(19) United States
(12) Patent Application Publication
(10) Pub. No.: US 2025/0101034 A1
(43) Pub. Date: Mar. 27, 2025

(54) COMPOSITIONS USEFUL FOR MODULATING SPLICING

(71) Applicant: SKYHAWK THERAPEUTICS, INC., Waltham, MA (US)

(72) Inventors: Brian LUCAS, Arlington, MA (US); Hasane RATNI, Habsheim (FR); Ingo KONETZKI, Basel (CH); Michael LUZZIO, Noank, CT (US)

(21) Appl. No.: 18/707,167

(22) PCT Filed: Nov. 4, 2022
(86) PCT No.: PCT/US2022/079347

§ 371 Date: May 3, 2024

Related U.S. Application Data
(60) Provisional application No. 63/275,808, filed on Nov. 4, 2021.

(51) Int. Cl. C07D 495/04

(52) U.S. Cl. CPC C07D 495/04

(57) ABSTRACT

Described herein are compounds that modulate splicing of a pre-mRNA, encoded by genes, and methods of treating diseases and conditions associated with gene expression or activity of proteins encoded by genes.

This application claims the benefit of U.S. Provisional Application No. 63/275,808 filed on Nov. 4, 2021, the disclosure of which is hereby incorporated by reference in its entirety.

### BACKGROUND

Spinocerebellar Ataxia 3 (SCA3 or Machado-Joseph Disease) is a rare, inherited, neurodegenerative, autosomal dominant disease. It is characterized by progressive degeneration of the brainstem, cerebellum and spinal cord, however, neurons in other areas of the brain are also affected. Presenting features include gait problems, speech difficulties, clumsiness, and often visual blurring and diplopia; saccadic eye movements become slow and ophthalmoparesis develops, resulting initially in up-gaze restriction. Ambulation becomes increasingly difficult, leading to the need for assistive devices 10 to 15 years following onset. Late in the disease course, individuals are wheelchair bound and have severe dysarthria, dysphagia, facial and temporal atrophy. The disease progresses relentlessly until death occurs at any time from 6 to approximately 30 years after onset through pulmonary complications.

SCA3 is caused by CAG tri-nucleotide repeats in exon 10 of the Ataxin 3 (ATXN3) gene. ATXN3 encodes for a deubiquinase with wide-ranging functions, but it does not appear to be an essential gene. Disease causing variants of the ATXN3 gene have approximately 40 to over 200 CAG tri-nucleotide repeats in exon 10. Expanded CAG repeats in the ATXN3 gene are translated into expanded polyglutamine repeats (polyQ) in the ataxin-3 protein and this toxic Ataxin 3 protein is associated with aggregates. The polyglutamine expanded ataxin-3 protein in these aggregates is ubiquinated and the aggregates contain other proteins, including heat shock proteins and transcription factors. Aggregates are frequently observed in the brain tissue of SCA3 patients. There are currently no treatments for SCA3.

### SUMMARY

In one aspect, described herein is a compound of Formula (I), or a pharmaceutically acceptable salt or a pharmaceutically acceptable solvate thereof.

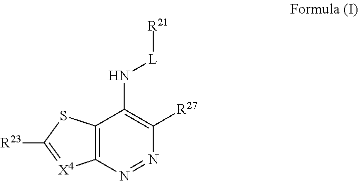

wherein X⁴, L, R²¹, R²³ and R²⁷ are as defined herein.

Also provided herein are pharmaceutical compositions comprising a compound disclosed herein, or a pharmaceutically acceptable salt or a pharmaceutically acceptable solvate thereof, and a pharmaceutically acceptable excipient or carrier.

In some aspects, described herein, is a method of modulating splicing of a Ataxin3 (ATXN3) pre-mRNA, comprising contacting a small molecule splicing modulator compound disclosed herein (SMSM) to the ATXN3 pre-mRNA with a splice site sequence or cells comprising the ATXN3 pre-mRNA, wherein the SMSM binds to the ATXN3 pre-mRNA and modulates splicing of the ATXN3 pre-mRNA in a cell of a subject to produce a spliced product of the ATXN3 pre-mRNA.

In some aspects, described herein, is a method of treating, preventing, delaying of progress, or ameliorating symptoms of a disease or a condition associated with Ataxin 3 (ATXN3) expression level or activity level in a subject in need thereof, comprising administering a therapeutically effective amount of a small molecule splicing modulator compound disclosed herein (SMSM), wherein the SMSM binds to a pre-mRNA encoded by ATXN3 and modulates splicing of the ATXN3 pre-mRNA in a cell of the subject to produce a spliced product of the ATXN3 pre-mRNA, wherein the amount of full length ATXN3 is reduced.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods, and materials are described below.

### Definitions

The term “small molecule splicing modulator” or “SMSM” denotes a small molecule compound that binds to a cell component (e.g., DNA, RNA, pre-mRNA, protein, RNP, snRNA, carbohydrates, lipids, co-factors, nutrients, and/or metabolites) and modulates splicing. For example, a SMSM can bind to a polynucleotide, e.g., an RNA (e.g., a pre-mRNA) with an aberrant splice site, resulting in steric modulation of the polynucleotide. For example, a SMSM can bind to a protein, e.g., a spliceosome protein or a ribonuclear protein, resulting in steric modulation of the protein. For example, a SMSM can bind to a spliceosome component, e.g., a spliceosome protein or snRNA resulting in steric modulation of the spliceosome protein or snRNA. For example, a SMSM is a compound of Formula (I). The term “small molecule splicing modulator” or “SMSM” specifically excludes compounds consisting of oligonucleotides.

“Steric alteration,” “steric modification,” or “steric modulation” herein refers to changes in the spatial orientation of chemical moieties with respect to each other. A person of ordinary skill in the art would recognize steric mechanisms include, but are not limited to, steric hindrance, steric shielding, steric attraction, chain crossing, steric repulsions, steric inhibition of resonance, and steric inhibition of protonation.

Any open valency appearing on a carbon, oxygen, sulfur or nitrogen atom in the structures herein indicates the presence of a hydrogen, unless indicated otherwise.

The definitions described herein apply irrespective of whether the terms in question appear alone or in combination. It is contemplated that the definitions described herein can be appended to form chemically relevant combinations, such as e.g., “heterocycloalkylaryl,” “haloalkylheteroaryl,” “arylalkylheterocycloalkyl,” or “alkoxyalkyl.” The last member of the combination is the radical which is binding to the rest of the molecule. The other members of the combination are attached to the binding radical in reversed order in respect of the literal sequence, e.g., the combination arylalkylheterocycloalkyl refers to a heterocycloalkyl-radical which is substituted by an alkyl which is substituted by an aryl.

When indicating the number of substituents, the term “one or more” refers to the range from one substituent to the highest possible number of substitutions, i.e., replacement of one hydrogen up to replacement of all hydrogens by substituents.

The term “optional” or “optionally” denotes that a subsequently described event or circumstance can but need not occur, and that the description includes instances where the event or circumstance occurs and instances in which it does not.

The term “substituent” denotes an atom or a group of atoms replacing a hydrogen atom on the parent molecule.

The term “substituted” denotes that a specified group bears one or more substituents. Where any group can carry multiple substituents and a variety of possible substituents is provided, the substituents are independently selected and need not to be the same. The term “unsubstituted” means that the specified group bears no substituents. The term “optionally substituted” means that the specified group is unsubstituted or substituted by one or more substituents, independently chosen from the group of possible substituents. When indicating the number of substituents, the term “one or more” means from one substituent to the highest possible number of substitutions, i.e., replacement of one hydrogen up to replacement of all hydrogens by substituents.

The terms “compound(s) of this disclosure,” “compound(s) of the present disclosure,” “small molecule steric modulator,” “small molecule splicing modulator,” “steric modulator,” “splicing modulator,” “compounds that modify splicing,” and “compounds modifying splicing” are interchangeably used herein and refer to compounds as disclosed herein and stereoisomers, tautomers, solvates, and salts (e.g., pharmaceutically acceptable salts) thereof.

The following abbreviations are used throughout the specification: acetic acid (AcOH); ethyl acetate (EtOAc); butyl alcohol (n-BuOH); 1,2-dichloroethane (DCE); dichloromethane (CH₂Cl₂, DCM); diisopropylethylamine (Diipea); dimethylformamide (DMF); hydrogen chloride (HCl); methanol (MeOH); methoxymethyl bromide (MOMBr); N-methyl-2-pyrrolidone (NMP); methyl Iodide (Mel); n-propanol (n-PrOH); p-methoxybenzyl (PMB); triethylamine (Et₃N); [1,1′-Bis(diphenylphosphino)ferrocene]dichloropalladium(II); (Pd(dppf)Cl₂); sodium ethane thiolate (EtSNa); sodium acetate (NaOAc); sodium hydride (NaH); sodium hydroxide (NaOH); tetrahydropyran (THP); tetrahydrofuran (THF).

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

The term “oxo” refers to the =O substituent.

“Carboxyl” refers to —COOH.

“Cyano” refers to —CN.

The term “thioxo” refers to the =S substituent.

“Amidinyl” refers to a radical of the formula —C(═NR^{a})—N(R^{a})₂ wherein each R^{a} is independently a hydrogen, a C₁-C₆ alkyl, C₁-C₆ haloalkyl, C₃-C₆cycloalkyl, or 3-6 membered heterocycloalkyl. In some embodiments, an amidinyl is C(═NH)NH₂. In some embodiments, an amidinyl is C(═NH)NH(C₁-C₆ alkyl).

The term “halo,” “halogen,” and “halide” are used interchangeably herein and denote fluoro, chloro, bromo, or iodo.

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted as described below. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

The term “alkoxy” refers to a radical of the formula —OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy.

The term “alkylamino” refers to a radical of the formula —NHR or —NRR where each R is, independently, an alkyl radical as defined above. Unless stated otherwise specifically in the specification, an alkylamino group may be optionally substituted as described below.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)=CH₂, —CH═CHCH₃, —C(CH₃)=CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “aromatic” refers to a planar ring having a delocalized R-electron system containing 4n+2π electrons, where n is an integer. Aromatics can be optionally substituted. The term “aromatic” includes both aryl groups (e.g., phenyl, naphthalenyl) and heteroaryl groups (e.g., pyridinyl, furanyl, quinolinyl).

The term “aryl” refers to a radical derived from a hydrocarbon ring system comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group is partially reduced to form a cycloalkyl group defined herein. In some embodiments, an aryl group is fully reduced to form a cycloalkyl group defined herein.

The term “haloalkyl” denotes an alkyl group wherein at least one of the hydrogen atoms of the alkyl group has been replaced by same or different halogen atoms, particularly fluoro atoms. Examples of haloalkyl include monofluoro-, difluoro- or trifluoro-methyl, -ethyl or -propyl, for example, 3,3,3-trifluoropropyl, 2-fluoroethyl, 2,2,2-trifluoroethyl, fluoromethyl, or trifluoromethyl. The term “perhaloalkyl” denotes an alkyl group where all hydrogen atoms of the alkyl group have been replaced by the same or different halogen atoms. Exemplary haloalkyl groups further include trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Cyanoalkyl” refers to an alkyl radical, as defined above, that is substituted by one or more cyano groups. In some embodiments, the alkyl is substituted with one cyano group. In some embodiments, the alkyl is substituted with one, two, or three cyano groups. Aminoalkyl include, for example, cyanomethyl, cyanoethyl, cyanopropyl, cyanobutyl, or cyanopentyl.

The term “haloalkoxy” denotes an alkoxy group wherein at least one of the hydrogen atoms of the alkoxy group has been replaced by same or different halogen atoms, particularly fluoro atoms. Examples of haloalkoxyl include monofluoro-, difluoro- or trifluoro-methoxy, -ethoxy or -propoxy, for example, 3,3,3-trifluoropropoxy, 2-fluoroethoxy, 2,2,2-trifluoroethoxy, fluoromethoxy, or trifluoromethoxy. The term “perhaloalkoxy” denotes an alkoxy group where all hydrogen atoms of the alkoxy group have been replaced by the same or different halogen atoms. Examples of haloalkoxyl further include trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

The term “bicyclic ring system” denotes two rings which are fused to each other via a common single or double bond (annelated bicyclic ring system), via a sequence of three or more common atoms (bridged bicyclic ring system) or via a common single atom (spiro bicyclic ring system). Bicyclic ring systems can be saturated, partially unsaturated, unsaturated, or aromatic. Bicyclic ring systems can comprise heteroatoms selected from N, O, and S.

The terms “carbocyclic” or “carbocycle” refer to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

The term “cycloalkyl” refers to a monocyclic or polycyclic non-aromatic radical, wherein each of the atoms forming the ring (i.e., skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopropyl, cyclobutyl, cyclopentyl or cyclohexyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “bridged” refers to any ring structure with two or more rings that contains a bridge connecting two bridgehead atoms. The bridgehead atoms are defined as atoms that are the part of the skeletal framework of the molecule and which are bonded to three or more other skeletal atoms. In some embodiments, the bridgehead atoms are C, N, or P. In some embodiments, the bridge is a single atom or a chain of atoms that connects two bridgehead atoms. In some embodiments, the bridge is a valence bond that connects two bridgehead atoms. In some embodiments, the bridged ring system is cycloalkyl. In some embodiments, the bridged ring system is heterocycloalkyl.

The term “fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with one or more N, S, and O atoms. The non-limiting examples of fused heterocyclyl or heteroaryl ring structures include 6-5 fused heterocycle, 6-6 fused heterocycle, 5-6 fused heterocycle, 5-5 fused heterocycle, 7-5 fused heterocycle, and 5-7 fused heterocycle.

The term “fluoroalkyl” refers to an alkyl in which one or more hydrogen atoms are replaced by a fluorine atom. In one aspect, a fluoroalkyl is a C₁-C₆ fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g., —NH—, —N(alkyl)-, or —N(aryl)-), sulfur (e.g., —S—, —S(═O)—, or —S(═O)₂—), or combinations thereof. In some embodiments, a heteroalkyl is attached to the rest of the molecule at a carbon atom of the heteroalkyl. In some embodiments, a heteroalkyl is attached to the rest of the molecule at a heteroatom of the heteroalkyl. In some embodiments, a heteroalkyl is a C₁-C₆ heteroalkyl. Representative heteroalkyl groups include, but are not limited to —OCH₂OMe, —OCH₂CH₂OH, —OCH₂CH₂OMe, or —OCH₂CH₂OCH₂CH₂NH₂. In some embodiments, a heteroalkyl contains one skeletal heteroatom. In some embodiments, a heteroalkyl contains 1-3 skeletal heteroatoms.

The term “heteroalkylene” refers to an alkyl radical as described above where one or more carbon atoms of the alkyl is replaced with a 0, N or S atom. “Heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —OCH₂CH₂O—, —OCH₂CH₂OCH₂CH₂O—, or —OCH₂CH₂OCH₂CH₂OCH₂CH₂O—.

The term “heterocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. In some embodiments, a heterocycloalkyl is monocyclic. In some embodiments, a heterocycloalkyl is bicyclic. In some embodiments, a heterocycloalkyl is partially saturated. In some embodiments, a heterocycloalkyl is fully saturated. The nitrogen, carbon, or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides, and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heterocycle” or “heterocyclic” refers to heteroaromatic rings (also known as heteroaryls) and heterocycloalkyl rings (also known as heteroalicyclic groups) that includes at least one heteroatom selected from nitrogen, oxygen and sulfur, wherein each heterocyclic group has from 3 to 12 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 12 atoms in its ring system and aromatic heterocyclic groups include rings having 5 to 12 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3 h-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The term “heteroaryl” refers to an aryl group that includes one or more ring heteroatoms selected from nitrogen, oxygen, and sulfur. The heteroaryl can be monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group is partially reduced to form a heterocycloalkyl group defined herein. In some embodiments, a heteroaryl group is fully reduced to form a heterocycloalkyl group defined herein.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄ alkyl), —C(═O)NH₂, —C(═O)NH(C₁-C₄ alkyl), —C(═O)N(C₁-C₄ alkyl)₂, —S(═O)₂NH₂, —S(═O)₂NH(C₁-C₄ alkyl), —S(═O)₂N(C₁-C₄ alkyl)₂, C₁-C₄ alkyl, C₃-C₆ cycloalkyl, C₁-C₄ fluoroalkyl, C₁-C₄ heteroalkyl, C₁-C₄ alkoxy, C₁-C₄ fluoroalkoxy, —SC₁—C₄ alkyl, —S(═O)C₁-C₄ alkyl, and —S(═O)₂(C₁-C₄ alkyl). In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

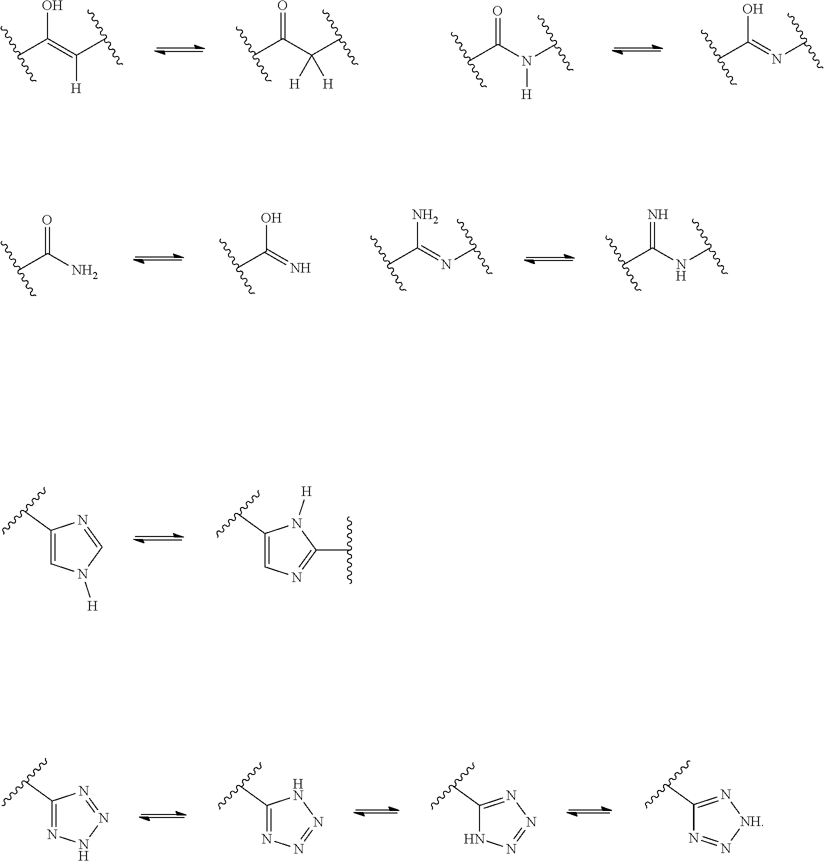

The terms “administer,” “administering,” “administration,” and the like, as used herein, refer to the methods that may be used to enable delivery of compounds or compositions to the desired site of biological action. These methods include but are not limited to oral routes (p.o.), intraduodenal routes (i.d.), parenteral injection (including intravenous (i.v.), subcutaneous (s.c.), intraperitoneal (i.p.), intramuscular (i.m.), intravascular or infusion (inf.)), topical (top.) and rectal (p.r.) administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient and are intended to include treatment regimens in which the agents are administered by the same or different route of administration or at the same or different time.

The term “subject” or “patient” encompasses mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In one embodiment, a non-human animal is a mouse.

The term “pharmaceutically acceptable” denotes an attribute of a material which is useful in preparing a pharmaceutical composition that is generally safe, non toxic, and neither biologically nor otherwise undesirable and is acceptable for veterinary as well as human pharmaceutical use. “Pharmaceutically acceptable” can refer a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The terms “pharmaceutically acceptable excipient”, “pharmaceutically acceptable carrier” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents, excipients, preservatives or lubricants used in formulating pharmaceutical products.

The term “pharmaceutically acceptable salts” denotes salts which are not biologically or otherwise undesirable. Pharmaceutically acceptable salts include both acid and base addition salts. A “pharmaceutically acceptable salt” can refer to a formulation of a compound that does not cause significant irritation to an organism to which it is administered and/or does not abrogate the biological activity and properties of the compound. In some embodiments, pharmaceutically acceptable salts are obtained by reacting a SMSM compound of the present disclosure with acids. Pharmaceutically acceptable salts are also obtained by reacting a compound of the present disclosure with a base to form a salt.

As used herein, a “small molecular weight compound” can be used interchangeably with “small molecule” or “small organic molecule.” Small molecules refer to compounds other than peptides or oligonucleotides; and typically have molecular weights of less than about 2000 Daltons, e.g., less than about 900 Daltons.

### Small Molecule Splicing Modulators (SMSMs)

It has now been found that compounds of this invention, and pharmaceutically acceptable compositions thereof, are effective as agents for use in treating, preventing, or ameliorating a disease or a condition associated with a target RNA. The present invention provides the unexpected discovery that certain small chemical molecules can modify splicing events in pre-mRNA molecules, herein referred to as small molecule splicing modulators (SMSMs). These SMSMs can modulate specific splicing events in specific pre-mRNA molecules. The small molecules of this invention are different from and are not related to antisense or antigene oligonucleotides.

In one aspect, a SMSM described herein is a compound of Formula (I), or a pharmaceutically acceptable salt thereof:

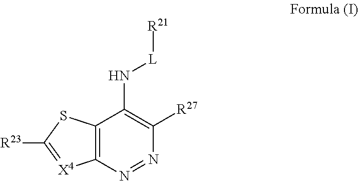

wherein,
X⁴ is selected from the group consisting of N, and CR²⁴;
L is absent or selected from the group consisting of C₁₋₆ alkylene, C₂₋₆ alkenylene, and C₂₋₆ alkynylene, wherein the C₁₋₆ alkylene, C₂₋₆ alkenylene, and C₂₋₆ alkynylene are each optionally substituted by 1, 2, 3, or 4 independently selected R²⁰ groups;
R²¹ is selected from the group consisting of 5 membered heteroaryl, and 5 membered heterocycloalkyl, each of which is unsubstituted or substituted with 1, 2, or 3, independently selected R^{1A} groups; each R^{1A} is independently selected from halo, CN, NO₂, C₁₋₆ alkyl, C₂₋₆ alkenyl, C₂₋₆ alkynyl, C₁₋₆ haloalkyl, C₁₋₆ alkoxy, —C(═O)OH, —C(═O)C₁₋₆ alkyl, —C(═O)C₁₋₆ haloalkyl, and —C(═O)C₁₋₆ alkoxy;
R²³ is selected from the group consisting of H, azido, halo, CN, NO₂, C₁₋₆ alkyl, C₂₋₆ alkenyl, C₂₋₆ alkynyl, C₁₋₆ heteroalkyl, —(C₁₋₆ alkylene)-C₃₋₁₀ cycloalkyl, —(C₁₋₆ alkylene)-4-10 membered heterocycloalkyl, —(C₁₋₆ heteroalkylene)-C₃₋₁₀ cycloalkyl, —(C₁₋₆ heteroalkylene)-4-10 membered heterocycloalkyl, C₃₋₁₀ cycloalkyl, C₆₋₁₀ aryl, 5-10 membered heteroaryl, 4-10 membered heterocycloalkyl, OR^{a3}, SR^{a3}, C(═O)R^{b3}, C(═O)OR^{b3}, NR^{c3}R^{d3}, C(═O)NR^{c3}R^{d3}, —OC(═O)NR^{c3}R^{d3}, NR^{c3}C(═O)R^{b3}, NR^{c3}C(═O)OR^{b3} NR^{c3}C(═O)NR^{c3}R^{d3} NR^{c3}S(═O)₂R^{b3}, NR^{c3}S(═O)₂NR^{c3}R^{d3}, S(O)NR^{c3}R^{d3}, and S(O)₂NR^{c3}R^{d3}, wherein the C₁₋₆ alkyl, C₂₋₆ alkenyl, C₂₋₆ alkynyl, C₁₋₆ heteroalkyl, C₁₋₆ alkylene, C₁₋₆ heteroalkylene, C₃₋₁₀ cycloalkyl, C₆₋₁₀ aryl, 5-10 membered heteroaryl, and 4-10 membered heterocycloalkyl are each optionally substituted by 1, 2, 3, or 4 independently selected R²⁰ groups;
R²⁴ is selected from the group consisting of H, azido, halo, CN, NO₂, C₁₋₆ alkyl, C₂₋₆ alkenyl, C₂₋₆ alkynyl, C₃₋₁₀ cycloalkyl, C₆₋₁₀ aryl, 5-10 membered heteroaryl, 4-10 membered heterocycloalkyl, OR^{a4}, C(═O)R^{b4}, C(═O)OR^{b4}, NR^{c4}R^{d4}, C(═O)NR^{c4}R^{d4}, —OC(═O)NR^{c4}R^{d4}, NR^{c4}C(═O)R^{b4}, NR^{c4}C(═O)OR^{b4}, NR^{c4}C(═O)NR^{c4}R^{d4}, NR^{c4}S(═O)₂R^{b4}, NR^{c4}S(═O)₂NR^{c4}R^{d4} S(O)NR^{c4}R^{d4}, and S(O)₂NR^{c4}R^{d4}, wherein the C₁₋₆ alkyl, C₃₋₁₀ cycloalkyl, C₂₋₆ alkenyl, C₂₋₆ alkynyl, C₆₋₁₀ aryl, 5-10 membered heteroaryl, and 4-10 membered heterocycloalkyl are each optionally substituted by 1, 2, 3, or 4 independently selected R²⁰ groups;
R²⁷ is selected from the group consisting of H, halogen, alkyl, heteroalkyl, CN, NO₂, OR^{a7}, C(═O)R^{b7}, C(═O)OR^{b7}, NR^{c7}R^{d7}, C(═O)NR^{c7}R^{d7}, —OC(═O)NR^{c7}R^{d7}, NR^{c7}C(═O)R^{b7} NR^{c7}C(═O)OR^{b7} NR^{c7}C(═O)NR^{c7}R^{d7} NR^{c7}S(═O)₂R^{b7}, and NR^{c7}S(═O)₂NR^{c7}R^{d7};
each R^{a3}, R^{b3}, R^{c3}, R^{d3}, R^{a4}, R^{b4}, R^{c4}, R^{d4}, R^{a7}, R^{b7}, R⁷, and R^{d7} is independently selected from the group consisting of H, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, 4-10 membered heterocycloalkyl, wherein the C₁₋₆ alkyl, C₂₋₆ alkenyl, C₂₋₆ alkynyl, C₃₋₁₀ cycloalkyl, —(C₁₋₆ alkylene)-C₃₋₁₀ cycloalkyl, C₆₋₁₀ aryl, 5-10 membered heteroaryl, and 4-10 membered heterocycloalkyl are each optionally substituted by 1, 2, 3, or 4 independently selected R²⁰ groups;
   or R^{c3} and R^{d3} together with the N atom to which they are connected, come together to form a 5-10 membered heteroaryl or 4-10 membered heterocycloalkyl ring, each optionally substituted by 1, 2, 3, or 4 independently selected R²⁰ groups;
   or R^{c4} and R^{d4} together with the N atom to which they are connected, come together to form a 5-10 membered heteroaryl or 4-10 membered heterocycloalkyl ring, each optionally substituted by 1, 2, 3, or 4 independently selected R²⁰ groups;
   or R^{c7} and R^{d7} together with the N atom to which they are connected, come together to form a 5-10 membered heteroaryl or 4-10 membered heterocycloalkyl ring, each optionally substituted by 1, 2, 3, or 4 independently selected R²⁰ groups; and
each R²⁰ is independently selected from the group consisting of OH, SH, CN, NO₂, halo, oxo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, phenyl, 5-6 membered heteroaryl, 4-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, di(C₁₋₄ alkyl)aminocarbonylamino, and amidinyl.

In some embodiments of a compound of Formula (I) or a pharmaceutically acceptable salt thereof,
X⁴ is selected from the group consisting of N and CR²⁴;
L is absent or selected from the group consisting of alkylene, alkenylene, and each of which is unsubstituted or substituted with 1, 2, 3, or 4 independently selected R²⁰ groups;
R²¹ is selected from the group consisting of 5 membered aryl, 5 membered heteroaryl, and 5 membered heterocycloalkyl, each of which is unsubstituted or substituted with 1, 2, or 3, independently selected R^{1A} groups; wherein each R^{1A} is independently selected from halo, CN, NO₂, alkyl, alkenyl, C₂₋₆ alkynyl, alkoxy, —C(═O)OH, an ether group, or an ester group, each of which is unsubstituted or substituted;
R²³ is selected from the group consisting of H, oxo, azido, halo, CN, NO₂, alkyl, heteroalkyl, alkenyl, alkynyl, cycloalkyl, aryl, heteroaryl, heterocycloalkyl, OR^{a3}, SR^{a3}, C(═O)R^{b3}, C(═O)OR^{b3}, NR^{c3}R^{d3}, C(═O)NR^{c3}R^{d3}, —OC(═O)NR^{c3}R^{d3}, NR^{c3}C(═O)R^{b3}, NR^{c3}C(═O)OR^{b3}, NR^{c3}C(═O)NR^{c3}R^{d3} NR^{c3}S(═O)₂R^{b3}, NR^{c3}S(═O)₂NR^{c3}R^{d3}, S(O)NR^{c3}R^{d3}, and S(O)₂NR^{c3}R^{d3}, wherein the alkyl, cycloalkyl, aryl, heteroaryl, and heterocycloalkyl are each unsubstituted or substituted with 1, 2, 3, or 4 independently selected R²⁰ groups;
R²⁴ is selected from the group consisting of H, oxo, azido, halo, CN, NO₂, alkyl, heteroalkyl, alkenyl, alkynyl, cycloalkyl, aryl, heteroaryl, heterocycloalkyl, OR^{a4}, C(═O)R^{b4}, C(═O)OR^{b4}, NR^{c4}R^{d4}, C(═O)NR^{c4}R^{d4}, —OC(═O)NR^{c4}R^{d4}, NR^{c4}C(═O)R^{b4}, NR^{c4}C(═O)OR^{b4}, NR^{c4}C(═O)NR^{c4}R^{d4}, NR^{c4}S(═O)₂R^{b4}, NR^{c4}S(═O)₂NR^{c4}R^{d4}, S(O)NR^{c4}R^{d4}, and S(O)₂NR^{c4}R^{d4}, wherein the alkyl, heteroalkyl, alkenyl, alkynyl, cycloalkyl, aryl, heteroaryl, and heterocycloalkyl are each unsubstituted or substituted with 1, 2, 3, or 4 independently selected R²⁰ groups;
R²⁷ is selected from the group consisting of H, alkyl, heteroalkyl, CN, NO₂, OR^{a7}, C(═O)R^{b7}, C(═O)OR^{b7}, NR^{c7}R^{d7}, C(═O)NR^{c7}R^{d7}, —OC(═O)NR^{c7}R^{d7}, NR^{c7}C(═O)R^{b7}, NR^{c7}C(═O)OR^{b7}, NR^{c7}C(═O)NR^{c7}R^{d7}, NR^{c7}S(═O)₂R^{b7}, and NR^{c7}S(═O)₂NR^{c7}R^{d7};
each R^{a3}, R^{b3}, R^{c3}, R^{d3}, R^{a4}, R^{b4}, R^{c4}, and R^{d4}, is independently selected from the group consisting of H, alkyl, alkenyl, alkynyl, alkoxy, cycloalkyl, aryl, heteroaryl, heterocycloalkyl, each of which is unsubstituted or substituted with 1, 2, 3, or 4 independently selected R²⁰ groups;
each R^{c3} and R^{d3} together with the N atom to which they are connected, come together to form a heteroaryl or heterocycloalkyl ring, each of which is unsubstituted or substituted with 1, 2, 3, or 4 independently selected R²⁰ groups;
or each R^{c4} and R^{d4} together with the N atom to which they are connected, come together to form a heteroaryl or heterocycloalkyl ring, each of which is unsubstituted or substituted with 1, 2, 3, or 4 independently selected R²⁰ groups; and
each R²⁰ is independently selected from the group consisting of OH, SH, CN, NO₂, halo, oxo, alkyl, alkenyl, alkynyl, alkoxy, cycloalkyl, aryl, heteroaryl, heterocycloalkyl, amino, carbamyl, or carbamoyl.

In some embodiments, X⁴ is N. In some embodiments, X⁴ is CR²⁴. In some embodiments, R²⁴ is selected from the group consisting of H, azido, halo, CN, NO₂, C₁₋₆ alkyl, C₂₋₆ alkenyl, C₂₋₆ alkynyl, C₃₋₁₀ cycloalkyl, C₆₋₁₀ aryl, 5-10 membered heteroaryl, 4-10 membered heterocycloalkyl, OR^{a4}, C(═O)R^{b4}, C(═O)OR^{b4}, NR^{c4}R^{d4}, C(═O)NR^{c4}R^{d4}, —OC(═O)NR^{c4}R^{d4}, NR^{c4}C(═O)R^{b4}, NR^{c4}C(═O)OR^{b4}, NR^{c4}C(═O)NR^{c4}R^{d4}, NR^{c4}S(═O)₂R^{b4}, NR^{c4}S(═O)₂NR^{c4}R^{d4} S(O)NR^{c4}R^{d4}, and S(O)₂NR^{c4}R^{d4}, wherein the C₁₋₆ alkyl, C₃₋₁₀ cycloalkyl, C₆₋₁₀ aryl, 5-10 membered heteroaryl, and 4-10 membered heterocycloalkyl are each optionally substituted by 1, 2, 3, or 4 independently selected R²⁰ groups. In some embodiments, R²⁴ is selected from the group consisting of H, halo, CN, NO₂, C₁₋₆ alkyl, C₂₋₆ alkenyl, C₂₋₆ alkynyl, C₃₋₁₀ cycloalkyl, 4-10 membered heterocycloalkyl, OH, C₁₋₆ alkoxyl, and C₁₋₆ haloalkyl. In some embodiments, R²⁴ is selected from the group consisting of H, halo, CN, C₁₋₆ alkyl, C₂₋₆ alkynyl, C₃₋₆ cycloalkyl, 4-6 membered heterocycloalkyl, OH, C₁₋₆ alkoxyl, and C₁₋₆ haloalkyl. In some embodiments, X⁴ is CR²⁴, wherein R²⁴ is selected from the group consisting of hydrogen, OH, halo, CN, substituted or unsubstituted C₁₋₆ alkyl, substituted or unsubstituted C₁₋₆ alkoxyl, substituted or unsubstituted C₃₋₆ cycloalkyl, substituted or unsubstituted C₂₋₄ alkenyl, and substituted or unsubstituted C₂₋₄ alkynyl. In some embodiments, R²⁴ is hydrogen. In some embodiments, R²⁴ is halogen. In some embodiments, R²⁴ is —Br. In some embodiments, R²⁴ is —F. In some embodiments, R²⁴ is —Cl. In some embodiments, R²⁴ is —CN. In some embodiments, R²⁴ is OH. In some embodiments, R²⁴ is C₁₋₄ alkyl. In some embodiments, R²⁴ is C₁₋₄ haloalkyl. In some embodiments, R²⁴ is C₁₋₄ alkoxyl. In some embodiments, R²⁴ is methyl. In some embodiments, R²⁴ is ethyl. In some embodiments, R²⁴ is cycloalkyl. In some embodiments, R²⁴ is cyclopropyl. In some embodiments, R²⁴ is C₂₋₄ alkenyl. In some embodiments, R²⁴ is C₂₋₄ alkynyl. In some embodiments, R²⁴ is ethynyl. In some embodiment, R²⁴ is propynyl.

In some embodiments, L is absent. In some embodiments, L is alkylene, which is unsubstituted or substituted with 1, 2, 3, or 4 independently selected R²⁰ groups. In some embodiments, L is C₁₋₆alkylene. In some embodiments, L is C₁₋₃alkylene. In some embodiments, L is —CH₂—.

In some embodiments, R²¹ is unsubstituted or substituted 5 membered aryl. In some embodiments, R²¹ is unsubstituted or substituted 5 membered heteroaryl. In some embodiments, R²¹ is unsubstituted or substituted 5 membered heterocycloalkyl. In some embodiments, R²¹ is unsubstituted. In some embodiments, R²¹ is substituted with 1, 2, or 3, independently selected R^{1A} groups; wherein each R^{1A} is independently selected from halo, CN, NO₂, alkyl, alkenyl, C₂₋₆ alkynyl, alkoxy, —C(═O)OH, an ether group, or an ester group, each of which is unsubstituted or substituted. In some embodiments, R²¹ is substituted with 1, 2, or 3 substituents independently selected R^{1A} groups; wherein each R^{1A} is independently selected from halo, C₁₋₆alkyl, C₁₋₆haloalkyl, and C₁₋₆alkoxy. In some embodiments, R²¹ is substituted with 1, 2, or 3 substituents independently selected R^{1A} groups; wherein each R^{1A} is independently selected from halo, C₁₋₃alkyl, C₁₋₃haloalkyl, and C₁₋₃alkoxy. In some embodiments, R²¹ is

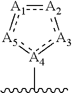

wherein [symbol] represents a single or a double bond; each of A₁, A₂, A₃, and A₅ is independently selected from the group consisting of O, S, N, NH, NR^{1A}, CH, CR^{1A}, CH₂, and CHR^{1A}; and A₄ is selected from the group consisting of N, C, CH and CR^{1A}. In some embodiments, R²¹ is

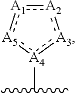

wherein [symbol] represents a single or a double bond; each of A₁, A₂, A₃, and A₅ is independently selected from the group consisting of O, S, N, NH, NR^{1A} C, CH, CR^{1A}, CH₂, and CHR^{1A}; and A4 is selected from the group consisting of N, C, CH and CR^{1A}.

In some embodiments, R²¹ is selected from the group consisting of

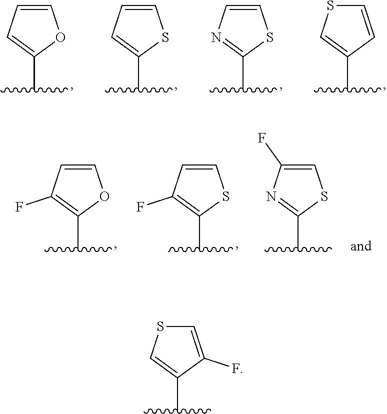

In some embodiments, R²¹ is 5 membered heteroaryl. In some embodiments, R²¹ is furanyl, or thiazolyl each of which is substituted or unsubstituted.

In some embodiments, R²¹ is unsubstituted furanyl. In some embodiments, R²¹ is substituted furanyl. In some embodiments, R²¹ is unsubstituted thiazolyl. In some embodiments, R²¹ is substituted thiazolyl.

In some embodiments, R²¹ is

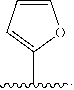 [SMILES: *c1ccco1]

In some embodiments, R²¹ is

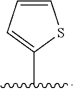 [SMILES: *c1cccs1]

In some embodiments, R²¹ is

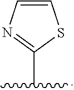 [SMILES: *c1nccs1]

In some embodiments, R²¹ is

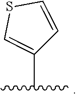 [SMILES: *c1ccsc1]

In some embodiments, R²¹ is

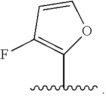 [SMILES: *c1occc1F]

In some embodiments, R²¹ is

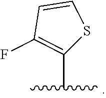 [SMILES: *c1sccc1F]

In some embodiments, R²¹ is

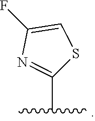 [SMILES: *c1nc(F)cs1]

In some embodiments, R²¹ is

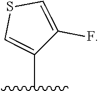 [SMILES: *c1cscc1F]

In some embodiments, R²¹ is

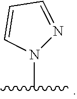 [SMILES: *n1cccn1]

In some embodiments, R²¹ is

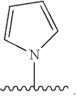 [SMILES: *n1cccc1]

In some embodiments, R²¹ is

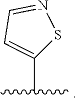 [SMILES: *c1ccns1]

In some embodiments, R²¹ is

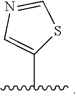 [SMILES: *c1cncs1]

In some embodiments, R²¹ is a

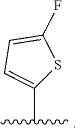 [SMILES: *c1ccc(F)s1]

In some embodiments, R²¹ is

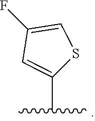 [SMILES: *c1cc(F)cs1]

In some embodiments, R²¹ is

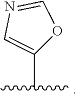 [SMILES: *c1cnco1]

In some embodiments, R²¹ is

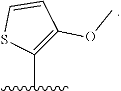 [SMILES: *c1sccc1OC]

In some embodiments, R²¹ is

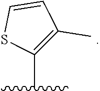 [SMILES: *c1sccc1C]

In some embodiments, R²¹ is

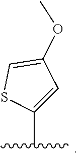 [SMILES: *c1cc(OC)cs1]

In some embodiments, R²¹ is

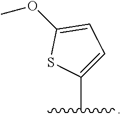 [SMILES: *c1ccc(OC)s1]

In some embodiments, R²¹ is

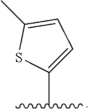 [SMILES: *c1ccc(C)s1]

In some embodiments, R²³ is H.

In some embodiments, R²³ is substituted or unsubstituted C₁₋₆ alkyl. In some embodiments, R²³ is C₁₋₆ alkyl, wherein C₁₋₆ alkyl is substituted with 1, 2, or 3 independently selected R²⁰ groups.

In some embodiments, R²³ is substituted or unsubstituted C₁₋₆ alkenyl. In some embodiments, R²³ is C₁₋₆ alkenyl, wherein C₁₋₆ alkenyl is substituted with 1, 2, or 3 independently selected R²⁰ groups.

In some embodiments, R²³ is substituted or unsubstituted C₁₋₆ alkynyl. In some embodiments, R²³ is C₁₋₆ alkynyl, wherein C₁₋₆ alkynyl is substituted with 1, 2, or 3 independently selected R²⁰ groups.

In some embodiments, R²³ is substituted or unsubstituted C₁₋₆ alkyl or substituted or unsubstituted C₁₋₆ heteroalkyl. In some embodiments, R²³ is substituted or unsubstituted C₁₋₆ heteroalkyl. In some embodiments, the C₁₋₆ heteroalkyl is —CH₂CH(NH₂)CH₂—S(═O)₂—CH₃ or —CH₂CH(NH₂)CH₂—S(═O)—CH₃. In some embodiments, R²³ is —CH₂CH(NH₂)CH₂—S(═O)₂—CH₃. In some embodiments, R²³ is —CH₂CH(NH₂)CH₂—S(═O)—CH₃. In some embodiments, R²³ is CH₂CHNH₂CH₃. In some embodiments, R²³ is CH₂CHNH₂CH₂OH. In some embodiments, R²³ is CH₂CHNH₂CH₂CH₃. In some embodiments, R²³ is CH₂CHNH₂CH₂CH₂OH. In some embodiments, R²³ is CH₂CHNH₂CH₂CH₂F. In some embodiments, R²³ is CH₂CHNH₂CH₂CHF₂. In some embodiments, R²³ is CH₂CHNH₂CH₂CH(CH₃)₂.

In some embodiments, R²³ is substituted or unsubstituted —(C₁₋₆ alkylene)-C₃₋₁₀ cycloalkyl. In some embodiments, R²³ is —(C₁₋₆ alkylene)-C₃₋₁₀ cycloalkyl, wherein —(C₁₋₆ alkylene)-C₃₋₁₀ cycloalkyl is substituted with 1, 2, or 3 independently selected R²⁰ groups. In some embodiments, the C₁₋₆ alkylene is C₁₋₃ alkylene. In some embodiments, the C₁₋₆ alkylene is CH₂. In some embodiments, the C₃₋₁₀ cycloalkyl is an optionally substituted a 3-6 membered ring. In some embodiments, the C₃₋₁₀ cycloalkyl is an optionally substituted a 3 membered ring. In some embodiments, the C₃₋₁₀ cycloalkyl is an optionally substituted a 4 membered ring. In some embodiments, the C₃₋₁₀ cycloalkyl is an optionally substituted a 5 membered ring. In some embodiments, the C₃₋₁₀ cycloalkyl is an optionally substituted a 6 membered ring. In some embodiments, the —C₃₋₁₀ cycloalkyl is

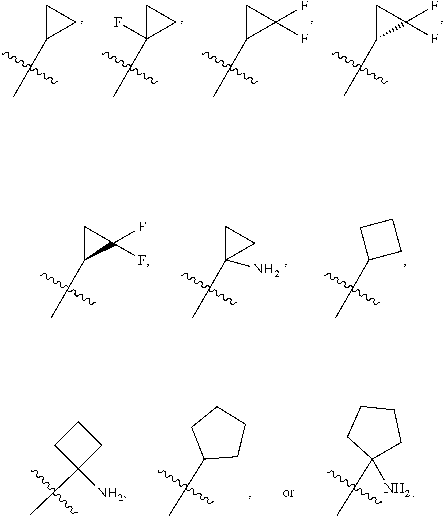

In some embodiments, R²³ is substituted or unsubstituted —(C₁₋₆ alkylene)-4-10 membered heterocycloalkyl. In some embodiments, R²³ is —(C₁₋₆ alkylene)-4-10 membered heterocycloalkyl, wherein —(C₁₋₆ alkylene)-4-10 membered heterocycloalkyl is substituted with 1, 2, or 3 independently selected R²⁰ groups. In some embodiments, the C₁₋₆ alkylene is C₁₋₃ alkylene. In some embodiments, the C₁₋₆ alkylene is CH₂. In some embodiments, the 4-10 membered heterocycloalkyl is an optionally substituted a 4-6 membered ring. In some embodiments, the 4-10 membered heterocycloalkyl is an optionally substituted a 4 membered ring. In some embodiments, the 4-10 membered heterocycloalkyl is an optionally substituted a 5 membered ring. In some embodiments, the 4-10 membered heterocycloalkyl is an optionally substituted a 6 membered ring. n some embodiments, the 4-10 membered heterocycloalkyl contains 0-1 oxygen and 0-2 nitrogen atoms. In some embodiments, the -4-10 membered heterocycloalkyl is

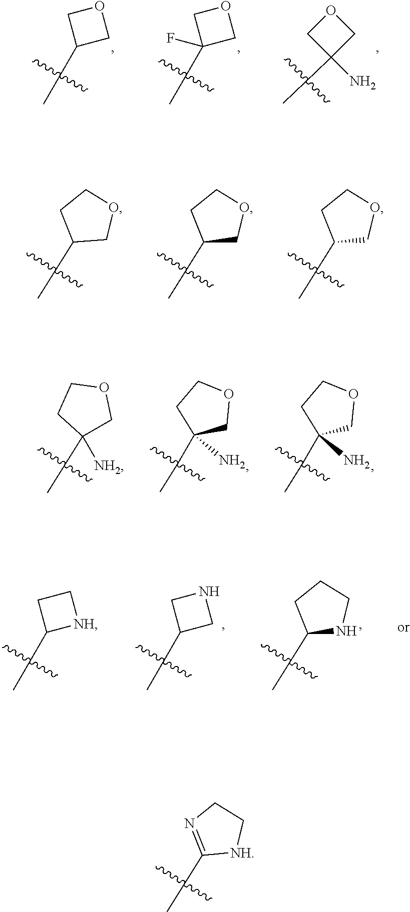

In some embodiments, R²³ is substituted or unsubstituted —(C₁₋₆ heteroalkylene)-C₃₋₁₀ cycloalkyl. In some embodiments, R²³ is —(C₁₋₆ heteroalkylene)-C₃₋₁₀ cycloalkyl, wherein —(C₁₋₆ heteroalkylene)-C₃₋₁₀ cycloalkyl is substituted with 1, 2, or 3 independently selected R²⁰ groups. In some embodiments, the heteroalkylene is C₁₋₃ heteroalkylene. In some embodiments, the C₃₋₁₀ cycloalkyl is an optionally substituted a 3-6 membered ring. In some embodiments, the C₃₋₁₀ cycloalkyl is an optionally substituted a 3 membered ring. In some embodiments, the C₃₋₁₀ cycloalkyl is an optionally substituted a 4 membered ring. In some embodiments, the C₃₋₁₀ cycloalkyl is an optionally substituted a 5 membered ring. In some embodiments, the C₃₋₁₀ cycloalkyl is an optionally substituted a 6 membered ring. In some embodiments, the heteroalkylene is C₁₋₃ heteroalkylene. In some embodiments, the —C₃₋₁₀ cycloalkyl is

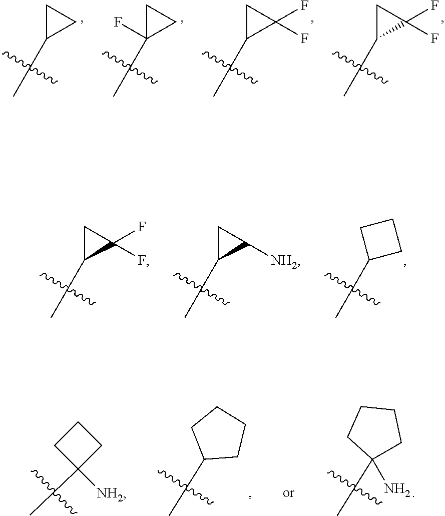

In some embodiments, R²³ is substituted or unsubstituted —(C₁₋₆ heteroalkylene)-4-10 membered heterocycloalkyl. In some embodiments, R²³ is —(C₁₋₆ heteroalkylene)-4-10 membered heterocycloalkyl, wherein —(C₁₋₆ heteroalkylene)-4-10 membered heterocycloalkyl is substituted with 1, 2, or 3 independently selected R²⁰ groups. In some embodiments, the heteroalkylene is C₁₋₃ heteroalkylene. In some embodiments, the 4-10 membered heterocycloalkyl is an optionally substituted a 4-6 membered ring. In some embodiments, the 4-10 membered heterocycloalkyl is an optionally substituted a 4 membered ring. In some embodiments, the 4-10 membered heterocycloalkyl is an optionally substituted a 5 membered ring. In some embodiments, the 4-10 membered heterocycloalkyl is an optionally substituted a 6 membered ring. n some embodiments, the 4-10 membered heterocycloalkyl contains 0-1 oxygen and 0-2 nitrogen atoms. In some embodiments, the -4-10 membered heterocycloalkyl is,

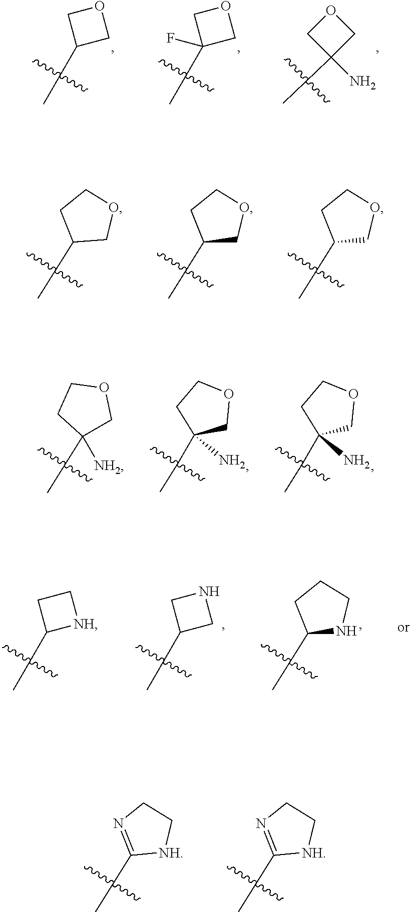

In some embodiments, R²³ is any one selected from the group consisting of

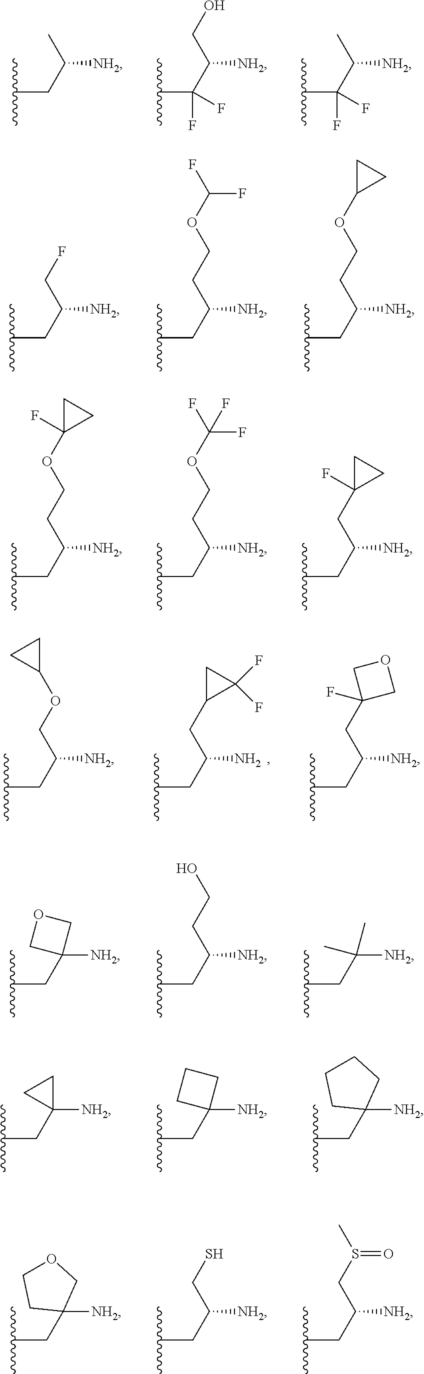

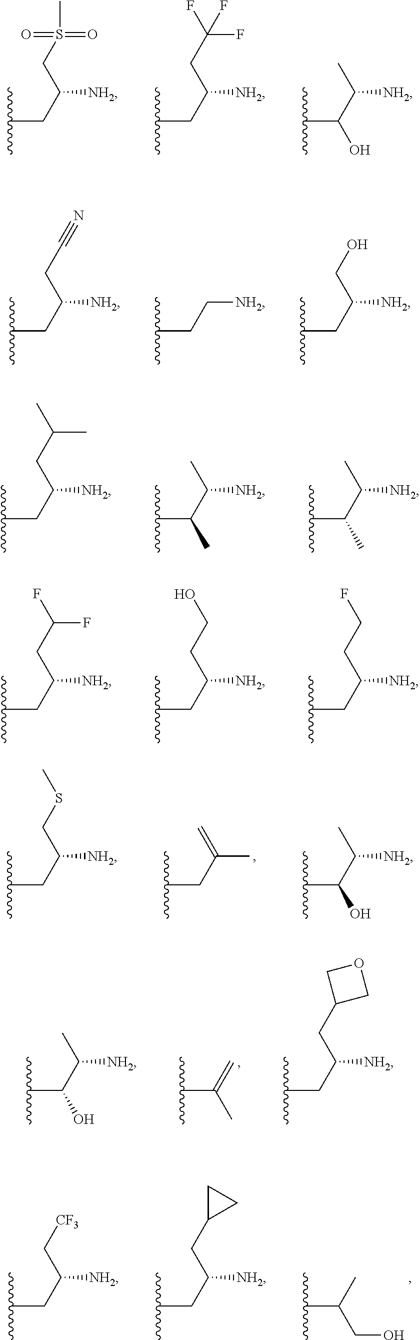

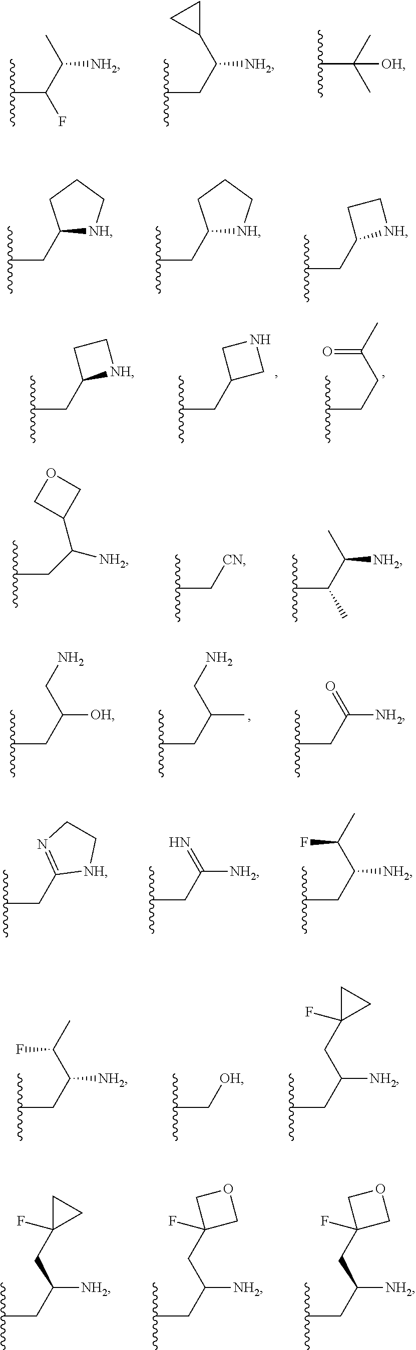

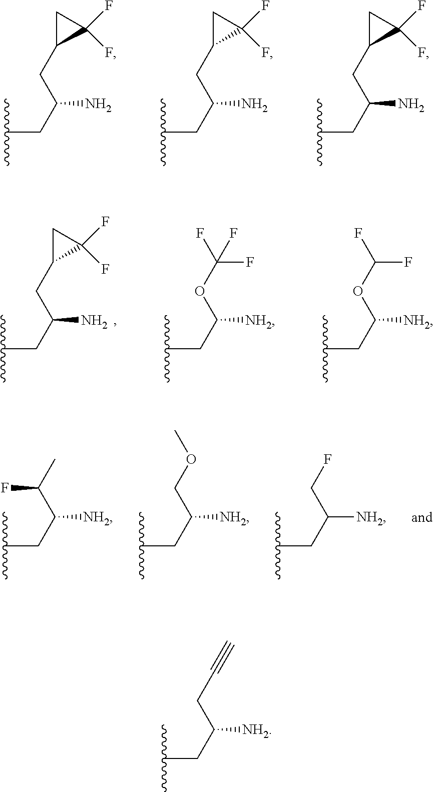

In some embodiments, each R²⁰ is independently selected from the group consisting of OH, SH, CN, NO₂, halo, oxo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, phenyl, 5-6 membered heteroaryl, 4-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, di(C₁₋₄ alkyl)aminocarbonylamino, and amidinyl. In some embodiments, each R²⁰ is independently selected from the group consisting of OH, SH, CN, NO₂, halo, oxo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —C₁₋₄ haloalkoxy, C₃₋₆ cycloalkyl, 4-6 membered heterocycloalkyl, amino, C₁₋₄ alkylamino, di(C₁₋₄ alkyl)amino, carbamyl, and amidinyl. In some embodiments, each R²⁰ is independently selected from the group consisting of OH, SH, CN, NO₂, halo, oxo, C₁₋₄ alkyl, C₁₋₄ haloalkyl, C₁₋₄ hydroxyalkyl, C₁₋₄ alkoxy, —C₁₋₄ haloalkoxy, C₃₋₆ cycloalkyl, 4-6 membered heterocycloalkyl, amino, carbamyl C₁₋₄ alkylamino, di(C₁₋₄ alkyl)amino, and amidinyl. In some embodiments, R²⁰ is OH. In some embodiments, R²⁰ is NH₂. In some embodiments, R²⁰ is SH. In some embodiments, R²⁰ is CN. In some embodiments, R²⁰ is F. In some embodiments, R²⁰ is carbamyl.

In some embodiments, X⁴ is N. In some embodiments, X⁴ is CH. In some embodiments, X⁴ is CR²⁴, wherein R²⁴ is selected from the group consisting of halo, CN, and substituted or unsubstituted C₁₋₆ alkyl. In some embodiments, X⁴ is CCl. In some embodiments, X⁴ is CBr. In some embodiments, X⁴ is CF. In some embodiments, X⁴ is CCN. In some embodiments, X⁴ is CCH₃. In some embodiments, X⁴ is C-cyclopropyl. In some embodiments, X⁴ is CR²⁴, wherein R²⁴ is selected from the group consisting of hydrogen, OH, halo, CN, substituted or unsubstituted C₁₋₆ alkyl, substituted or unsubstituted C₁₋₆ alkoxyl, substituted or unsubstituted C₃₋₁₀ cycloalkyl, substituted or unsubstituted C₂₋₄ alkenyl, and substituted or unsubstituted C₂₋₄ alkynyl.

In some embodiments, R²⁷ is hydrogen. In some embodiments, R²⁷ is halogen. In some embodiments, R²⁷ is C₁₋₆ alkyl. In some embodiments, R²⁷ is heteroalkyl. In some embodiments, R²⁷ is CN.

In some embodiments, each R^{a3}, R^{b3}, R^{c3}, R^{d3}, R^{a4}, R^{b4}, R^{c4}, R^{d4}, R^{a7}, R^{b7}, R^{c7}, and R^{d7} is independently selected from the group consisting of H, C₁₋₆ alkyl, C₁₋₆ hydroxyalkyl, and C₁₋₆ haloalkyl. In some embodiments, each R^{a3}, R^{b3}, R^{c3}, R^{d3}, R^{a4}, R^{b4}, R^{c4}, R^{d4}, R^{a7}, R^{b7}, R^{C7}, and R^{d7} is independently selected from the group consisting of H and C₁₋₆ alkyl. In some embodiments, each R^{a3}, R^{b3}, R^{c3}, R^{d3}, R^{a4}, R^{b4}, R⁴, R^{d4}, R^{a7}, R^{b7}, R^{c7}, and R^{d7} is independently selected from the group consisting of H and C₁₋₃ alkyl. In some embodiments, each R^{a3}, R^{b3}, R^{c3}, R^{d3}, R^{a4}, R^{b4}, R^{c4}, R^{d4}, R^{a7}, R^{b7}, R^{c7}, and R^{d7} is hydrogen.

In some embodiments, the compound is of the Formula (II):

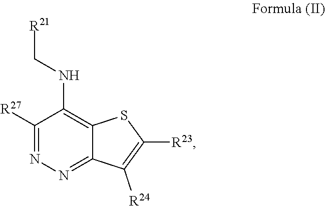

or a pharmaceutically acceptable salt or a pharmaceutically acceptable solvate thereof.

In some embodiments, R²³ is C₁₋₆ alkyl, wherein the C₁₋₆ alkyl is substituted with 1, 2, or 3 independently selected R²⁰ groups. In some embodiments, R²³ is C₁₋₆ heteroalkyl, wherein the C₁₋₆ heteroalkyl is substituted with 1, 2, or 3 independently selected R²⁰ groups. In some embodiments, C₁₋₆ alkyl is substituted with 1 R²⁰ group. In some embodiments, C₁₋₆ alkyl is substituted with 2 independently selected R²⁰ groups. In some embodiments, C₁₋₆ alkyl is substituted with 3 independently selected R²⁰ groups. In some embodiments, C₁₋₆ heteroalkyl is substituted with 1 R²⁰ group. In some embodiments, C₁₋₆ heteroalkyl is substituted with 2 independently selected R²⁰ groups. In some embodiments, C₁₋₆ heteroalkyl is substituted with 3 independently selected R²⁰ groups. In some embodiments, the C₁₋₆ heteroalkyl is —CH₂CH₂CH₂—S—CH₃.

In some embodiments, R²³ is methylene substituted with 1, 2, or 3 independently selected R²⁰ groups. In some embodiments, R²⁰ is methyl, ethyl, NH₂, CH₂OH, CH₂CH₂OH, CH₂CH₂F, CH₂CHF₂, or CH₂CH(CH₃)₂. In some embodiments, R²⁰ is NH₂ and methyl. In some embodiments, R²⁰ is NH₂ and CH₂OH. In some embodiments, R²⁰ is NH₂ and CH₂CH(CH₃)₂. In some embodiments, R²⁰ is NH₂ and CH₂CHF₂. In some embodiments, R²⁰ is NH₂ and CH₂CH₂F. In some embodiments, R²⁰ is NH₂ and CH₂CH₂OH. In some embodiments, R²⁰ is NH₂ and ethyl.

In some embodiments, the compound is of the Formula (IIIa):

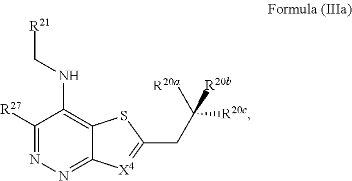

wherein each R^{20a}, R^{20b}, and R^{20c} is independently selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₁₋₄ heteroalkyl, phenyl, 5-6 membered heteroaryl, 4-6 membered heterocycloalkyl, —(C₁₋₃ alkylene)-C₃₋₁₀ cycloalkyl, —(C₁₋₃ alkylene)-4-10 membered heterocycloalkyl, —(C₁₋₃ heteroalkylene)-C₃₋₁₀ cycloalkyl, —(C₁₋₃ heteroalkylene)-4-10 membered heterocycloalkyl, amidinyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein:
each of the cycloalkyl and heterocycloalkyl is optionally substituted by 1, 2, 3, or 4 substituents independently selected from halo, CN, SH, —CN, oxo, NO₂, OH, C₁₋₄ alkyl, C₂₋₄ alkenyl, C₂₋₄ alkynyl, C₁₋₄ haloalkyl, C₁₋₄ cyanoalkyl, C₁₋₄ aminoalkyl, C₁₋₄ hydroxyalkyl, C₁₋₄ alkoxy, and amino,
or R^{20a} and R^{20b} are taken together to form a ═NH or =N(C₁₋₄ alkyl),
   or a pharmaceutically acceptable salt or a pharmaceutically acceptable solvate thereof.

In some embodiments of the compound of the Formula (IIIa) or a pharmaceutically acceptable salt or a pharmaceutically acceptable solvate thereof, each R^{20a}, R^{20b}, and R^{20c} is independently selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, phenyl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂. In some embodiments, R^{20c} is NH₂. In some embodiments, R^{20b} is hydrogen. In some embodiments, R^{20a} and R^{20b} are taken together to form a ═NH or =N(C₁₋₄ alkyl). In some embodiments, R^{20a} is 4-6 membered heterocycloalkyl. In some embodiments, R^{20a} is —(C₁₋₃ alkylene)-C₃₋₁₀ cycloalkyl. In some embodiments, R^{20a} is —(C₁₋₃ alkylene)-4-10 membered heterocycloalkyl. In some embodiments, R^{20a} is —(C₁₋₃ heteroalkylene)-C₃₋₁₀ cycloalkyl. In some embodiments, R^{20a} is —(C₁₋₃ heteroalkylene)-4-10 membered heterocycloalkyl. In some embodiments, the —C₃₋₁₀ cycloalkyl is

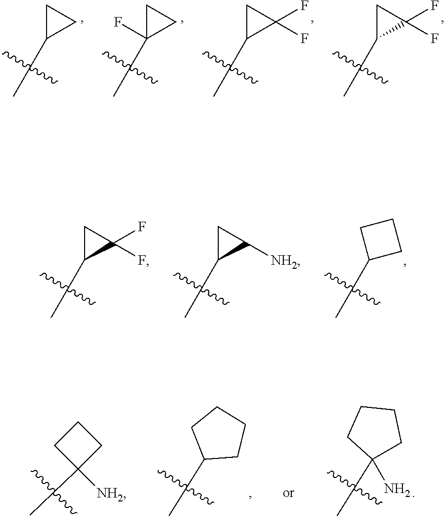

In some embodiments, the —C₃₋₁₀ cycloalkyl is a 3-5 membered ring, which is optionally substituted. In some embodiments, the -4-10 membered heterocycloalkyl is

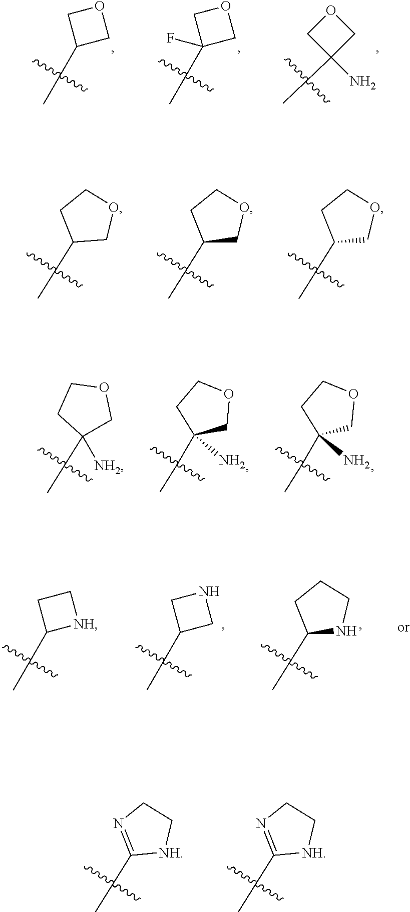

In some embodiments, the -4-10 membered heterocycloalkyl is a 4-5 membered ring, which is optionally substituted. In some embodiments, R^{20a} is C₁₋₄ heteroalkyl.

In some embodiments, R²⁷ is hydrogen. In some embodiments, R²⁷ is halogen. In some embodiments, R²⁷ is C₁₋₆ alkyl. In some embodiments, R²⁷ is heteroalkyl. In some embodiments, R²⁷ is CN.

In some embodiments, the compound is of the Formula (IIIb):

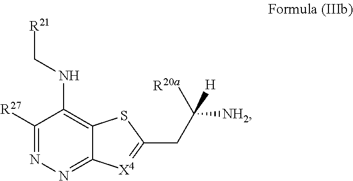

wherein R^{20a} is selected from the group consisting of H, OH, SH, CN, NO₂, halo, oxo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₁₋₄ heteroalkyl, phenyl, 5-6 membered heteroaryl, 4-6 membered heterocycloalkyl, —(C₁₋₃ alkylene)-C₃₋₁₀ cycloalkyl, —(C₁₋₃ alkylene)-4-10 membered heterocycloalkyl, —(C₁₋₃ heteroalkylene)-C₃₋₁₀ cycloalkyl, —(C₁₋₃ heteroalkylene)-4-10 membered heterocycloalkyl, amidinyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein each of the cycloalkyl and heterocycloalkyl is optionally substituted by 1, 2, 3, or 4 substituents independently selected from halo, CN, SH, —CN, oxo, NO₂, OH, C₁₋₄ alkyl, C₂₋₄ alkenyl, C₂₋₄ alkynyl, C₁₋₄ haloalkyl, C₁₋₄ cyanoalkyl, C₁₋₄ aminoalkyl, C₁₋₄ hydroxyalkyl, C₁₋₄ alkoxy, and amino,
   or a pharmaceutically acceptable salt or a pharmaceutically acceptable solvate thereof.

In some embodiments of the compound of the Formula (IIIb) or a pharmaceutically acceptable salt or a pharmaceutically acceptable solvate thereof: R^{20a} is selected from the group consisting of OH, SH, CN, NO₂, halo, oxo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, phenyl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂. In some embodiments, R^{20a} is 4-6 membered heterocycloalkyl. In some embodiments, R^{20a} is —(C₁₋₃ alkylene)-C₃₋₁₀ cycloalkyl. In some embodiments, R^{20a} is —(C₁₋₃ alkylene)-4-10 membered heterocycloalkyl. In some embodiments, R^{20a} is —(C₁₋₃ heteroalkylene)-C₃₋₁₀ cycloalkyl. In some embodiments, R^{20a} is —(C₁₋₃ heteroalkylene)-4-10 membered heterocycloalkyl. In some embodiments, the —C₃₋₁₀ cycloalkyl is

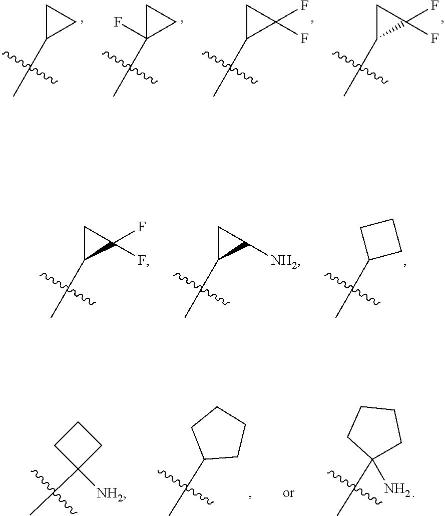

In some embodiments, the —C₃₋₁₀ cycloalkyl is a 3-5 membered ring, which is optionally substituted. In some embodiments, the -4-10 membered heterocycloalkyl is

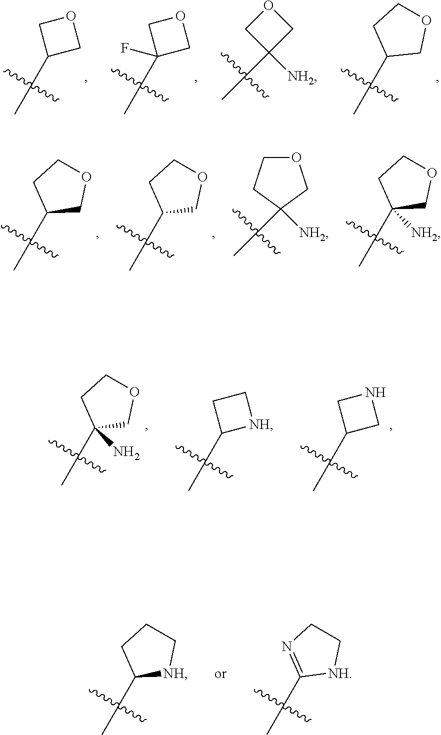

In some embodiments, the -4-10 membered heterocycloalkyl is a 4-5 membered ring, which is optionally substituted. In some embodiments, R^{20a} is C₁₋₄ heteroalkyl. In some embodiments, R^{20a} is C₁₋₄ alkyl. In some embodiments, R^{20a} is optionally substituted C₁₋₄ heteroalkyl. In some embodiments, R^{20a} is optionally substituted C₁₋₄ alkyl.

In some embodiments, R²⁷ is hydrogen. In some embodiments, R²⁷ is halogen. In some embodiments, R²⁷ is C₁₋₆ alkyl. In some embodiments, R²⁷ is heteroalkyl. In some embodiments, R²⁷ is CN.

In some embodiments, the compound is of the Formula (IVa):

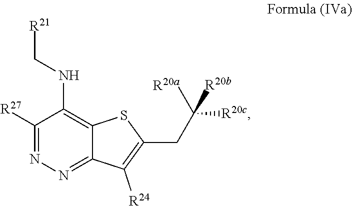

wherein each R^{20a}, R^{20b}, and R^{20c} is independently selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₁₋₄ heteroalkyl, phenyl, 5-6 membered heteroaryl, 4-6 membered heterocycloalkyl, —(C₁₋₃ alkylene)-C₃₋₁₀ cycloalkyl, —(C₁₋₃ alkylene)-4-10 membered heterocycloalkyl, —(C₁₋₃ heteroalkylene)-C₃₋₁₀ cycloalkyl, —(C₁₋₃ heteroalkylene)-4-10 membered heterocycloalkyl, amidinyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein:
each of the cycloalkyl and heterocycloalkyl is optionally substituted by 1, 2, 3, or 4 substituents independently selected from halo, CN, SH, —CN, oxo, NO₂, OH, C₁₋₄ alkyl, C₂₋₄ alkenyl, C₂₋₄ alkynyl, C₁₋₄ haloalkyl, C₁₋₄ cyanoalkyl, C₁₋₄ aminoalkyl, C₁₋₄ hydroxyalkyl, C₁₋₄ alkoxy, and amino,
or R^{20a} and R^{20b} are taken together to form a ═NH or =N(C₁₋₄ alkyl),
   or a pharmaceutically acceptable salt or a pharmaceutically acceptable solvate thereof.

In some embodiments of the compound of the Formula (IVa) or a pharmaceutically acceptable salt or a pharmaceutically acceptable solvate thereof, each R^{20a}, R^{20b}, and R^{20c} is independently selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, phenyl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂. In some embodiments, R^{20c} is NH₂. In some embodiments, R^{20b} is hydrogen. In some embodiments, R^{20a} is 4-6 membered heterocycloalkyl. In some embodiments, R^{20a} is —(C₁₋₃ alkylene)-C₃₋₁₀ cycloalkyl. In some embodiments, R^{20a} is —(C₁₋₃ alkylene)-4-10 membered heterocycloalkyl. In some embodiments, R^{20a} is —(C₁₋₃ heteroalkylene)-C₃₋₁₀ cycloalkyl. In some embodiments, R^{20a} is —(C₁₋₃ heteroalkylene)-4-10 membered heterocycloalkyl. In some embodiments, the —C₃₋₁₀ cycloalkyl is

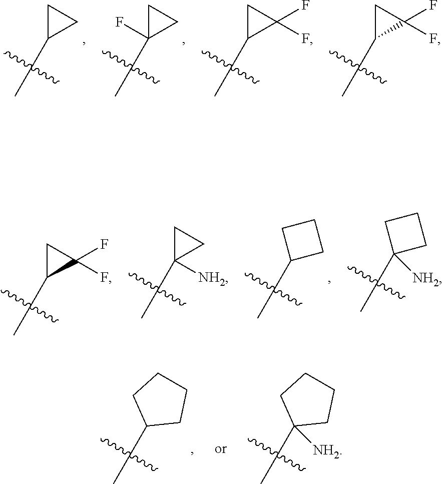

In some embodiments, the —C₃₋₁₀ cycloalkyl is a 3-5 membered ring, which is optionally substituted. In some embodiments, the -4-10 membered heterocycloalkyl is

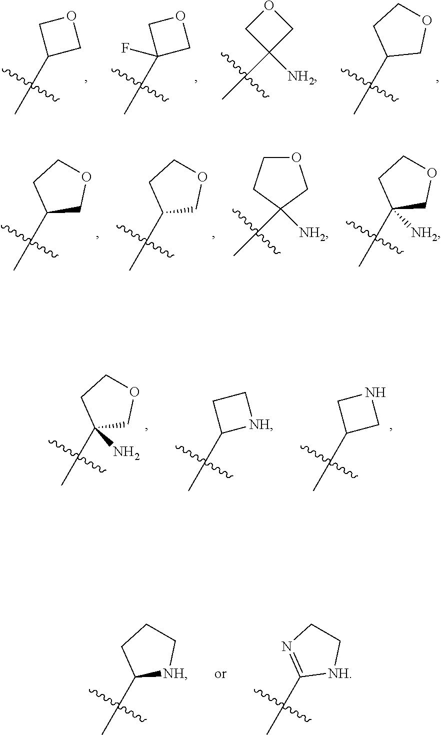

In some embodiments, the -4-10 membered heterocycloalkyl is a 4-5 membered ring, which is optionally substituted. In some embodiments, R^{20a} is C₁₋₄ heteroalkyl. In some embodiments, R^{20a} is C₁₋₄ alkyl. In some embodiments, R^{20a} is optionally substituted C₁₋₄ heteroalkyl. In some embodiments, R^{20a} is optionally substituted C₁₋₄ alkyl.

In some embodiments, R²⁷ is hydrogen. In some embodiments, R²⁷ is halogen. In some embodiments, R²⁷ is C₁₋₆ alkyl. In some embodiments, R²⁷ is heteroalkyl. In some embodiments, R²⁷ is CN.

In some embodiments, the compound is of the Formula (IVb):

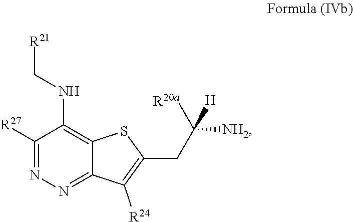

wherein R^{20a} is selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₁₋₄ heteroalkyl, phenyl, 5-6 membered heteroaryl, 4-6 membered heterocycloalkyl, —(C₁₋₃ alkylene)-C₃₋₁₀ cycloalkyl, —(C₁₋₃ alkylene)-4-10 membered heterocycloalkyl, —(C₁₋₃ heteroalkylene)-C₃₋₁₀ cycloalkyl, —(C₁₋₃ heteroalkylene)-4-10 membered heterocycloalkyl, amidinyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein each of the cycloalkyl and heterocycloalkyl is optionally substituted by 1, 2, 3, or 4 substituents independently selected from halo, CN, SH, —CN, oxo, NO₂, OH, C₁₋₄ alkyl, C₂₋₄ alkenyl, C₂₋₄ alkynyl, C₁₋₄ haloalkyl, C₁₋₄ cyanoalkyl, C₁₋₄ aminoalkyl, C₁₋₄ hydroxyalkyl, C₁₋₄ alkoxy, and amino,
   or a pharmaceutically acceptable salt or a pharmaceutically acceptable solvate thereof.

In some embodiments of the compound of the Formula (IVb) or a pharmaceutically acceptable salt or a pharmaceutically acceptable solvate thereof, R^{20a} is selected from the group consisting of OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, phenyl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂. In some embodiments, R^{20a} is 4-6 membered heterocycloalkyl. In some embodiments, R^{20a} is —(C₁₋₃ alkylene)-C₃₋₁₀ cycloalkyl. In some embodiments, R^{20a} is —(C₁₋₃ alkylene)-4-10 membered heterocycloalkyl. In some embodiments, R^{20a} is —(C₁₋₃ heteroalkylene)-C₃₋₁₀ cycloalkyl. In some embodiments, R^{20a} is —(C₁₋₃ heteroalkylene)-4-10 membered heterocycloalkyl. In some embodiments, the —C₃₋₁₀ cycloalkyl is

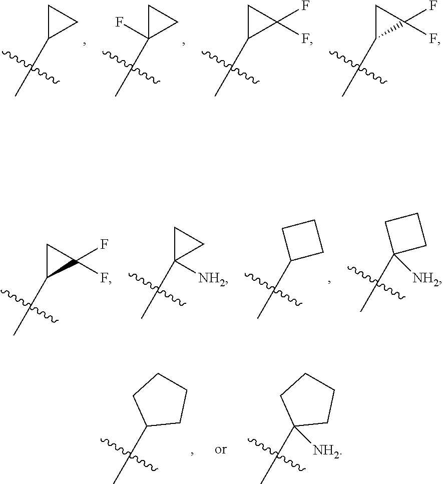

In some embodiments, the —C₃₋₁₀ cycloalkyl is a 3-5 membered ring, which is optionally substituted. In some embodiments, the -4-10 membered heterocycloalkyl is

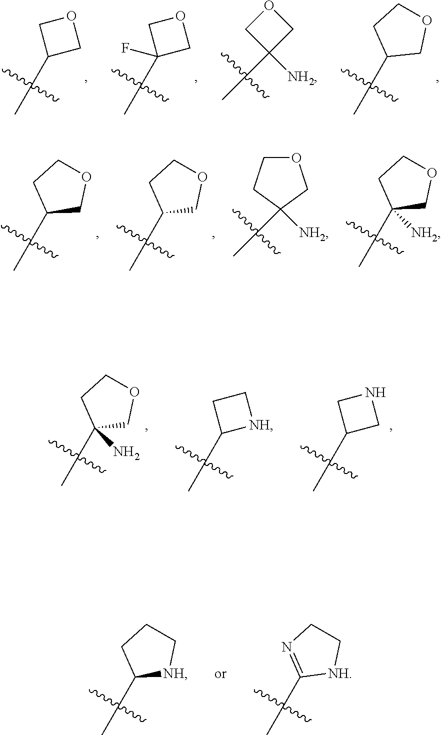

In some embodiments, the -4-10 membered heterocycloalkyl is a 4-5 membered ring, which is optionally substituted. In some embodiments, R^{20a} is C₁₋₄ heteroalkyl. In some embodiments, R^{20a} is C₁₋₄ alkyl. In some embodiments, R^{20a} is optionally substituted C₁₋₄ heteroalkyl. In some embodiments, R^{20a} is optionally substituted C₁₋₄ alkyl.

In some embodiments, R²⁴ is C₁₋₆ alkyl. In some embodiments, R²⁴ is methyl. In some embodiments, R²⁴ is halo. In some embodiments, R²⁴ is fluoro, bromo, or chloro. In some embodiments, R²⁴ is hydrogen. In some embodiments, R²⁴ is CN. In some embodiments, R²⁴ is C₃₋₁₀ cycloalkyl.

In some embodiments, R²⁷ is hydrogen. In some embodiments, R²⁷ is halogen. In some embodiments, R²⁷ is C₁₋₆ alkyl. In some embodiments, R²⁷ is heteroalkyl. In some embodiments, R²⁷ is CN.

In some embodiments, the compound is of the Formula (IVc):

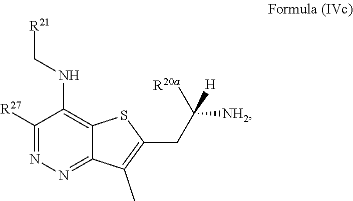

wherein R^{20a} is selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₁₋₄ heteroalkyl, phenyl, 5-6 membered heteroaryl, 4-6 membered heterocycloalkyl, —(C₁₋₃ alkylene)-C₃₋₁₀ cycloalkyl, —(C₁₋₃ alkylene)-4-10 membered heterocycloalkyl, —(C₁₋₃ heteroalkylene)-C₃₋₁₀ cycloalkyl, —(C₁₋₃ heteroalkylene)-4-10 membered heterocycloalkyl, amidinyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein each of the cycloalkyl and heterocycloalkyl is optionally substituted by 1, 2, 3, or 4 substituents independently selected from halo, CN, SH, —CN, oxo, NO₂, OH, C₁₋₄ alkyl, C₂₋₄ alkenyl, C₂₋₄ alkynyl, C₁₋₄ haloalkyl, C₁₋₄ cyanoalkyl, C₁₋₄ aminoalkyl, C₁₋₄ hydroxyalkyl, C₁₋₄ alkoxy, and amino,
   or a pharmaceutically acceptable salt or a pharmaceutically acceptable solvate thereof.

In some embodiments of the compound of the Formula (IVc) or a pharmaceutically acceptable salt or a pharmaceutically acceptable solvate thereof, R^{20a} is selected from the group consisting of OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, phenyl, 5-6 membered heteroaryl, 5-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂. In some embodiments, R^{20a} is 4-6 membered heterocycloalkyl. In some embodiments, R^{20a} is —(C₁₋₃ alkylene)-C₃₋₁₀ cycloalkyl. In some embodiments, R^{20a} is —(C₁₋₃ alkylene)-4-10 membered heterocycloalkyl. In some embodiments, R^{20a} is —(C₁₋₃ heteroalkylene)-C₃₋₁₀ cycloalkyl. In some embodiments, R^{20a} is —(C₁₋₃ heteroalkylene)-4-10 membered heterocycloalkyl. In some embodiments, the —C₃₋₁₀ cycloalkyl is

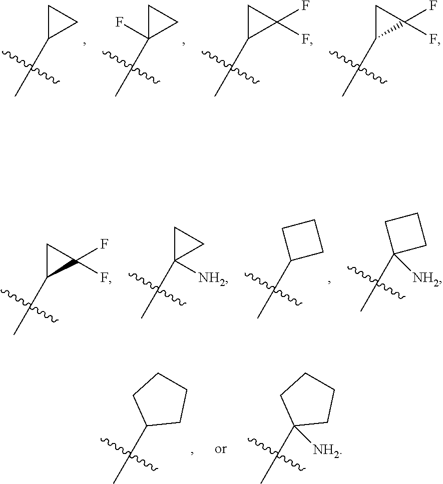

In some embodiments, the —C₃₋₁₀ cycloalkyl is a 3-5 membered ring, which is optionally substituted. In some embodiments, the -4-10 membered heterocycloalkyl is

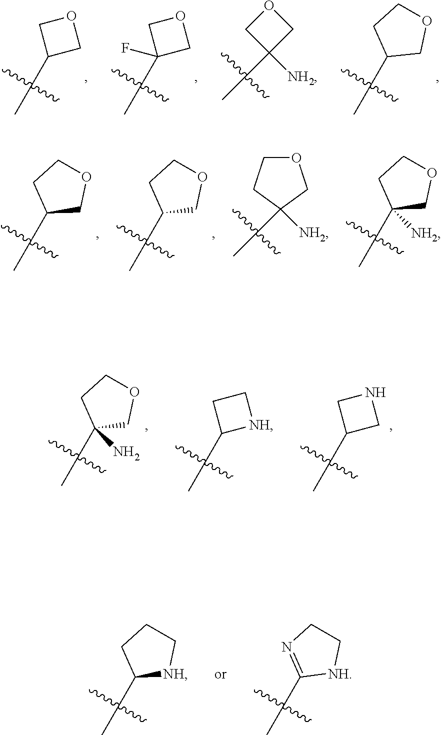

In some embodiments, the -4-10 membered heterocycloalkyl is a 4-5 membered ring, which is optionally substituted. In some embodiments, R^{20a} is C₁₋₄ heteroalkyl. In some embodiments, R^{20a} is C₁₋₄ alkyl. In some embodiments, R^{20a} is optionally substituted C₁₋₄ heteroalkyl. In some embodiments, R^{20a} is optionally substituted C₁₋₄ alkyl.

In some embodiments, R²⁷ is hydrogen. In some embodiments, R²⁷ is halogen. In some embodiments, R²⁷ is C₁₋₆ alkyl. In some embodiments, R²⁷ is heteroalkyl. In some embodiments, R²⁷ is CN.

In some embodiments, the compound is of the Formula (IVd):

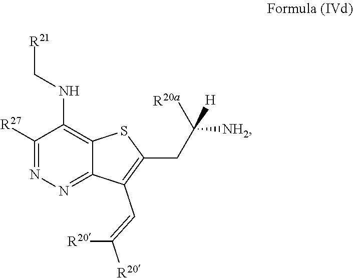

wherein R^{20a} is selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₁₋₄ heteroalkyl, phenyl, 5-6 membered heteroaryl, 4-6 membered heterocycloalkyl, —(C₁₋₃ alkylene)-C₃₋₁₀ cycloalkyl, —(C₁₋₃ alkylene)-4-10 membered heterocycloalkyl, —(C₁₋₃ heteroalkylene)-C₃₋₁₀ cycloalkyl, —(C₁₋₃ heteroalkylene)-4-10 membered heterocycloalkyl, amidinyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein each of the cycloalkyl and heterocycloalkyl is optionally substituted by 1, 2, 3, or 4 substituents independently selected from halo, CN, SH, —CN, oxo, NO₂, OH, C₁₋₄ alkyl, C₂₋₄ alkenyl, C₂₋₄ alkynyl, C₁₋₄ haloalkyl, C₁₋₄ cyanoalkyl, C₁₋₄ aminoalkyl, C₁₋₄ hydroxyalkyl, C₁₋₄ alkoxy, and amino,
wherein each of R^{20′} is independently H or R²⁰ as disclosed herein,
   or a pharmaceutically acceptable salt or a pharmaceutically acceptable solvate thereof.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂. In some embodiments, R^{20a} is 4-6 membered heterocycloalkyl. In some embodiments, R^{20a} is —(C₁₋₃ alkylene)-C₃₋₁₀ cycloalkyl. In some embodiments, R^{20a} is —(C₁₋₃ alkylene)-4-10 membered heterocycloalkyl. In some embodiments, R^{20a} is —(C₁₋₃ heteroalkylene)-C₃₋₁₀ cycloalkyl. In some embodiments, R^{20a} is —(C₁₋₃ heteroalkylene)-4-10 membered heterocycloalkyl. In some embodiments, the —C₃₋₁₀ cycloalkyl is

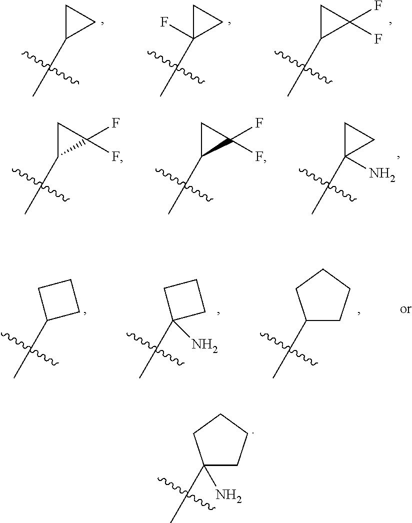

In some embodiments, the —C₃₋₁₀ cycloalkyl is a 3-5 membered ring, which is optionally substituted. In some embodiments, the -4-10 membered heterocycloalkyl is

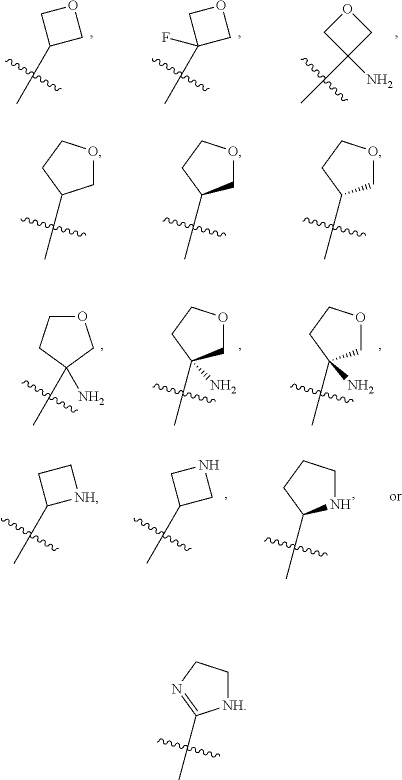

In some embodiments, the -4-10 membered heterocycloalkyl is a 4-5 membered ring, which is optionally substituted. In some embodiments, R^{20a} is C₁₋₄ heteroalkyl. In some embodiments, R^{20a} is C₁₋₄ alkyl. In some embodiments, R^{20a} is optionally substituted C₁₋₄ heteroalkyl. In some embodiments, R^{20a} is optionally substituted C₁₋₄ alkyl.

In some embodiments, R²⁷ is hydrogen. In some embodiments, R²⁷ is halogen. In some embodiments, R²⁷ is C₁₋₆ alkyl. In some embodiments, R²⁷ is heteroalkyl. In some embodiments, R²⁷ is CN.

In some embodiments, the compound is of the Formula (IVe):

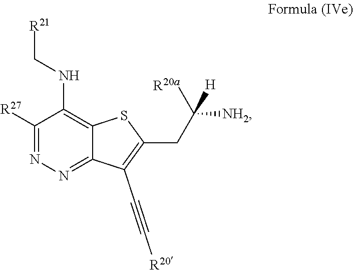

wherein R^{20a} is selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₁-4 heteroalkyl, phenyl, 5-6 membered heteroaryl, 4-6 membered heterocycloalkyl, —(C₁₋₃ alkylene)-C₃₋₁₀ cycloalkyl, —(C₁₋₃ alkylene)-4-10 membered heterocycloalkyl, —(C₁₋₃ heteroalkylene)-C₃₋₁₀ cycloalkyl, —(C₁₋₃ heteroalkylene)-4-10 membered heterocycloalkyl, amidinyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein each of the cycloalkyl and heterocycloalkyl is optionally substituted by 1, 2, 3, or 4 substituents independently selected from halo, CN, SH, —CN, oxo, NO₂, OH, C₁₋₄ alkyl, C₂₋₄ alkenyl, C₂₋₄ alkynyl, C₁₋₄ haloalkyl, C₁₋₄ cyanoalkyl, C₁₋₄ aminoalkyl, C₁₋₄ hydroxyalkyl, C₁₋₄ alkoxy, and amino,
wherein R^{20′} is H or R²⁰ as disclosed herein,
   or a pharmaceutically acceptable salt or a pharmaceutically acceptable solvate thereof.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂. In some embodiments, R^{20a} is 4-6 membered heterocycloalkyl. In some embodiments, R^{20a} is —(C₁₋₃ alkylene)-C₃₋₁₀ cycloalkyl. In some embodiments, R^{20a} is —(C₁₋₃ alkylene)-4-10 membered heterocycloalkyl. In some embodiments, R^{20a} is —(C₁₋₃ heteroalkylene)-C₃₋₁₀ cycloalkyl. In some embodiments, R^{20a} is —(C₁₋₃ heteroalkylene)-4-10 membered heterocycloalkyl. In some embodiments, the —C₃₋₁₀ cycloalkyl is

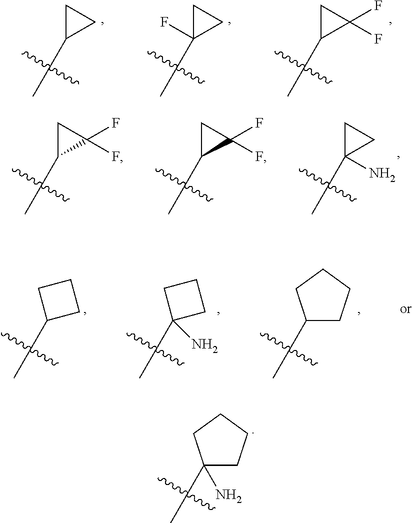

In some embodiments, the —C₃₋₁₀ cycloalkyl is a 3-5 membered ring, which is optionally substituted. In some embodiments, the -4-10 membered heterocycloalkyl is

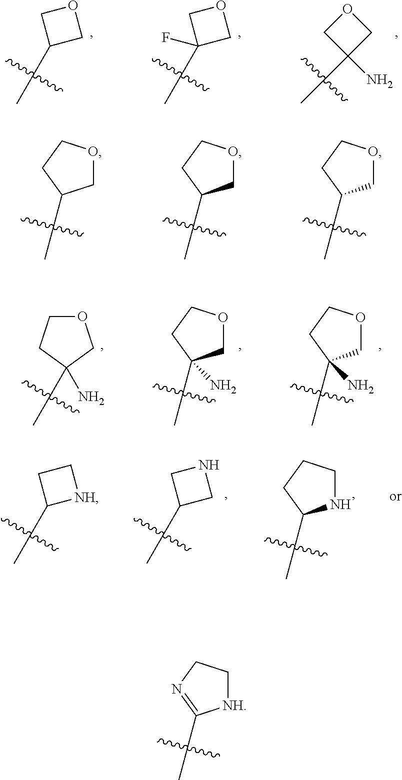

In some embodiments, the -4-10 membered heterocycloalkyl is a 4-5 membered ring, which is optionally substituted. In some embodiments, R^{20a} is C₁₋₄ heteroalkyl. In some embodiments, R^{20a} is C₁₋₄ alkyl. In some embodiments, R^{20a} is optionally substituted C₁₋₄ heteroalkyl. In some embodiments, R^{20a} is optionally substituted C₁₋₄ alkyl.

In some embodiments, R²⁷ is hydrogen. In some embodiments, R²⁷ is halogen. In some embodiments, R²⁷ is C₁₋₆ alkyl. In some embodiments, R²⁷ is heteroalkyl. In some embodiments, R²⁷ is CN.

In some embodiments, the compound is of the Formula (IVf):

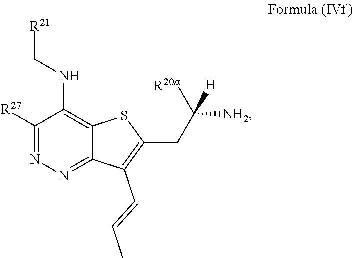

wherein R^{20a} is selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₁₋₄ heteroalkyl, phenyl, 5-6 membered heteroaryl, 4-6 membered heterocycloalkyl, —(C₁₋₃ alkylene)-C₃₋₁₀ cycloalkyl, —(C₁₋₃ alkylene)-4-10 membered heterocycloalkyl, —(C₁₋₃ heteroalkylene)-C₃₋₁₀ cycloalkyl, —(C₁₋₃ heteroalkylene)-4-10 membered heterocycloalkyl, amidinyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein each of the cycloalkyl and heterocycloalkyl is optionally substituted by 1, 2, 3, or 4 substituents independently selected from halo, CN, SH, —CN, oxo, NO₂, OH, C₁₋₄ alkyl, C₂₋₄ alkenyl, C₂₋₄ alkynyl, C₁₋₄ haloalkyl, C₁₋₄ cyanoalkyl, C₁₋₄ aminoalkyl, C₁₋₄ hydroxyalkyl, C₁₋₄ alkoxy, and amino,
   or a pharmaceutically acceptable salt or a pharmaceutically acceptable solvate thereof.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂. In some embodiments, R^{20a} is 4-6 membered heterocycloalkyl. In some embodiments, R^{20a} is —(C₁₋₃ alkylene)-C₃₋₁₀ cycloalkyl. In some embodiments, R^{20a} is —(C₁₋₃ alkylene)-4-10 membered heterocycloalkyl. In some embodiments, R^{20a} is —(C₁₋₃ heteroalkylene)-C₃₋₁₀ cycloalkyl. In some embodiments, R^{20a} is —(C₁₋₃ heteroalkylene)-4-10 membered heterocycloalkyl. In some embodiments, the —C₃₋₁₀ cycloalkyl is

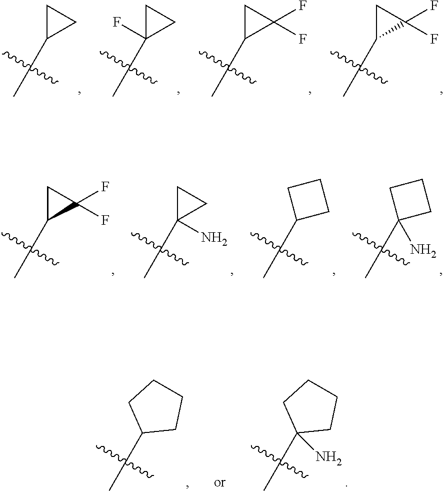

In some embodiments, the —C₃₋₁₀ cycloalkyl is a 3-5 membered ring, which is optionally substituted. In some embodiments, the -4-10 membered heterocycloalkyl is

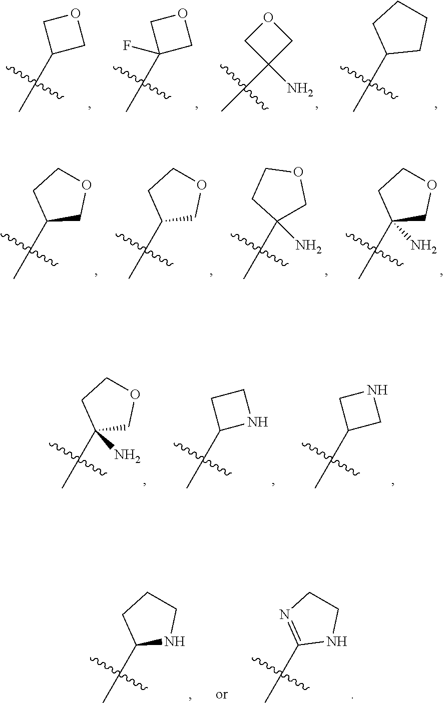

In some embodiments, the -4-10 membered heterocycloalkyl is a 4-5 membered ring, which is optionally substituted. In some embodiments, R^{20a} is C₁₋₄ heteroalkyl. In some embodiments, R^{20a} is C₁₋₄ alkyl. In some embodiments, R^{20a} is optionally substituted C₁₋₄ heteroalkyl. In some embodiments, R^{20a} is optionally substituted C₁₋₄ alkyl.

In some embodiments, R²⁷ is hydrogen. In some embodiments, R²⁷ is halogen. In some embodiments, R²⁷ is C₁₋₆ alkyl. In some embodiments, R²⁷ is heteroalkyl. In some embodiments, R²⁷ is CN.

In some embodiments, the compound is of the Formula (IVg):

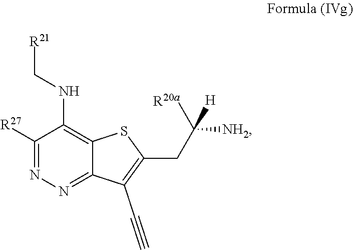

wherein R^{20a} is selected from the group consisting of H, OH, SH, CN, NO₂, halo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, C₁₋₄ heteroalkyl, phenyl, 5-6 membered heteroaryl, 4-6 membered heterocycloalkyl, —(C₁₋₃ alkylene)-C₃₋₁₀ cycloalkyl, —(C₁₋₃ alkylene)-4-10 membered heterocycloalkyl, —(C₁₋₃ heteroalkylene)-C₃₋₁₀ cycloalkyl, —(C₁₋₃ heteroalkylene)-4-10 membered heterocycloalkyl, amidinyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, and di(C₁₋₄ alkyl)aminocarbonylamino, wherein each of the cycloalkyl and heterocycloalkyl is optionally substituted by 1, 2, 3, or 4 substituents independently selected from halo, CN, SH, —CN, oxo, NO₂, OH, C₁₋₄ alkyl, C₂₋₄ alkenyl, C₂₋₄ alkynyl, C₁₋₄ haloalkyl, C₁₋₄ cyanoalkyl, C₁₋₄ aminoalkyl, C₁₋₄ hydroxyalkyl, C₁₋₄ alkoxy, and amino,
   or a pharmaceutically acceptable salt or a pharmaceutically acceptable solvate thereof.

In some embodiments, R^{20a} is methyl. In some embodiments, R^{20a} is ethyl. In some embodiments, R^{20a} is CH₂OH. In some embodiments, R^{20a} is CH₂CH₂OH. In some embodiments, R^{20a} is CH₂CH₂F. In some embodiments, R^{20a} is CH₂CHF₂. In some embodiments, R^{20a} is CH₂CH(CH₃)₂. In some embodiments, R^{20a} is 4-6 membered heterocycloalkyl. In some embodiments, R^{20a} is —(C₁₋₃ alkylene)-C₃₋₁₀ cycloalkyl. In some embodiments, R^{20a} is —(C₁₋₃ alkylene)-4-10 membered heterocycloalkyl. In some embodiments, R^{20a} is —(C₁₋₃ heteroalkylene)-C₃₋₁₀ cycloalkyl. In some embodiments, R^{20a} is —(C₁₋₃ heteroalkylene)-4-10 membered heterocycloalkyl. In some embodiments, the —C₃₋₁₀ cycloalkyl is

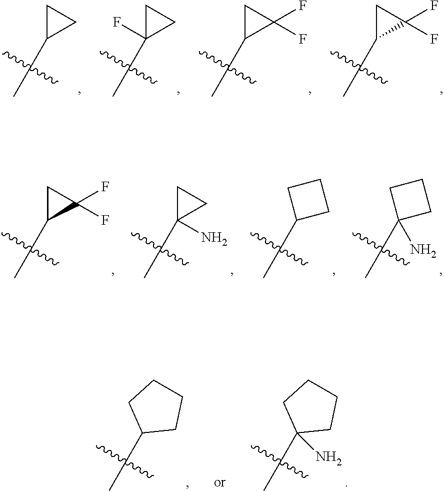

In some embodiments, the —C₃₋₁₀ cycloalkyl is a 3-5 membered ring, which is optionally substituted. In some embodiments, the -4-10 membered heterocycloalkyl is

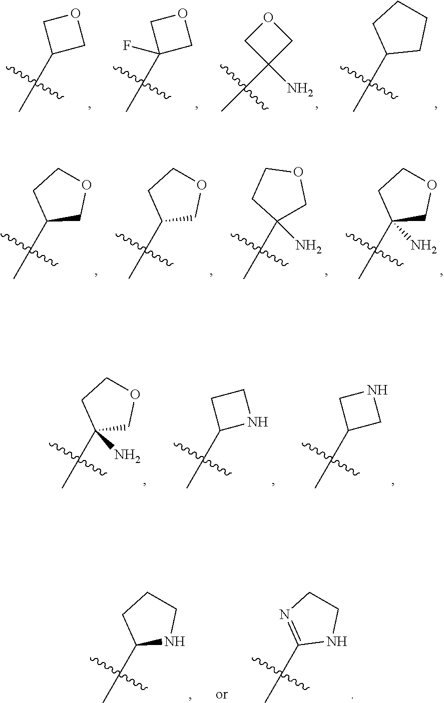

In some embodiments, the -4-10 membered heterocycloalkyl is a 4-5 membered ring, which is optionally substituted. In some embodiments, R^{20a} is C₁₋₄ heteroalkyl. In some embodiments, R^{20a} is C₁₋₄ alkyl. In some embodiments, R^{20a} is optionally substituted C₁₋₄ heteroalkyl. In some embodiments, R^{20a} is optionally substituted C₁₋₄ alkyl.

In some embodiments, R²⁷ is hydrogen. In some embodiments, R²⁷ is halogen. In some embodiments, R²⁷ is C₁₋₆ alkyl. In some embodiments, R²⁷ is heteroalkyl. In some embodiments, R²⁷ is CN.

In some embodiments, R^{20′} is H. In some embodiments, R^{20′} is selected from R²⁰.

In some embodiments, the compound is selected from Table 1.

In some embodiments, a SMSM described herein, possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis, trans, syn, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof (See, for example, Jean Jacques, Andre Collet, Samuel H. Wilen, “Enantiomers, Racemates and Resolutions”, John Wiley and Sons, Inc., 1981.) In one aspect, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) to facilitate transmittal across a cell membrane where water solubility is detrimental to mobility, but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In one aspect, prodrugs are designed to alter the metabolic stability or the transport characteristics of a drug, to mask side effects or toxicity, to improve the flavor of a drug or to alter other characteristics or properties of a drug. By virtue of knowledge of pharmacokinetic, pharmacodynamic processes and drug metabolism in vivo, once a pharmaceutically active compound is known, the design of prodrugs of the compound is possible. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Rooseboom et al., Pharmacological Reviews, 56:53-102, 2004; Aesop Cho, “Recent Advances in Oral Prodrug Discovery”, Annual Reports in Medicinal Chemistry, Vol. 41, 395-407, 2006; T. Higuchi and V. Stella, Pro-drugs as Novel Delivery Systems, Vol. 14 of the A.C.S. Symposium Series).

In some cases, some of the herein-described compounds may be a prodrug for another derivative or active compound.

In some embodiments, sites on the aromatic ring portion of compounds described herein are susceptible to various metabolic reactions Therefore incorporation of appropriate substituents on the aromatic ring structures will reduce, minimize or eliminate this metabolic pathway. In specific embodiments, the appropriate substituent to decrease or eliminate the susceptibility of the aromatic ring to metabolic reactions is, by way of example only, a halogen, or an alkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine and chlorine, such as, for example, ²H, ³H, ¹³C, ¹⁴C, ¹⁵N, ¹⁸O, ¹⁷O, ³⁵S, ¹⁸F, ³⁶Cl. In one aspect, isotopically labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent and may be formed during the process of crystallization with pharmaceutically acceptable solvents such as water, ethanol, and the like. Hydrates are formed when the solvent is water, or alcoholates are formed when the solvent is alcohol. In some embodiments, solvates of compounds described herein are conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

In some embodiments, a SMSM has a molecular weight of at most about 2000 Daltons, 1500 Daltons, 1000 Daltons or 900 Daltons. In some embodiments, a SMSM has a molecular weight of at least 100 Daltons, 200 Daltons, 300 Daltons, 400 Daltons or 500 Daltons. In some embodiments, a SMSM does not comprise a phosphodiester linkage. In some embodiments, a SMSM is a compound with a structure set forth in Table 1 below.

TABLE 1
Exemplary SMSM compounds

| Compound | Structure | IUPAC Name |
|---|---|---|
| 1 | 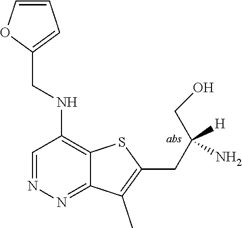 [SMILES: Cc1c(C[C@@H](N)CO)sc2c(NCc3ccco3)cnnc12] | (2R)-2-amino-3-(4-{[(furan-2- yl)methyl]amino}-7-methylthieno[3,2- c]pyridazin-6-yl)propan-1-ol |
| 2 | 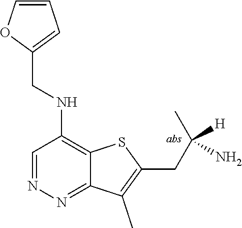 [SMILES: Cc1c(C[C@H](C)N)sc2c(NCc3ccco3)cnnc12] | 6-[(2S)-2-aminopropyl]-N-[(furan-2- yl)methyl]-7-methylthieno[3,2- c]pyridazin-4-amine |
| 3 | 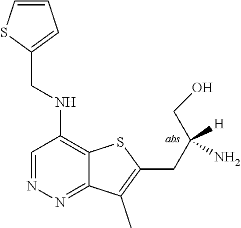 [SMILES: Cc1c(C[C@@H](N)CO)sc2c(NCc3cccs3)cnnc12] | (2R)-2-amino-3-(7-methyl-4- {[(thiophen-2- yl)methyl]amino}thieno[3,2- c]pyridazin-6-yl)propan-1-ol |
| 4 | 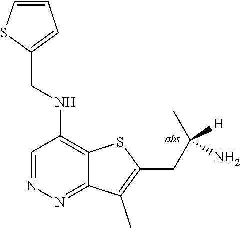 [SMILES: Cc1c(C[C@H](C)N)sc2c(NCc3cccs3)cnnc12] | 6-[(2S)-2-aminopropyl]-7-methyl-N- [(thiophen-2-yl)methyl]thieno[3,2- c]pyridazin-4-amine |
| 5 | 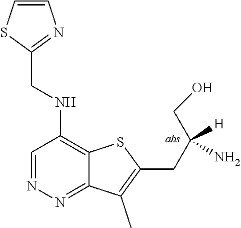 [SMILES: Cc1c(C[C@@H](N)CO)sc2c(NCc3nccs3)cnnc12] | (2R)-2-amino-3-(7-methyl-4-{[(1,3- thiazol-2-yl)methyl]amino}thieno[3,2- c]pyridazin-6-yl)propan-1-ol |
| 6 | 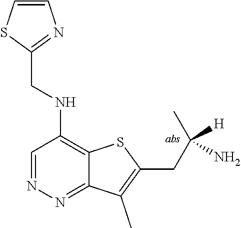 [SMILES: Cc1c(C[C@H](C)N)sc2c(NCc3nccs3)cnnc12] | 6-[(2S)-2-aminopropyl]-7-methyl-N- [(1,3-thiazol-2-yl)methyl]thieno[3,2- c]pyridazin-4-amine |
| 7 | 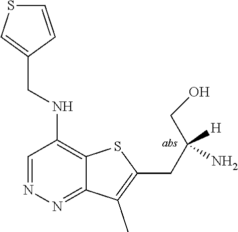 [SMILES: Cc1c(C[C@@H](N)CO)sc2c(NCc3ccsc3)cnnc12] | (2R)-2-amino-3-(7-methyl-4- {[(thiophen-3- yl)methyl]amino}thieno[3,2- c]pyridazin-6-yl)propan-1-ol |
| 8 | 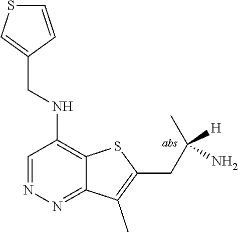 [SMILES: Cc1c(C[C@H](C)N)sc2c(NCc3ccsc3)cnnc12] | 6-[(2S)-2-aminopropyl]-7-methyl-N- [(thiophen-3-yl)methyl]thieno[3,2- c]pyridazin-4-amine |
| 9 | 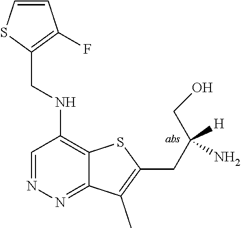 [SMILES: Cc1c(C[C@@H](N)CO)sc2c(NCc3sccc3F)cnnc12] | (2R)-2-amino-3-(4-{[(3- fluorothiophen-2-yl)methyl]amino}-7- methylthieno[3,2-c]pyridazin-6- yl)propan-1-ol |
| 10 | 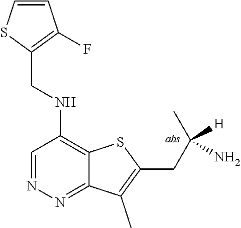 [SMILES: Cc1c(C[C@H](C)N)sc2c(NCc3sccc3F)cnnc12] | 6-[(2S)-2-aminopropyl]-N-[(3- fluorothiophen-2-yl)methyl]-7- methylthieno[3,2-c]pyridazin-4-amine |
| 11 | 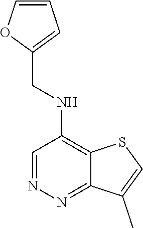 [SMILES: Cc1csc2c(NCc3ccco3)cnnc12] | N-[(furan-2-yl)methyl]-7- methylthieno[3,2-c]pyridazin-4-amine |
| 12 | 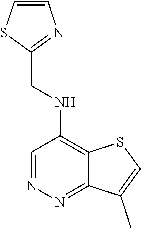 [SMILES: Cc1csc2c(NCc3nccs3)cnnc12] | 7-methyl-N-[(1,3-thiazol-2- yl)methyl]thieno[3,2-c]pyridazin-4- amine |
| 13 | 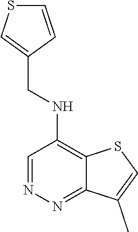 [SMILES: Cc1csc2c(NCc3ccsc3)cnnc12] | 7-methyl-N-[(thiophen-3- yl)methyl]thieno[3,2-c]pyridazin-4- amine |
| 14 | 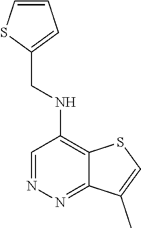 [SMILES: Cc1csc2c(NCc3cccs3)cnnc12] | 7-methyl-N-[(thiophen-2- yl)methyl]thieno[3,2-c]pyridazin-4- amine |
| 15 | 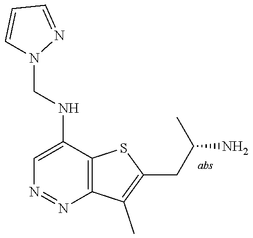 [SMILES: Cc1c(C[C@H](C)N)sc2c(NCn3cccn3)cnnc12] | 6-[(2S)-2-aminopropyl]-7-methyl-N- [(1H-pyrazol-1-yl)methyl]thieno[3,2- c]pyridazin-4-amine |
| 16 | 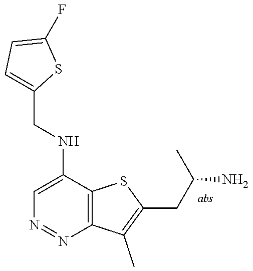 [SMILES: Cc1c(C[C@H](C)N)sc2c(NCc3ccc(F)s3)cnnc12] | 6-[(2S)-2-aminopropyl]-N-[(5- fluorothiophen-2-yl)methyl]-7- methylthieno[3,2-c]pyridazin-4-amine |
| 17 | 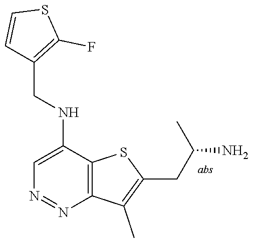 [SMILES: Cc1c(C[C@H](C)N)sc2c(NCc3ccsc3F)cnnc12] | 6-[(2S)-2-aminopropyl]-N-[(2- fluorothiophen-3-y1)methyl]-7- methylthieno[3,2-c]pyridazin-4-amine |
| 18 | 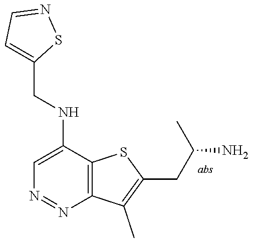 [SMILES: Cc1c(C[C@H](C)N)sc2c(NCc3ccns3)cnnc12] | 6-[(2S)-2-aminopropyl]-7-methyl-N- [(1,2-thiazol-5-yl)methyl]thieno[3,2- c]pyridazin-4-amine |
| 19 | 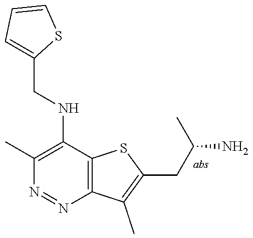 [SMILES: Cc1nnc2c(C)c(C[C@H](C)N)sc2c1NCc1cccs1] | 6-[(2S)-2-aminopropyl]-3,7-dimethyl- N-[(thiophen-2-yl)methyl]thieno[3,2- c]pyridazin-4-amine |
| 20 | 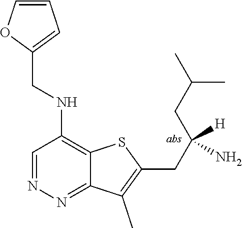 [SMILES: Cc1c(C[C@@H](N)CC(C)C)sc2c(NCc3ccco3)cnnc12] | 6-[(2S)-2-amino-4-methylpentyl]-N- [(furan-2-y1)methyl]-7- methylthieno[3,2-c]pyridazin-4-amine |
| 21 | 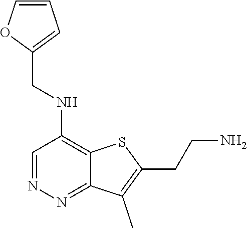 [SMILES: Cc1c(CCN)sc2c(NCc3ccco3)cnnc12] | 6-(2-aminoethyl)-N-[(furan-2- yl)methyl]-7-methylthieno[3,2- c]pyridazin-4-amine |
| 22 | 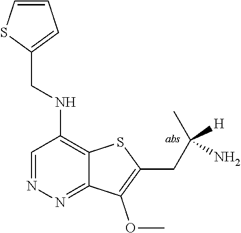 [SMILES: COc1c(C[C@H](C)N)sc2c(NCc3cccs3)cnnc12] | 6-[(2S)-2-aminopropyl]-7-methoxy-N- [(thiophen-2-yl)methyl]thieno[3,2- c]pyridazin-4-amine |
| 23 | 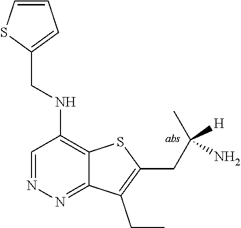 [SMILES: CCc1c(C[C@H](C)N)sc2c(NCc3cccs3)cnnc12] | 6-[(2S)-2-aminopropyl]-7-ethyl-N- [(thiophen-2-yl)methyl]thieno[3,2- c]pyridazin-4-amine |
| 24 | 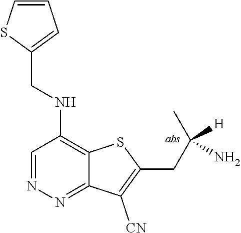 [SMILES: C[C@H](N)Cc1sc2c(NCc3cccs3)cnnc2c1C#N] | 6-[(2S)-2-aminopropyl]-4-{[(thiophen- 2-yl)methyl]amino}thieno[3,2- c]pyridazine-7-carbonitrile |
| 25 | 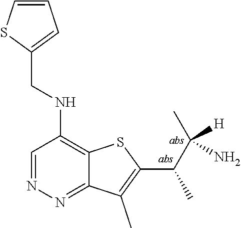 [SMILES: Cc1c([C@@H](C)[C@H](C)N)sc2c(NCc3cccs3)cnnc12] | 6-[(2S,3S)-3-aminobutan-2-yl]-7- methyl-N-[(thiophen-2- yl)methyl]thieno[3,2-c]pyridazin-4- amine |
| 26 | 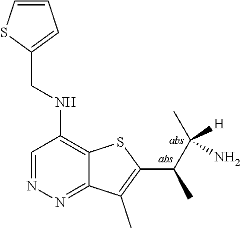 [SMILES: Cc1c([C@H](C)[C@H](C)N)sc2c(NCc3cccs3)cnnc12] | 6-[(2R,3S)-3-aminobutan-2-yl]-7- methyl-N-[(thiophen-2- yl)methyl]thieno[3,2-c]pyridazin-4- amine |
| 27 | 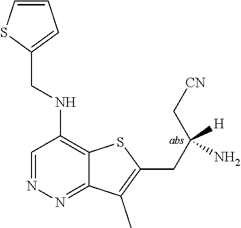 [SMILES: Cc1c(C[C@@H](N)CC#N)sc2c(NCc3cccs3)cnnc12] | (3S)-3-amino-4-(7-methyl-4- {[(thiophen-2- yl)methyl]amino}thieno[3,2- c]pyridazin-6-yl)butanenitrile |
| 28 | 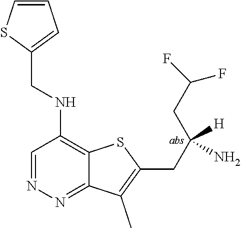 [SMILES: Cc1c(C[C@@H](N)CC(F)F)sc2c(NCc3cccs3)cnnc12] | 6-[(2S)-2-amino-4,4-difluorobutyl]-7- methyl-N-[(thiophen-2- yl)methyl]thieno[3,2-c]pyridazin-4- amine |
| 29 | 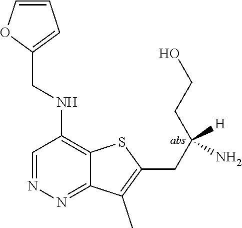 [SMILES: Cc1c(C[C@@H](N)CCO)sc2c(NCc3ccco3)cnnc12] | (3S)-3-amino-4-(4-{[(furan-2- yl)methyl]amino}-7-methylthieno[3,2- c]pyridazin-6-yl)butan-1-ol |
| 30 | 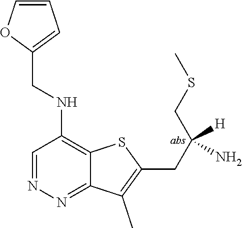 [SMILES: CSC[C@H](N)Cc1sc2c(NCc3ccco3)cnnc2c1C] | 6-[(2R)-2-amino-3- (methylsulfanyl)propyl]-N-[(furan-2- yl)methyl]-7-methylthieno[3,2- c]pyridazin-4-amine |
| 31 | 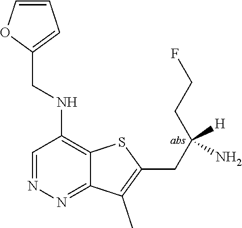 [SMILES: Cc1c(C[C@@H](N)CCF)sc2c(NCc3ccco3)cnnc12] | 6-[(2S)-2-amino-4-fluorobutyl]-N- [(furan-2-yl)methyl]-7- methylthieno[3,2-c]pyridazin-4-amine |
| 32 | 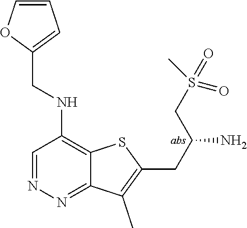 [SMILES: Cc1c(C[C@@H](N)CS(C)(=O)=O)sc2c(NCc3ccco3)cnnc12] | 6-[(2R)-2-amino-3- methanesulfonylpropyl]-N-[(furan-2- yl)methyl]-7-methylthieno[3,2- c]pyridazin-4-amine |
| 33 | 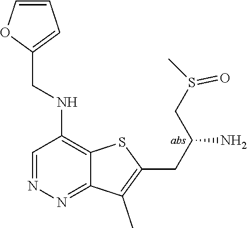 [SMILES: Cc1c(C[C@@H](N)CS(C)=O)sc2c(NCc3ccco3)cnnc12] | 6-[(2R)-2-amino-3- methanesulfinylpropyl]-N-[(furan-2- yl)methyl]-7-methylthieno[3,2- c]pyridazin-4-amine |
| 34 | 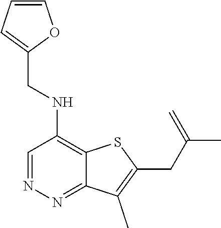 [SMILES: C=C(C)Cc1sc2c(NCc3ccco3)cnnc2c1C] | N-[(furan-2-yl)methyl]-7-methyl-6-(2- methylprop-2-en-1-yl)thieno[3,2- c]pyridazin-4-amine |
| 35 | 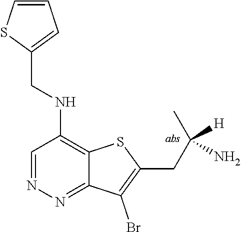 [SMILES: C[C@H](N)Cc1sc2c(NCc3cccs3)cnnc2c1Br] | 6-[(2S)-2-aminopropyl]-7-bromo-N- [(thiophen-2-yl)methyl]thieno[3,2- c]pyridazin-4-amine |
| 36 | 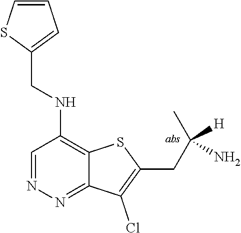 [SMILES: C[C@H](N)Cc1sc2c(NCc3cccs3)cnnc2c1Cl] | 6-[(2S)-2-aminopropyl]-7-chloro-N- [(thiophen-2-yl)methyl]thieno[3,2- c]pyridazin-4-amine |
| 37 | 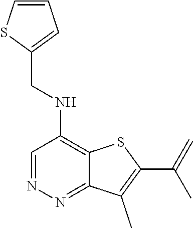 [SMILES: C=C(C)c1sc2c(NCc3cccs3)cnnc2c1C] | 7-methyl-6-(prop-1-en-2-yl)-N- [(thiophen-2-yl)methyl]thieno[3,2- c]pyridazin-4-amine |
| 38 | 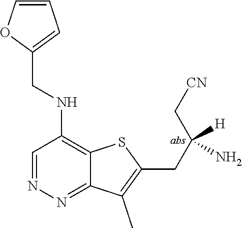 [SMILES: Cc1c(C[C@@H](N)CC#N)sc2c(NCc3ccco3)cnnc12] | (3S)-3-amino-4-(4-{[(furan-2- yl)methyl]amino}-7-methylthieno[3,2- c]pyridazin-6-yl)butanenitrile |
| 39 | 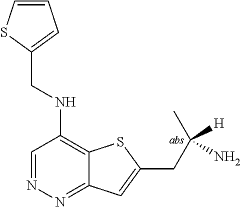 [SMILES: C[C@H](N)Cc1cc2nncc(NCc3cccs3)c2s1] | 6-[(2S)-2-aminopropyl]-N-[(thiophen- 2-yl)methyl]thieno[3,2-c]pyridazin-4- amine |
| 40 | 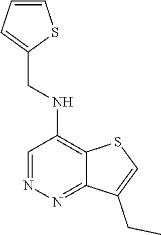 [SMILES: CCc1csc2c(NCc3cccs3)cnnc12] | 7-ethyl-N-[(thiophen-2- yl)methyl]thieno[3,2-c]pyridazin-4- amine |
| 41 and | 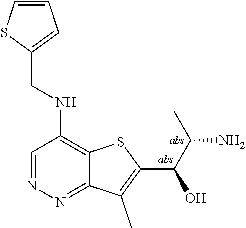 [SMILES: Cc1c([C@H](O)[C@H](C)N)sc2c(NCc3cccs3)cnnc12] | (1S,2S)-2-amino-1-(7-methyl-4- {[(thiophen-2- yl)methyl]amino}thieno[3,2- c]pyridazin-6-yl)propan-1-ol |
| 42 | 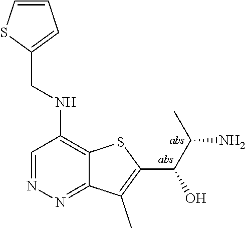 [SMILES: Cc1c([C@@H](O)[C@H](C)N)sc2c(NCc3cccs3)cnnc12] | (1R,2S)-2-amino-1-(7-methyl-4- {[(thiophen-2- yl)methyl]amino}thieno[3,2- c]pyridazin-6-yl)propan-1-ol |
| 43 | 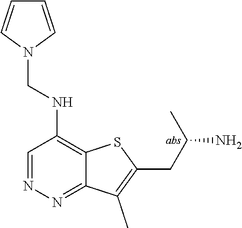 [SMILES: Cc1c(C[C@H](C)N)sc2c(NCn3cccc3)cnnc12] | 6-[(2S)-2-aminopropyl]-7-methyl-N- [(1H-pyrrol-1-yl)methyl]thieno[3,2- c]pyridazin-4-amine |
| 44 | 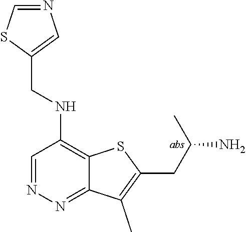 [SMILES: Cc1c(C[C@H](C)N)sc2c(NCc3cncs3)cnnc12] | 6-[(2S)-2-aminopropyl]-7-methyl-N- [(1,3-thiazol-5-yl)methyl]thieno[3,2- c]pyridazin-4-amine |
| 45 | 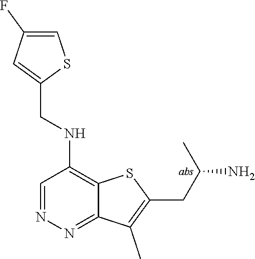 [SMILES: Cc1c(C[C@H](C)N)sc2c(NCc3cc(F)cs3)cnnc12] | 6-[(2S)-2-aminopropyl]-N-[(4- fluorothiophen-2-yl)methyl]-7- methylthieno[3,2-c]pyridazin-4-amine |
| 46 | 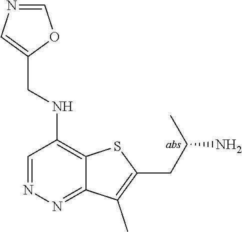 [SMILES: Cc1c(C[C@H](C)N)sc2c(NCc3cnco3)cnnc12] | 6-[(2S)-2-aminopropyl]-7-methyl-N- [(1,3-oxazol-5-yl)methyl]thieno[3,2- c]pyridazin-4-amine |
| 47 | 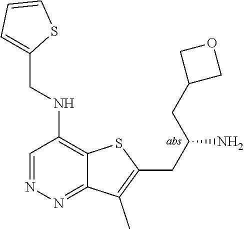 [SMILES: Cc1c(C[C@@H](N)CC2COC2)sc2c(NCc3cccs3)cnnc12] | 6-[(2S)-2-amino-3-(oxetan-3- yl)propyl]-7-methyl-N-[(thiophen-2- yl)methyl]thieno[3,2-c]pyridazin-4- amine |
| 48 | 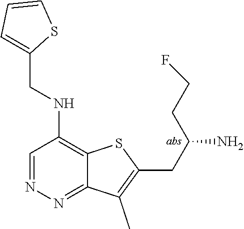 [SMILES: Cc1c(C[C@@H](N)CCF)sc2c(NCc3cccs3)cnnc12] | 6-[(2S)-2-amino-4-fluorobutyl]-7- methyl-N-[(thiophen-2- yl)methyl]thieno[3,2-c]pyridazin-4- amine |
| 49 | 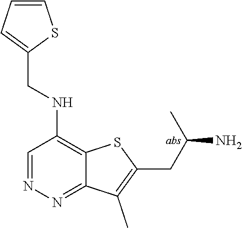 [SMILES: Cc1c(C[C@@H](C)N)sc2c(NCc3cccs3)cnnc12] | 6-[(2R)-2-aminopropyl]-7-methyl-N- [(thiophen-2-yl)methyl]thieno[3,2- c]pyridazin-4-amine |
| 50 | 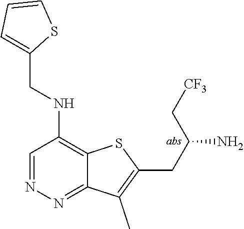 [SMILES: Cc1c(C[C@@H](N)CC(F)(F)F)sc2c(NCc3cccs3)cnnc12] | 6-[(2S)-2-amino-4,4,4-trifluorobutyl]- 7-methyl-N-[(thiophen-2- yl)methyl]thieno[3,2-c]pyridazin-4- amine |
| 51 | 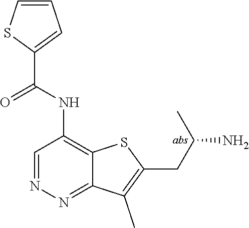 [SMILES: Cc1c(C[C@H](C)N)sc2c(NC(=O)c3cccs3)cnnc12] | N-{6-[(2S)-2-aminopropyl]-7- methylthieno[3,2-c]pyridazin-4- yl}thiophene-2-carboxamide |
| 52 | 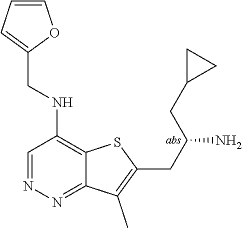 [SMILES: Cc1c(C[C@@H](N)CC2CC2)sc2c(NCc3ccco3)cnnc12] | 6-[(2S)-2-amino-3-cyclopropylpropyl]- N-[(furan-2-yl)methyl]-7- methylthieno[3,2-c]pyridazin-4-amine |
| 53 | 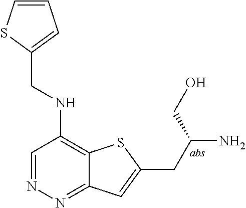 [SMILES: N[C@@H](CO)Cc1cc2nncc(NCc3cccs3)c2s1] | (2R)-2-amino-3-(4-{[(thiophen-2- yl)methyl]amino}thieno[3,2- c]pyridazin-6-yl)propan-1-ol |
| 54 | 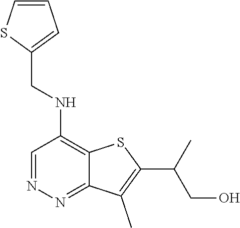 [SMILES: Cc1c(C(C)CO)sc2c(NCc3cccs3)cnnc12] | 2-(7-methyl-4-{[(thiophen-2- yl)methyl]amino}thieno[3,2- c]pyridazin-6-yl)propan-1-ol |
| 55 | 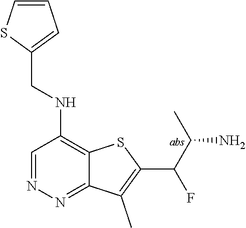 [SMILES: Cc1c(C(F)[C@H](C)N)sc2c(NCc3cccs3)cnnc12] | 6-[(2S)-2-amino-1-fluoropropyl]-7- methyl-N-[(thiophen-2- yl)methyl]thieno[3,2-c]pyridazin-4- amine |
| 56 | 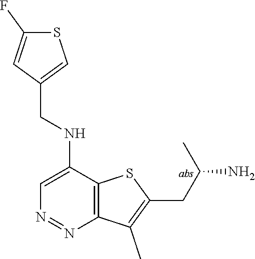 [SMILES: Cc1c(C[C@H](C)N)sc2c(NCc3csc(F)c3)cnnc12] | 6-[(2S)-2-aminopropyl]-N-[(5- fluorothiophen-3-yl)methyl]-7- methylthieno[3,2-c]pyridazin-4-amine |
| 57 | 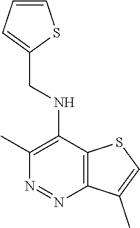 [SMILES: Cc1nnc2c(C)csc2c1NCc1cccs1] | 3,7-dimethyl-N-[(thiophen-2- yl)methyl]thieno[3,2-c]pyridazin-4- amine |
| 58 | 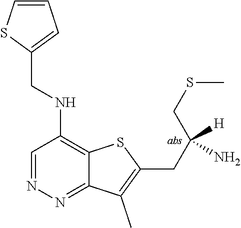 [SMILES: CSC[C@H](N)Cc1sc2c(NCc3cccs3)cnnc2c1C] | 6-[(2R)-2-amino-3- (methylsulfanyl)propyl]-7-methyl-N- [(thiophen-2-yl)methyl]thieno[3,2- c]pyridazin-4-amine |
| 59 | 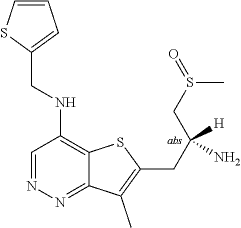 [SMILES: Cc1c(C[C@@H](N)CS(C)=O)sc2c(NCc3cccs3)cnnc12] | 6-[(2R)-2-amino-3- methanesulfinylpropyl]-7-methyl-N- [(thiophen-2-yl)methyl]thieno[3,2- c]pyridazin-4-amine |
| 60 | 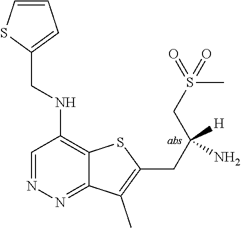 [SMILES: Cc1c(C[C@@H](N)CS(C)(=O)=O)sc2c(NCc3cccs3)cnnc12] | 6-[(2R)-2-amino-3- methanesulfonylpropyl]-7-methyl-N- [(thiophen-2-yl)methyl]thieno[3,2- c]pyridazin-4-amine |
| 61 | 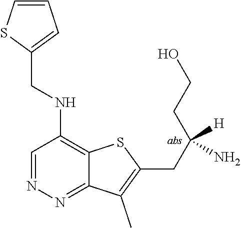 [SMILES: Cc1c(C[C@@H](N)CCO)sc2c(NCc3cccs3)cnnc12] | (3S)-3-amino-4-(7-methyl-4- {[(thiophen-2- yl)methyl]amino}thieno[3,2- c]pyridazin-6-yl)butan-1-ol |
| 62 | 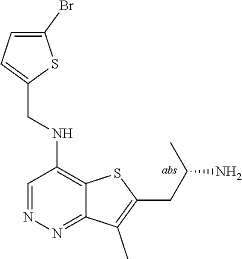 [SMILES: Cc1c(C[C@H](C)N)sc2c(NCc3ccc(Br)s3)cnnc12] | 6-[(2S)-2-aminopropyl]-N-[(5- bromothiophen-2-yl)methyl]-7- methylthieno[3,2-c]pyridazin-4-amine |
| 63 | 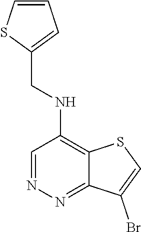 [SMILES: Brc1csc2c(NCc3cccs3)cnnc12] | 7-bromo-N-[(thiophen-2- yl)methyl]thieno[3,2-c]pyridazin-4- amine |
| 64 | 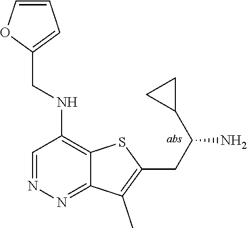 [SMILES: Cc1c(C[C@@H](N)C2CC2)sc2c(NCc3ccco3)cnnc12] | 6-[(2R)-2-amino-2-cyclopropylethyl]- N-[(furan-2-yl)methyl]-7- methylthieno[3,2-c]pyridazin-4-amine |
| 65 | 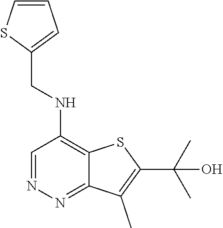 [SMILES: Cc1c(C(C)(C)O)sc2c(NCc3cccs3)cnnc12] | 2-(7-methyl-4-{[(thiophen-2- yl)methyl]amino}thieno[3,2- c]pyridazin-6-yl)propan-2-ol |
| 66 | 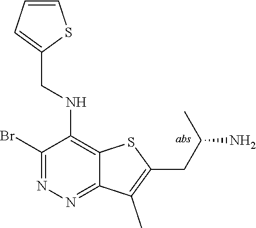 [SMILES: Cc1c(C[C@H](C)N)sc2c(NCc3cccs3)c(Br)nnc12] | 6-[(2S)-2-aminopropyl]-3-bromo-7- methyl-N-[(thiophen-2- yl)methyl]thieno[3,2-c]pyridazin-4- amine |
| 67 | 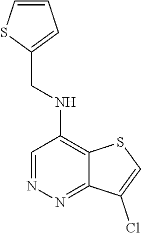 [SMILES: Clc1csc2c(NCc3cccs3)cnnc12] | 7-chloro-N-[(thiophen-2- yl)methyl]thieno[3,2-c]pyridazin-4- amine |
| 68 | 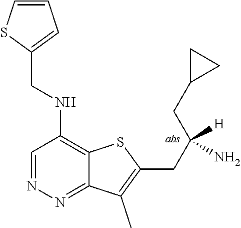 [SMILES: Cc1c(C[C@@H](N)CC2CC2)sc2c(NCc3cccs3)cnnc12] | 6-[(2S)-2-amino-3-cyclopropylpropyl]- 7-methyl-N-[(thiophen-2- yl)methyl]thieno[3,2-c]pyridazin-4- amine |
| 69 | 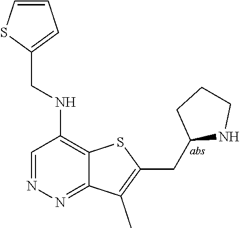 [SMILES: Cc1c(C[C@H]2CCCN2)sc2c(NCc3cccs3)cnnc12] | 7-methyl-6-{[(2R)-pyrrolidin-2- yl]methyl}-N-[(thiophen-2- yl)methyl]thieno[3,2-c]pyridazin-4- amine |
| 70 | 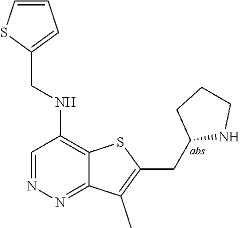 [SMILES: Cc1c(C[C@@H]2CCCN2)sc2c(NCc3cccs3)cnnc12] | 7-methyl-6-{[(2S)-pyrrolidin-2- yl]methyl}-N-[(thiophen-2- yl)methyl]thieno[3,2-c]pyridazin-4- amine |
| 71 | 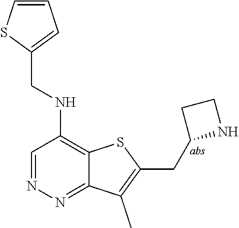 [SMILES: Cc1c(C[C@@H]2CCN2)sc2c(NCc3cccs3)cnnc12] | 6-{[(2S)-azetidin-2-y1]methyl}-7- methyl-N-[(thiophen-2- yl)methyl]thieno[3,2-c]pyridazin-4- amine |
| 72 | 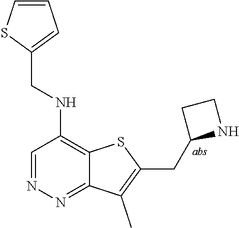 [SMILES: Cc1c(C[C@H]2CCN2)sc2c(NCc3cccs3)cnnc12] | 6-{[(2R)-azetidin-2-yl]methyl}-7- methyl-N-[(thiophen-2- yl)methyl]thieno[3,2-c]pyridazin-4- amine |
| 73 | 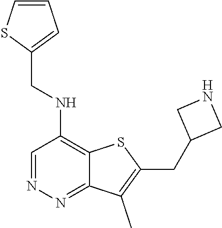 [SMILES: Cc1c(CC2CNC2)sc2c(NCc3cccs3)cnnc12] | 6-[(azetidin-3-yl)methyl]-7-methyl-N- [(thiophen-2-yl)methyl]thieno[3,2- c]pyridazin-4-amine |
| 74 | 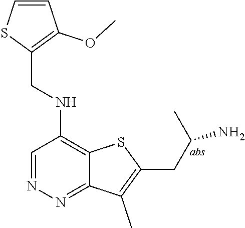 [SMILES: COc1ccsc1CNc1cnnc2c(C)c(C[C@H](C)N)sc12] | 6-[(2S)-2-aminopropyl]-N-[(3- methoxythiophen-2-yl)methyl]-7- methylthieno[3,2-c]pyridazin-4-amine |
| 75 | 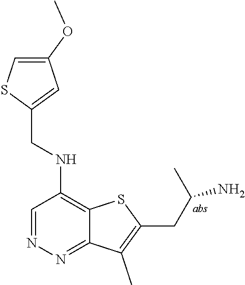 [SMILES: COc1csc(CNc2cnnc3c(C)c(C[C@H](C)N)sc23)c1] | 6-[(2S)-2-aminopropyl]-N-[(4- methoxythiophen-2-yl)methyl]-7- methylthieno[3,2-c]pyridazin-4-amine |
| 76 | 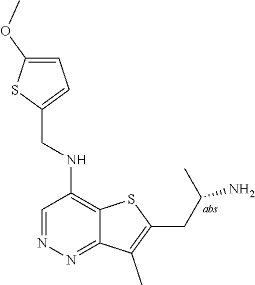 [SMILES: COc1ccc(CNc2cnnc3c(C)c(C[C@H](C)N)sc23)s1] | 6-[(2S)-2-aminopropyl]-N-[(5- methoxythiophen-2-yl)methyl]-7- methylthieno[3,2-c]pyridazin-4-amine |
| 77 | 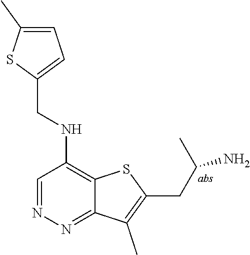 [SMILES: Cc1ccc(CNc2cnnc3c(C)c(C[C@H](C)N)sc23)s1] | 6-[(2S)-2-aminopropyl]-7-methyl-N- [(5-methylthiophen-2- yl)methyl]thieno[3,2-c]pyridazin-4- amine |
| 78 | 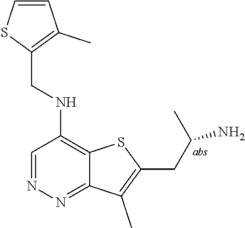 [SMILES: Cc1ccsc1CNc1cnnc2c(C)c(C[C@H](C)N)sc12] | 6-[(2S)-2-aminopropyl]-7-methyl-N- [(3-methylthiophen-2- yl)methyl]thieno[3,2-c]pyridazin-4- amine |
| 79 | 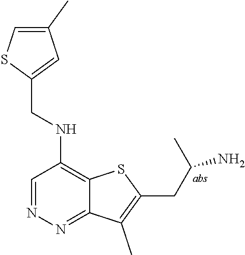 [SMILES: Cc1csc(CNc2cnnc3c(C)c(C[C@H](C)N)sc23)c1] | 6-[(2S)-2-aminopropyl]-7-methyl-N- [(4-methylthiophen-2- yl)methyl]thieno[3,2-c]pyridazin-4- amine |
| 80 | 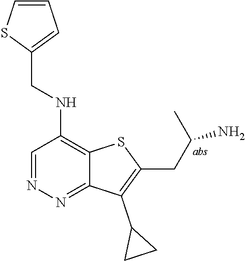 [SMILES: C[C@H](N)Cc1sc2c(NCc3cccs3)cnnc2c1C1CC1] | 6-[(2S)-2-aminopropyl]-7-cyclopropyl- N-[(thiophen-2-yl)methyl]thieno[3,2- c]pyridazin-4-amine |
| 81 | 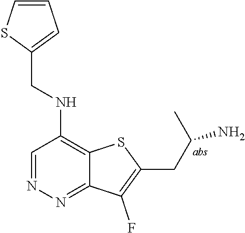 [SMILES: C[C@H](N)Cc1sc2c(NCc3cccs3)cnnc2c1F] | 6-[(2S)-2-aminopropyl]-7-fluoro-N- [(thiophen-2-yl)methyl]thieno[3,2- c]pyridazin-4-amine |
| 82 | 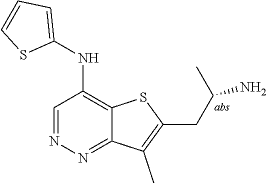 [SMILES: Cc1c(C[C@H](C)N)sc2c(Nc3cccs3)cnnc12] | 6-[(2S)-2-aminopropyl]-7-methyl-N- (thiophen-2-yl)thieno[3,2-c]pyridazin- 4-amine |
| 83 | 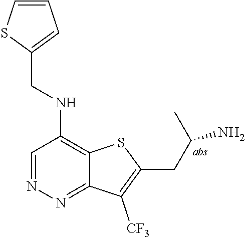 [SMILES: C[C@H](N)Cc1sc2c(NCc3cccs3)cnnc2c1C(F)(F)F] | 6-[(2S)-2-aminopropyl]-N-[(thiophen- 2-yl)methyl]-7- (trifluoromethyl)thieno[3,2- c]pyridazin-4-amine |
| 84 | 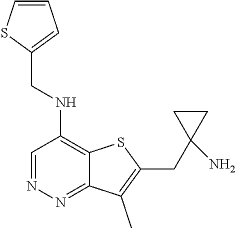 [SMILES: Cc1c(CC2(N)CC2)sc2c(NCc3cccs3)cnnc12] | 6-[(1-aminocyclopropyl)methyl]-7- methyl-N-[(thiophen-2- yl)methyl]thieno[3,2-c]pyridazin-4- amine |
| 85 | 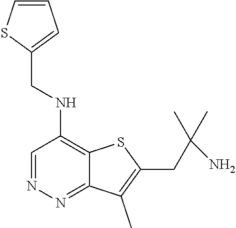 [SMILES: Cc1c(CC(C)(C)N)sc2c(NCc3cccs3)cnnc12] | 6-(2-amino-2-methylpropyl)-7-methyl- N-[(thiophen-2-yl)methyl]thieno[3,2- c]pyridazin-4-amine |
| 86 | 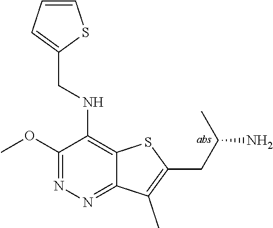 [SMILES: COc1nnc2c(C)c(C[C@H](C)N)sc2c1NCc1cccs1] | 6-[(2S)-2-aminopropyl]-3-methoxy-7- methyl-N-[(thiophen-2- yl)methyl]thieno[3,2-c]pyridazin-4- amine |
| 87 | 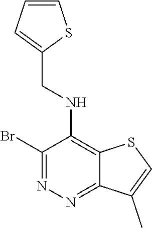 [SMILES: Cc1csc2c(NCc3cccs3)c(Br)nnc12] | 3-bromo-7-methyl-N-[(thiophen-2- yl)methyl]thieno[3,2-c]pyridazin-4- amine |
| 88 | 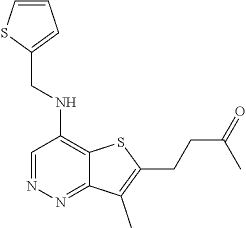 [SMILES: CC(=O)CCc1sc2c(NCc3cccs3)cnnc2c1C] | 4-(7-methyl-4-{[(thiophen-2- yl)methyl]amino}thieno[3,2- c]pyridazin-6-yl)butan-2-one |
| 89 | 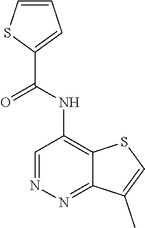 [SMILES: Cc1csc2c(NC(=O)c3cccs3)cnnc12] | N-{7-methylthieno[3,2-c]pyridazin-4- yl}thiophene-2-carboxamide |
| 90 | 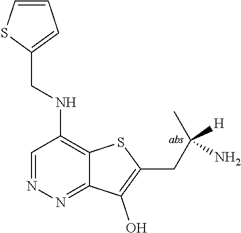 [SMILES: C[C@H](N)Cc1sc2c(NCc3cccs3)cnnc2c1O] | 6-[(2S)-2-aminopropyl]-4-{[(thiophen- 2-yl)methyl]amino}thieno[3,2- c]pyridazin-7-ol |
| 91 | 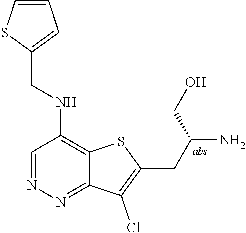 [SMILES: N[C@@H](CO)Cc1sc2c(NCc3cccs3)cnnc2c1Cl] | (2R)-2-amino-3-(7-chloro-4- {[(thiophen-2- yl)methyl]amino}thieno[3,2- c]pyridazin-6-yl)propan-1-ol |
| 92 | 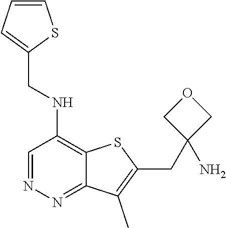 [SMILES: Cc1c(CC2(N)COC2)sc2c(NCc3cccs3)cnnc12] | 6-[(3-aminooxetan-3-yl)methyl]-7- methyl-N-[(thiophen-2- yl)methyl]thieno[3,2-c]pyridazin-4- amine |
| 93 | 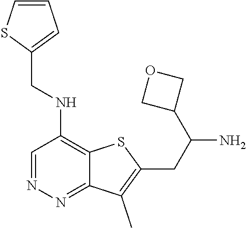 [SMILES: Cc1c(CC(N)C2COC2)sc2c(NCc3cccs3)cnnc12] | 6-[2-amino-2-(oxetan-3-yl)ethyl]-7- methyl-N-[(thiophen-2- yl)methyl]thieno[3,2-c]pyridazin-4- amine |
| 94 | 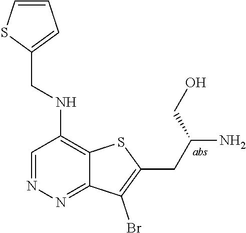 [SMILES: N[C@@H](CO)Cc1sc2c(NCc3cccs3)cnnc2c1Br] | (2R)-2-amino-3-(7-bromo-4- {[(thiophen-2- yl)methyl]amino}thieno[3,2- c]pyridazin-6-yl)propan-1-ol |
| 95 | 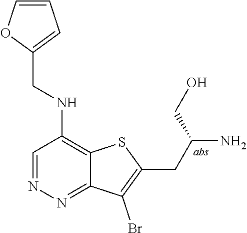 [SMILES: N[C@@H](CO)Cc1sc2c(NCc3ccco3)cnnc2c1Br] | (2R)-2-amino-3-(7-bromo-4-{[(furan- 2-yl)methyl]amino}thieno[3,2- c]pyridazin-6-yl)propan-1-ol |
| 96 | 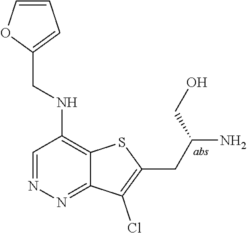 [SMILES: N[C@@H](CO)Cc1sc2c(NCc3ccco3)cnnc2c1Cl] | (2R)-2-amino-3-(7-chloro-4-{[(furan- 2-yl)methyl]amino}thieno[3,2- c]pyridazin-6-yl)propan-1-ol |
| 97 | 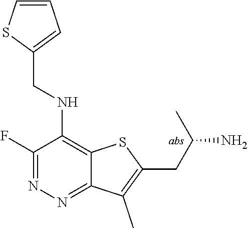 [SMILES: Cc1c(C[C@H](C)N)sc2c(NCc3cccs3)c(F)nnc12] | 6-[(2S)-2-aminopropyl]-3-fluoro-7- methyl-N-[(thiophen-2- yl)methyl]thieno[3,2-c]pyridazin-4- amine |
| 98 | 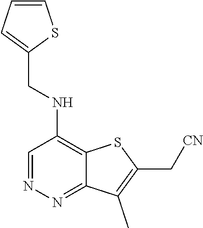 [SMILES: Cc1c(CC#N)sc2c(NCc3cccs3)cnnc12] | 2-(7-methyl-4-{[(thiophen-2- yl)methyl]amino}thieno[3,2- c]pyridazin-6-yl)acetonitrile |
| 99 | 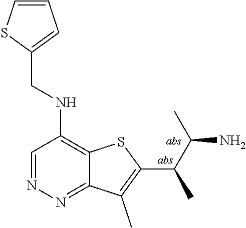 [SMILES: Cc1c([C@H](C)[C@@H](C)N)sc2c(NCc3cccs3)cnnc12] | 6-[(2R,3R)-3-aminobutan-2-yl]-7- methyl-N-[(thiophen-2- yl)methyl]thieno[3,2-c]pyridazin-4- amine |
| 100 | 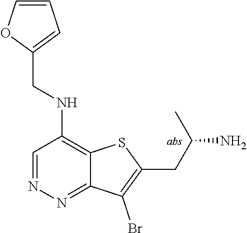 [SMILES: C[C@H](N)Cc1sc2c(NCc3ccco3)cnnc2c1Br] | 6-[(2S)-2-aminopropyl]-7-bromo-N- [(furan-2-y1)methyl]thieno[3,2- c]pyridazin-4-amine |
| 101 | 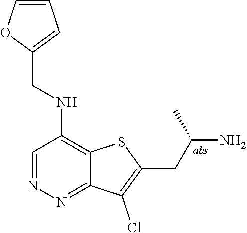 [SMILES: C[C@H](N)Cc1sc2c(NCc3ccco3)cnnc2c1Cl] | 6-[(2S)-2-aminopropyl]-7-chloro-N- [(furan-2-yl)methyl]thieno[3,2- c]pyridazin-4-amine |
| 102 | 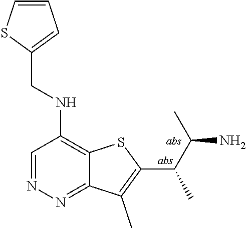 [SMILES: Cc1c([C@@H](C)[C@@H](C)N)sc2c(NCc3cccs3)cnnc12] | 6-[(2S,3R)-3-aminobutan-2-yl]-7- methyl-N-[(thiophen-2- yl)methyl]thieno[3,2-c]pyridazin-4- amine |
| 103 | 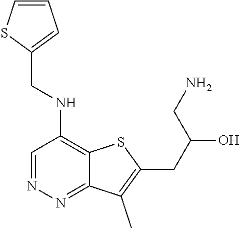 [SMILES: Cc1c(CC(O)CN)sc2c(NCc3cccs3)cnnc12] | 1-amino-3-(7-methyl-4-{[(thiophen-2- yl)methyl]amino}thieno[3,2- c]pyridazin-6-yl)propan-2-ol |
| 104 | 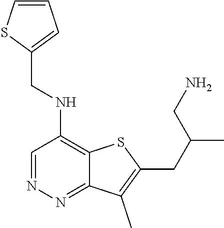 [SMILES: Cc1c(CC(C)CN)sc2c(NCc3cccs3)cnnc12] | 6-(3-amino-2-methylpropyl)-7-methyl- N-[(thiophen-2-yl)methyl]thieno[3,2- c]pyridazin-4-amine |
| 105 | 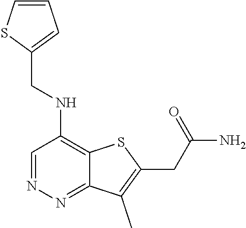 [SMILES: Cc1c(CC(N)=O)sc2c(NCc3cccs3)cnnc12] | 2-(7-methyl-4-{[(thiophen-2- yl)methyl]amino}thieno[3,2- c]pyridazin-6-yl)acetamide |
| 106 | 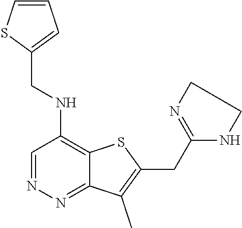 [SMILES: Cc1c(CC2=NCCN2)sc2c(NCc3cccs3)cnnc12] | 6-[(4,5-dihydro-1H-imidazol-2- yl)methyl]-7-methyl-N-[(thiophen-2- yl)methyl]thieno[3,2-c]pyridazin-4- amine |
| 107 | 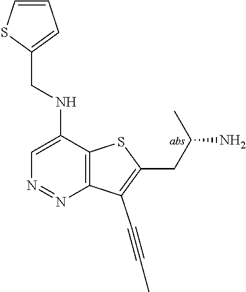 [SMILES: CC#Cc1c(C[C@H](C)N)sc2c(NCc3cccs3)cnnc12] | 6-[(2S)-2-aminopropyl]-7-(prop-1-yn- 1-yl)-N-[(thiophen-2- yl)methyl]thieno[3,2-c]pyridazin-4- amine |
| 108 | 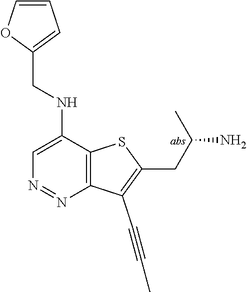 [SMILES: CC#Cc1c(C[C@H](C)N)sc2c(NCc3ccco3)cnnc12] | 6-[(2S)-2-aminopropyl]-N-[(furan-2- yl)methyl]-7-(prop-1-yn-1- yl)thieno[3,2-c]pyridazin-4-amine |
| 109 | 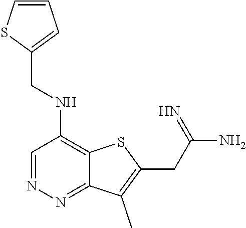 [SMILES: Cc1c(CC(=N)N)sc2c(NCc3cccs3)cnnc12] | 2-(7-methyl-4-{[(thiophen-2- yl)methyl]amino}thieno[3,2- c]pyridazin-6-yl)ethanimidamide |
| 110 | 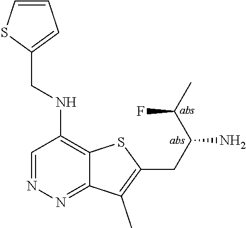 [SMILES: Cc1c(C[C@@H](N)[C@H](C)F)sc2c(NCc3cccs3)cnnc12] | 6-[(2R,3S)-2-amino-3-fluorobutyl]-7- methyl-N-[(thiophen-2- yl)methyl]thieno[3,2-c]pyridazin-4- amine |
| 111 | 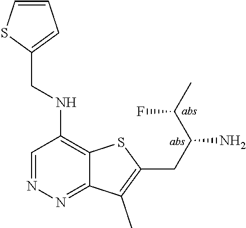 [SMILES: Cc1c(C[C@@H](N)[C@@H](C)F)sc2c(NCc3cccs3)cnnc12] | 6-[(2R,3R)-2-amino-3-fluorobutyl]-7- methyl-N-[(thiophen-2- yl)methyl]thieno[3,2-c]pyridazin-4- amine |
| 112 | 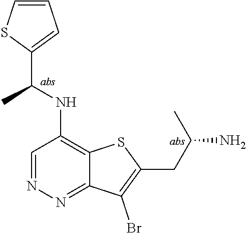 [SMILES: C[C@H](N)Cc1sc2c(N[C@@H](C)c3cccs3)cnnc2c1Br] | 6-[(2S)-2-aminopropyl]-7-bromo-N- [(1S)-1-(thiophen-2- yl)ethyl]thieno[3,2-c]pyridazin-4- amine |
| 113 | 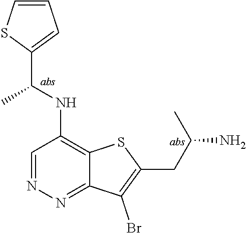 [SMILES: C[C@H](N)Cc1sc2c(N[C@H](C)c3cccs3)cnnc2c1Br] | 6-[(2S)-2-aminopropyl]-7-bromo-N- [(1R)-1-(thiophen-2- yl)ethyl]thieno[3,2-c]pyridazin-4- amine |
| 114 | 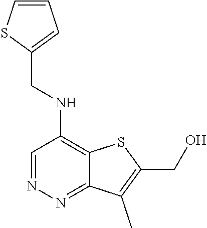 [SMILES: Cc1c(CO)sc2c(NCc3cccs3)cnnc12] | (7-methyl-4-{[(thiophen-2- yl)methyl]amino}thieno[3,2- c]pyridazin-6-yl)methanol |
| 115 | 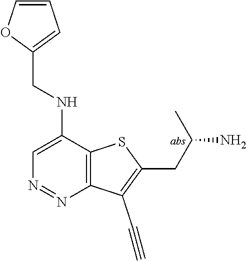 [SMILES: C#Cc1c(C[C@H](C)N)sc2c(NCc3ccco3)cnnc12] | 6-[(2S)-2-aminopropyl]-7-ethynyl-N- [(furan-2-y1)methyl]thieno[3,2- c]pyridazin-4-amine |
| 116 | 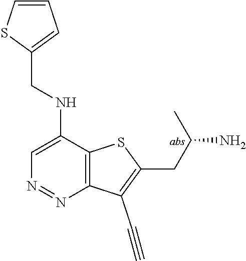 [SMILES: C#Cc1c(C[C@H](C)N)sc2c(NCc3cccs3)cnnc12] | 6-[(2S)-2-aminopropyl]-7-ethynyl-N- [(thiophen-2-yl)methyl]thieno[3,2- c]pyridazin-4-amine |
| 117 | 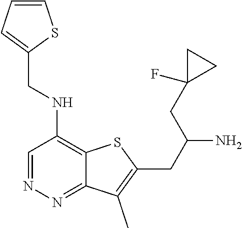 [SMILES: Cc1c(CC(N)CC2(F)CC2)sc2c(NCc3cccs3)cnnc12] | 6-[2-amino-3-(1- fluorocyclopropyl)propyl]-7-methyl-N- [(thiophen-2-yl)methyl]thieno[3,2- c]pyridazin-4-amine |
| 118 | 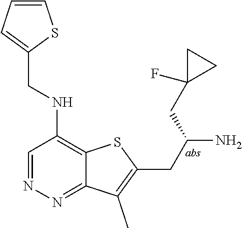 [SMILES: Cc1c(C[C@@H](N)CC2(F)CC2)sc2c(NCc3cccs3)cnnc12] | 6-[(2S)-2-amino-3-(1- fluorocyclopropyl)propyl]-7-methyl-N- [(thiophen-2-yl)methyl]thieno[3,2- c]pyridazin-4-amine |
| 119 | 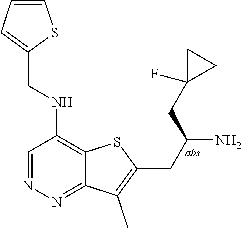 [SMILES: Cc1c(C[C@H](N)CC2(F)CC2)sc2c(NCc3cccs3)cnnc12] | 6-[(2R)-2-amino-3-(1- fluorocyclopropyl)propyl]-7-methyl-N- [(thiophen-2-yl)methyl]thieno[3,2- c]pyridazin-4-amine |
| 120 | 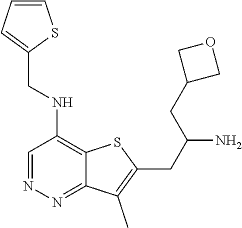 [SMILES: Cc1c(CC(N)CC2COC2)sc2c(NCc3cccs3)cnnc12] | 6-[2-amino-3-(oxetan-3-yl)propyl]-7- methyl-N-[(thiophen-2- yl)methyl]thieno[3,2-c]pyridazin-4- amine |
| 121 | 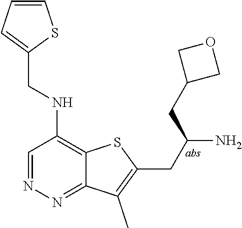 [SMILES: Cc1c(C[C@H](N)CC2COC2)sc2c(NCc3cccs3)cnnc12] | 6-[(2R)-2-amino-3-(oxetan-3- yl)propyl]-7-methyl-N-[(thiophen-2- yl)methyl]thieno[3,2-c]pyridazin-4- amine |
| 122 | 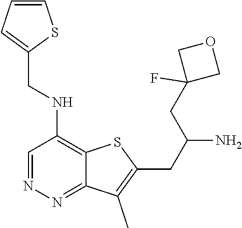 [SMILES: Cc1c(CC(N)CC2(F)COC2)sc2c(NCc3cccs3)cnnc12] | 6-[2-amino-3-(3-fluorooxetan-3- yl)propyl]-7-methyl-N-[(thiophen-2- yl)methyl]thieno[3,2-c]pyridazin-4- amine |
| 123 | 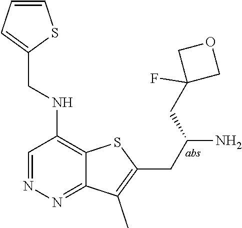 [SMILES: Cc1c(C[C@@H](N)CC2(F)COC2)sc2c(NCc3cccs3)cnnc12] | 6-[(2S)-2-amino-3-(3-fluorooxetan-3- yl)propyl]-7-methyl-N-[(thiophen-2- yl)methyl]thieno[3,2-c]pyridazin-4- amine |
| 124 | 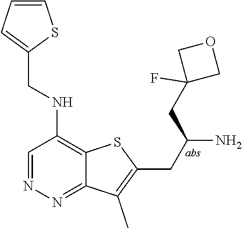 [SMILES: Cc1c(C[C@H](N)CC2(F)COC2)sc2c(NCc3cccs3)cnnc12] | 6-[(2R)-2-amino-3-(3-fluorooxetan-3- yl)propyl]-7-methyl-N-[(thiophen-2- yl)methyl]thieno[3,2-c]pyridazin-4- amine |
| 125 | 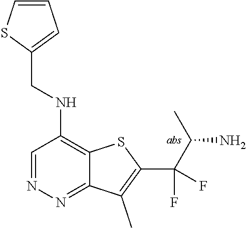 [SMILES: Cc1c(C(F)(F)[C@H](C)N)sc2c(NCc3cccs3)cnnc12] | 6-[(2S)-2-amino-1,1-difluoropropyl]-7- methyl-N-[(thiophen-2- yl)methyl]thieno[3,2-c]pyridazin-4- amine |
| 126 | 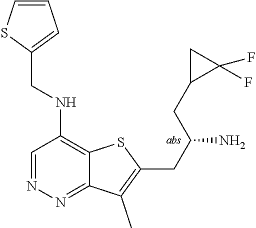 [SMILES: Cc1c(C[C@@H](N)CC2CC2(F)F)sc2c(NCc3cccs3)cnnc12] | 6-[(2S)-2-amino-3-(2,2- difluorocyclopropyl)propyl]-7-methyl- N-[(thiophen-2-yl)methyl]thieno[3,2- c]pyridazin-4-amine |
| 127 | 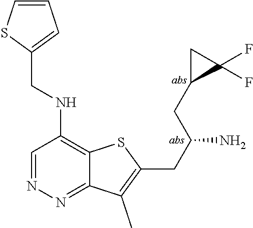 [SMILES: Cc1c(C[C@@H](N)C[C@H]2CC2(F)F)sc2c(NCc3cccs3)cnnc12] | 6-[(2S)-2-amino-3-[(1S)-2,2- difluorocyclopropyl]propyl]-7-methyl- N-[(thiophen-2-yl)methyl]thieno[3,2- c]pyridazin-4-amine |
| 128 | 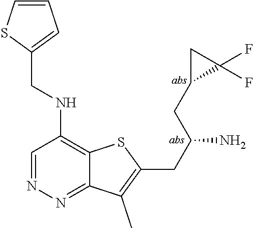 [SMILES: Cc1c(C[C@@H](N)C[C@@H]2CC2(F)F)sc2c(NCc3cccs3)cnnc12] | 6-[(2S)-2-amino-3-[(1R)-2,2- difluorocyclopropyl]propyl]-7-methyl- N-[(thiophen-2-yl)methyl]thieno[3,2- c]pyridazin-4-amine |
| 129 & | 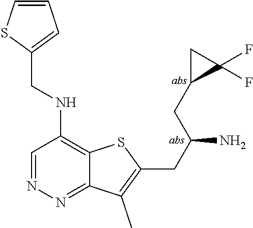 [SMILES: Cc1c(C[C@H](N)C[C@H]2CC2(F)F)sc2c(NCc3cccs3)cnnc12] | 6-[(2R)-2-amino-3-[(1S)-2,2- difluorocyclopropyl]propyl]-7-methyl- N-[(thiophen-2-yl)methyl]thieno[3,2- c]pyridazin-4-amine |
| 130 | 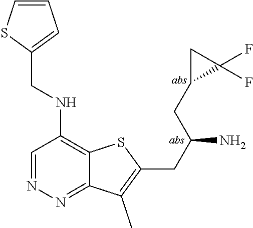 [SMILES: Cc1c(C[C@H](N)C[C@@H]2CC2(F)F)sc2c(NCc3cccs3)cnnc12] | 6-[(2R)-2-amino-3-[(1R)-2,2- difluorocyclopropyl]propyl]-7-methyl- N-[(thiophen-2-yl)methyl]thieno[3,2- c]pyridazin-4-amine |
| 131 | 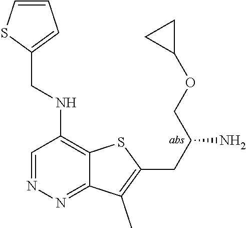 [SMILES: Cc1c(C[C@@H](N)COC2CC2)sc2c(NCc3cccs3)cnnc12] | 6-[(2R)-2-amino-3- cyclopropoxypropyl]-7-methyl-N- [(thiophen-2-yl)methyl]thieno[3,2- c]pyridazin-4-amine |
| 132 | 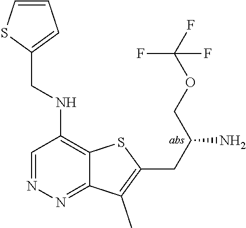 [SMILES: Cc1c(C[C@@H](N)COC(F)(F)F)sc2c(NCc3cccs3)cnnc12] | 6-[(2R)-2-amino-3- (trifluoromethoxy)propyl]-7-methyl-N- [(thiophen-2-yl)methyl]thieno[3,2- c]pyridazin-4-amine |
| 133 | 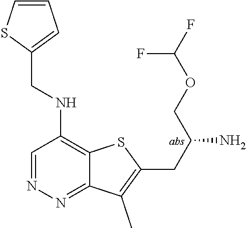 [SMILES: Cc1c(C[C@@H](N)COC(F)F)sc2c(NCc3cccs3)cnnc12] | 6-[(2R)-2-amino-3- (difluoromethoxy)propyl]-7-methyl-N- [(thiophen-2-y1)methyl]thieno[3,2- c]pyridazin-4-amine |
| 134 | 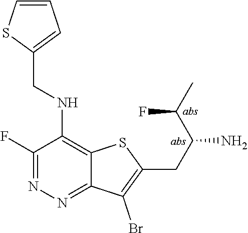 [SMILES: C[C@H](F)[C@H](N)Cc1sc2c(NCc3cccs3)c(F)nnc2c1Br] | 6-[(2R,3S)-2-amino-3-fluorobutyl]-7- bromo-3-fluoro-N-[(thiophen-2- yl)methyl]thieno[3,2-c]pyridazin-4- amine |
| 135 | 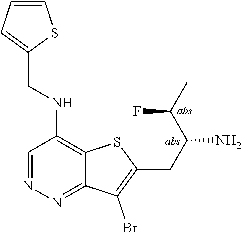 [SMILES: C[C@H](F)[C@H](N)Cc1sc2c(NCc3cccs3)cnnc2c1Br] | 6-[(2R,3S)-2-amino-3-fluorobutyl]-7- bromo-N-[(thiophen-2- yl)methyl]thieno[3,2-c]pyridazin-4- amine |
| 136 | 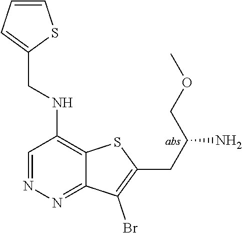 [SMILES: COC[C@H](N)Cc1sc2c(NCc3cccs3)cnnc2c1Br] | 6-[(2R)-2-amino-3-methoxypropyl]-7- bromo-N-[(thiophen-2- yl)methyl]thieno[3,2-c]pyridazin-4- amine |
| 137 | 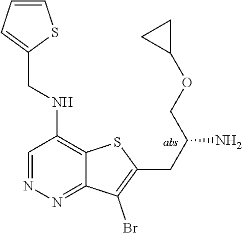 [SMILES: N[C@@H](COC1CC1)Cc1sc2c(NCc3cccs3)cnnc2c1Br] | 6-[(2R)-2-amino-3- cyclopropoxypropyl]-7-bromo-N- [(thiophen-2-yl)methyl]thieno[3,2- c]pyridazin-4-amine |
| 138 | 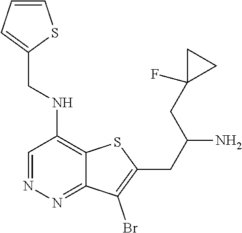 [SMILES: NC(Cc1sc2c(NCc3cccs3)cnnc2c1Br)CC1(F)CC1] | 6-[2-amino-3-(1- fluorocyclopropyl)propyl]-7-bromo-N- [(thiophen-2-yl)methyl]thieno[3,2- c]pyridazin-4-amine |
| 139 | 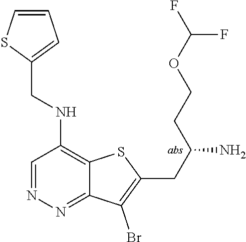 [SMILES: N[C@@H](CCOC(F)F)Cc1sc2c(NCc3cccs3)cnnc2c1Br] | 6-[(2S)-2-amino-4- (difluoromethoxy)butyl]-7-methyl-N- [(thiophen-2-yl)methyl]thieno[3,2- c]pyridazin-4-amine |
| 140 | 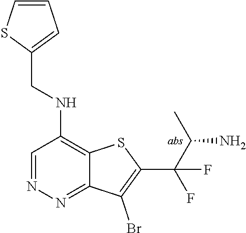 [SMILES: C[C@H](N)C(F)(F)c1sc2c(NCc3cccs3)cnnc2c1Br] | 6-[(2S)-2-amino-1,1-difluoropropyl]-7- bromo-N-[(thiophen-2- yl)methyl]thieno[3,2-c]pyridazin-4- amine |
| 141 | 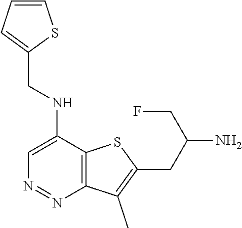 [SMILES: Cc1c(CC(N)CF)sc2c(NCc3cccs3)cnnc12] | 6-(2-amino-3-fluoropropyl)-7-methyl- N-[(thiophen-2-yl)methyl]thieno[3,2- c]pyridazin-4-amine |
| 142 | 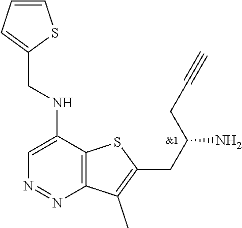 [SMILES: C#CC[C@H](N)Cc1sc2c(NCc3cccs3)cnnc2c1C] | 6-[(2S)-2-aminopent-4-yn-1-yl]-7- methyl-N-[(thiophen-2- yl)methyl]thieno[3,2-c]pyridazin-4- amine |
| 143 | 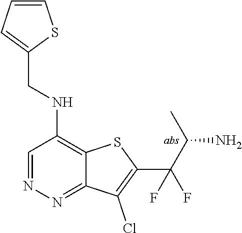 [SMILES: C[C@H](N)C(F)(F)c1sc2c(NCc3cccs3)cnnc2c1Cl] | 6-[(2S)-2-amino-1,1-difluoropropyl]-7- chloro-N-[(thiophen-2- yl)methyl]thieno[3,2-c]pyridazin-4- amine |
| 144 | 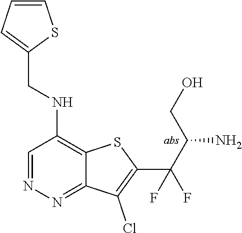 [SMILES: N[C@@H](CO)C(F)(F)c1sc2c(NCc3cccs3)cnnc2c1Cl] | (2S)-2-amino-3-(7-chloro-4- {[(thiophen-2- yl)methyl]amino}thieno[3,2- c]pyridazin-6-yl)-3,3-difluoropropan-1- ol |
| 145 | 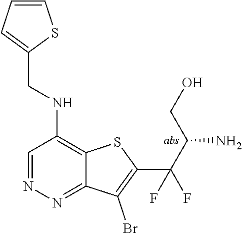 [SMILES: N[C@@H](CO)C(F)(F)c1sc2c(NCc3cccs3)cnnc2c1Br] | (2S)-2-amino-3-(7-bromo-4- {[(thiophen-2- yl)methyl]amino}thieno[3,2- c]pyridazin-6-yl)-3,3-difluoropropan-1- ol |
| 146 | 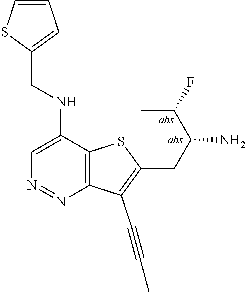 [SMILES: CC#Cc1c(C[C@@H](N)[C@H](C)F)sc2c(NCc3cccs3)cnnc12] | 6-[(2R,3S)-2-amino-3-fluorobutyl]-7- (prop-1-yn-1-yl)-N-[(thiophen-2- yl)methyl]thieno[3,2-c]pyridazin-4- amine |

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition can be a mixture of a SMSM described herein with one or more other chemical components (i.e., pharmaceutically acceptable ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

The compositions described herein can be administered to the subject in a variety of ways, including parenterally, intravenously, intradermally, intramuscularly, colonically, rectally, or intraperitoneally. In some embodiments, the small molecule splicing modulator, or a pharmaceutically acceptable salt thereof is administered by intraperitoneal injection, intramuscular injection, subcutaneous injection, or intravenous injection of the subject. In some embodiments, the pharmaceutical compositions can be administered parenterally, intravenously, intramuscularly or orally. The oral agents comprising a small molecule splicing modulator can be in any suitable form for oral administration, such as liquid, tablets, capsules, or the like. The oral formulations can be further coated or treated to prevent or reduce dissolution in stomach. The compositions of the present invention can be administered to a subject using any suitable methods known in the art. Suitable formulations for use in the present invention and methods of delivery are generally well known in the art. For example, the small molecule splicing modulators described herein can be formulated as pharmaceutical compositions with a pharmaceutically acceptable diluent, carrier, or excipient. The compositions may contain pharmaceutically acceptable auxiliary substances as required to approximate physiological conditions including pH adjusting and buffering agents, tonicity adjusting agents, wetting agents and the like, such as, for example, sodium acetate, sodium lactate, sodium chloride, potassium chloride, calcium chloride, sorbitan monolaurate, triethanolamine oleate, etc.

In some embodiments, the pharmaceutical formulation is in the form of a tablet. In other embodiments, pharmaceutical formulations containing a SMSM described herein are in the form of a capsule. In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a SMSM described herein can be formulated for use as an aerosol, a mist, or a powder. For buccal or sublingual administration, the compositions may take the form of tablets, lozenges, or gels formulated in a conventional manner. In some embodiments, a SMSM described herein can be prepared as transdermal dosage forms. In some embodiments, a SMSM described herein can be formulated into a pharmaceutical composition suitable for intramuscular, subcutaneous, or intravenous injection. In some embodiments, a SMSM described herein can be administered topically and can be formulated into a variety of topically administrable compositions, such as solutions, suspensions, lotions, gels, pastes, medicated sticks, balms, creams, or ointments. In some embodiments, a SMSM described herein can be formulated in rectal compositions such as enemas, rectal gels, rectal foams, rectal aerosols, suppositories, jelly suppositories, or retention enemas.

In some embodiments, disclosed herein is a pharmaceutical composition comprising a compound of the disclosure or a pharmaceutically acceptable salt or a pharmaceutically acceptable solvate thereof, and a pharmaceutically acceptable excipient or carrier.

### Splicing Modulation of Target Gene Products

The present invention contemplates use of small molecules with favorable drug properties that modulate the activity of splicing of a target RNA. Provided herein are small molecule splicing modulators (SMSMs) that modulate splicing of a polynucleotide. In some embodiments, the SMSMs bind and modulate target RNA. In some embodiments, provided herein is a library of SMSMs that bind and modulate one or more target RNAs. In some embodiments, the target RNA is mRNA. In some embodiments, the target RNA is a noncoding RNA. In some embodiments, the target RNA is a pre-mRNA. In some embodiments, the target RNA is hnRNA. In some embodiments, the small molecules modulate splicing of the target RNA. In some embodiments, a small molecule provided herein modulates splicing at a sequence of the target RNA. In some embodiments, a small molecule provided herein modulates splicing at a cryptic splice site sequence of the target RNA. In some embodiments, a small molecule provided herein modulates splicing at an alternative splice site sequence of the target RNA. In some embodiments, a small molecule provided herein modulates splicing at a native splice site sequence of the target RNA. In some embodiments, a small molecule provided herein binds to a target RNA. In some embodiments, a small molecule provided herein binds to a splicing complex or a component thereof. In some embodiments, a small molecule provided herein binds to a target RNA and a splicing complex or a component thereof. In some embodiments, a small molecule provided herein modulates binding affinity of a splicing complex component to a target RNA such as a pre-mRNA. In some embodiments, a small molecule provided herein modulates binding affinity of a splicing complex component to a target RNA such as a pre-mRNA at a splice site sequence. In some embodiments, a small molecule provided herein modulates binding affinity of a splicing complex component to a target RNA such as a pre-mRNA upstream of a splice site sequence or downstream of a splice site sequence.

Described herein are compounds modifying splicing of gene products, such as Ataxin 3 pre-mRNA for use in the treatment, prevention, and/or delay of progression of diseases or conditions.

In some embodiments, described herein, is a method of treating, preventing, delaying of progress, or ameliorating symptoms of a disease or a condition associated with Ataxin 3 (ATXN3) expression level or activity level in a subject in need thereof, comprising administering a therapeutically effective amount of a small molecule splicing modulator (SMSM), wherein the SMSM binds to a pre-mRNA encoded by ATXN3 and modulates splicing of the ATXN3 pre-mRNA in a cell of the subject to produce a spliced product of the ATXN3 pre-mRNA.

In some embodiments, described herein is a method of treating, preventing, delaying of progress, or ameliorating symptoms of a disease or a condition associated with Ataxin 3 (ATXN3) expression level or activity level in a subject in need thereof, comprising administering a therapeutically effective amount of a compound or salt of Formula (I). In some embodiments, described herein is a method of modulating splicing of a Ataxin3 (ATXN3) pre-mRNA, comprising contacting a compound or salt of Formula (I) to the ATXN3 pre-mRNA with a splice site sequence or cells comprising the ATXN3 pre-mRNA, wherein the compound binds to the ATXN3 pre-mRNA and modulates splicing of the ATXN3 pre-mRNA in a cell of a subject to produce a spliced product of the ATXN3 pre-mRNA. In some embodiments, described herein is use of a compound of Formula (I), or a pharmaceutically acceptable salt thereof, in the manufacture of a medicament for the treatment of a condition or disease associated with Ataxin 3 (ATXN3) expression level or activity level.

In some embodiments, the spliced product of the ATXN3 pre-mRNA undergoes non-sense mediated decay (NMD) and/or nuclear retention. In some embodiments, the nonsense-mediated decay (NMD) and/or nuclear retention of the spliced product of the ATXN3 pre-mRNA is promoted. In some embodiments, the nonsense-mediated decay (NMD) and/or nuclear retention of the spliced product of the ATXN3 pre-mRNA is increased compared to a spliced product of the ATXN3 pre-mRNA produced in the absence of the SMSM.

In some embodiments, described herein is a method of modulating splicing of a Ataxin3 (ATXN3) pre-mRNA, comprising contacting a small molecule splicing modulator (SMSM) to the ATXN3 pre-mRNA with a splice site sequence or cells comprising the ATXN3 pre-mRNA, wherein the SMSM binds to the ATXN3 pre-mRNA and modulates splicing of the ATXN3 pre-mRNA in a cell of a subject to produce a spliced product of the ATXN3 pre-mRNA.

In some embodiments, described herein, is a method of modulating splicing of Ataxin 3 (ATXN3) pre-mRNA, comprising contacting a small molecule splicing modulator (SMSM) to the ATXN3 pre-mRNA with a splice site sequence or cells comprising the ATXN3 pre-mRNA, wherein the SMSM binds to the ATXN3 pre-mRNA and modulates splicing of the ATXN3 pre-mRNA in a cell of a subject to produce a spliced product of the ATXN3 pre-mRNA, wherein the splice site sequence comprises UCCUAU/guaagauucugu.

In some embodiments, described herein, is a method of treating, preventing, delaying of progress, or ameliorating symptoms of a disease or condition associated with Ataxin 3 (ATXN3) expression level or activity level in a subject in need thereof, comprising administering a therapeutically effective amount of a small molecule splicing modulator (SMSM) to the subject, wherein the SMSM binds to a ATXN3 pre-mRNA with a splice site sequence and modulates splicing of the ATXN3 pre-mRNA in a cell of the subject, wherein a spliced product of the ATXN3 pre-mRNA undergoes nonsense-mediated decay (NMD), and wherein the splice site sequence comprises UCCUAU/guaagauucugu.

In some embodiments, the modulating splicing comprises modulating alternative splicing. In some embodiments, the modulating splicing comprises promoting exon skipping. In some embodiments, the modulating splicing comprises promoting exon inclusion. In some embodiments, the modulating splicing comprises modulating nonsense-mediated mRNA decay (NMD). In some embodiments, the modulating NMD comprises promoting NMD. In some embodiments, the modulating splicing comprises modulating nuclear retention of the spliced product of the pre-mRNA. In some embodiments, the modulating intron retention comprises promoting nuclear retention of the spliced product of the pre-mRNA.

In some embodiments, the splice site sequence is a native splice site sequence. In some embodiments, the native splice site is a canonical splice site. In some embodiments, the native splice site is an alternative splice site. In some embodiments, the alternative splice site comprises a 5′ splice site sequence. In some embodiments, the alternative splice site sequence comprises UCCUAU/guaagauucugu. In some embodiments, the SMSM induces splicing at the alternative splice site. In some embodiments, the splicing at the alternative splice site results in a frameshift in a downstream exon in the spliced product. In some embodiments, the downstream exon comprises an in-frame stop codon that is not in frame in the absence of splicing at the alternative splice site. In some embodiments, the in-frame stop codon in the downstream exon is at least 50 or at least 60 base pairs upstream of the 3′ end of the downstream exon. In some embodiments, the in-frame stop codon in the downstream exon is at least 50 or at least 60 base pairs upstream of a final exon-exon junction.

In some embodiments, the splicing of the pre-mRNA at the alternative splice site promotes NMD of the spliced product of the ATXN3 pre-mRNA. In some embodiments, the spliced product comprises an alternative exon. In some embodiments, the SMSM promotes inclusion of the alternative exon in the spliced product. In some embodiments, the alternative exon comprises a poison exon. In some embodiments, the SMSM promotes inclusion of the poison exon in the spliced product. In some embodiments, the poison exon comprises an in-frame stop codon. In some embodiments, the in-frame stop codon is a premature termination codon. In some embodiments, the in-frame stop codon is at least 50 or 60 base pairs upstream of the 3′ end of the poison exon. In some embodiments, the in-frame stop codon is less than 60 base pairs upstream of the 3′ end of the poison exon and wherein the exon immediately downstream of the poison exon is not the last exon in the pre-mRNA. In some embodiments, the sum of (a) the number of base pairs in the exon immediately downstream of the poison exon and (b) the number of base pairs between the premature termination codon in the poison exon and the 3′ end of the poison exon is at least 50 or at least 60.

In some embodiments, the cells comprise primary cells. In some embodiments, the cells comprise disease cells. In some embodiments, the SMSM modulates proliferation or survival of the cells. In some embodiments, the SMSM modulates the expression level of a protein encoded by the spliced product of the pre-mRNA in the cells.

TABLE 2
Exemplary targets for exon inclusion

| Gene | ATXN3 |
|---|---|
| Exon Coordinates | Chr14: 92093746-92093831 |
| Splicing Event Region | chr14: 92093319-92096092 |
| Strand | — |
| Target site | Exon 4 |
| Exon length | 86 |
| SEQ ID NO: | 1 |
| 5′ ss sequence | UCCUAU/guaagauucugu |
| (−6~+12) | |
| 5′ ss-U1 duplex | -1U-C loop |
| structure | |
| Disease | Spinocerebellar |
| | Ataxia Type 3 |

### Methods of Treatment

The compositions and methods described herein can be used for treating a human disease or disorder associated with aberrant splicing, such as aberrant pre-mRNA splicing. The compositions and methods described herein can be used for treating a human disease or disorder by modulating mRNA, such as pre-mRNA. In some embodiments, the compositions and methods described herein can be used for treating a human disease or disorder by modulating splicing of a nucleic acid even when that nucleic acid is not aberrantly spliced in the pathogenesis of the disease or disorder being treated.

In some embodiments, an effective amount in the context of the administration of a SMSM or a pharmaceutically acceptable salt thereof, or composition or medicament thereof refers to an amount of a SMSM or a pharmaceutically acceptable salt thereof to a patient which has a therapeutic effect and/or beneficial effect. In certain specific embodiments, an effective amount in the context of the administration of a SMSM or a pharmaceutically acceptable salt thereof, or composition or medicament thereof to a patient results in one, two or more of the following effects: (i) reduces or ameliorates the severity of a disease; (ii) delays onset of a disease; (iii) inhibits the progression of a disease; (iv) reduces hospitalization of a subject; (v) reduces hospitalization length for a subject; (vi) increases the survival of a subject; (vii) improves the quality of life of a subject; (viii) reduces the number of symptoms associated with a disease; (ix) reduces or ameliorates the severity of a symptom associated with a disease; (x) reduces the duration of a symptom associated with a disease associated; (xi) prevents the recurrence of a symptom associated with a disease; (xii) inhibits the development or onset of a symptom of a disease; and/or (xiii) inhibits of the progression of a symptom associated with a disease. In some embodiments, an effective amount of a SMSM or a pharmaceutically acceptable salt thereof is an amount effective to restore the amount of an RNA transcript of a gene to the amount of the RNA transcript detectable in healthy patients or cells from healthy patients. In other embodiments, an effective amount of a SMSM or a pharmaceutically acceptable salt thereof is an amount effective to restore the amount an RNA isoform and/or protein isoform of a gene to the amount of the RNA isoform and/or protein isoform detectable in healthy patients or cells from healthy patients.

In some embodiments, an effective amount of a SMSM or a pharmaceutically acceptable salt thereof is an amount effective to decrease the aberrant amount of an RNA transcript of a gene which associated with a disease. In some embodiments, an effective amount of a SMSM or a pharmaceutically acceptable salt thereof is an amount effective to decrease the amount of the aberrant expression of an isoform of a gene. In some embodiments, an effective amount of a SMSM or a pharmaceutically acceptable salt thereof is an amount effective to result in a substantial change in the amount of an RNA transcript (e.g., an mRNA transcript), alternative splice variant, or isoform.

In some embodiments, an effective amount of a SMSM or a pharmaceutically acceptable salt thereof is an amount effective to increase the amount of an RNA transcript (e.g., an mRNA transcript) of a gene that is beneficial for the prevention and/or treatment of a disease. In some embodiments, an effective amount of a SMSM or a pharmaceutically acceptable salt thereof is an amount effective to increase the amount of an alternative splice variant of an RNA transcript of a gene that is beneficial for the prevention and/or treatment of a disease. In some embodiments, an effective amount of a SMSM or a pharmaceutically acceptable salt thereof is an amount effective to increase the amount of an isoform of a gene that is beneficial for the prevention and/or treatment of a disease.

In some embodiments, an effective amount of a SMSM or a pharmaceutically acceptable salt thereof is an amount effective to decrease the amount of an RNA transcript (e.g., an mRNA transcript) which causes or is related to the symptoms of the condition or disease. In particular embodiments, the SMSM decreases the amount of an RNA transcript that causes or relates to the symptoms of the condition or disease by modulating one or more splicing elements of the RNA transcript. In some embodiments, the SMSM promotes skipping of one or more exons. In some embodiments, the SMSM promotes inclusion of one or more exons. In some embodiments, the SMSM promotes inclusion of one or more exons and/or introns that relate to nonsense-mediated mRNA decay (NMD). In some embodiments, the one or more exons harbor a premature termination codon. In particular embodiments, the premature stop codon is an in-frame codon that does not cause frameshift of the downstream exon(s). In some embodiments, inclusion of the one or more exons causes a reading frameshift in a downstream exon, for example, in the immediately downstream exon, introducing a premature termination codon.

A method of treating a disease or a condition in a subject in need thereof can comprise administering to the subject a therapeutically effective amount of a compound described herein or a pharmaceutically acceptable salt thereof. In some embodiments, the present disclosure relates to a method for the treatment, prevention and/or delay of progression of a disease or a condition associated with a gene listed in Table 2,

Non-limiting examples of effective amounts of a SMSM or a pharmaceutically acceptable salt thereof are described herein. For example, the effective amount may be the amount required to prevent and/or treat a disease associated with the aberrant amount of an mRNA transcript of gene in a human subject. In general, the effective amount will be in a range of from about 0.001 mg/kg/day to about 500 mg/kg/day for a patient having a weight in a range of between about 1 kg to about 200 kg. The typical adult subject is expected to have a median weight in a range of between about 70 and about 100 kg.

In one embodiment, a SMSM described herein can be used in the preparation of medicaments for the treatment of diseases or conditions described herein. In addition, a method for treating any of the diseases or conditions described herein in a subject in need of such treatment, can involve administration of pharmaceutical compositions that include at least one SMSM described herein or a pharmaceutically acceptable salt, thereof, in a therapeutically effective amount to a subject.

In certain embodiments, a SMSM described herein can be administered for prophylactic and/or therapeutic treatments. In certain therapeutic applications, the compositions are administered to a patient already suffering from a disease or a condition, in an amount sufficient to cure or at least partially arrest at least one of the symptoms of the disease or the condition. Amounts effective for this use depend on the severity and course of the disease or the condition, previous therapy, the patient's health status, weight, and response to the drugs, and the judgment of the treating physician. Therapeutically effective amounts are optionally determined by methods including, but not limited to, a dose escalation clinical trial. In prophylactic applications, compositions containing a SMSM described herein can be administered to a patient susceptible to or otherwise at risk of a particular disease, disorder, or condition. In certain embodiments, the dose of drug being administered may be temporarily reduced or temporarily suspended for a certain length of time (i.e., a “drug holiday”). Doses employed for adult human treatment typically range of 0.01 mg-5000 mg per day or from about 1 mg to about 1000 mg per day. In some embodiments, a desired dose is conveniently presented in a single dose or in divided doses.

For combination therapies described herein, dosages of the co-administered compounds can vary depending on the type of co-drug(s) employed, on the specific drug(s) employed, on the disease or the condition being treated and so forth. In additional embodiments, when co-administered with one or more other therapeutic agents, the compound provided herein is administered either simultaneously with the one or more other therapeutic agents, or sequentially. If administration is simultaneous, the multiple therapeutic agents can be, by way of example only, provided in a single, unified form, or in multiple forms.

### Methods of Administering

The compositions described herein can be administered to the subject in a variety of ways, including parenterally, intravenously, intradermally, intramuscularly, colonically, rectally or intraperitoneally. In some embodiments, the small molecule splicing modulator (SMSM) or a pharmaceutically acceptable salt thereof is administered by intraperitoneal injection, intramuscular injection, subcutaneous injection, or intravenous injection of the subject. In some embodiments, the pharmaceutical compositions can be administered parenterally, intravenously, intramuscularly or orally. The oral agents comprising a small molecule splicing modulator can be in any suitable form for oral administration, such as liquid, tablets, capsules, or the like. The oral formulations can be further coated or treated to prevent or reduce dissolution in stomach. The compositions of the present invention can be administered to a subject using any suitable methods known in the art. Suitable formulations for use in the present invention and methods of delivery are generally well known in the art. For example, the small molecule splicing modulators described herein can be formulated as pharmaceutical compositions with a pharmaceutically acceptable diluent, carrier, or excipient. The compositions may contain pharmaceutically acceptable auxiliary substances as required to approximate physiological conditions including pH adjusting and buffering agents, tonicity adjusting agents, wetting agents and the like, such as, for example, sodium acetate, sodium lactate, sodium chloride, potassium chloride, calcium chloride, sorbitan monolaurate, triethanolamine oleate, etc.

Pharmaceutical formulations described herein can be administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the pharmaceutical compositions described herein are administered orally. In some embodiments, the pharmaceutical compositions described herein are administered topically. In such embodiments, the pharmaceutical compositions described herein are formulated into a variety of topically administrable compositions, such as solutions, suspensions, lotions, gels, pastes, shampoos, scrubs, rubs, smears, medicated sticks, medicated bandages, balms, creams, or ointments. In some embodiments, the pharmaceutical compositions described herein are administered topically to the skin. In some embodiments, the pharmaceutical compositions described herein are administered by inhalation. In some embodiments, the pharmaceutical compositions described herein are formulated for intranasal administration. Such formulations include nasal sprays, nasal mists, and the like. In some embodiments, the pharmaceutical compositions described herein are formulated as eye drops. In some embodiments, the pharmaceutical compositions described herein are: (a) systemically administered to the mammal; and/or (b) administered orally to the mammal; and/or (c) intravenously administered to the mammal; and/or (d) administered by inhalation to the mammal; and/or (e) administered by nasal administration to the mammal; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal. In some embodiments, the pharmaceutical compositions described herein are administered orally to the mammal. In certain embodiments, a SMSM described herein is administered in a local rather than systemic manner. In some embodiments, a SMSM described herein is administered topically. In some embodiments, a SMSM described herein is administered systemically.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

SMSMs suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It must be stable under the conditions of manufacture and storage and must be preserved against contamination from microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition.

### Dosing and Schedules

The SMSMs utilized in the methods of the invention can be, e.g., administered at dosages that may be varied depending upon the requirements of the subject, the severity of the condition being treated and/or imaged, and/or the SMSM being employed. For example, dosages can be empirically determined considering the type and stage of disease diagnosed in a particular subject and/or the type of imaging modality being used in conjunction with the SMSMs. The dose administered to a subject, in the context of the present invention should be sufficient to affect a beneficial diagnostic or therapeutic response in the subject. The size of the dose also can be determined by the existence, nature, and extent of any adverse side-effects that accompany the administration of a SMSM in a particular subject.

Within the scope of the present description, the effective amount of a SMSM or a pharmaceutically acceptable salt thereof for use in the manufacture of a medicament, the preparation of a pharmaceutical kit or in a method for preventing and/or treating a disease in a human subject in need thereof, is intended to include an amount in a range of from about 1 μg to about 50 grams.

The compositions of the present invention can be administered as frequently as necessary, including hourly, daily, weekly, or monthly.

In any of the aforementioned aspects are further embodiments comprising single administrations of an effective amount of a SMSM described herein, including further embodiments in which (i) the compound is administered once; (ii) the compound is administered to the mammal multiple times over the span of one day; (iii) continually; or (iv) continuously.

In any of the aforementioned aspects are further embodiments comprising multiple administrations of the effective amount of a SMSM described herein, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of a SMSM described herein is temporarily suspended or the dose of the compound being administered is temporarily reduced; at the end of the drug holiday, dosing of the compound is resumed. In one embodiment, the length of the drug holiday varies from 2 days to 1 year.

### Combination Therapies

In certain instances, it is appropriate to administer at least one SMSM described herein in combination with another therapeutic agent. For example, a compound SMSM described herein can be co-administered with a second therapeutic agent, wherein SMSM and the second therapeutic agent modulate different aspects of the disease, disorder or condition being treated, thereby providing a greater overall benefit than administration of either therapeutic agent alone.

In some embodiments, a SMSM may be administered in combination with one or more other SMSMs.

A SMSM may be administered to a subject in need thereof prior to, concurrent with, or following the administration of chemotherapeutic agents. For instance, SMSMs may be administered to a subject at least 8 hours, 7 hours, 6 hours, 5 hours, 4 hours, 3 hours, 2 hours, 1.5 hours, 1 hour, or 30 minutes before the starting time of the administration of chemotherapeutic agent(s). In certain embodiments, they may be administered concurrent with the administration of chemotherapeutic agent(s). In other words, in these embodiments, SMSMs are administrated at the same time when the administration of chemotherapeutic agent(s) starts. In other embodiments, SMSMs may be administered following the starting time of administration of chemotherapeutic agent(s) (e.g., at least 30 minutes, 1 hour, 2 hours, 3 hours, 4 hours, 5 hours, 6 hours, 7 hours, or 8 hours after the starting time of administration of chemotherapeutic agents). Alternatively, SMSMs may be administered at least 30 minutes, 1 hour, 2 hours, 3 hours, 4 hours, 5 hours, 6 hours, 7 hours, or 8 hours after the completion of administration of chemotherapeutic agents. Generally, these SMSMs are administered for a sufficient period of time so that the disease or the condition is prevented or reduced. Such sufficient period of time may be identical to, or different from, the period during which chemotherapeutic agent(s) are administered. In certain embodiments, multiple doses of SMSMs are administered for each administration of a chemotherapeutic agent or a combination of multiple chemotherapeutic agents.

In certain embodiments, an appropriate dosage of a SMSM is combined with a specific timing and/or a particular route to achieve the optimum effect in preventing or reducing the disease or the condition. For instance, a SMSM may be administered to a human orally at least 1 hour, 2 hours, 3 hours, 4 hours, 5 hours, 6 hours, 7 hours 8 hours, 9 hours, 10 hours, 11 hours, or 12 hours; or at least 1 day, 2 days, 3 days, 4 days, 5 days, 6 days, 7 days, 8 days, 9 days, 10 days, 11 days, 12 days; or at least 1 week, 2 weeks, 3 weeks or 4 weeks; or at least 1 month, 2 months, 3 months, 4 months, 5 months, 6 months, 7 months, 8 months, 9 months, 10 months, 11 months, or 12 months; prior to or after the beginning or the completion, of the administration of a chemotherapeutic agent or a combination of chemotherapeutic agents.

### Subjects

The subjects that can be treated with the SMSMs and methods described herein can be any subject that produces mRNA that is subject to alternative splicing, e.g., the subject may be a eukaryotic subject, such as a plant or an animal. In some embodiments, the subject is a mammal, e.g., human. In some embodiments, the subject is a human. In some embodiments, the subject is a non-human animal. In some embodiments, the subject is a fetus, an embryo, or a child. In some embodiments, the subject is a non-human primate such as chimpanzee, and other apes and monkey species; farm animals such as cattle, horses, sheep, goats, swine; domestic animals such as rabbits, dogs, and cats; laboratory animals including rodents, such as rats, mice and guinea pigs, and the like.

In some embodiments, the subject is prenatal (e.g., a fetus), a child (e.g., a neonate, an infant, a toddler, a preadolescent), an adolescent, a pubescent, or an adult (e.g., an early adult, a middle-aged adult, a senior citizen).

### Methods of Making Compounds

Compounds described herein can be synthesized using standard synthetic techniques or using methods known in the art in combination with methods described herein. Unless otherwise indicated, conventional methods of mass spectroscopy, NMR, HPLC, protein chemistry, biochemistry, recombinant DNA techniques and pharmacology can be employed. Compounds can be prepared using standard organic chemistry techniques such as those described in, for example, March's Advanced Organic Chemistry, 6th Edition, John Wiley and Sons, Inc. Alternative reaction conditions for the synthetic transformations described herein may be employed such as variation of solvent, reaction temperature, reaction time, as well as different chemical reagents and other reaction conditions. The starting materials can be available from commercial sources or can be readily prepared. By way of example only, provided are schemes for preparing the SMSMs described herein.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3 527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

In the reactions described, it may be necessary to protect reactive functional groups, for example hydroxy, amino, imino, thio or carboxy groups, where these are desired in the final product, in order to avoid their unwanted participation in reactions. A detailed description of techniques applicable to the creation of protecting groups and their removal are described in Greene and Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure).

SMSMs can be made using known techniques and further chemically modified, in some embodiments, to facilitate intranuclear transfer to, e.g., a splicing complex component, a spliceosome or a pre-mRNA molecule. One of ordinary skill in the art will appreciate the standard medicinal chemistry approaches for chemical modifications for intranuclear transfer (e.g., reducing charge, optimizing size, and/or modifying lipophilicity).

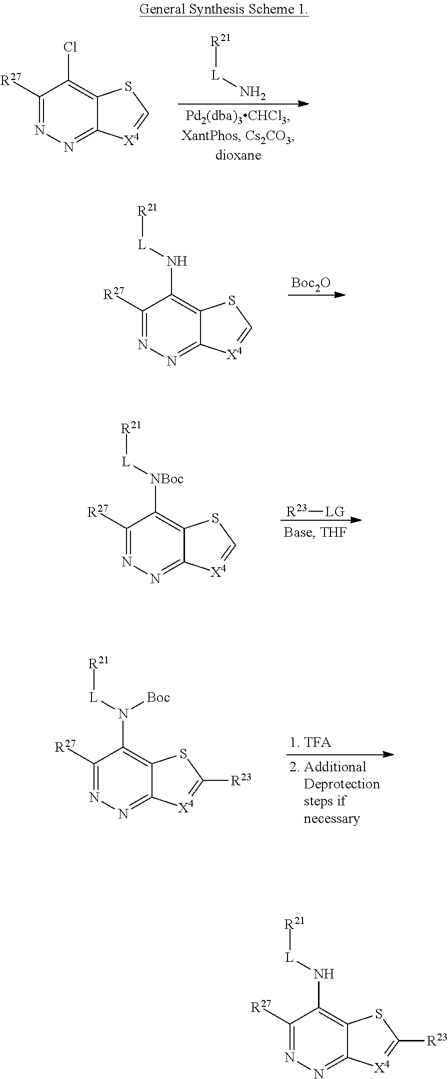

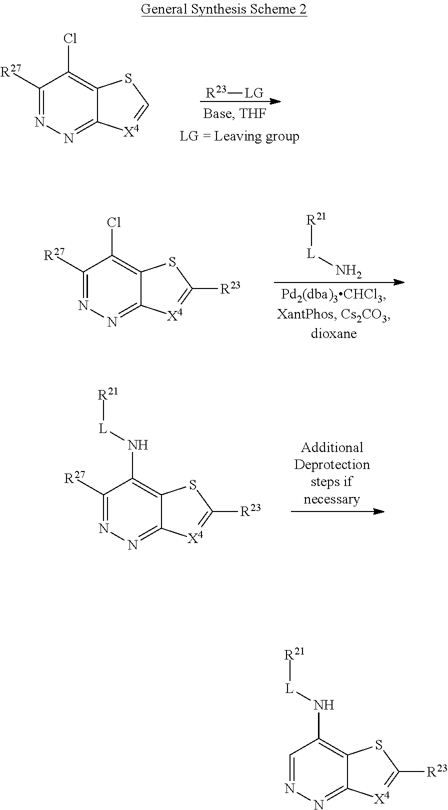

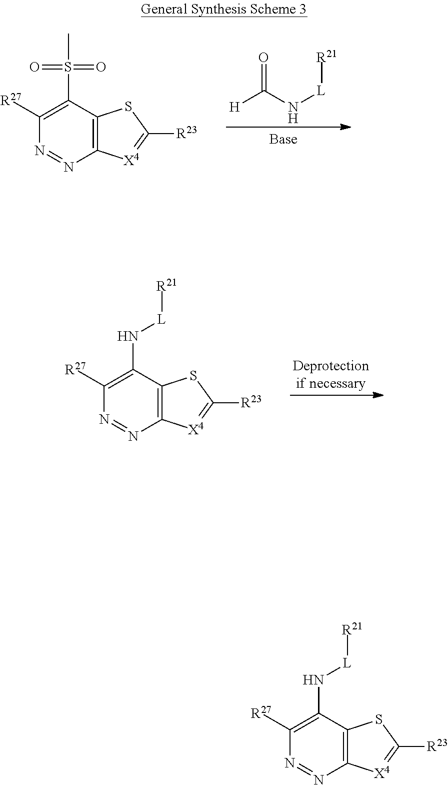

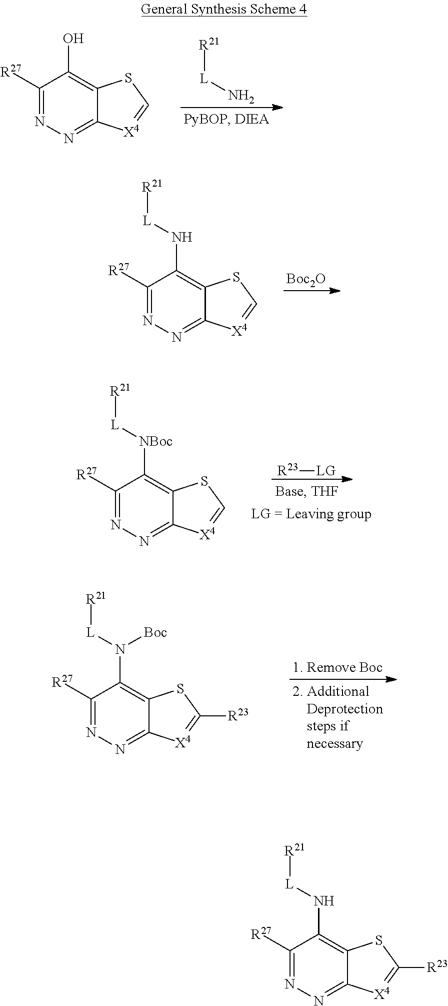

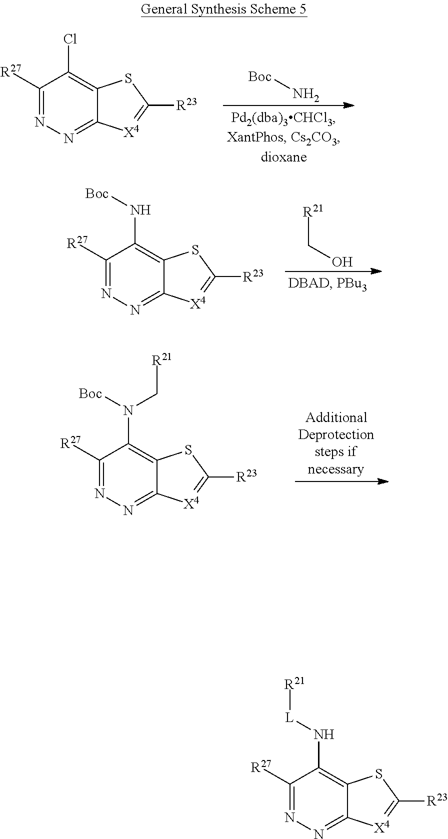

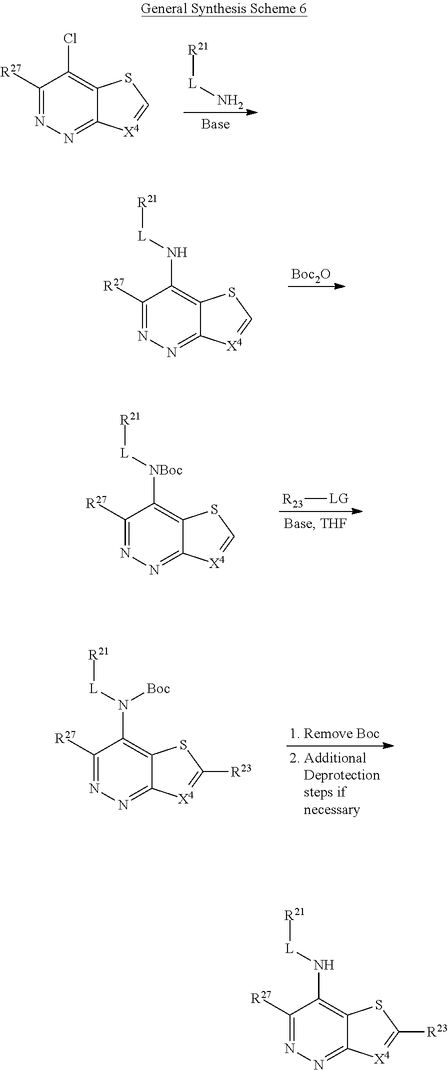

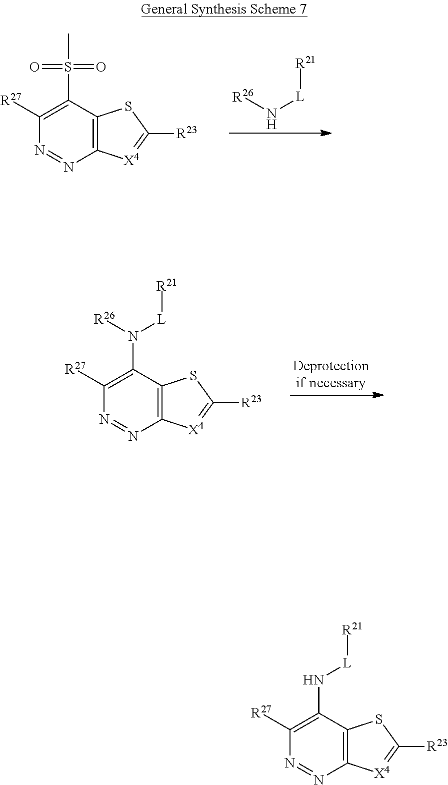

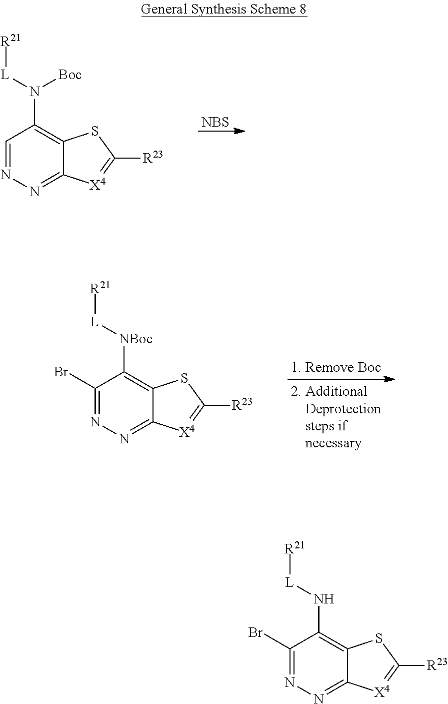

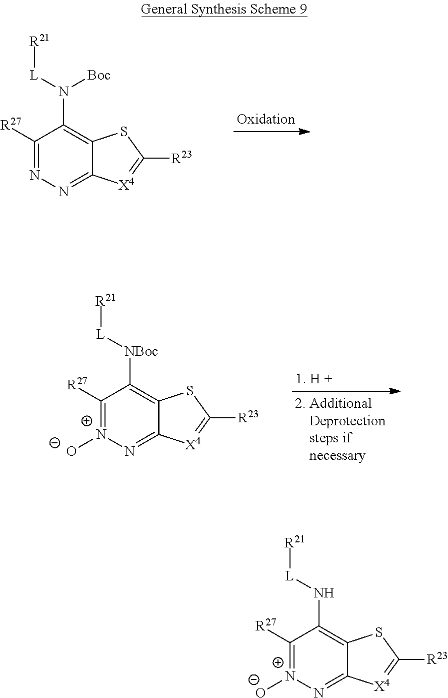

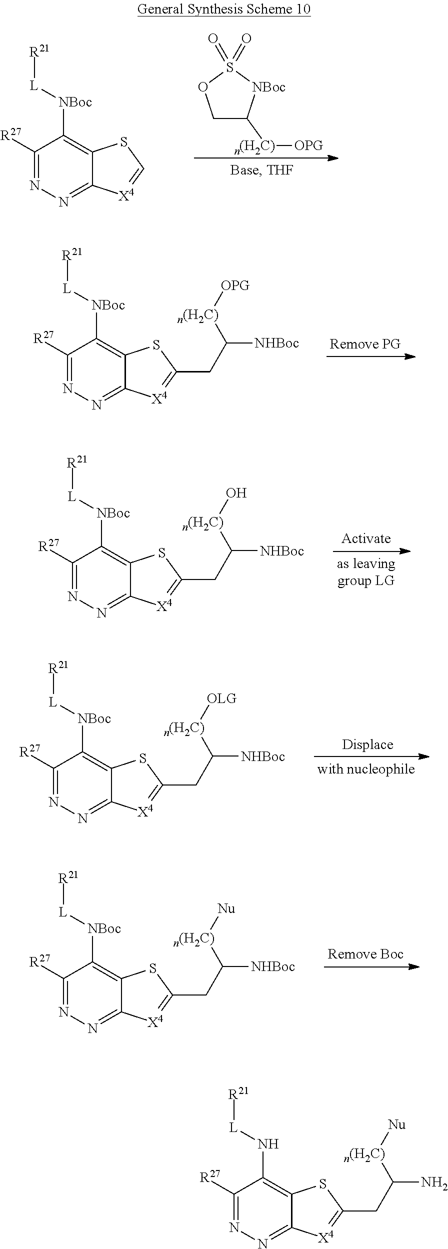

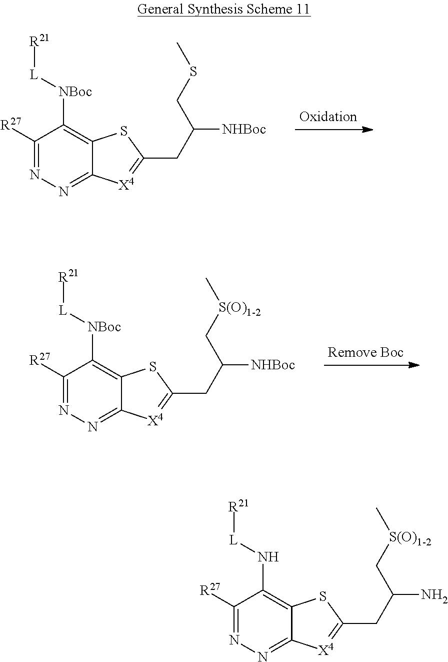

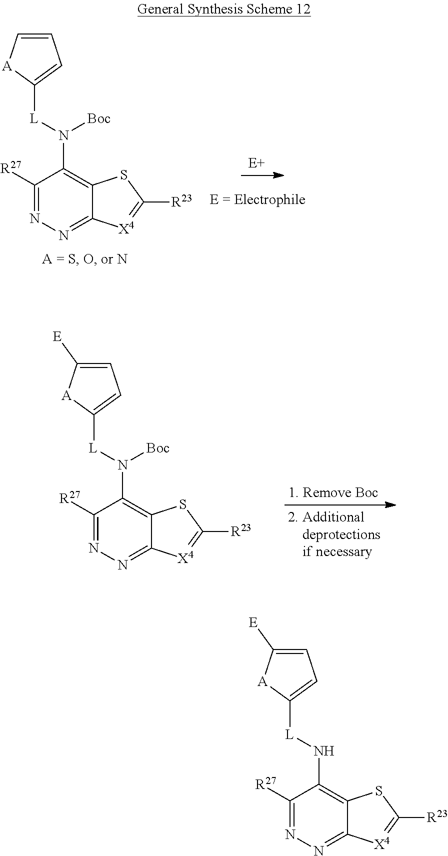

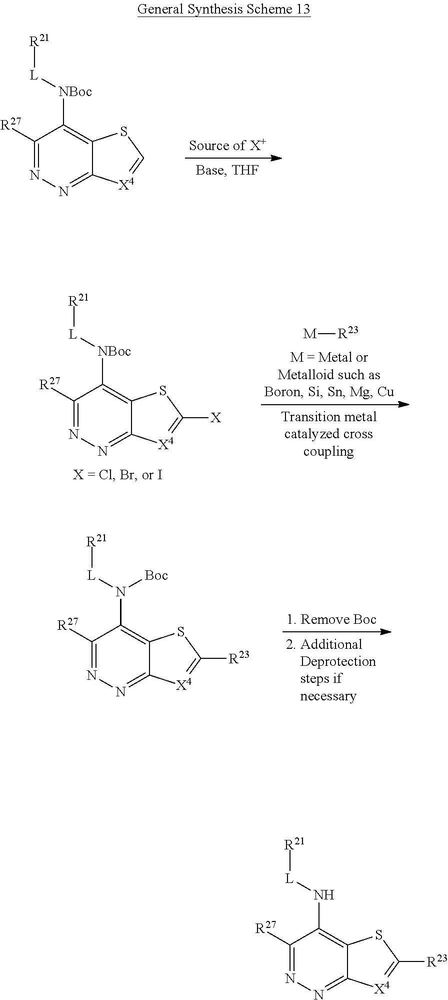

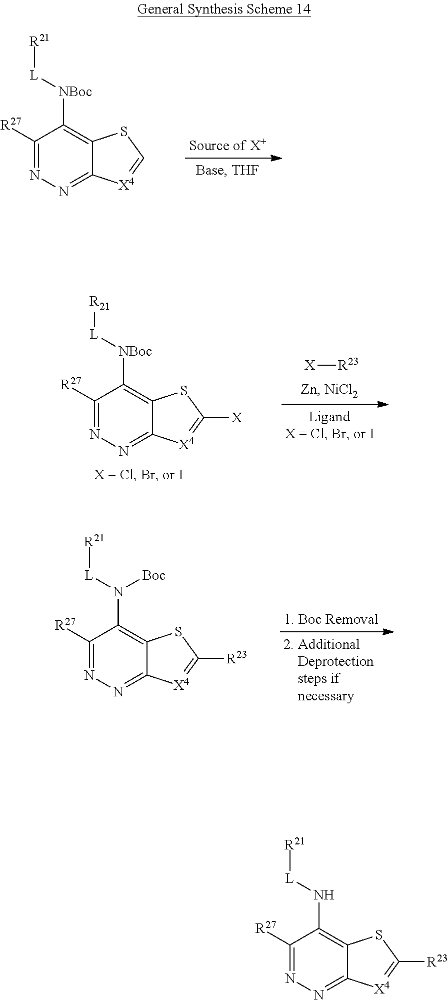

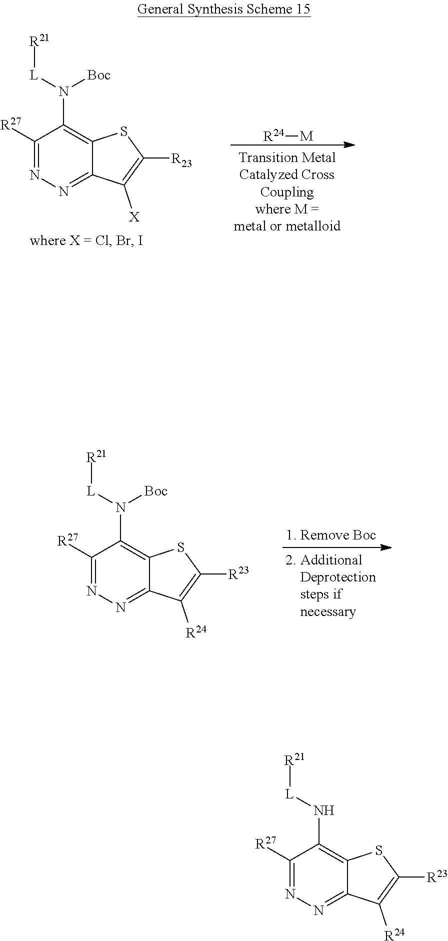

### EXAMPLES

These examples are provided for illustrative purposes only and not to limit the scope of the claims provided herein. The starting materials and reagents used for the synthesis of the compounds described herein may be synthesized or can be obtained from commercial sources, such as, but not limited to, Sigma-Aldrich, Acros Organics, Fluka, and Fisher Scientific.

### Example 1: Synthesis of Common Building Blocks

### Synthesis of 7-methylthieno[3,2-c]pyridazin-4-ol (BB-1)

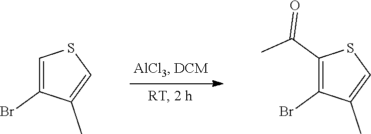

Step 1. Synthesis of 1-(3-bromo-4-methylthiophen-2-yl)ethanone: To a stirred mixture of AlCl₃ (8.28 g, 62.126 mmol, 1.1 equiv) and 3-bromo-4-methylthiophene (10.0 g, 56.478 mmol, 1 equiv) in DCM (200 mL) was added acetyl chloride (4.88 g, 62.126 mmol, 1.1 equiv) dropwise at 0° C. under nitrogen atmosphere. The resulting mixture was stirred for 2 h at room temperature under nitrogen atmosphere. The reaction was quenched by the addition of water (100 mL). The resulting mixture was extracted with CH₂Cl₂ (2×100 mL). The combined organic layers were washed with brine (1×100 mL) and dried over anhydrous Na₂SO₄. After filtration, the filtrate was concentrated under reduced pressure. The residue was purified by silica gel column chromatography, eluted with PE/EA (10:1) to afford 1-(3-bromo-4-methylthiophen-2-yl)ethanone (6 g, 48.5%) as a brown oil.

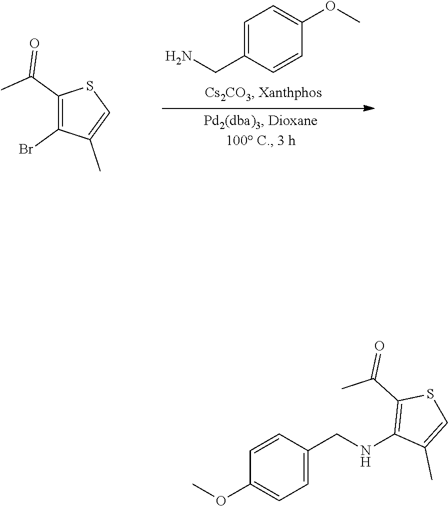

Step 2. Synthesis of 1-(3-{[(4-methoxyphenyl)methyl]amino}-4-methylthiophen-2-yl)ethanone: Into a 250 mL 3-necked round-bottom flask were added 1-(3-bromo-4-methylthiophen-2-yl)ethanone (6 g, 27.400 mmol, 1 equiv), Pd₂(dba)₃ (2.51 g, 2.740 mmol, 0.1 equiv), (4-methoxyphenyl)methanamine (4.136 g, 43.930 mmol, 1.1 equiv), XantPhos (3.17 g, 5.48 mmol, 0.2 equiv), Cs₂CO₃ (17.856 g, 54.801 mmol, 2 equiv) and dioxane (50 mL). The resulting mixture was stirred for 3 h at 100° C. under nitrogen atmosphere. The reaction was quenched by the addition of water (50 mL). The resulting mixture was extracted with EtOAc (3×100 mL). The combined organic layers were washed with brine (1×50 mL) and dried over anhydrous Na₂SO₄. After filtration, the filtrate was concentrated under reduced pressure. The residue was purified by silica gel column chromatography, eluted with PE/EA (10:1) to afford 1-(3-{[(4-methoxyphenyl)methyl]amino}-4-methylthiophen-2-yl)ethanone (3 g, 39.77%) as a brown solid.

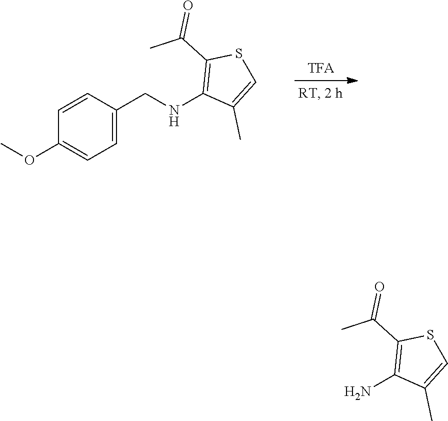

Step 3. Synthesis of 1-(3-amino-4-methylthiophen-2-yl)ethanone: Into a 50 mL 3-necked round-bottom flask were added 1-(3-{[(4-methoxyphenyl)methyl]amino}-4-methylthiophen-2-yl)ethanone (3 g, 10.894 mmol, 1 equiv) and TFA (30 mL). The resulting mixture was stirred for 2 h at room temperature under nitrogen atmosphere. The resulting mixture was concentrated under reduced pressure. The residue was purified by silica gel column chromatography, eluted with PE/EA (5:1) to afford 1-(3-amino-4-methylthiophen-2-yl)ethanone (1.5 g, 88.76%) as a brown solid.

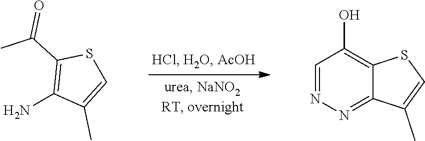

Step 4. Synthesis of 7-methylthieno[3,2-c]pyridazin-4-ol: To a stirred solution of 1-(3-amino-4-methylthiophen-2-yl)ethanone (1.5 g, 9.659 mmol, 1 equiv) in AcOH (3 mL) was added HCl (6 mL) in H₂O (12 mL) dropwise at 0° C. under nitrogen atmosphere. To the above mixture was added NaNO₂ (0.84 g, 12.169 mmol, 1.26 equiv) dropwise over 15 min at 0° C. The resulting mixture was stirred for additional 1 h at 0° C. To the above mixture was added urea (0.07 g, 1.173 mmol, 0.12 equiv) in portions over 1 h at room temperature. The resulting mixture was stirred for additional overnight at room temperature. The precipitated solids were collected by filtration and washed with water (2×20 mL) to afford 7-methylthieno[3,2-c]pyridazin-4-ol (800 mg, 49.69%) as a brown solid.

### Synthesis of 4-chloro-7-methylthieno[3,2-c]pyridazine (BB-2)

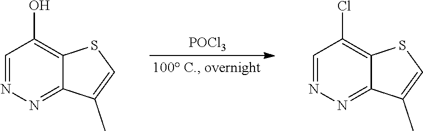

Into a 50 mL round-bottom flask were added 7-methylthieno[3,2-c]pyridazin-4-ol (800 mg, 4.813 mmol, 1 equiv) and POCl₃ (8 mL, 57.231 mmol, 11.89 equiv) at room temperature. The resulting mixture was stirred for overnight at 105° C. under nitrogen atmosphere. The reaction was poured into Water/Ice (50 mL). The aqueous layer was extracted with CH₂Cl₂ (2×50 mL). The combined organic layers were washed with brine (1×50 mL), dried over anhydrous Na₂SO₄. After filtration, the filtrate was concentrated under reduced pressure. This resulted in 4-chloro-7-methylthieno[3,2-c]pyridazine (600 mg, 67.51%) as a brown solid.

### Synthesis of 7-bromothieno[3,2-c]pyridazin-4-ol (BB-3)

### Step 1

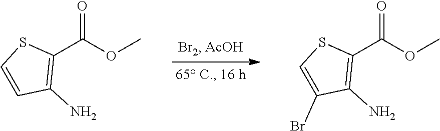

To a stirred mixture of methyl 3-aminothiophene-2-carboxylate (260 g, 1654.05 mmol, 1 equiv) in AcOH (2600 mL) was added Br₂ (528.66 g, 3308.09 mmol, 2 equiv) dropwise at room temperature under nitrogen atmosphere. The resulting mixture was stirred for overnight at 65° C. under nitrogen atmosphere. The reaction was quenched with water at 0° C. The mixture was neutralized to pH 7 with saturated NaHCO₃ (aq.). The aqueous layer was extracted with EA (5×500 mL). The resulting mixture was washed with 500 mL of brine, dried with Na₂SO₄, filtered, and concentrated under reduced pressure. The residue was purified by silica gel column chromatography, eluted with PE/EA (8:1) to afford methyl 3-amino-4-bromothiophene-2-carboxylate (120 g, 30.73%) as a yellow solid. LC-MS (ES, m/z): [M+H]⁺=236

### Step 2

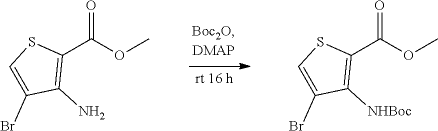 [SMILES: COC(=O)c1scc(Br)c1N ; COC(=O)c1scc(Br)c1NC(=O)OC(C)(C)C]

To a stirred mixture of methyl 3-amino-4-bromothiophene-2-carboxylate (120 g, 508.302 mmol, 1 equiv) and DMAP (12.42 g, 101.66 mmol, 0.2 equiv) in DCM (1000 mL) was added (Boc)₂O (332.81 g, 1524.90 mmol, 3 equiv) dropwise at room temperature under nitrogen atmosphere. The resulting mixture was stirred for overnight at room temperature under nitrogen atmosphere.

The resulting mixture was diluted with water (1000 mL) and extracted with DCM (5×1 L). The resulting mixture was washed with 500 mL of brine, dried with Na₂SO₄, filtered, and oncentrated under reduced pressure. The residue was purified by silica gel column chromatography, eluted with PE/EA (10:1) to afford methyl 4-bromo-3-[(tert-butoxycarbonyl)amino]thiophene-2-carboxylate (170 g, 99.48%) as a yellow solid. LC-MS (ES, m/z): [M+H]⁺=336

### Step 3

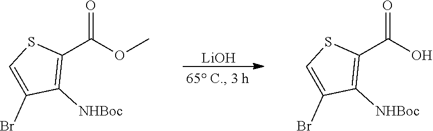 [SMILES: COC(=O)c1scc(Br)c1NC(=O)OC(C)(C)C ; "LiOH" ; CC(C)(C)OC(=O)Nc1c(Br)csc1C(=O)O]

To a stirred mixture of methyl 4-bromo-3-[(tert-butoxycarbonyl)amino]thiophene-2-carboxylate (170 g, 505.651 mmol, 1 equiv) in MeOH (630 mL), THE (630 mL) and H₂O (756 mL) was added lithium hydroxide (96.88 g, 4045.20 mmol, 8 equiv) dropwise at room temperature under nitrogen atmosphere. The resulting mixture was stirred for 3 h at 65° C. under nitrogen atmosphere. The mixture was allowed to cool down to room temperature. The mixture was acidified to pH 5 with citric acid. The aqueous layer was extracted with EA (4×1 L). The resulting mixture was washed with 500 mL of brine, dried with Na₂SO₄. The residue was purified by silica gel column chromatography, eluted with PE/EA (7:1) to afford 4-bromo-3-[(tert-butoxycarbonyl)amino]thiophene-2-carboxylic acid (130 g, 79.80%) as a light yellow oil. LC-MS (ES, m/z): [M+H]⁺=322

### Step 4

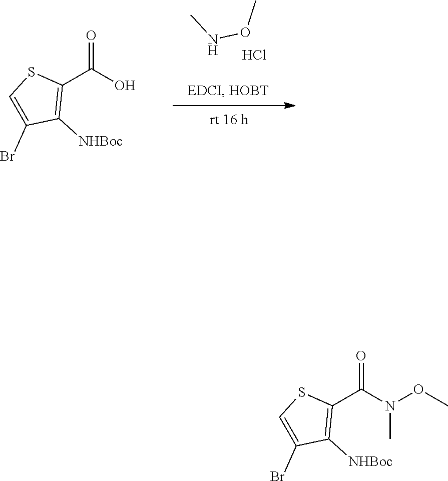

To a stirred mixture of 4-bromo-3-[(tert-butoxycarbonyl)amino]thiophene-2-carboxylic acid (120 g, 372.474 mmol, 1 equiv) and N,O-dimethylhydroxylamine hydrochloride (29.58 g, 484.21 mmol, 1.3 equiv) in DCM (1200 mL) were added EDCI (85.68 g, 446.96 mmol, 1.2 equiv), HOBT (60.40 g, 446.96 mmol, 1.2 equiv), DIEA (96.28 g, 744.94 mmol, 2 equiv) dropwise at room temperature under nitrogen atmosphere. The resulting mixture was stirred overnight at room temperature under nitrogen atmosphere. The reaction was quenched with water at room temperature. The aqueous layer was extracted with DCM (3×800 mL). The resulting mixture was washed with 500 mL of brine, dried with Na₂SO₄, filtered, and concentrated under reduced pressure. The residue was purified by silica gel column chromatography, eluted with PE/EA (5:1) to afford tert-butyl N-{4-bromo-2-[methoxy(methyl)carbamoyl]thiophen-3-yl}carbamate (110 g, 80.86%) as a light yellow solid. LC-MS [M+H]⁺=365.

### Step 5

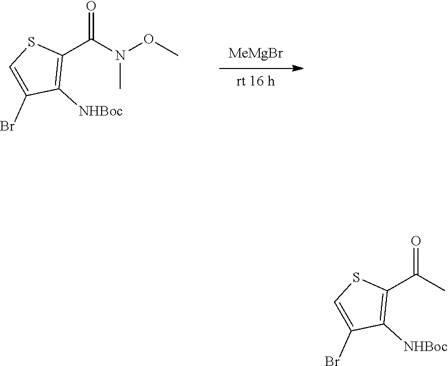 [SMILES: "MeMgBr" ; CON(C)C(=O)c1scc(Br)c1NC(=O)OC(C)(C)C ; CC(=O)c1scc(Br)c1NC(=O)OC(C)(C)C]

To a stirred mixture of tert-butyl N-{4-bromo-2-[methoxy(methyl)carbamoyl]thiophen-3-yl}carbamate (270 g, 739.24 mmol, 1 equiv) in THE (2700 mL) was added bromo(methyl)magnesium (3M in 2-MeTHF, 1480 mL, 4438 mmol, 6.0 equiv) dropwise at 0° C. under nitrogen atmosphere. The resulting mixture was stirred for overnight at room temperature under nitrogen atmosphere. The reaction was quenched with water/ice and NH₄Cl at 0° C. The aqueous layer was extracted with EA (3×600 mL). The resulting mixture was washed with 500 mL brine, dried with Na₂SO₄. The resulting mixture was concentrated under reduced pressure. The residue was purified by silica gel column chromatography, eluted with PE/EA (5:1) to afford tert-butyl N-(2-acetyl-4-bromothiophen-3-yl)carbamate (125 g, 52.81%) as a light yellow solid. LC-MS (ES, m/z): [M+H]⁺=320.

### Step 6

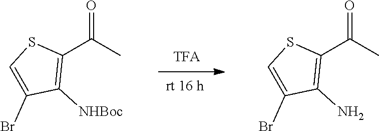

A mixture of tert-butyl N-(2-acetyl-4-bromothiophen-3-yl)carbamate (125 g, 390.381 mmol, 1 equiv) and trifluoroacetaldehyde (2500 mL, 255.04 mmol, 8.17 equiv) in DCM (2500 mL) was stirred for overnight at room temperature under nitrogen atmosphere. The reaction was quenched with water at room temperature. The aqueous layer was extracted with DCM (5×500 mL). The resulting mixture was washed with 500 mL of brine, dried with Na₂SO₄, filtered, and concentrated under reduced pressure. This resulted in 1-(3-amino-4-bromothiophen-2-yl)ethanone (75 g, 87.30%) as a brown yellow solid. The crude mixture was used in the next step directly without further purification. LC-MS (ES, m/z): [M+H]⁺=220

### Step 7

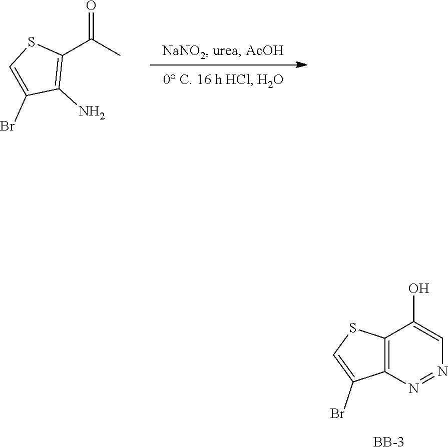

To a stirred mixture of 1-(3-amino-4-bromothiophen-2-yl)ethanone (30 g, 136.31 mmol, 1 equiv), concentrated HCl (52 mL) in AcOH (32 mL) and H₂O (76 mL, 4218.70 mmol, 30.95 equiv) were added NaNO₂ (23.5 g, 340.60 mmol, 2.50 equiv) and urea (1.88 g, 31.35 mmol, 0.23 equiv) in portions at 0° C. under nitrogen atmosphere. The resulting mixture was stirred overnight at room temperature under nitrogen atmosphere. The precipitated solids were collected by filtration and washed with EA (50 mL) to afford 7-bromothieno[3,2-c]pyridazin-4-ol (23 g, 73.02%) as a brown solid. The crude mixture was used in the next step directly without further purification. LC-MS (ES, m/z): [M+H]⁺=231

### Step 8

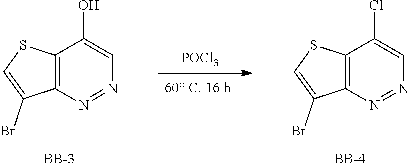

To a stirred mixture of 7-bromothieno[3,2-c]pyridazin-4-ol (2 g, 8.655 mmol, 1 equiv) and phosphorus oxychloride (2.65 g, 17.310 mmol, 2 equiv) in MeCN (20 mL) was added DIEA (2.24 g, 17.310 mmol, 2 equiv) dropwise at 0° C. under nitrogen atmosphere. The resulting mixture was stirred for overnight at 60° C. under nitrogen atmosphere. The reaction was quenched with water at room temperature. The aqueous layer was extracted with EA (200 mL). The resulting mixture was washed with 500 mL brine, dried with Na₂SO₄, filtered, and concentrated under reduced pressure. The residue was purified by silica gel column chromatography, eluted with PE/EA (6:1) to afford 7-bromo-4-chlorothieno[3,2-c]pyridazine (800 mg, 37.04%) as a brown solid.

### Synthesis of 7-chlorothieno[3,2-c]pyridazin-4-ol (BB-5)

### Step 1

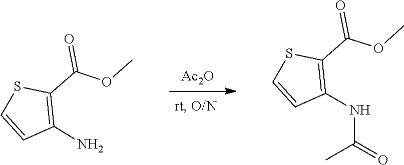

Into a 500 mL 3-necked round-bottom flask were added methyl 3-aminothiophene-2-carboxylate (140 g, 890.642 mmol, 1 equiv) and acetic anhydride (210 mL, 146.931 mmol, 2.31 equiv) at room temperature for overnight. The resulting mixture was filtered, the filter cake was washed with PE (3×300 mL). The filtrate was concentrated under reduced pressure. The reaction was quenched with water (500 mL) at room temperature. The product was precipitated by the addition of PE. to afford methyl 3-acetamidothiophene-2-carboxylate (144 g, 81.16%) as a white solid.

### Step 2

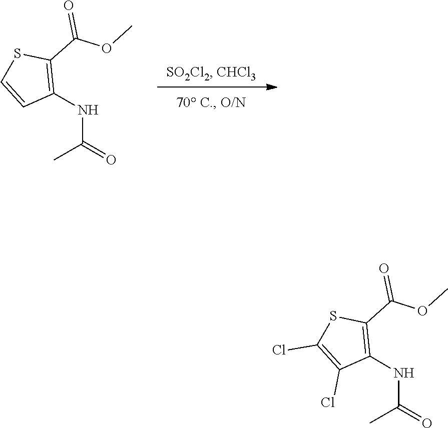

Into a 1000 mL 3-necked round-bottom flask were added methyl 3-acetamidothiophene-2-carboxylate (140 g, 702.741 mmol, 1 equiv), SO₂Cl₂ (140.28 mL, 1735.770 mmol, 2.47 equiv) and CHCl₃ (300 mL, 3719.528 mmol, 5.29 equiv) at room temperature. The solution was stirred for overnight at 70° C. The resulting mixture was concentrated under vacuum. The product was precipitated by the addition of PE. to afford 3-amino-4,5-dichlorothiophene-2-carboxylic acid (130 g, 87.24%) as a yellow solid.

### Step 3

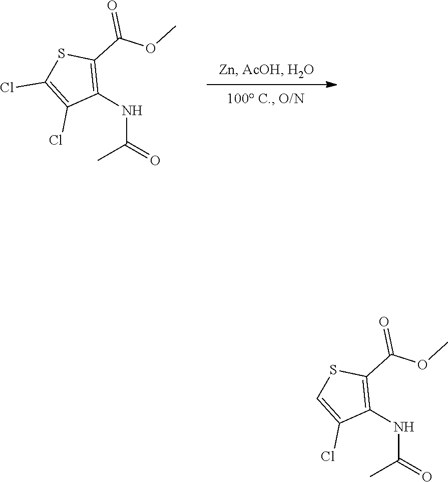

Into a 1000 mL 3-necked round-bottom flask were added methyl 4,5-dichloro-3-acetamidothiophene-2-carboxylate (70 g, 261.087 mmol, 1 equiv), H₂O (210 mL, 11656.953 mmol, 44.65 equiv), AcOH (70 mL, 1221.608 mmol, 4.68 equiv) and Zn (68.28 g, 1044.348 mmol, 4 equiv) at room temperature. A solution was stirred for overnight at 100° C. The resulting mixture was filtered, the filter cake was washed with CH₂Cl₂ (3×200 mL). The filtrate was concentrated under reduced pressure. The resulting mixture was concentrated under vacuum. to afford methyl 4-chloro-3-acetamidothiophene-2-carboxylate (50 g, 81.96%) as a white solid.

### Step 4

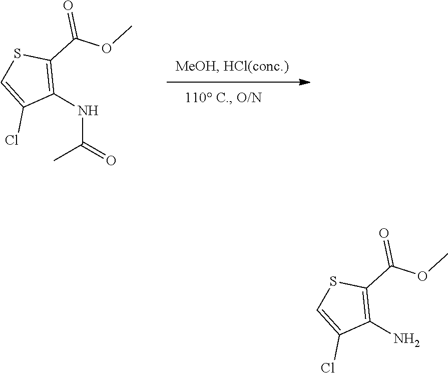

Into a 1000 mL 3-necked round-bottom flask were added methyl 4-chloro-3-acetamidothiophene-2-carboxylate (70 g, 299.568 mmol, 1 equiv), MeOH (210 mL, 5186.755 mmol, 17.31 equiv) and HCl (210 mL, 6911.684 mmol, 23.07 equiv) at room temperature. The solution was stirred for overnight at 110° C. The mixture was acidified to pH 9 with NaOH. The residue was purified by trituration with PE (1000 mL). This resulted in methyl 3-amino-4-chlorothiophene-2-carboxylate (50 g, 87.10%) as a black solid.

### Step 5

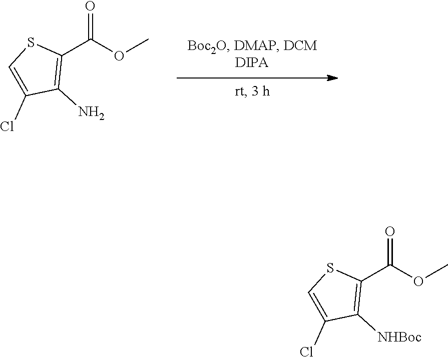 [SMILES: COC(=O)c1scc(Cl)c1N ; COC(=O)c1scc(Cl)c1NC(=O)OC(C)(C)C]

Into a 1000 mL round-bottom flask were added methyl 3-amino-4-chlorothiophene-2-carboxylate (50 g, 260.919 mmol, 1 equiv), DMAP (6.38 g, 52.184 mmol, 0.2 equiv), DCM (249.96 mL, 3932.049 mmol, 15.07 equiv), DIPA (52.81 g, 521.838 mmol, 2 equiv) and Boc₂O (85.42 g, 391.378 mmol, 1.50 equiv) at room temperature for 3 h. The reaction was quenched by the addition of water (10 mL) at room temperature. The aqueous layer was extracted with CH₂Cl₂ (3×200 mL). The residue was purified by silica gel column chromatography, eluted with PE/EA (4:1) to afford methyl 3-[(tert-butoxycarbonyl)amino]-4-chlorothiophene-2-carboxylate (39 g, 51.23%) as a yellow solid

### Step 6

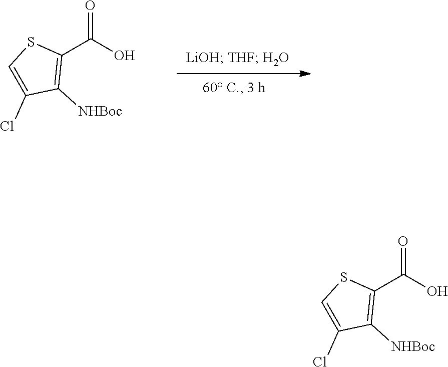 [SMILES: CC(C)(C)OC(=O)Nc1c(Cl)csc1C(=O)O ; CC(C)(C)OC(=O)Nc1c(Cl)csc1C(=O)O]

Into a 1000 mL 3-necked round-bottom flask were added methyl 3-[(tert-butoxycarbonyl)amino]-4-chlorothiophene-2-carboxylate (70 g, 239.931 mmol, 1.00 equiv), THF (80 mL, 987.421 mmol, 4.12 equiv), H₂O (250 mL, 13877.324 mmol, 57.84 equiv) and lithium hydroxide (10 g, 417.537 mmol, 1.74 equiv) at 60° C. for 3 h. The mixture was acidified to pH 7 with conc. HCl. The aqueous layer was extracted with EtOAc (3×300 mL). The residue was purified by silica gel column chromatography, eluted with PE/EA (10:1) to afford 3-[(tert-butoxycarbonyl)amino]-4-chlorothiophene-2-carboxylic acid (65 g, 97.55%) as a white solid.

### Step 7

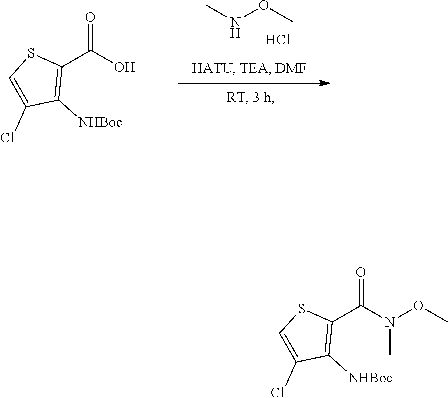

Into a 250 mL round-bottom flask were added 3-[(tert-butoxycarbonyl)amino]-4-chlorothiophene-2-carboxylic acid (36 g, 129.627 mmol, 1 equiv), N,O-dimethylhydroxylamine (8.71 g, 142.590 mmol, 1.1 equiv), hydrogen chloride (5.20 g, 142.590 mmol, 1.1 equiv), HATU (64.08 g, 168.515 mmol, 1.3 equiv), TEA (39.35 g, 388.881 mmol, 3 equiv) and DMF (100 mL, 1292.154 mmol, 9.97 equiv) at room temperature for 3 h. The resulting mixture was concentrated under vacuum. The residue was purified by silica gel column chromatography, eluted with PE/EA (10:1) to afford tert-butyl N-{4-chloro-2-[methoxy(methyl)carbamoyl]thiophen-3-yl}carbamate (18.2 g, 43.77%) as a yellow solid.

### Step 8

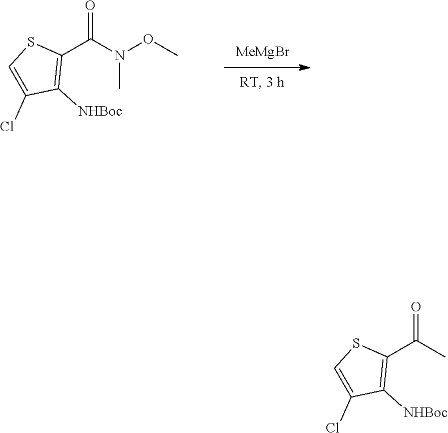 [SMILES: "MeMgBr" ; CON(C)C(=O)c1scc(Cl)c1NC(=O)OC(C)(C)C ; CC(=O)c1scc(Cl)c1NC(=O)OC(C)(C)C]

Into a 500 mL round-bottom flask were added tert-butyl N-{4-chloro-2-[methoxy(methyl)carbamoyl]thiophen-3-yl}carbamate (17 g, 52.994 mmol, 1 equiv), THE (169.98 mL, 2098.032 mmol, 39.59 equiv) and 1M bromo(methyl)magnesium (425.01 mL, 425.012 mmol, 8.02 equiv) at room temperature for 3 h. The reaction was quenched with water (10 mL) at room temperature. The aqueous layer was extracted with EtOAc (3×100 mL). The residue was purified by silica gel column chromatography, eluted with PE/EA (4:1) to afford tert-butyl N-(2-acetyl-4-chlorothiophen-3-yl)carbamate (13 g, 88.96%) as a red solid.

### Step 9

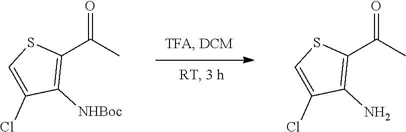

Into a 250 mL round-bottom flask were added tert-butyl N-(2-acetyl-4-chlorothiophen-3-yl)carbamate (14 g, 50.771 mmol, 1 equiv), DCM (96.92 mL, 1524.653 mmol, 30.03 equiv) and trifluoroacetaldehyde (30 mL, 306.047 mmol, 6.49 equiv) at room temperature for 3 h. The resulting mixture was concentrated under vacuum. The mixture was acidified to pH 8 with saturated NaHCO₃ (aq.). The aqueous layer was extracted with EtOAc (3×100 mL). The resulting mixture was concentrated under reduced pressure. The residue was purified by silica gel column chromatography, eluted with PE/EA (1:3) to afford 1-(3-amino-4-chlorothiophen-2-yl)ethanone (8 g, 89.72%) as a yellow solid.

### Step 10

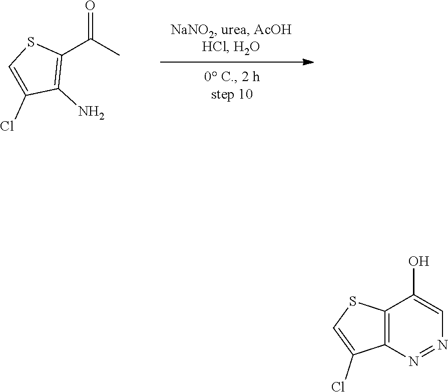

Into a 100 mL round-bottom flask were added 1-(3-amino-4-chlorothiophen-2-yl)ethanone (8 g, 45.550 mmol, 1.00 equiv), AcOH (8.01 mL, 139.838 mmol, 3.07 equiv), HCl (12.00 mL, 394.918 mmol, 8.67 equiv) and H₂O (16.00 mL, 888.225 mmol, 19.50 equiv) at 0° C. The resulting mixture was stirred for 15 min at 0° C. Into the vial was added NaNO₂ (3.14 g, 45.550 mmol, 1.00 equiv) at 0° C. with stirring for 1 h. Next, urea (410.33 mg, 6.832 mmol, 0.15 equiv) was added at 0° C. and stirred for 2 h. The resulting mixture was concentrated under vacuum. The residue was purified by reverse flash chromatography with the following conditions: column, C₁₈; mobile phase, MeCN in Water (10 mmol/L NH₄HCO₃), 0 to 10% gradient in 20 min; detector, UV 254 nm. This resulted in 7-chlorothieno[3,2-c]pyridazin-4-ol (5.6 g, 65.88%) as a red solid.

### Synthesis of tert-butyl (S)-(1-(7-methyl-4-(methylsulfonyl)thieno[3,2-c]pyridazin-6-yl)propan-2-yl)carbamate (BB-7)

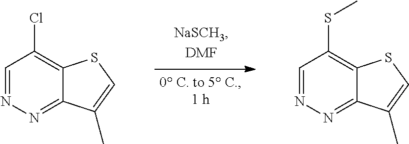

### Step 1. 7-methyl-4-(methylthio)thieno[3,2-c]pyridazine

To a stirred solution of 4-chloro-7-methylthieno[3,2-c]pyridazine (4 g, 21.664 mmol, 1 equiv) in DMF (50 mL) was added (methylsulfanyl)sodium (3.04 g, 43.328 mmol, 2 equiv) in portions at 0-5° C. The resulting mixture was stirred for 2 h at 0-5° C. and then quenched with water/ice (100 mL) thereby maintaining the temperature between 0 and 5° C. The precipitate was filtered off and washed with water providing the product as off-white solid (3 g, 71%). LCMS: m/z 197.0 [M+H]⁺

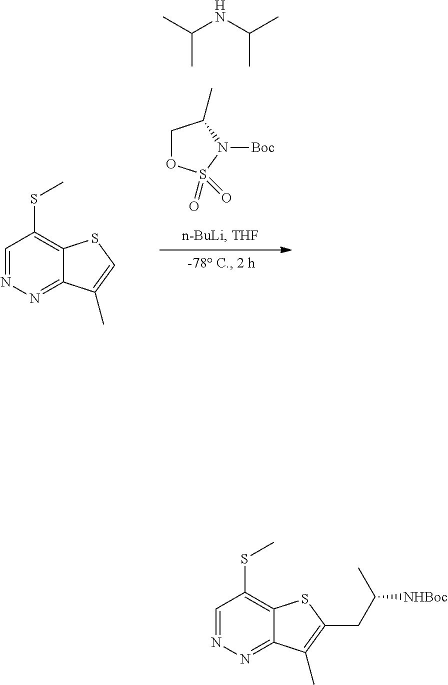

### Step 2: tert-butyl (S)-(1-(7-methyl-4-(methylthio)thieno[3,2-c]pyridazin-6-yl)propan-2-yl)carbamate

An aliquot of n-butyllithium solution (2.5 M in hexane, 8.5 mL) was added dropwise under an inert atmosphere at −60° C. to diisopropylamine (2.09 g, 20.633 mmol, 1.5 equiv) in THF (20 mL) and the solution was stirred at this temperature for 15 min. 7-methyl-4-(methylthio)thieno[3,2-c]pyridazine (2.7 g, 13.755 mmol, 1.00 equiv) in THE (58 mL) was then added dropwise and the mixture was stirred for another 20 min at −60° C. Next, a solution of tert-butyl tert-butyl 1,2,3-oxathiazolidine-3-carboxylate 2,2-dioxide (3.26 g, 13.755 mmol, 1 equiv) in THE (20 mL) was added dropwise and the mixture was stirred for further 60 min at −78° C. The reaction was quenched with water and extracted with ethyl acetate. The combined organic layers were dried and concentrated, and the residue was purified by reverse flash chromatography under the following conditions: C₁₈ column, mobile phase: 10% to 70% MeCN in 10 M aqueous NH4HCO3 solution, detector: UV 254 nm). Light yellow oil (2.2 g, 45%). LCMS: m/z 354.1 [M+H]⁺

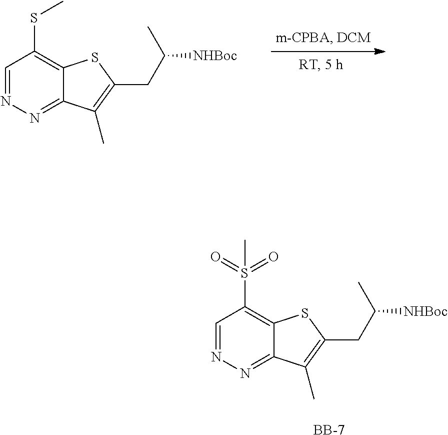

### Step 3: tert-butyl (S)-(1-(7-methyl-4-(methylsulfonyl)thieno[3,2-c]pyridazin-6-yl)propan-2-yl)carbamate (BB-7)

To a stirred mixture of tert-butyl (S)-(1-(7-methyl-4-(methylthio)thieno[3,2-c]pyridazin-6-yl)propan-2-yl)carbamate (2 g, 5.658 mmol, 1 equiv) in DCM (30 mL) was added m-CPBA (2.44 g, 14.145 mmol, 2.5 equiv) in portions at 0-5° C. The resulting mixture was stirred for 5 h at room temperature and then concentrated in vacuo. The residue was purified by reverse flash chromatography (C18 column, mobile phase: 10% to 70% MeCN in 10 M aqueous NH4HCO3 solution, detector: UV 254 nm). White solid (800 mg, 37%).

### Synthesis of tert-butyl (S)-(1-(4-chloro-7-methylthieno[3,2-c]pyridazin-6-yl)propan-2-yl)carbamate (BB-8)

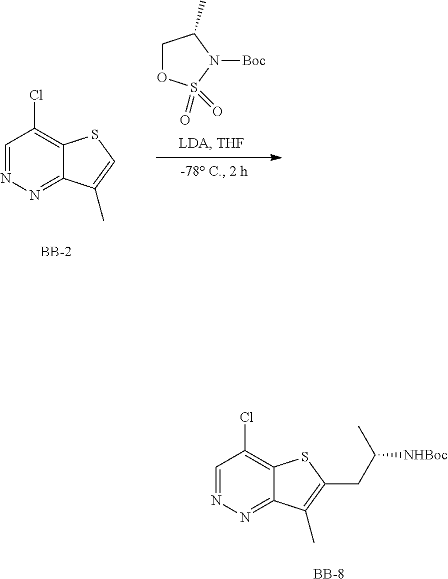

LDA (in 2M THF, 0.41 mL, 0.813 mmol, 1.5 equiv) was added dropwise at −78° C. under a nitrogen atmosphere to 4-chloro-7-methylthieno[3,2-c]pyridazine (100 mg, 0.542 mmol, 1 equiv) in THF (1 mL). The reaction mixture was stirred at −78° C. for 30 min, tert-butyl (S)-4-methyl-1,2,3-oxathiazolidine-3-carboxylate 2,2-dioxide (154.20 mg, 0.650 mmol, 1.2 equiv) in THF (1 mL) was then added dropwise and stirring was continued for another 30 mins at this temperature. The reaction mixture was quenched with water, and extracted with a blend of diethylether and EtOAc. The combined organic layers were washed with brine, dried over anhydrous Na2SO4, and concentrated. The crude product was purified by Prep-HPLC (C18 column, mobile phase A: 0.5% aqueous NH4HCO3 solution, mobile phase B: 30-80% acetonitrile) affording the title compound as purple solid (37 mg, 20%).

### Synthesis of 4-Chloro-3,7-dimethylthieno[3,2-c]pyridazine (BB-9)

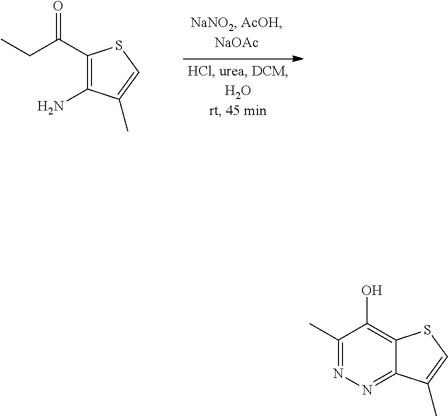

### Step 1. 3,7-Dimethylthieno[3,2-c]pyridazin-4-ol

To a stirred solution of 1-(3-amino-4-methylthiophen-2-yl)propan-1-one (6.5 g, 38.4 mmol, 1 equiv) in AcOH (1.85 g, 30.7 mmol, 0.8 equiv) and water (20 mL) was added at 0° C. dropwise concentrated aqueous HCl (37%, 8.8 mL. The reaction mixture was stirred at this temperature for 15 min, NaNO₂ (3.44 g, 49.9 mmol, 1.3 equiv) was added and stirring was continued for 1.25 h at 0° C. Urea (0.23 g, 3.8 mmol, 0.1 equiv) was then added and the mixture was stirred for additional 1 h at 0° C. Finally, NaOAc (12.60 g, 153.6 mmol, 4 equiv) and water (35 mL) were added at 0° C. and the resulting mixture was stirred for 45 min at room temperature. The precipitate was filtered off, washed with water and purified through silica gel chromatography (PE/EA=1:3) to afford the product (4.8 g, 69%) as a white solid.

### Step 2. 4-Chloro-3,7-dimethylthieno[3,2-c]pyridazine

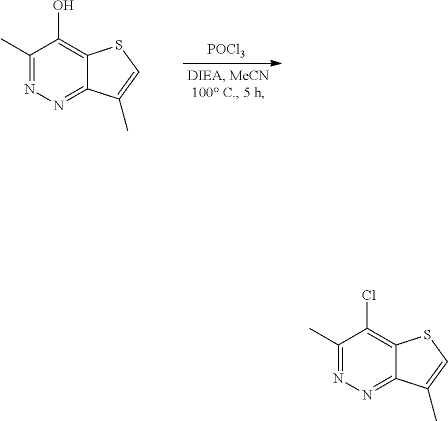

3,7-Dimethylthieno[3,2-c]-100-yridazine-4-ol (160 mg, 0.888 mmol, 1 equiv), POCl₃ (272 mg, 1.776 mmol, 2 equiv), DIEA (229 mg, 1.776 mmol, 2 equiv) and can (3 mL) were stirred for 5 h at 100° C. After hydrolysis and aqueous work up, the crude product was purified by Prep-HPLC (C₁₈, Mobile Phase A: Water/0.05% FA, Mobile Phase B: Acetonitrile 15-70%) providing the product as white solid (60 mg, 34%).

### Synthesis of 4-chloro-7-ethylthieno[3,2-c]pyridazine (BB-10)

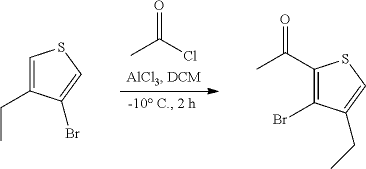

### Step 1. 1-(3-bromo-4-ethylthiophen-2-yl)ethan-1-one

A mixture of acetyl chloride (8.22 g, 104.7 mmol, 1 equiv), DCM (500 mL), AlCl₃ (27.91 g, 209.3 mmol, 2 equiv) and 3-bromo-4-ethylthiophene (20 g, 104.7 mmol, 1 equiv) was stirred for 2 h at −10° C. The mixture was acidified to pH 2 with HCl (aq.) and extracted with DCM. The combined organic layers were washed with brine, dried over anhydrous Na₂SO₄ and filtered. The solvents were removed under reduced pressure affording the product as yellow oil (20 g, 82%).

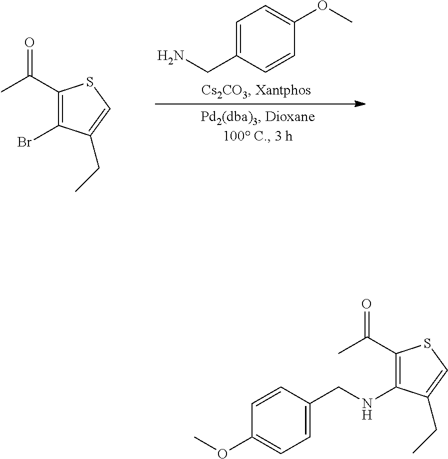

### Step 2. 1-(4-ethyl-3-((4-methoxybenzyl)amino)thiophen-2-yl)ethan-1-one

A mixture of 1-(3-bromo-4-ethylthiophen-2-yl)ethan-1-one (20 g, 85.8 mmol, 1 equiv), (4-methoxyphenyl)methanamine (11.77 g, 85.8 mmol, 1 equiv), Cs₂CO₃ (55.91 g, 171.6 mmol, 2 equiv), Xantphos (9.93 g, 17.2 mmol, 0.2 equiv) and Pd₂(dba)₃ (7.86 g, 8.6 mmol, 0.1 equiv) in dioxane (200 mL) was stirred for 3 h at 100° C. The mixture was allowed to cool to room temperature and filtered. The filter was rinsed with EtOAc and the filtrate concentrated under reduced pressure. The residue was purified by silica gel column chromatography (PE/EA=5:1) to afford the product (12 g, 48%) as yellow solid.

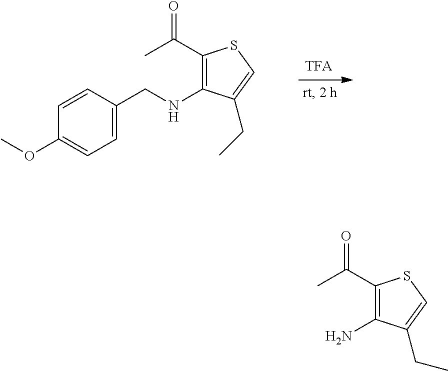 [SMILES: CCc1csc(C(C)=O)c1NCc1ccc(OC)cc1 ; CCc1csc(C(C)=O)c1N ; "TFA"]

### Step 3. 1-(3-amino-4-ethylthiophen-2-yl)ethan-1-one

1-(4-ethyl-3-((4-methoxybenzyl)amino)thiophen-2-yl)ethan-1-one (10 g, 34.6 mmol, 1 equiv) in trifluoroacetaldehyde (50 mL) was stirred for 2 h at room temperature. The excess of the aldehyde was removed under reduced pressure and the residue was purified by silica gel column chromatography (PE/EA=5:1) affording the product (5 g, 85.50%) as yellow solid.

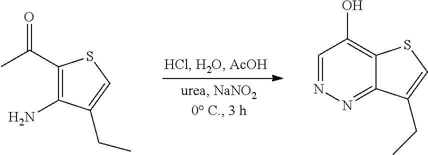

### Step 4. 7-ethylthieno[3,2-102-yridazinezin-4-ol

A mixture of 1-(3-amino-4-ethylthiophen-2-yl)ethan-1-one (5 g, 29.5 mmol, 1 equiv), AcOH (5 mL, 87.3 mmol, 2.95 equiv), conc. aqueous HCl (37%, 7.5 mL, 246.8 mmol, 8.36 equiv), water (15.00 mL, 832.6 mmol, 28.18 equiv), NaNO₂ (2.65 g, 38.4 mmol, 1.3 equiv), urea (0.18 g, 3.0 mmol, 0.1 equiv) and NaOAc (9.69 g, 118.2 mmol, 4 equiv) was stirred for 3 h at 0° C. The precipitated solid was filtered off, washed with water and further purified by silica gel column chromatography (DCM/MeOH=10:1) affording the product (4 g, 75%) as a brown solid.

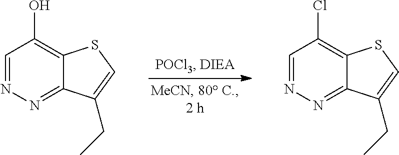

### Step 5. 4-chloro-7-ethylthieno[3,2-c]pyridazine (BB-10)

POCl₃ (6.81 g, 44.4 mmol, 2 equiv) was added at room temperature to a solution of 7-ethylthieno[3,2-102-yridazinezin-4-ol (4 g, 22.2 mmol, 1 equiv) and DIEA (5.74 g, 44.4 mmol, 2 equiv) in MeCN (20.0 mL). The resulting mixture was stirred for 2 h at 80° C., concentrated under reduced pressure and diluted with water. The mixture was extracted with DCM and the combined organic layers were washed with brine, and dried over anhydrous Na₂SO₄. The solvent was evaporated and the residue purified by silica gel column chromatography (PE/EA=10:1). Yellow solid (1.5 g, 34%).

Synthesis of tert-butyl N-{6-bromo-7-methylthieno[3,2-c]-102-yridazine-4-yl}-N-(thiophen-2-ylmethyl)carbamate (BB-11) is described in the specific examples of General Methods section below.

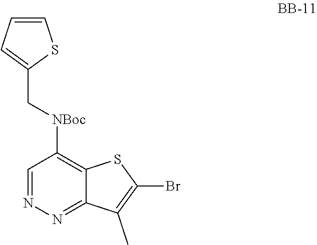 [SMILES: Cc1c(Br)sc2c(N(Cc3cccs3)C(=O)OC(C)(C)C)cnnc12]

### Synthesis of 4-chloro-3-fluoro-7-methylthieno[3,2-c]pyridazine (BB-12)

### Step 1

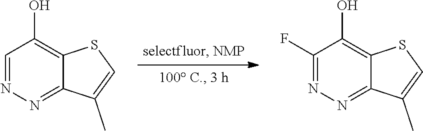

To a stirred mixture of 7-methylthieno[3,2-c]pyridazin-4-ol (3 g, 18.051 mmol, 1 equiv) in NMP (75 mL) was added Selectfluor (19.2 g, 54.197 mmol, 3.00 equiv) at room temperature under air atmosphere. The resulting mixture was stirred for 3 h at 100° C. under nitrogen atmosphere. The resulting mixture was filtered, the filter cake was washed with MeCN (1×30 mL). The filtrate was concentrated under reduced pressure. The residue was purified by reversed-phase flash chromatography with the following conditions: column, C₁₈; mobile phase, MeCN in Water (10 mmol/L NH₄HCO₃), 5% to 70% gradient in 10 min; detector, UV 254 nm. This resulted in 3-fluoro-7-methylthieno[3,2-c]pyridazin-4-ol (1.1 g, 33.09%) as a brown solid. LC-MS (ES, m/z): [M+H]⁺=185

### Step 2

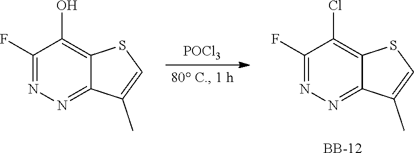

A mixture of 3-fluoro-7-methylthieno[3,2-c]pyridazin-4-ol (100 mg, 0.543 mmol, 1 equiv) and phosphorus oxychloride (0.5 mL) was combined at room temperature under air atmosphere. The resulting mixture was stirred for 1 h at 80° C. under air atmosphere. The resulting mixture was concentrated under vacuum. The reaction was quenched by the addition of water (30 mL) at room temperature. The mixture basified to pH7 with NH₃·H₂O. The aqueous layer was extracted with CH₂Cl₂ (3×30 mL). The resulting mixture was washed with 1×30 mL of water. The residue was purified by reversed-phase flash chromatography with the following conditions: column, Cis; mobile phase, MeCN in Water (10 mmol/L NH₄HCO₃), 20% to 80% gradient in 10 min; detector, UV 254 nm. This resulted in 4-chloro-3-fluoro-7-methylthieno[3,2-c]pyridazine (90 mg, 81.81%) as a light brown solid. LC-MS (ES, m/z): [M+H]⁺=203

Synthesis of tert-butyl N-{7-methylthieno[3,2-c]pyridazin-4-yl}-N-(thiophen-2-ylmethyl)carbamate (BB-13) is described in the specific examples of General Methods section below.

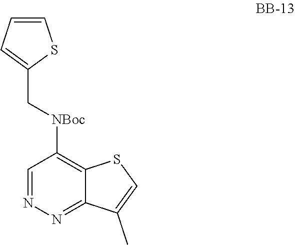 [SMILES: Cc1csc2c(N(Cc3cccs3)C(=O)OC(C)(C)C)cnnc12]

Synthesis of 6-[(2S)-2-aminopropyl]-N-[(3-methoxythiophen-2-yl)methyl]-7-methylthieno[3,2-c]pyridazin-4-amine (BB-14)) is described in the specific examples of General Methods section below.

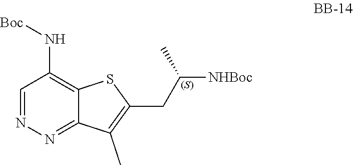

Synthesis of 4-chlorothieno[3,2-c]pyridazine (BB-15) was performed as described in WO2020/161208.

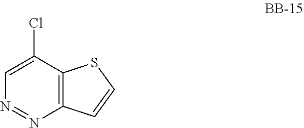

### Synthesis of 3-bromo-7-methylthieno[3,2-c]pyridazin-4-ol (BB-17)

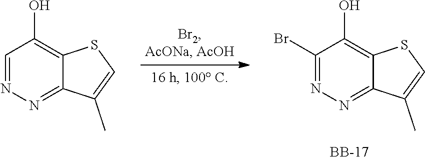

Into a 250 mL 3-necked round-bottom flask were added 7-methylthieno[3,2-c]pyridazin-4-ol (10 g, 60.168 mmol, 1 equiv), AcONa (14.81 g, 180.504 mmol, 3 equiv), AcOH (100 mL) and Br₂ (19.23 g, 120.336 mmol, 2 equiv) at room temperature. The resulting mixture was stirred for overnight at 100° C. The mixture was allowed to cool down to room temperature and quenched with water/ice (200 mL) at 0° C. The precipitated solids were collected by filtration and washed with water (3×100 mL). The resulting solid was dried in an oven under reduced pressure. This resulted in 3-bromo-7-methylthieno[3,2-c]pyridazin-4-ol (8 g, 54.25%) as a red solid.

### General Procedure for the Synthesis of Cyclic Sulfamidates AA:

Cyclic Sulfamidates can be synthesized by the following general method starting from the appropriate amino alcohol.

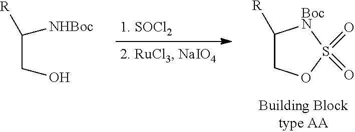

Additional synthetic procedures for making cyclic sulfamidates and/or literature references for known cyclic sulfamidates are provided below.

Reference for tert-butyl (S)-4-methyl-1,2,3-oxathiazolidine-3-carboxylate 2,2-dioxide (AA-1) Bower, J. F., Szetzo, P.; Gallagher, T. Org. Lett. 2007, 9, 3283-3286.

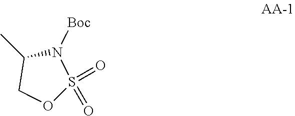

Reference for tert-butyl (R)-4-methyl-1,2,3-oxathiazolidine-3-carboxylate 2,2-dioxide (AA-2) WO2017/182495.

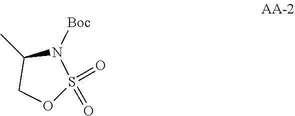

Reference for tert-butyl (R)-4-(fluoromethyl)-1,2,3-oxathiazolidine-3-carboxylate 2,2-dioxide (AA-3), WO2020/167628.

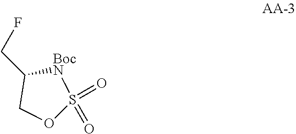 [SMILES: CC(C)(C)OC(=O)N1[C@@H](CF)COS1(=O)=O]

Reference for tert-butyl (S)-4-(((tert-butyldimethylsilyl)oxy)methyl)-1,2,3-oxathiazolidine-3-carboxylate 2,2-dioxide (AA-4) Hebeisen, P.; Weiss, U. I Alker, A. I Ataempfli, A. Tetrahedron Lett., 2011, 52, 5229-5233.

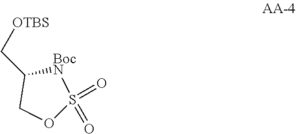 [SMILES: CC(C)(C)OC(=O)N1[C@@H](CO[Si](C)(C)C(C)(C)C)COS1(=O)=O]

Reference for tert-butyl (S)-4-(methoxymethyl)-1,2,3-oxathiazolidine-3-carboxylate 2,2-dioxide WO2017/080979.

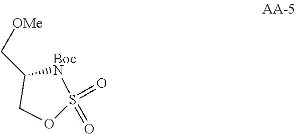 [SMILES: COC[C@H]1COS(=O)(=O)N1C(=O)OC(C)(C)C]

Reference for tert-butyl (S)-4-ethyl-1,2,3-oxthiazolidine-3-carboxylate 2,2-dioxide (AA-6) Baig, N.; Kanimozhi, C. K.; Sudhir, V. S.1 Chandrasekhan, S., Synlett, 2009, 8, 1227-1232.

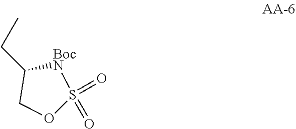 [SMILES: CC[C@H]1COS(=O)(=O)N1C(=O)OC(C)(C)C]

### Synthesis of Tert-butyl (R)-4-((S)-1-fluoroethyl)-1,2,3-oxathiazolidine-3-carboxylate 2,2-dioxide (AA-7)

### Step 1: Synthesis of 3-(tert-butyl) 4-methyl (4S,5R)-5-methyl-1,2,3-oxathiazolidine-3,4-dicarboxylate

### 2,2-dioxide

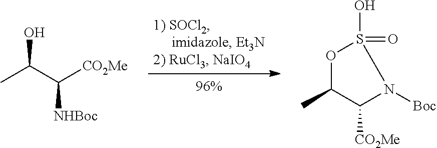

To a solution of imidazole (3.62 g, 4 Eq, 53.2 mmol), triethylamine (3.36 g, 4.63 mL, 2.5 Eq, 33.2 mmol) in DCM (60 mL) at −60° C. was added dropwise thionyl chloride (1.74 g, 1.07 mL, 1.10 Eq, 14.6 mmol) followed by methyl (tert-butoxycarbonyl)-L-threoninate (3.10 g, 1 Eq, 13.3 mmol) in DCM (30.00 mL) keeping the reaction mixture below −55° C. The turbid mixture was allowed to warm to rt slowly and stirred 30 minutes. The reaction was quenched with 0.5N HCl (150 mL) and the water layer was extracted with DCM (2×75 mL). The combined organic layers were washed with half brine and half water, dried over sodium sulfate, filtered, and concentrated in vacuo.

The crude mixture was redissolved in MeCN (50 mL), cooled to 0° C., and treated with sodium periodate (3.27 g, 1.15 Eq, 15.3 mmol) followed by ruthenium trichloride (276 mg, 88.6 μL, 0.1 Eq, 1.33 mmol) and water (50 mL). The reaction was stirred for 1 h at 0° C. and diluted with water and TBME and filtered through a pad of celite. The water layer was extracted with TBME two times. The combined organic layers were washed with brine, dried over sodium sulfate, filtered and concentrated in vacuo to afford 3-(tert-butyl) 4-methyl (4S,5R)-5-methyl-1,2,3-oxathiazolidine-3,4-dicarboxylate 2,2-dioxide (3.770 g, 12.77 mmol, 96.1%). 1H NMR (299 MHz, CDCl₃) δ 5.06-4.72 (m, 1H), 4.51 (dd, J=5.8, 1.4 Hz, 1H), 3.87 (s, 3H), 1.73 (d, J=6.4 Hz, 3H), 1.57 (s, 9H).

### Step 2: Synthesis of methyl (2R,3S)-2-((tert-butoxycarbonyl)amino)-3-fluorobutanoate

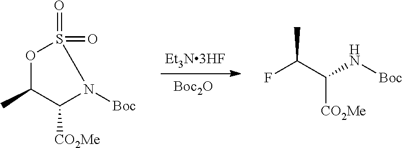 [SMILES: "Boc2O" ; COC(=O)[C@@H]1[C@@H](C)OS(=O)(=O)N1C(=O)OC(C)(C)C ; COC(=O)[C@@H](NC(=O)OC(C)(C)C)[C@H](C)F]

To a solution of 3-(tert-butyl) 4-methyl (4S,5R)-5-methyl-1,2,3-oxathiazolidine-3,4-dicarboxylate 2,2-dioxide (5.770 g, 1 Eq, 19.54 mmol) in THE (100.00 mL) was added triethylamine trihydrofluoride (20.47 g, 20.7 mL, 6.50 Eq, 127.0 mmol) and the reaction mixture was refluxed for 16 h. The reaction was neutralized with a saturated solution of sodium bicarbonate until the pH tested basic. Next, boc anhydride (4.264 g, 1 eq, 19.54 mmol) was added in one portion. The reaction mixture was stirred for 30 minutes at room temperature and extracted with EtOAc twice. The combined organic layers were washed with water and brine, dried over sodium sulfate, filtered and concentrated in vacuo. The crude material was purified by column chromatography (Heptanes/EtOAc 90/10) to afford methyl (2R,3S)-2-((tert-butoxycarbonyl)amino)-3-fluorobutanoate (3.150 g, 13.39 mmol, 68.53%). ¹H NMR (299 MHz, CDCl₃) δ 5.38 (s, 1H), 4.88 (ddd, J=47.2, 6.4, 3.7 Hz, 1H), 4.47 (dd, J=22.2, 9.0 Hz, 1H), 3.82 (s, 3H), 1.51-1.36 (m, 12H).

### Step 3: Synthesis of tert-butyl ((2R,3S)-3-fluoro-1-hydroxybutan-2-yl)carbamate

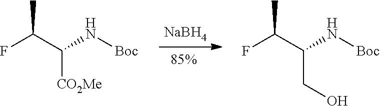 [SMILES: COC(=O)[C@@H](NC(=O)OC(C)(C)C)[C@H](C)F ; "85%" ; "NaBH4" ; C[C@H](F)[C@@H](CO)NC(=O)OC(C)(C)C]

To a solution of methyl (2R,3S)-2-((tert-butoxycarbonyl)amino)-3-fluorobutanoate (3.08 g, 1 Eq, 13.1 mmol) in EtOH (65.00 mL) at 0° C. was added NaBH₄ (1.24 g, 2.5 Eq, 32.7 mmol). The mixture was stirred at 0° C. for 8 h. The mixture was poured into 100 mL of water. The water layer was extracted with DCM (3×100 mL). The combined organic layers were washed with brine, dried over sodium sulfate, filtered and concentrated in vacuo. The crude material was purified by column chromatography (500 mL silica, Heptanes/EA, 65/35 to afford tert-butyl ((2R,3S)-3-fluoro-1-hydroxybutan-2-yl)carbamate (2.30 g, 11.1 mmol, 84.8%). ¹H NMR (299 MHz, CDCl3) δ 5.12 (s, 1H), 4.80 (dt, J=48.0, 6.1 Hz, 1H), 4.04-3.88 (m, 1H), 3.85-3.59 (m, 2H), 1.97 (s, 1H), 1.56-1.33 (m, 12H).

### Step 4: Tert-butyl (R)-4-((S)-1-fluoroethyl)-1,2,3-oxathiazolidine-3-carboxylate 2,2-dioxide

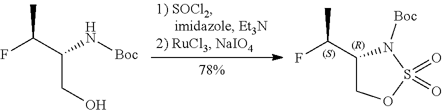

To a solution of imidazole (3.02 g, 4 Eq, 44.4 mmol), triethylamine (2.81 g, 3.87 mL, 2.5 Eq, 27.7 mmol) in DCM (60.00 mL) at −60° C. was added dropwise thionyl chloride (1.45 g, 891 μL, 1.1 Eq, 12.2 mmol) followed by tert-butyl ((2R,3S)-3-fluoro-1-hydroxybutan-2-yl)carbamate (2.30 g, 1 Eq, 11.1 mmol) in DCM (30.00 mL) while keeping the temperature of the reaction mixture below −55° C. The turbid mixture was allowed to warm to rt slowly and stirred for 30 minutes. The reaction was quenched with 0.5N HCl (150 mL) and the phases were separated. The water layer was extracted with DCM (2×75 mL). The combined organic layers washed brine (100 mL), dried over sodium sulfate, filtered, and concentrated in vacuo. To a solution of the crude material in MeCN (40.00 mL) at 0° C. was added sodium periodate (2.73 g, 1.15 Eq, 12.8 mmol) followed by ruthenium trichloride (230 mg, 74.0 μL, 0.1 Eq, 1.11 mmol) and water (40.00 mL). The reaction was stirred 1 h at 0° C. and diluted with water (50 mL) and TBME (150 mL) and filtered through a pad of celite. The celite cake was washed with 50 mL of TBME. The water layer was extracted with TBME twice (2×75 mL). The combined organic layers were washed with brine (100 mL), dried over sodium sulfate, filtered and concentrated in vacuo. The crude material was purified by column chromatography (500 mL silica, Hept/EtOAc, 80/20 to 75/25) to afford tert-butyl (R)-4-((S)-1-fluoroethyl)-1,2,3-oxathiazolidine-3-carboxylate 2,2-dioxide (2.32 g, 8.62 mmol, 77.6%) as a white solid. ¹H NMR (299 MHz, CDCl₃) δ 4.95 (d of quintets, J=47.5, 6.3 Hz, 1H), 4.79-4.56 (m, 2H), 4.33 (dtd, J=13.7, 5.5, 2.5 Hz, 1H), 1.58 (s, 9H), 1.43 (d, J=24.1, 6.4, 1.1 Hz, 3H).

### Synthesis of tert-butyl (R)-4-((R)-1-fluoroethyl)-1,2,3-oxathiazolidine-3-carboxylate 2,2-dioxide (AA-8)

### Step 1. Synthesis of tert-butyl ((2R,3S)-1,3-dihydroxybutan-2-yl)carbamate

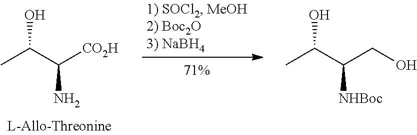

To a suspension of L-allothreonine (22.00 g, 97.00% Wt, 1 Eq, 179.1 mmol) in MeOH (500.00 mL) at 0° C., thionyl chloride (85.25 g, 52.30 mL, 4 Eq, 716.6 mmol) was added in a slow stream. The reaction mixture was allowed to warm to rt and refluxed for 3 h and concentrated in vacuo. The crude material was diluted with DCM (500.00 mL) and Boc₂O (39.10 g, 41.2 mL, 1 Eq, 179.1 mmol) was added followed by triethylamine (54.38 g, 74.9 mL, 3 Eq, 537.4 mmol). The reaction mixture was stirred at rt for 90 minutes, washed with water twice and also brine, dried over sodium sulfate, filtered and concentrated in vacuo to afford methyl (tert-butoxycarbonyl)-L-allothreoninate (39.140 g, 167.80 mmol, 93.66%). To a solution of methyl (tert-butoxycarbonyl)-L-allothreoninate (39.140 g, 1 Eq, 167.80 mmol) in EtOH (650.00 mL) at 0° C. was added NaBH4 (12.70 g, 2 Eq, 335.59 mmol). The reaction mixture was stirred at 0° C. overnight.

The reaction was quenched with water (400 mL) and diluted with DCM (500 mL). The mixture was stirred vigorously for 5 minutes. The phases were separated and the water layer was thoroughly extracted with DCM/EtOH 10% three times (3×750 mL). The combined organic layers were washed with brine (500 mL) dried over sodium sulfate, filtered and concentrated in vacuo to afford tert-butyl ((2R,3S)-1,3-dihydroxybutan-2-yl)carbamate (26.11 g, 127.2 mmol, 75.81%). ¹H NMR (299 MHz, CDCl₃) δ 5.57-5.13 (m, 1H), 4.10-3.93 (m, 2H), 3.87-3.66 (m, 1H), 3.51 (s, 1H), 2.62 (s, 2H), 1.47 (s, 9H), 1.31 (d, J=6.4 Hz, 3H).

### Step 2, Synthesis of Tert-butyl ((2R,3S)-1-(benzyloxy)-3-hydroxybutan-2-yl)carbamate

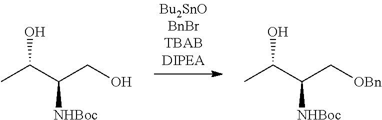 [SMILES: C[C@H](O)[C@@H](CO)NC(=O)OC(C)(C)C ; C[C@H](O)[C@@H](COCc1ccccc1)NC(=O)OC(C)(C)C]

A mixture of dibutyltin oxide (2.814 g, 0.1 Eq, 11.30 mmol), tetrabutylammonium bromide (10.93 g, 0.30 Eq, 33.91 mmol), DIPEA (58.44 g, 78.8 mL, 4 Eq, 452.1 mmol) and benzyl bromide (77.33 g, 53.78 mL, 4 Eq, 452.1 mmol) and tert-butyl ((2R,3S)-1,3-dihydroxybutan-2-yl)carbamate (23.2 g, 113 mmol) was stirred neat at 76° C. for 18 h under vigorous stirring. The reaction was cooled to room temperature and diluted with EtOAc and water. The water layer was extracted twice with EtOAc. The combined organic layers were washed with brine, dried over sodium sulfate, filtered and concentrated in vacuo. Purification by column chromatography (H/EA, 70/30) afforded tert-butyl ((2R,3S)-1-(benzyloxy)-3-hydroxybutan-2-yl)carbamate. ¹H NMR (299 MHz, CDCl₃) δ 7.51-7.31 (m, 5H), 5.28 (s, 1H), 4.64-4.48 (m, 2H), 3.96-3.77 (m, 2H), 3.73-3.56 (m, 2H), 2.92 (d, J=8.2 Hz, 1H), 1.47 (s, 9H), 1.25 (d, 3H).

### Step 3. Synthesis of tert-butyl (4R,5S)-4-((benzyloxy)methyl)-5-methyl-1,2,3-oxathiazolidine-3-carboxylate 2,2-dioxide

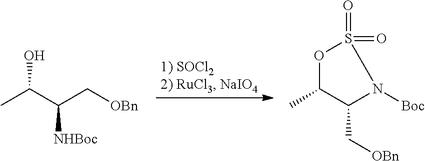 [SMILES: C[C@H](O)[C@@H](COCc1ccccc1)NC(=O)OC(C)(C)C ; C[C@@H]1OS(=O)(=O)N(C(=O)OC(C)(C)C)[C@@H]1COCc1ccccc1]

To a solution of imidazole (18.81 g, 4 Eq, 276.3 mmol) and triethylamine (17.47 g, 24.1 mL, 2.5 Eq, 172.7 mmol) in DCM (550.00 mL) at −60° C. was added thionyl chloride (9.448 g, 5.796 mL, 1.15 Eq, 79.42 mmol) dropwise keeping the reaction mixture below −55° C. Next, a solution of tert-butyl ((2R,3S)-1-(benzyloxy)-3-hydroxybutan-2-yl)carbamate (20.40 g, 1 Eq, 69.06 mmol) in DCM (200.00 mL) was added dropwise keeping the reaction mixture below −55° C. and the reaction mixture was allowed to warm to rt slowly. The reaction was quenched with 0.5N HCl (1 L). The phases were separated and the acidic layer was extracted with DCM (2×500 mL). The combined organic layers were washed with brine (300 mL), dried over sodium sulfate, filtered and concentrated in vacuo. To a solution of the crude material in acetonitrile (200.00 mL) at 0° C. was added sodium periodate (16.99 g, 1.15 Eq, 79.42 mmol) followed by ruthenium trichloride (1.433 g, 0.1 Eq, 6.906 mmol) and water (200.00 mL). The mixture was stirred at 0° C. for 45 minutes and monitored by NMR for full conversion. The reaction was diluted with water (200 mL) and TBME (300 mL), and filtered through a pad of celite. The water layer was extracted with TBME (3×400 mL). The combined organic layers were washed with brine (300 mL), dried over sodium sulfate, filtered and concentrated in vacuo. The crude material was purified over a short plug of silica, eluting with TBME to afford tert-butyl (4R,5S)-4-((benzyloxy)methyl)-5-methyl-1,2,3-oxathiazolidine-3-carboxylate 2,2-dioxide (24.38 g, 68.21 mmol, 98.77%). ¹H NMR (299 MHz, CDCl₃) δ 7.48-7.31 (m, 5H), 5.24-4.97 (m, 1H), 4.58 (s, 2H), 4.39-4.26 (m, 1H), 3.85 (dd, J=10.2, 7.4 Hz, 1H), 3.67 (dd, J=10.3, 2.9 Hz, 1H), 1.81-1.40 (m, 12H).

### Step 5. Synthesis of tert-butyl ((2R,3R)-1-(benzyloxy)-3-fluorobutan-2-yl)carbamate

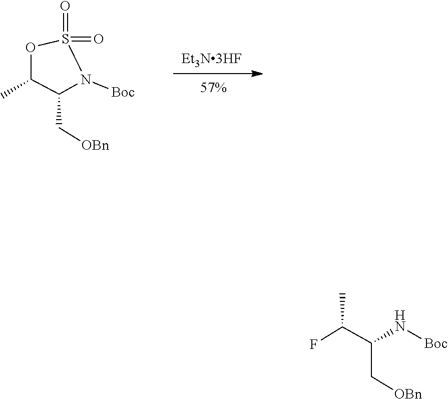 [SMILES: C[C@@H]1OS(=O)(=O)N(C(=O)OC(C)(C)C)[C@@H]1COCc1ccccc1 ; C[C@@H](F)[C@@H](COCc1ccccc1)NC(=O)OC(C)(C)C]

To a solution of tert-butyl (4R,5S)-4-((benzyloxy)methyl)-5-methyl-1,2,3-oxathiazolidine-3-carboxylate 2,2-dioxide (16.30 g, 1 Eq, 45.60 mmol) in toluene (250.00 mL) was added triethylamine trihydrofluoride (47.79 g, 48.3 mL, 6.5 Eq, 296.4 mmol) and the reaction was refluxed and stopped after 2.5 hours. The reaction was cooled to rt and the phases were separated. The bottom layer extracted twice with toluene (2×250 mL). The combined organic layers were washed with saturated sodium bicarbonate and brine, dried over sodium sulfate, filtered and concentrated in vacuo to afford 9 g of crude material. The crude was purified by column chromatography (Heptane/EA, 90/10) to afford tert-butyl ((2R,3R)-1-(benzyloxy)-3-fluorobutan-2-yl)carbamate (6.360 g, 21.39 mmol, 46.90%). Additional material could be obtained as follows: further elution of the chromatography column afforded des-boc sulfamidate (Heptane/EA, 65/35). The initial aqueous layer was diluted with EtOAc (250 mL) and basified with a saturated sodium bicarbonate solution. The water layer was extracted twice with EtOAc (2×500 mL) and the combined organic layers washed with saturated sodium bicarbonate and brine, dried over sodium sulfate, filtered and concentrated in vacuo to afford 3.8 g of a mixture of compounds. The mixture was reprotected with Boc₂O and 1.57 g of additional desired product was isolated after column chromatography together with 1.7 g of de-boc sulfamidate which gave 2.36 g of boc-protected sulfamidate that was reconverted again to the desired product as described above. In total, 11.5 g of material were obtained starting from 24.4 g of starting material (57% yield). ¹H NMR (299 MHz, CDCl₃) δ 7.44-7.29 (m, 5H), 5.07-4.83 (m, 1H), 4.76 (d, J=9.3 Hz, 1H), 4.55 (d, J=2.0 Hz, 2H), 3.97-3.76 (m, 1H), 3.61-3.38 (m, 2H), 1.47 (s, 9H), 1.37 (dd, J=24.4, 6.4 Hz, 3H).

### Step 6. Tert-butyl ((2R,3R)-3-fluoro-1-hydroxybutan-2-yl)carbamate

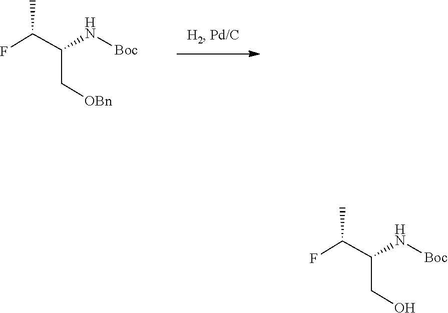 [SMILES: C[C@@H](F)[C@@H](COCc1ccccc1)NC(=O)OC(C)(C)C ; C[C@@H](F)[C@@H](CO)NC(=O)OC(C)(C)C]

To a solution of tert-butyl ((2R,3R)-1-(benzyloxy)-3-fluorobutan-2-yl)carbamate (11.50 g, 1 Eq, 38.67 mmol) in EtOAc (250.00 mL) was added PdOH₂ (1.20 g, 0.221 Eq, 8.55 mmol) and the reaction mixture was stirred under an Hydrogen gas overnight. The reaction was deemed complete by NMR. The mixture was filtered over celite and the cake was washed twice with EtOAc (2×200 mL) and concentrated in vacuo. The crude material was purified over column chromatography (Heptanes/EA, 60/40) tp afford a total 5.80 g of tert-butyl ((2R,3R)-3-fluoro-1-hydroxybutan-2-yl)carbamate ¹H NMR (299 MHz, CDCl₃) δ 5.24-4.56 (m, 2H), 3.90-3.48 (m, 3H), 2.09 (d, J=14.2 Hz, 1H), 1.48 (s, 9H), 1.40 (dd, J=24.7, 6.4 Hz, 3H).

### Step 7. Synthesis of tert-butyl (R)-4-((R)-1-fluoroethyl)-1,2,3-oxathiazolidine-3-carboxylate 2,2-dioxide

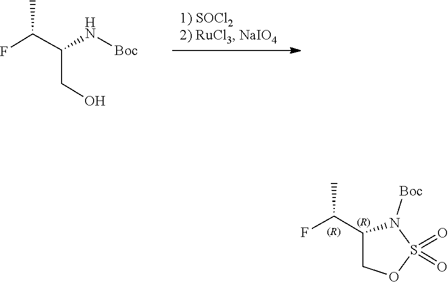

To a solution of imidazole (7.62 g, 4 Eq, 112 mmol), triethylamine (7.08 g, 9.75 mL, 2.5 Eq, 70.0 mmol) in DCM (160 mL) at −60° C. was added dropwise thionyl chloride (3.99 g, 2.45 mL, 1.2 Eq, 33.6 mmol) followed by tert-butyl ((2R,3R)-3-fluoro-1-hydroxybutan-2-yl)carbamate (5.80 g, 1 Eq, 28.0 mmol) in DCM (60 mL) keeping the reaction mixture below −55° C. The turbid mixture was allowed to warm to rt slowly and stirred for 30 minutes. The reaction was quenched with 0.5N HCl (500 mL). The phashes were separated and the water layer was extracted with DCM (2×200 mL). The combined organic layers were washed with brine (250 mL), dried over sodium sulfate, filtered, and concentrated in vacuo.

To a solution of the crude material in acetonitrile (60 mL) at 0° C. was added sodium periodate (6.88 g, 1.15 Eq, 32.2 mmol) followed by ruthenium trichloride (580 mg, 187 μL, 0.1 Eq, 2.80 mmol) and water (60 mL). The mixture was stirred at 0° C. for 45 minutes. Full conversion was noted by NMR. The reaction was diluted with water (200 mL) and TBME (300 mL), filtered over celite. The water layer was extracted with TBME (3×400 mL). The combined organic layers were washed with brine (300 mL), dried over sodium sulfate, filtered and concentrated in vacuo. Crude material purified by column chromatography (500 mL silica, Hept/EtOAc, 80/20 to 75/25) to afford tert-butyl (R)-4-((R)-1-fluoroethyl)-1,2,3-oxathiazolidine-3-carboxylate 2,2-dioxide (5.46 g, 20.3 mmol, 72.5%) as a white solid.

¹H NMR (299 MHz, CDCl₃ δ 5.23-4.88 (m, 1H), 4.69-4.64 (m, 2H), 4.61-4.51 (m, 1H), 1.58 (s, 9H), 1.48 (dd, J=24.4, 6.5 Hz, 3H).

Reference for tert-butyl (S)-4-(cyclopropylmethyl)-1,2,3-oxathiazolidine-3-carboxylate 2,2-dioxide (AA-9) WO2022/042657.

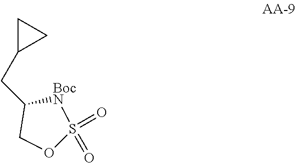 [SMILES: CC(C)(C)OC(=O)N1[C@@H](CC2CC2)COS1(=O)=O]

### Synthesis of tert-butyl 4-[(1-fluorocyclopropyl)methyl]-2,2-dioxo-1,2lambda6,3-oxathiazolidine-3-carboxylate (AA-10)

### Step 1

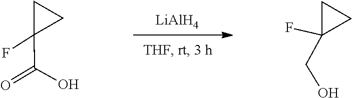 [SMILES: O=C(O)C1(F)CC1 ; "LiAlH4" ; OCC1(F)CC1]

To a stirred solution of 1-fluorocyclopropane-1-carboxylic acid (15 g, 144.120 mmol, 1 equiv) in THF (150 mL) was added LiAlH₄ in THF (78 mL, 158.532 mmol, 2M) dropwise at 0° C. under nitrogen atmosphere. The resulting mixture was stirred for 3 h at room temperature under nitrogen atmosphere. The reaction was quenched by the addition of water (2 mL) and 1N NaOH (2 mL) at 0° C. The resulting mixture was filtered, the filter cake was washed with THF (3×100 mL), dried over anhydrous Na₂SO₄, filtered, and concentrated under reduced pressure (temperature not to surpass 20° C.) to afford 1-fluorocyclopropyl)methanol (12 g, 92.42%) as a light yellow oil.

### Step 2

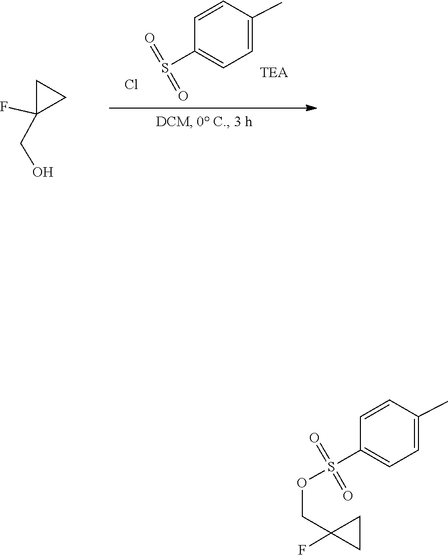

A solution of (1-fluorocyclopropyl)methanol (14 g, 155.388 mmol, 1 equiv) in DCM (150 mL) was treated with TEA (31.45 g, 310.776 mmol, 2 equiv) for 10 min at −10° C. under nitrogen atmosphere followed by the addition of P-toluenesulfonyl chloride (59.25 g, 310.776 mmol, 2 equiv) in DCM (20 mL) dropwise −10° C. The resulting mixture was stirred for additional 3 h at 0° C. The reaction was quenched with 50 mL water at room temperature. The resulting mixture was extracted with DCM (3×500 mL). The combined organic layers were washed with brine (1×10 mL), dried over anhydrous Na₂SO₄. After filtration, the filtrate was concentrated under reduced pressure. The residue was purified by silica gel column chromatography, eluted with PE/EA (5:1) to afford (1-fluorocyclopropyl)methyl 4-methylbenzenesulfonate (17 g, 44.79%) as a colorless oil.

¹H NMR (400 MHz, DMSO-d6) δ 7.84-7.79 (m, 2H), 7.52-7.46 (m, 2H), 4.38 (d, J=22.8 Hz, 2H), 2.43 (s, 3H), 1.11-1.01 (m, 2H), 0.82-0.74 (m, 2H).

### Step 3

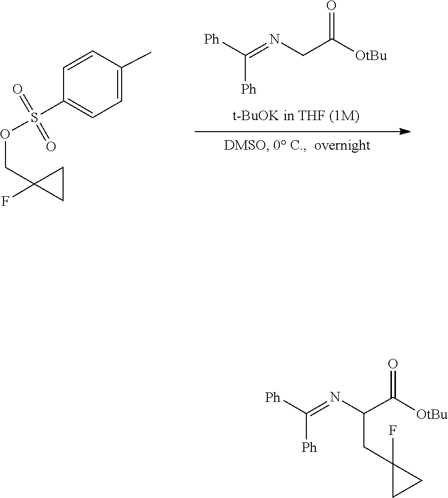 [SMILES: Cc1ccc(S(=O)(=O)OCC2(F)CC2)cc1 ; CC(C)(C)OC(=O)CN=C(c1ccccc1)c1ccccc1 ; CC(C)(C)OC(=O)C(CC1(F)CC1)N=C(c1ccccc1)c1ccccc1]

To a stirred solution of (1-fluorocyclopropyl)methyl 4-methylbenzenesulfonate (9 g, 36.843 mmol, 1 equiv) in DMSO (50 mL) was added t-BuOK (4.42 g, 39.422 mmol, 1.07 equiv) in DMSO (50 mL) dropwise at 15° C. under nitrogen atmosphere. The resulting mixture was stirred for additional 30 min at room temperature. To the above mixture was added tert-butyl 2-[(diphenylmethylidene)amino]acetate (12.41 g, 42.001 mmol, 1.14 equiv) in DMSO (50 mL) dropwise in 5 min at −15° C. The resulting mixture was stirred for 16 h at room temperature. The reaction was quenched with water (200 mL) at room temperature. The resulting mixture was extracted with EtOAc (3×400 mL) and the combined organic layers were washed with brine (2×100 mL) and dried over anhydrous Na₂SO₄. After filtration, the filtrate was concentrated under reduced pressure. The residue was purified by silica gel column chromatography, eluted with PE/EA (5:1) to afford tert-butyl 2-[(diphenylmethylidene)amino]-3-(1-fluorocyclopropyl)propanoate (12 g, 88.64%) as a light yellow solid.

### Step 4

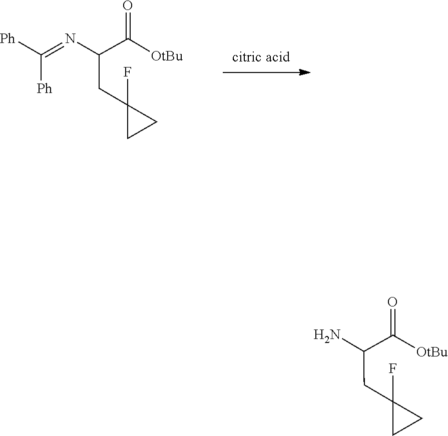 [SMILES: CC(C)(C)OC(=O)C(CC1(F)CC1)N=C(c1ccccc1)c1ccccc1 ; CC(C)(C)OC(=O)C(N)CC1(F)CC1]

To a stirred solution of tert-butyl 2-[(diphenylmethylidene)amino]-3-(1-fluorocyclopropyl)propanoate (14 g, 38.099 mmol, 1 equiv) in THF (170 mL) was added 2-hydroxypropane-1,2,3-tricarboxylic acid (731.97 mg, 3.810 mmol, 0.1 equiv) in H₂O (80 mL) dropwise at 0° C. under nitrogen atmosphere. The resulting mixture was stirred for additional 2 h at room temperature. The resulting mixture was concentrated under reduced pressure. The residue was dissolved in H₂O (200 mL) and the mixture was acidified to pH 2 with 1M HCl. The resulting mixture was washed with 2×100 mL of EtOAc and the residue was basified to pH 8 with saturated K₂CO₃ (aq.). The resulting mixture was extracted with EtOAc (3×200 mL). The combined organic layers were washed with brine (2×50 mL), dried over anhydrous Na₂SO₄. After filtration, the filtrate was concentrated under reduced pressure. This resulted in tert-butyl 2-amino-3-(1-fluorocyclopropyl)propanoate (5.5 g, 71.02%) as a light yellow solid.

### Step 5

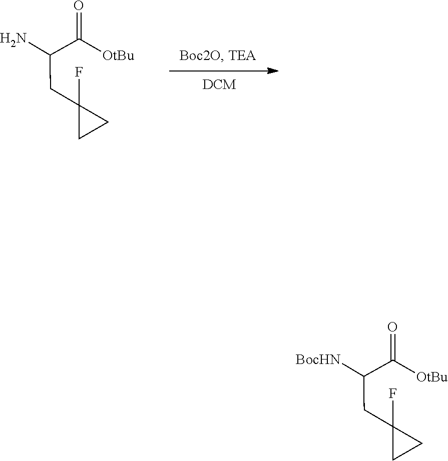 [SMILES: CC(C)(C)OC(=O)C(N)CC1(F)CC1 ; CC(C)(C)OC(=O)NC(CC1(F)CC1)C(=O)OC(C)(C)C]

To a stirred mixture of tert-butyl 2-amino-3-(1-fluorocyclopropyl)propanoate (4.6 g, 22.631 mmol, 1 equiv) in DCM (50 mL) was added (Boc)₂O (7.41 g, 33.947 mmol, 1.5 equiv) and TEA (3.44 g, 33.947 mmol, 1.5 equiv) dropwise at 0° C. under nitrogen atmosphere. The resulting mixture was stirred for additional overnight at room temperature. The resulting mixture was concentrated under reduced pressure. The residue was purified by silica gel column chromatography, eluted with PE/EA (1:1) to afford tert-butyl 2-[(tert-butoxycarbonyl)amino]-3-(1-fluorocyclopropyl)propanoate (5.68 g, 82.73%) as a colour-less oil.

¹H NMR (300 MHz, DMSO-d6) δ 7.13 (d, J=8.1 Hz, 1H), 4.06 (dtd, J=14.2, 8.0, 7.1, 4.0 Hz, 1H), 2.32 (td, J=15.7, 5.1 Hz, 1H), 1.85 (ddd, J=28.1, 14.9, 9.5 Hz, 1H), 1.40 (d, J=2.5 Hz, 18H), 0.99-0.42 (m, 4H).

### Step 6

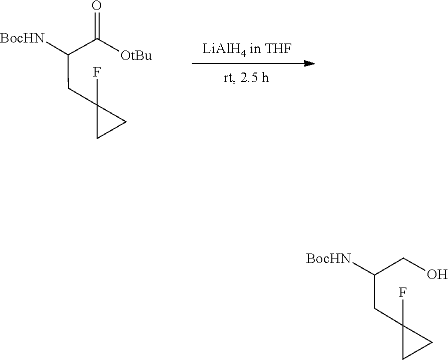 [SMILES: CC(C)(C)OC(=O)NC(CC1(F)CC1)C(=O)OC(C)(C)C ; CC(C)(C)OC(=O)NC(CO)CC1(F)CC1]

To a stirred solution tert-butyl 2-[(tert-butoxycarbonyl)amino]-3-(1-fluorocyclopropyl)propanoate (2.5 g, 8.241 mmol, 1 equiv) in THF (50 mL) was added a mixture of LiAlH₄ (0.63 g, 16.482 mmol, 2 equiv) in 16 mL THE dropwise at 0° C. under nitrogen atmosphere. The resulting mixture was stirred for additional 2.5 h at room temperature. The reaction was quenched by the addition of water (1 mL), 1 mol/L NaOH (1 mL) at 0° C. The precipitated solids were collected by filtration and washed with THE (2×50 mL). The resulting mixture was concentrated under reduced pressure. The residue was purified by silica gel column chromatography, eluted with PE/EA (1:1) to afford tert-butyl N-[1-(1-fluorocyclopropyl)-3-hydroxypropan-2-yl]carbamate (1.1 g, 57.22%) as an off-white solid. ¹H NMR (300 MHz, Chloroform-d) δ 5.01 (s, 1H), 4.00-3.86 (m, 1H), 3.80 (d, J=4.4 Hz, 2H), 2.03-1.86 (m, 2H), 1.47 (s, 9H), 1.14-1.01 (m, 2H), 0.62 (ddt, J=8.2, 5.7, 3.4 Hz, 2H).

### Step 7

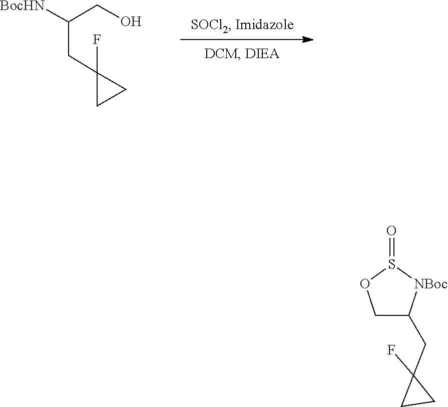 [SMILES: CC(C)(C)OC(=O)NC(CO)CC1(F)CC1 ; CC(C)(C)OC(=O)N1C(CC2(F)CC2)COS1=O]

To a solution of imidazole (2.07 g, 30.348 mmol, 4 equiv) in DCM (53 mL) were added SOCl₂ (0.83 mL, 11.380 mmol, 1.5 equiv) followed by DIEA (2.64 mL, 15.174 mmol, 2 equiv) dropwise at 0° C. To the above mixture was added tert-butyl N-[1-(1-fluorocyclopropyl)-3-hydroxypropan-2-yl]carbamate (1.77 g, 7.587 mmol, 1 equiv) dissolved in DCM (10 mL) dropwise at 0° C. The resulting mixture was stirred for additional 1 h at room temperature. The resulting mixture was washed with 3×50 mL of conc. HCl (1M). The resulting mixture was concentrated under vacuum to afford tert-butyl 4-[(1-fluorocyclopropyl)methyl]-2-oxo-1,2lambda4,3-oxathiazolidine-3-carboxylate (2.1 g, 99.09%) as a yellow oil.

### Step 8

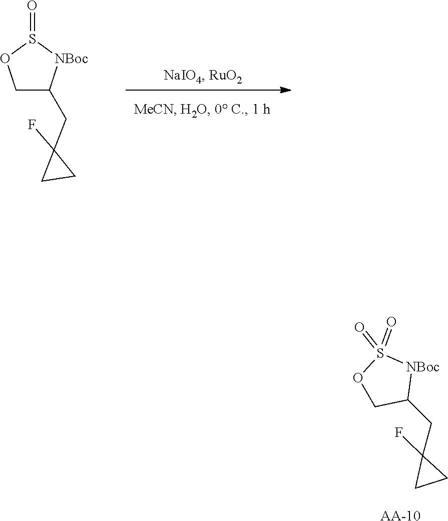 [SMILES: CC(C)(C)OC(=O)N1C(CC2(F)CC2)COS1=O ; CC(C)(C)OC(=O)N1C(CC2(F)CC2)COS1(=O)=O ; "AA-10"]

To a solution of tert-butyl 4-[(1-fluorocyclopropyl)methyl]-2-oxo-1,2lambda4,3-oxathiazolidine-3-carboxylate (2.1 g, 7.518 mmol, 1 equiv) in MeCN (28 mL) and H₂O (15 mL) was added NaIO₄ (1.93 g, 9.022 mmol, 1.2 equiv) and ruthenium(iv) oxide hydrate (22.72 mg, 0.150 mmol, 0.02 equiv) at 0° C. The resulting mixture was stirred for 1 h at 0° C. The resulting mixture was filtered through a Celite pad. The filtrate was extracted with EtOAc (3×40 mL). The combined organic layers were washed with brine (40 mL), dried over anhydrous Na₂SO₄. After filtration, the filtrate was concentrated under reduced pressure. The residue was purified by silica gel column chromatography, eluted with PE/EA (3:1) to afford tert-butyl 4-[(1-fluorocyclopropyl)methyl]-2,2-dioxo-1,2lambda6,3-oxathiazolidine-3-carboxylate (1.1 g, 49.54%) as a white solid.

### Synthesis of tert-butyl (4S)-4-(cyclopropoxymethyl)-2,2-dioxo-1,2lambda6,3-oxathiazolidine-3-carboxylate (AA-11)

### Step 1

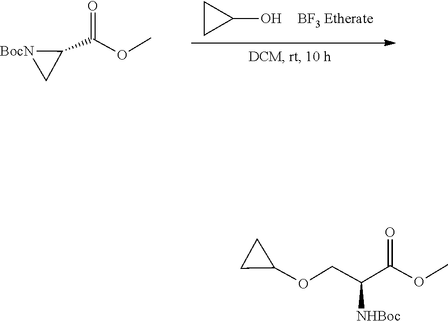

To a stirred mixture of 1-tert-butyl 2-methyl (2S)-aziridine-1,2-dicarboxylate vanadium (500 mg, 1.983 mmol, 1 equiv) and cyclopropanol (426.10 mg, 7.337 mmol, 3.7 equiv) in DCM (5 mL) was added BF₃·Et₂O (28.14 mg, 0.198 mmol, 0.1 equiv) dropwise at 0° C. under nitrogen atmosphere. The resulting mixture was stirred for overnight at room temperature under nitrogen atmosphere. The resulting mixture was diluted with water (5 mL). The resulting mixture was extracted with CH₂Cl₂ (3×50 mL). The combined organic layers were washed with brine (1×30 mL), dried over anhydrous Na₂SO₄. After filtration, the filtrate was concentrated under reduced pressure. The residue was purified by reversed-phase flash chromatography with the following conditions: column, C₁₈ silica gel; mobile phase, MeCN in Water (10 mmol/L NH₄HCO₃), 30% to 100% gradient in 15 min; detector, UV 190 nm. This resulted in methyl (2S)-2-[(tert-butoxycarbonyl)amino]-3-cyclopropoxypropanoate (200 mg, 38.90%) as a yellow oil.

### Step 2

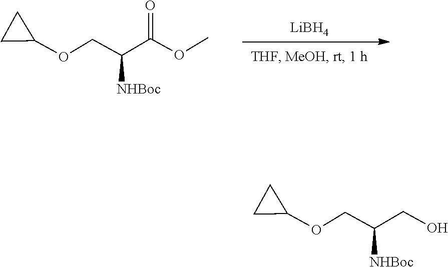 [SMILES: COC(=O)[C@H](COC1CC1)NC(=O)OC(C)(C)C ; "LiBH4" ; CC(C)(C)OC(=O)N[C@H](CO)COC1CC1]

To a stirred solution of methyl (2S)-2-[(tert-butoxycarbonyl)amino]-3-cyclopropoxypropanoate (1.8 g, 6.942 mmol, 1 equiv) in tetrahydrofuran (20 mL) was added LiBH₄ (453.57 mg, 20.82 mmol, 3 equiv) dropwise at 0° C. under nitrogen atmosphere. The resulting mixture was stirred for 1 h at room temperature under nitrogen atmosphere. The reaction was quenched by the addition of water/Ice (10 mL) at room temperature. The resulting mixture was extracted with EtOAc (3×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (4:1) to afford tert-butyl N-[(2R)-1-cyclopropoxy-3-hydroxypropan-2-yl]carbamate (1.3 g, 80.97%) as a colorless oil.

### Step 3

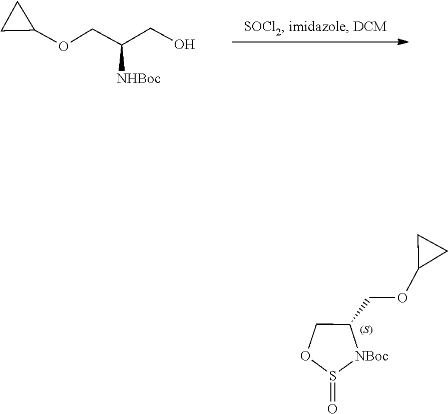

To a solution of Imidazole (1.05 g, 15.392 mmol, 4 equiv) in DCM (30 mL) were added SOCl₂ (0.42 mL, 5.772 mmol, 1.5 equiv) followed by DIEA (1.34 mL, 7.696 mmol, 2 equiv) dropwise at 0° C. To the above mixture was added tert-butyl N-[(2R)-1-cyclopropoxy-3-hydroxypropan-2-yl]carbamate (890 mg, 3.848 mmol, 1 equiv) dissolved in DCM (5 mL) dropwise at 0° C. The resulting mixture was stirred for additional 1 h at room temperature. The resulting mixture was washed with 3×30 mL of conc. HCl (0.5 M). The resulting mixture was concentrated under vacuum to afford tert-butyl (4S)-4-(cyclopropoxymethyl)-2-oxo-1,2lambda4,3-oxathiazolidine-3-carboxylate (1.06 g, 99.33%) as a yellow solid.

### Step 4

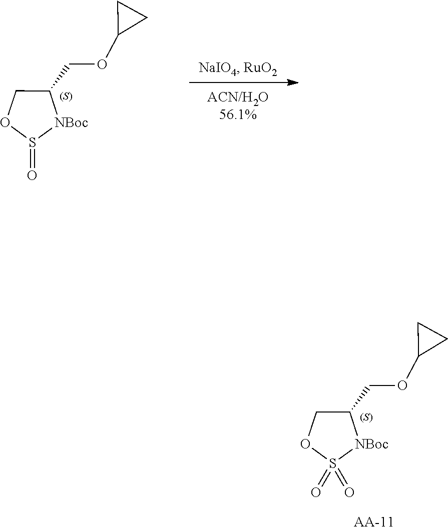

To a solution of tert-butyl (4S)-4-(cyclopropoxymethyl)-2-oxo-1,2lambda4,3-oxathiazolidine-3-carboxylate (1.06 g, 3.822 mmol, 1 equiv) in MeCN (13 mL) and H₂O (7 mL) was added NaIO₄ (0.98 g, 4.586 mmol, 1.2 equiv) and ruthenium(iv) oxide hydrate (11.55 mg, 0.076 mmol, 0.02 equiv) at 0° C. The resulting mixture was stirred for 1 h at 0° C. The resulting mixture was filtered through a Celite pad. The filtrate was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (3:1) to afford tert-butyl (4S)-4-(cyclopropoxymethyl)-2,2-dioxo-1,2lambda6,3-oxathiazolidine-3-carboxylate (AA-11, 0.63 g, 56.19%) as a colorless oil.

Tert-butyl (S)-4-(2,2,2-trifluoroethyl)-1,2,3-oxathiazolidine-3-carboxylate 2,2-dioxide (AA-13) was synthesized from tert-butyl (S)-(4,4,4-trifluoro-1-hydroxybutan-2-yl)carbamate (WO2012/116279) according to the general method

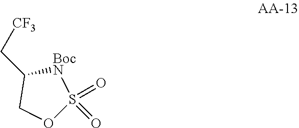 [SMILES: CC(C)(C)OC(=O)N1[C@@H](CC(F)(F)F)COS1(=O)=O]

Tert-butyl (S)-4-((trifluoromethoxy)methyl)-1,2,3-oxathiazolidine-3-carboxylate 2,2-dioxide (AA-14) was synthesized from tert-butyl (R)-(1-hydroxy-3-(trifluoromethoxy)propan-2-yl)carbamate (WO2021/087018) according to the general method

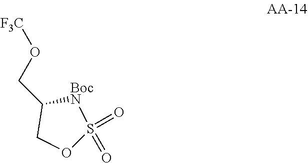 [SMILES: CC(C)(C)OC(=O)N1[C@@H](COC(F)(F)F)COS1(=O)=O]

Reference for Tert-butyl (S)-4-isobutyl-1,2,3-oxathiazolidine-3-carboxylate 2,2-dioxide (AA-16) WO2022/042657.

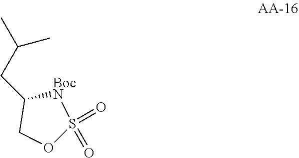 [SMILES: CC(C)C[C@H]1COS(=O)(=O)N1C(=O)OC(C)(C)C]

### Synthesis of tert-butyl (S)-4-(2,2-difluoroethyl)-1,2,3-oxathiazolidine-3-carboxylate (AA-18)

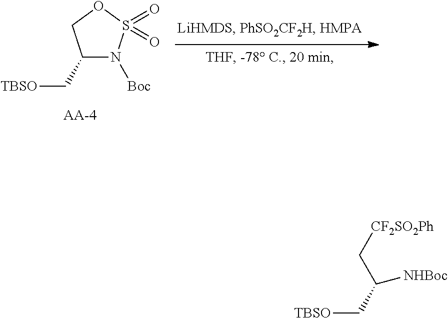

### Step 1: tert-butyl (S)-(1-((tert-butyldimethylsilyl)oxy)-4,4-difluoro-4-(phenylsulfonyl)butan-2-yl)carbamate

LiHMDS (1M solution in THF, 16.33 mL, 16.3 mmol, 1.2 equiv) was added slowly to a solution of tert-butyl (R)-4-(((tert-butyldimethylsilyl)oxy)methyl)-1,2,3-oxathiazolidine-3-carboxylate 2,2-dioxide (5 g, 13.6 mmol, 1 equiv) and (difluoromethane)sulfonylbenzene (2.13 mL, 15.0 mmol, 1.1 equiv) in THF (150 mL) and HMPA (6.90 mL, 39.5 mmol, 2.9 equiv) at −78° C. under N₂. The reaction mixture was stirred at this temperature for 20 min, and then quenched with 20% aqueous sulfuric acid (60 mL). The mixture was extracted with EtOAc and the combined organic layers were washed with saturated NaHCO₃ solution and then dried over Na₂SO₄. The sodium salts were filtered off and the filtrate was concentrated under reduced pressure. The residue was purified by silica gel column chromatography (PE/EA=3:1) affording the product as colorless oil (6.3 g, 97%).

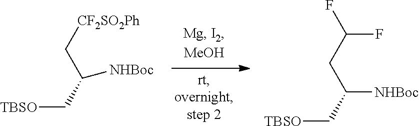

### Step 2: tert-butyl (S)-(1-((tert-butyldimethylsilyl)oxy)-4,4-difluorobutan-2-yl)carbamate

10 mL of methanol and 12 (0.17 g, 0.69 mmol, 0.1 equiv) were added to Mg (5.02 g, 206.4 mmol, 30 equiv) stored under a nitrogen atmosphere. The mixture was cooled to 0° C. and a solution of tert-butyl (S)-(1-((tert-butyldimethylsilyl)oxy)-4,4-difluoro-4-(phenylsulfonyl)butan-2-yl)carbamate (3.3 g, 6.9 mmol, 1 equiv) in 30 mL methanol was added. The reaction mixture was allowed to warm to 20° C. and stirred at this temperature overnight. The reaction was quenched with saturated ammonium chloride solution and extracted with EtOAc. The combined organic layers were washed with brine, dried over anhydrous sodium sulfate, and filtered. The solvents were evaporated, and the residue was purified by silica gel column chromatography (PE/EA=7:1) affording the product as colorless oil (1.47 g, 63%).

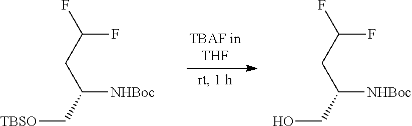 [SMILES: CC(C)(C)OC(=O)N[C@H](CO[Si](C)(C)C(C)(C)C)CC(F)F ; CC(C)(C)OC(=O)N[C@H](CO)CC(F)F]

### Step 3: tert-butyl (S)-(4,4-difluoro-1-hydroxybutan-2-yl)carbamate

To a solution of tert-butyl (S)-(1-((tert-butyldimethylsilyl)oxy)-4,4-difluorobutan-2-yl)carbamate (700 mg, 2.06 mmol, 1 equiv) in THE (7 mL) was added TBAF (1.65 mL, 1.65 mmol, 0.8 equiv) dropwise at 0° C. The resulting mixture was stirred for 1 h at room temperature and then concentrated under reduced pressure. The residue was purified by silica gel column chromatography (PE/EA=1:1) yielding the product as white solid (430 mg, 93%).

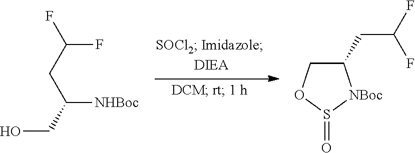 [SMILES: CC(C)(C)OC(=O)N[C@H](CO)CC(F)F ; CC(C)(C)OC(=O)N1[C@@H](CC(F)F)COS1=O]

### Step 4. tert-butyl (4S)-4-(2,2-difluoroethyl)-1,2,3-oxathiazolidine-3-carboxylate 2-oxide

A portion of SOCl₂ (0.21 mL, 2.86 mmol, 1.5 equiv) followed by DIEA (0.67 mL, 3.82 mmol, 2 equiv) were added dropwise at 0° C. to a solution of imidazole (520 mg, 7.64 mmol, 4 equiv) in DCM (13 mL). (S)-(4,4-difluoro-1-hydroxybutan-2-yl)carbamate (430 mg, 1.91 mmol, 1 equiv) dissolved in DCM (2 mL) was added dropwise at 0° C. and the resulting mixture was stirred for 1 h at room temperature. The mixture was washed with saturated NaHCO₃ solution and evaporated affording the product as yellow oil (500 mg, 97%).

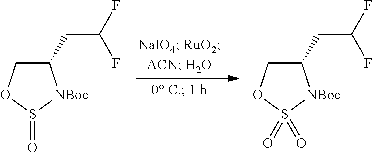 [SMILES: CC(C)(C)OC(=O)N1[C@@H](CC(F)F)COS1=O ; CC(C)(C)OC(=O)N1[C@@H](CC(F)F)COS1(=O)=O]

### Step 5: tert-butyl (S)-4-(2,2-difluoroethyl)-1,2,3-oxathiazolidine-3-carboxylate (AA-18)

To a solution of tert-butyl (4S)-4-(2,2-difluoroethyl)-1,2,3-oxathiazolidine-3-carboxylate 2-oxide (500 mg, 1.84 mmol, 1 equiv) in ACN (6.5 mL) and water (3.5 mL) were added NaIO₄ (591 mg, 2.76 mmol, 1.5 equiv) and ruthenium(iv) oxide hydrate (13.9 mg, 0.092 mmol, 0.05 equiv) at 0° C. The resulting mixture was stirred for 1 h at 0° C., and then filtered. The filtrate was extracted with EtOAc and the combined organic layers were washed with brine (20 mL) and dried over anhydrous Na₂SO₄. The solvents were removed under reduced pressure and the residue was purified by silica gel column chromatography (PE/EA=2:1). White solid (310 mg, 59%).

Reference for tert-butyl (S)-4-(2-((tert-butyldimethylsilyl)oxy)ethyl)-1,2,3-oxathiazolidine-3-carboxylate 2,2-dioxide (AA-19) Zhang, N.; Arnold, M. A.; Dakka, A.; Karp, G. M.; Luong, T. T.; Narasimhan, J.; Naryshkin, N. A.; Wang, J.; Zhang, X. WO 2020167628.

### Synthesis of tert-butyl 4-(oxetan-3-yl)-2,2-dioxo-1,2lambda6,3-oxathiazolidine-3-carboxylate (AA-20)

### Step 1

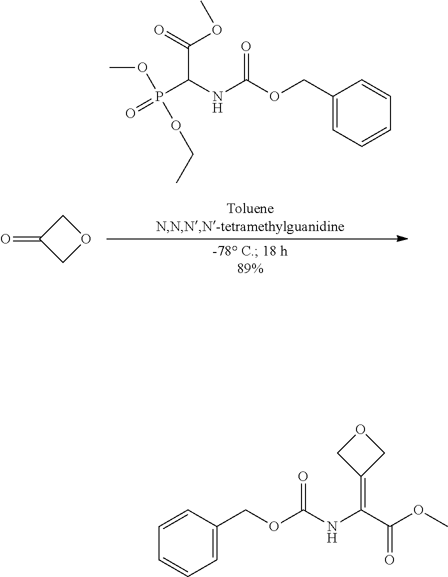 [SMILES: CCOP(=O)(OC)C(NC(=O)OCc1ccccc1)C(=O)OC ; O=C1COC1 ; COC(=O)C(NC(=O)OCc1ccccc1)=C1COC1]

To a stirred solution of methyl 2-{[(benzyloxy)carbonyl]amino}-2-(dimethoxyphosphoryl)acetate (10 g, 30.188 mmol, 1 equiv) in toluene (100 mL) under an nitrogen atmosphere was added 1,1,3,3-tetramethylguanidine (3.48 g, 30.188 mmol, 1 equiv) at −78° C. and stirred for 30 min. Then a solution of 3-oxetanone (2.18 g, 30.188 mmol, 1 equiv) in toluene (10 mL) was added to the reaction mixture at −78° C. The reaction mixture was warmed to room temperature and stirred for 18 h. After consumption of the starting material (monitored by TLC), the reaction mixture was diluted with water (250 mL) and extracted with EtOAc (2×250 mL) and 20% MeOH:CH₂Cl₂ (2×250 mL). The combined organic extracts were dried over sodium sulfate, filtered and concentrated in vacuo. The crude material was purified by column chromatography using 5-10% MeOH:CH₂Cl₂ to afford methyl 2-{[(benzyloxy)carbonyl]amino}-2-(oxetan-3-ylidene)acetate (7.45 g, 89.00%) as a white solid.

### Step 2

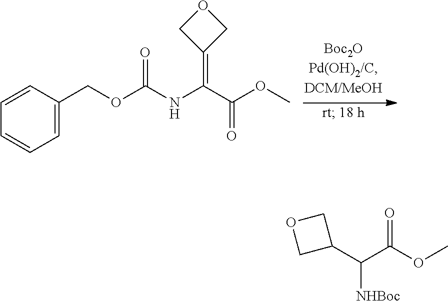 [SMILES: COC(=O)C(NC(=O)OCc1ccccc1)=C1COC1 ; COC(=O)C(NC(=O)OC(C)(C)C)C1COC1]

A solution of methyl 2-{[(benzyloxy)carbonyl]amino}-2-(oxetan-3-ylidene)acetate (4 g, 14.426 mmol, 1 equiv) in DCM/MeOH (1/1; 50 mL) was treated with Pd(OH)₂/C (800 mg, 5.697 mmol, 0.39 equiv) and Boc₂O (3.15 g, 14.426 mmol, 1 equiv). The resulting mixture was stirred for 18 h at room temperature. The reaction was monitored by LCMS. The residue was purified by reverse flash chromatography with the following conditions: column, C₁₈ silica gel; mobile phase, MeCN in Water (10 mmol/L NH₄HCO₃), 10% to 50% gradient in 10 min; detector, UV 254 nm. This resulted in methyl 2-[(tert-butoxycarbonyl)amino]-2-(oxetan-3-yl)acetate (1.3 g, 36.74%) as a yellow oil.

### Step 3

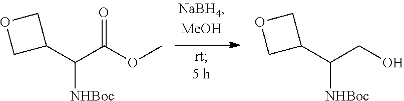 [SMILES: COC(=O)C(NC(=O)OC(C)(C)C)C1COC1 ; CC(C)(C)OC(=O)NC(CO)C1COC1]

A solution of methyl 2-[(tert-butoxycarbonyl)amino]-2-(oxetan-3-yl)acetate (1.3 g, 5.300 mmol, 1 equiv) in MeOH (13.00 mL) was treated with NaBH₄ (0.60 g, 15.900 mmol, 3 equiv) at 0° C. The resulting mixture was stirred for 5 h at room temperature. The reaction was monitored by LCMS. The residue was purified by silica gel column chromatography, eluted with PE/EA (1/1) to afford tert-butyl N-[2-hydroxy-1-(oxetan-3-yl)ethyl]carbamate (700 mg, 60.79%) as a yellow oil.

### Step 4

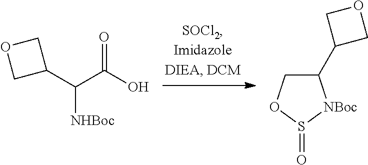 [SMILES: CC(C)(C)OC(=O)NC(C(=O)O)C1COC1 ; CC(C)(C)OC(=O)N1C(C2COC2)COS1=O]

To a solution of Imidazole (1.00 g, 14.728 mmol, 4 equiv) in DCM (24 mL) were added SOCl2 (0.40 mL, 5.523 mmol, 1.5 equiv) followed by DIEA (1.28 mL, 7.364 mmol, 2 equiv) dropwise at 0° C. To the above mixture was added tert-butyl N-[2-hydroxy-1-(oxetan-3-yl)ethyl]carbamate (800 mg, 3.682 mmol, 1 equiv) dissolved in DCM (4 mL) dropwise at 0° C. The resulting mixture was stirred for additional 1 h at room temperature. The resulting mixture was washed with 3×20 mL of conc. HCl (1M). The resulting mixture was concentrated under vacuum. The residue was purified by silica gel column chromatography, eluted with PE/EA (1:1) to afford tert-butyl 4-(oxetan-3-yl)-2-oxo-1,2lambda4,3-oxathiazolidine-3-carboxylate (780 mg, 80.45%) as a yellow oil.

### Step 5

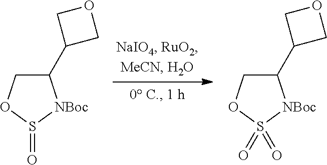 [SMILES: CC(C)(C)OC(=O)N1C(C2COC2)COS1=O ; CC(C)(C)OC(=O)N1C(C2COC2)COS1(=O)=O]

To a solution of tert-butyl 4-(oxetan-3-yl)-2-oxo-1,2lambda4,3-oxathiazolidine-3-carboxylate (780 mg, 2.962 mmol, 1 equiv) in MeCN (10 mL) and H₂O (6 mL) was added NaIO4 (760.32 mg, 3.554 mmol, 1.20 equiv) and ruthenium(iv) oxide hydrate (8.95 mg, 0.059 mmol, 0.02 equiv) at 0° C. The resulting mixture was stirred for 1 h at 0° C. The resulting mixture was filtered through a Celite pad. The filtrate was extracted with EtOAc (3×20 mL). The combined organic layers were washed with brine (20 mL), dried over anhydrous Na2SO4. After filtration, the filtrate was concentrated under reduced pressure. The residue was purified by silica gel column chromatography, eluted with PE/EA (1:1) to afford tert-butyl 4-(oxetan-3-yl)-2,2-dioxo-1,2lambda6,3-oxathiazolidine-3-carboxylate (470 mg, 56.80%) as a white solid.

### Synthesis of tert-butyl 5-methyl-2,2-dioxo-1,2lambda6,3-oxathiazinane-3-carboxylate (AA-21)

### Step 1

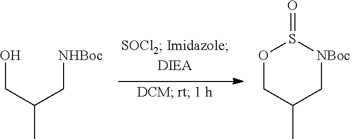

To a solution of Imidazole (719.44 mg, 10.568 mmol, 4 equiv) in DCM (15 mL) were added SOCl₂ (0.29 mL, 3.963 mmol, 1.5 equiv) followed by DIEA (0.92 mL, 5.284 mmol, 2 equiv) dropwise at 0° C. To the above mixture was added tert-butyl N-(3-hydroxy-2-methylpropyl)carbamate (500 mg, 2.642 mmol, 1 equiv) dissloved in DCM (3 mL) dropwise at 0° C. The resulting mixture was stirred for additional 1 h at room temperature. The resulting mixture was washed with 3×20 mL of saturated NaHCO₃ (aq.). The resulting mixture was concentrated under vacuum. The crude product was used in the next step directly without further purification.

### Step 2

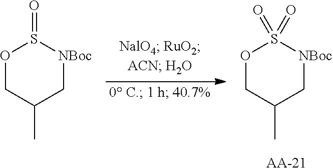

To a solution of tert-butyl 5-methyl-2-oxo-1,2lambda4,3-oxathiazinane-3-carboxylate (620 mg, 2.635 mmol, 1 equiv) in MeCN (8 mL) and H₂O (4 mL) was added NaIO₄ (845.38 mg, 3.952 mmol, 1.5 equiv) and ruthenium(iv) oxide hydrate (19.90 mg, 0.132 mmol, 0.05 equiv) at 0° C. The resulting mixture was stirred for 1 h at 0° C. The resulting mixture was filtered through a Celite pad. The filtrate was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (3:1) to afford tert-butyl 5-methyl-2,2-dioxo-1,2lambda6,3-oxathiazinane-3-carboxylate (270 mg, 40.78%) as a white solid. tert-butyl (S)-4-cyclopropyl-1,2,3-oxathiazolidine-3-carboxylate 2,2-dioxide (AA-22) was prepared according to the general method starting from commercially available (S)-2-amino-2-cyclopropylethanol hydrochloride.

The Table 3 below lists the General Synthesis method(s) used for the synthesis of final compounds, applicable building blocks, and selected spectroscopic data.

TABLE 3

| | General | | | | LC/ |
|---|---|---|---|---|---|
| | Synthesis | | Building | | MS |
| Compound | Method | Fragment | Block | NMR data | Ion |
| 1 | 1 | AA-4 | BB-2 | 1H NMR (300 MHz, DMSO- | 319.2 |
| | | | | d6) δ 8.61 (s, 1H), 7.63 − 7.48 | |
| | | | | (m, 2H), 6.40 (d, J = 1.6 Hz, | |
| | | | | 2H), 4.59 (d, J = 6.0 Hz, 2H), | |
| | | | | 3.31 − 3.13 (m, 2H), 3.10 − | |
| | | | | 2.90 (m, 2H), 2.76 (dd, J = | |
| | | | | 13.9, 7.4 Hz, 1H), 2.40 (d, J = | |
| | | | | 2.1 Hz, 3H). | |
| 2 | 1 | AA-1 | BB-2 | 1H NMR (300 MHz, DMSO- | 303.3 |
| | | | | d6) δ 8.62 (s, 1H), 7.62 − 7.51 | |
| | | | | (m, 2H), 6.41 (d, J = 1.8 Hz, | |
| | | | | 2H), 4.59 (d, J = 6.1 Hz, 2H), | |
| | | | | 3.17 (q, J = 6.4 Hz, 1H), 2.90 | |
| | | | | (d, J = 6.8 Hz, 2H), 2.40 (s, | |
| | | | | 3H), 1.14 − 1.02 (m, 3H). | |
| 3 | 2 | AA-4 | BB-2 | 1H NMR(300 MHz, DMSO- | 335 |
| | | | | d6) δ 8.55 (s, 1H), 7.69 (t, J = | |
| | | | | 6.1 Hz, 1H), 7.45 − 7.350 (m, | |
| | | | | 1H), 7.19 − 7.09 (m, 1H), | |
| | | | | 7.04 − 6.94 (m, 1H), 4.80 (d, J = | |
| | | | | 6.1 Hz, 2H), 3.39 − 3.29 (m, 5H), | |
| | | | | 3.07 − 2.97 (m, 2H), | |
| | | | | 2.84 − 2.74 (m, 1H), 2.40 (s, 3H). | |
| 4 | 3 | none | BB-7 | 1H NMR (300 MHz, DMSO- | 319 |
| | | | | d6) δ 8.55 (s, 1H), 7.69 (t, J = | |
| | | | | 6.2 Hz, 1H), 7.40 (dd, J = 5.1, | |
| | | | | 1.3 Hz, 1H), 7.14 (dd, J = 3.5, | |
| | | | | 1.2 Hz, 1H), 6.99 (dd, J = 5.1, | |
| | | | | 3.4 Hz, 1H), 4.80 (d, J = 6.1 | |
| | | | | Hz, 2H), 3.14 (h, J = 6.3 Hz, | |
| | | | | 1H), 2.88 (d, J = 6.4 Hz, 2H), | |
| | | | | 2.40 (s, 3H), 1.05 (d, J = 6.3 | |
| | | | | Hz, 3H). | |
| 5 | 2 | AA-4 | BB-2 | 1H NMR (300 MHz, DMSO- | 336.2 |
| | | | | d6) δ 8.51 (s, 1H), 7.87 (t, J = | |
| | | | | 6.3 Hz, 1H), 7.79 (d, J = 3.3 | |
| | | | | Hz, 1H), 7.64 (d, J = 3.2 Hz, | |
| | | | | 1H), 4.94 (d, J = 6.3 Hz, 2H), | |
| | | | | 4.75 (s, 1H), 3.02 (ddd, J = | |
| | | | | 28.9, 13.5, 5.3 Hz, 3H), 2.76 | |
| | | | | (dd, J = 14.1, 7.7 Hz, 1H), 2.41 | |
| | | | | (s, 3H | |
| 6 | 3 | none | BB-7 | 1H NMR (300 MHz, DMSO- | 320.2 |
| | | | | d6) δ 8.52 (s, 1H), 7.89 (t, J = | |
| | | | | 6.3 Hz, 1H), 7.78 (d, J = 3.2 | |
| | | | | Hz, 1H), 7.64 (d, J = 3.3 Hz, | |
| | | | | 1H), 4.94 (d, J = 6.3 Hz, 2H), | |
| | | | | 3.16 (d, J = 6.4 Hz, 1H), 2.93 − | |
| | | | | 2.86 (m, 2H), 2.41 (s, 3H), 1.06 | |
| | | | | (d, J = 6.2 Hz, 3H). | |
| 7 | 2 | AA-4 | BB-2 | 1H-NMR (300 MHz, Methanol- | 335 |
| | | | | d4) δ 8.42 (s, 1H), 7.47 − 7.37 | |
| | | | | (m, 1H), 7.38 − 7.28 (m, 1H), | |
| | | | | 7.18 − 7.08 (m, 1H), 4.71 (d, | |
| | | | | J = 1.0 Hz, 2H), 3.67 − 3.57 (m, | |
| | | | | 1H), 3.56 − 3.45 (m, 1H), | |
| | | | | 3.30 − 3.12 (m, 2H), 3.06 − 2.93 | |
| | | | | (m, 1H), 2.49 (s, 3H). | |
| 8 | 3 | none | BB-7 | 1H NMR (300 MHz, Methanol- | 319 |
| | | | | d4) δ 8.49 (s, 1H), 7.49 (dd, J = | |
| | | | | 5.0, 3.0 Hz, 1H), 7.40 (dd, J = | |
| | | | | 3.0, 1.3 Hz, 1H), 7.19 (dd, J = | |
| | | | | 5.0, 1.4 Hz, 1H), 4.78 (s, 2H), | |
| | | | | 3.52 − 3.39 (m, 1H), 3.24 − | |
| | | | | 3.03 (m, 2H), 2.56 (s, 3H), 1.34 | |
| | | | | (s, 1H), 1.30 (d, J = 6.4 Hz, | |
| | | | | 3H). | |
| 9 | 2 | AA-4 | BB-02 | 1H NMR (300 MHz, Methanol- | 353.2 |
| | | | | d4) δ 8.55 (s, 1H), 7.31 (dd, J = | |
| | | | | 5.6, 4.0 Hz, 1H), 6.87 (d, J = | |
| | | | | 5.6 Hz, 1H), 4.81 (d, J = 1.2 | |
| | | | | Hz, 2H), 3.73 (dd, J = 11.2, 3.9 | |
| | | | | Hz, 1H), 3.58 (dd, J = 11.2, 5.6 | |
| | | | | Hz, 1H), 3.49 − 3.39 (m, 1H), | |
| | | | | 3.29 (d, J = 7.3 Hz, 1H), 3.16 | |
| | | | | (dd, J = 14.6, 7.0 Hz, 1H), 2.52 | |
| | | | | (s, 3H). | |
| 10 | 2 using | AA-1 | BB-2 | (400 MHz, Methanol-d4) δ 8.52 | 337 |
| | Ruphos in | | | (s, 1H), 7.34 − 7.24 (m, 1H), | |
| | place of | | | 6.85 (d, J = 5.5 Hz, 1H), 4.79 | |
| | xantphos | | | (d, J = 1.2 Hz, 2H), 3.47 − 3.37 | |
| | | | | (m, 1H), 3.16 − 3.06 (m, 2H), | |
| | | | | 2.48 (s, 3H), 1.25 (d, J = 6.4 | |
| | | | | Hz, 3H). | |
| 11 | 1 | None | BB-2 | 1H NMR (300 MHz, Methanol- | 246.2 |
| | | | | d4) δ 8.59 (s, 1H), 7.58 (q, J = | |
| | | | | 1.2 Hz, 1H), 7.47 (t, J = 1.4 Hz, | |
| | | | | 1H), 6.38 (d, J = 1.4 Hz, 2H), | |
| | | | | 4.69 (s, 2H), 2.55 (d, J = 1.2 | |
| | | | | Hz, 3H). | |
| 12 | 1 | None | BB-2 | 1H NMR (300 MHz, Methanol- | 263.1 |
| | | | | d4) δ 8.52 (s, 1H), 7.80 (d, J = | |
| | | | | 3.3 Hz, 1H), 7.62 (q, J = 1.2 | |
| | | | | Hz, 1H), 7.55 (d, J = 3.3 Hz, | |
| | | | | 1H), 5.05 (s, 2H), 2.56 (d, J = | |
| | | | | 1.2 Hz, 3H). | |
| 15 | 5 | | BB-8 | 1H NMR (300 MHz, Methanol- | 303.2 |
| | | | | d4) δ 8.84 (s, 1H), 7.84 (d, J = | |
| | | | | 2.4 Hz, 1H), 7.55 (d, J = 1.8 | |
| | | | | Hz, 1H), 6.35 (t, J = 2.2 Hz, | |
| | | | | 1H), 5.82 (s, 2H), 3.45 − 3.34 | |
| | | | | (m, 1H), 3.19 − 2.99 (m, 2H), | |
| | | | | 2.51 (s, 3H), 1.23 (d, J = 6.4 | |
| | | | | Hz, 3H). | |
| 16 | 2 | AA-1 | BB-2 | 1H NMR (300 MHz, DMSO- | 337.1 |
| | | | | d6) δ 8.59 (s, 1H), 7.62 (t, J = | |
| | | | | 6.2 Hz, 1H), 6.86 (t, J = 3.8 Hz, | |
| | | | | 1H), 6.62 − 6.52 (m, 1H), | |
| | | | | 4.73 − 4.63 (m, 2H), 3.18 − 3.08 | |
| | | | | (m, 1H), 2.88 (d, J = 6.5 Hz, 2H), | |
| | | | | 2.40 (s, 3H), 2.18 (s, 2H), 1.05 | |
| | | | | (d, J = 6.2 Hz, 3H). | |
| 17 | 5 | | BB-8 | 1H NMR (300 MHz, Methanol- | 337.2 |
| | | | | d4) δ 8.43 (s, 1H), 6.78 − | |
| | | | | 6.68 (m, 1H), 6.59 (t, J = 1.9 | |
| | | | | Hz, 1H), 4.58 (d, J = 1.2 Hz, | |
| | | | | 2H), 3.40 − 3.27 (m, 1H) 3.15 − | |
| | | | | 2.95 (m, 2H), 2.49 (s, 3H), 1.21 | |
| | | | | (d, J = 6.4 Hz, 3H). | |
| 18 | 2 | AA-1 | BB-2 | 1H NMR (400 MHz, Methanol- | 320 |
| | | | | d4) δ 8.48 − 8.36 (m, 2H), | |
| | | | | 7.39 − 7.29 (m, 1H), 5.16 − 5.04 | |
| | | | | (m, 2H), 3.35 (d, J = 6.8 Hz, 1H), | |
| | | | | 3.13 − 2.97 (m, 2H), 2.49 (s, | |
| | | | | 3H), 1.20 (d, J = 6.4 Hz, 3H). | |
| 19 | 2 | AA-1 | BB-9 | 1H NMR (300 MHz, Methanol- | 333.2 |
| | | | | d4) δ 7.27 (dd, J = 5.1, 1.2 Hz, | |
| | | | | 1H), 7.01 (dq, J = 3.5, 1.1 Hz, | |
| | | | | 1H), 6.94 (dd, J = 5.1, 3.5 Hz, | |
| | | | | 1H), 5.09 (d, J = 1.0 Hz, 2H), | |
| | | | | 3.29 (d, J = 6.6 Hz, 1H), 3.10 − | |
| | | | | 2.90 (m, 2H), 2.64 (s, 3H), 2.44 | |
| | | | | (s, 3H), 1.19 (d, J = 6.4 Hz, | |
| | | | | 3H). | |
| 20 | 1 | AA-16 | BB-2 | 1H NMR (300 MHz, DMSO- | 345.1 |
| | | | | d6) δ 8.62 (s, 1H), 7.70 − 7.46 | |
| | | | | (m, 2H), 6.41 (q, J = 2.1, 1.7 | |
| | | | | Hz, 2H), 4.59 (d, J = 6.0 Hz, | |
| | | | | 2H), 3.07 − 2.80 (m, 4H), 2.40 | |
| | | | | (s, 3H), 1.79 (dt, J = 13.2, 6.6 | |
| | | | | Hz, 1H), 1.24 (dt, J = 8.4, 5.3 | |
| | | | | Hz, 2H), 0.85 (dd, J = 14.7, 6.6 | |
| | | | | Hz, 6H). | |
| 21 | 13 | | tert-butyl (furan- | 1 H NMR (300 MHz, DMSO- | 289.2 |
| | | | 2-ylmethyl)(7- | d6) δ 8.62 (s, 1H), 7.63 − 7.50 | |
| | | | methylthieno[3,2- | (m, 2H), 6.41 (d, J = 1.5 Hz, | |
| | | | c]pyridazin-4- | 2H), 4.60 (d, J = 6.1 Hz, 2H), | |
| | | | yl)carbamate | 3.29 − 3.18 (m, 1H), 3.01 (q, | |
| | | | | J = 7.0 Hz, 2H), 2.89 (d, J = 7.0 | |
| | | | | Hz, 1H), 2.40 (d, J = 3.2 Hz, | |
| | | | | 3H). | |
| 22 | | AA-1 | BB-4 | 1H NMR (400 MHz, Methanol- | 335 |
| | | | | d4) δ 8.50 (d, J = 12.4 Hz, 1H), | |
| | | | | 7.30 (dd, J = 5.1, 1.2 Hz, 1H), | |
| | | | | 7.11 (dd, J = 3.5, 1.2 Hz, 1H), | |
| | | | | 6.97 (dd, J = 5.1, 3.5 Hz, 1H), | |
| | | | | 4.11 (s, 2H), 3.37 − 3.32 (m, | |
| | | | | 1H), 3.14 − 2.88 (m, 2H), | |
| | | | | 1.24 − 1.13 (m, 3H). | |
| 23 | 2 | AA-1 | BB-10 | 1H NMR (400 MHz, DMSO- | 333.2 |
| | | | | d6) δ 8.54 (s, 1H), 7.67 (t, J = | |
| | | | | 6.2 Hz, 1H), 7.40 (dd, J = 5.0, | |
| | | | | 1.3 Hz, 1H), 7.20 − 7.11 (m, | |
| | | | | 1H), 6.98 (dd, J = 5.1, 3.4 Hz, | |
| | | | | 1H), 4.79 (d, J = 6.1 Hz, 2H), | |
| | | | | 3.15 (p, J = 6.4 Hz, 1H), 2.97 − | |
| | | | | 2.82 (m, 4H), 1.19 (t, J = 7.4 | |
| | | | | Hz, 3H), 1.15 − 1.03 (m, 3H). | |
| 24 | | AA-1 | BB-4 | 1H NMR (300 MHz, Methanol- | 330 |
| | | | | d4) δ 8.70 − 8.58 (m, 1H), 7.35 | |
| | | | | (dd, J = 5.1, 1.2 Hz, 1H), 7.15 | |
| | | | | (dd, J = 3.6, 1.1 Hz, 1H), 7.06 − | |
| | | | | 6.96 (m, 1H), 3.44 − 3.37 (m, | |
| | | | | 1H), 3.29 − 3.10 (m, 2H), 1.24 | |
| | | | | (d, J = 6.3 Hz, 3H). | |
| 25 | | tert-butyl (S)-(3-(4,4,5,5- | BB-11 | 1H NMR (400 MHz, DMSO- | 333 |
| | | tetramethyl-1,3,2- | | d6) δ 8.55 (d, J = 6.8 Hz, 1H), | |
| | | dioxaborolan-2-yl)but-3- | | 7.69 (dt, J = 12.4, 6.1 Hz, 1H), | |
| | | en-2-yl)carbamate | | 7.40 (dt, J = 5.0, 1.5 Hz, 1H), | |
| | | | | 7.14 (dd, J = 3.3, 1.5 Hz, 1H), | |
| | | | | 6.98 (td, J = 4.2, 3.4, 1.4 Hz, | |
| | | | | 1H), 4.79 (t, J = 5.4 Hz, 2H), | |
| | | | | 3.14 (t, J = 7.0 Hz, 1H), 3.04 − | |
| | | | | 2.81 (m, 1H), 2.41 (d, J = 2.8 | |
| | | | | Hz, 3H), 1.30 (d, J = 6.6 Hz, | |
| | | | | 3H), 0.95 (d, J = 6.2 Hz, 3H). | |
| 26 | | tert-butyl (S)-(3-(4,4,5,5- | BB-11 | 1H NMR (400 MHz, DMSO- | 333.1 |
| | | tetramethyl-1,3,2- | | d6) δ 8.55 (d, J = 2.2 Hz, 1H), | |
| | | dioxaborolan-2-yl)but-3- | | 7.69 (s, 1H), 7.48 − 7.31 (m, | |
| | | en-2-yl)carbamate | | 1H), 7.13 (d, J = 3.4 Hz, 1H), | |
| | | | | 6.98 (dd, J = 5.1, 3.4 Hz, 1H), | |
| | | | | 2.95 (dt, J = 19.7, 9.7 Hz, 3H), | |
| | | | | 2.38 (d, J = 9.3 Hz, 3H), 1.84 − | |
| | | | | 1.47 (m, 2H), 1.09 (dd, J = | |
| | | | | 11.9, 6.5 Hz, 3H). | |
| 27 | 10 | AA-4 | BB-2 | 1H NMR (400 MHz, DMSO- | 344 |
| | | | | d6) δ 8.56 (s, 1H), 7.71 (t, J = | |
| | | | | 6.2 Hz, 1H), 7.40 (dd, J = 5.1, | |
| | | | | 1.3 Hz, 1H), 7.13 (d, J = 3.5 | |
| | | | | Hz, 1H), 6.98 (dd, J = 5.1, 3.5 | |
| | | | | Hz, 1H), 4.79 (d, J = 6.2 Hz, | |
| | | | | 1H), 3.26 (s, 1H), 3.05 − 2.91 | |
| | | | | (m, 1H), 2.63 (d, J = 5.2 Hz, | |
| | | | | 1H), 2.59 (s, 1H), 2.57 (s, 2H), | |
| | | | | 2.41 (s, 2H). | |
| 28 | 1 | AA-18 | BB-2 | 1H-NMR 300 MHz, Methanol- | 369.1 |
| | | | | d4) δ 8.49 (s, 1H), 7.37 − | |
| | | | | 7.27 (m, 1H), 7.18 − 7.08 (m, | |
| | | | | 1H), 7.04 − 6.94 (m, 1H), | |
| | | | | 6.40 − 5.86 (m, 1H), 4.88 (s, 2H), | |
| | | | | 3.47 − 3.36 (m, 1H), 3.20 − | |
| | | | | 2.99 (m, 2H), 2.50 (s, 3H), | |
| | | | | 2.19 − 1.89 (m, 2H). | |
| 29 | 1 | AA-19 | BB-2 | 1H-NMR (400 MHz, DMSO- | 333.2 |
| | | | | d6) δ 8.61 (s, 1H), 7.60 (s, 1H), | |
| | | | | 7.53 (t, J = 5.8 Hz, 1H), 6.78 − | |
| | | | | 6.71 (m, 1H), 6.41 (d, J = 1.8 | |
| | | | | Hz, 2H), 4.59 (d, J = 6.0 Hz, | |
| | | | | 2H), 3.89 − 3.75 (m, 1H), 3.53 | |
| | | | | (t, J = 6.3 Hz, 2H), 3.46 − 3.42 | |
| | | | | (m, 1H), 3.17 − 2.79 (m, 3H), | |
| | | | | 2.40 (s, 3H), 1.68 − 1.36 (m, | |
| | | | | 2H). | |
| 30 | 10 | AA-4 | BB-2 | 1H NMR (300 MHz, DMSO- | 349.3 |
| | | | | d6) δ 8.61 (d, J = 1.6 Hz, 1H), | |
| | | | | 7.60 (t, J = 1.4 Hz, 1H), 7.52 (t, | |
| | | | | J = 6.4 Hz, 1H), 6.40 (d, J = 1.4 | |
| | | | | Hz, 2H), 4.59 (d, J = 6.0 Hz, | |
| | | | | 2H), 3.17 − 2.99 (m, 2H), | |
| | | | | 2.93 − 2.81 (m, 1H), 2.67 − 2.55 | |
| | | | | (m, 2H), 2.41 (s, 3H), 2.08 | |
| | | | | (d, J = 3.3 Hz, 3H). | |
| 31 | 10 | AA-19 | BB-2 | 1H-NMR (300 MHz, Methanol- | 335.2 |
| | | | | d4) δ 8.56 (s, 1H), 7.47 (t, J = | |
| | | | | 1.3 Hz, 1H), 6.38 (d, J = 1.4 | |
| | | | | Hz, 2H), 4.79 − 4.63 (m, 3H), | |
| | | | | 4.63 − 4.46 (m, 1H), 3.38 | |
| | | | | (d, J = 8.0 Hz, 1H), 3.14 − | |
| | | | | 3.04 (m, 2H), 2.50 (s, 3H), | |
| | | | | 2.10 − 1.66 (m, 2H). | |
| 32 | 11 | AA-4 | BB-2 | 1H NMR (400 MHz, DMSO- | 381.3 |
| | | | | d6) δ 8.62 (s, 1H), 7.59 (dd, J = | |
| | | | | 1.8, 0.9 Hz, 1H), 7.56 (t, J = 6.1 | |
| | | | | Hz, 1H), 6.40 (qd, J = 3.3, 1.3 | |
| | | | | Hz, 2H), 4.59 (d, J = 6.1 Hz, | |
| | | | | 2H), 3.54 − 3.43 (m, 1H), | |
| | | | | 3.27 − 3.09 (m, 3H), 3.00 | |
| | | | | (dd, J = 14.4, 7.7 Hz, 1H), 2.41 | |
| | | | | (s, 3H), 2.00 − 1.47 (m, 1H). | |
| 33 | 11 | AA-4 | BB-2 | 1H NMR (400 MHz, Methanol- | 365.2 |
| | | | | d4) δ 8.55 (s, 1H), 7.44 (t, J = | |
| | | | | 1.4 Hz, 1H), 6.36 (d, J = 1.4 | |
| | | | | Hz, 2H), 4.66 (s, 2H), 3.69 − | |
| | | | | 3.59 (m, 1H), 3.27 − 3.09 (m, | |
| | | | | 2H), 2.95 (d, J = 6.4 Hz, 1H), | |
| | | | | 2.90 − 2.79 (m, 1H), 2.66 | |
| | | | | (d, J = 11.8 Hz, 3H). | |
| 34 | 1 | 3-bromo-2-methylprop- | BB-2 | 1H NMR (400 MHz, Methanol- | 300 |
| | | 1-ene | | d4) δ 8.55 (s, 1H), 7.59 (q, J = | |
| | | | | 1.3 Hz, 1H), 7.44 (t, J = 1.3 Hz, | |
| | | | | 1H), 6.34 (d, J = 1.4 Hz, 2H), | |
| | | | | 5.23 (t, J = 7.4 Hz, 1H), 4.84 − | |
| | | | | 4.81 (m, 2H), 2.86 − 2.73 (m, | |
| | | | | 2H), 2.52 (d, J = 1.3 Hz, 3H), | |
| | | | | 1.77 (t, J = 1.1 Hz, 3H). | |
| 35 | 1 | AA-1 | BB-4 | 1H NMR (300 MHz, Methanol- | 385.1 |
| | | | | d4) δ 8.60 (s, 1H), 7.34 (dd, J = | |
| | | | | 5.1, 1.3 Hz, 1H), 7.15 (d, J = | |
| | | | | 3.4 Hz, 1H), 7.01 (dd, J = 5.1, | |
| | | | | 3.5 Hz, 1H), 4.91 (s, 2H), 3.46 | |
| | | | | (q, J = 6.7 Hz, 1H), 3.17 (d, J = | |
| | | | | 6.9 Hz, 2H), 1.24 (d, J = 6.4 | |
| | | | | Hz, 3H). | |
| 36 | 4 | AA-1 | BB-5 | 1H NMR (400 MHz, Methanol- | 339.2 |
| | | | | d4) δ 8.57 (d, J = 1.3 Hz, 1H), | |
| | | | | 7.32 (dd, J = 5.2, 1.2 Hz, 1H), | |
| | | | | 7.13 (d, J = 3.5 Hz, 1H), 6.98 | |
| | | | | (dd, J = 5.1, 3.6 Hz, 1H), 3.38 | |
| | | | | (q, J = 6.4 Hz, 1H), 3.12 (dd, | |
| | | | | J = 6.7, 1.9 Hz, 2H), 1.20 | |
| | | | | (dd, J = 6.3, 1.5 Hz, 3H). | |
| 37 | 13 | isopropenylboronic acid | BB-2 | 1H NMR (300 MHz, DMSO- | 302.2 |
| | | pinacol ester | | d6) δ 8.60 (s, 1H), 7.79 (t, J = | |
| | | | | 6.2 Hz, 1H), 7.41 (dd, J = 5.1, | |
| | | | | 1.3 Hz, 1H), 7.14 (dd, J = 3.5, | |
| | | | | 1.2 Hz, 1H), 6.99 (dd, J = 5.1, | |
| | | | | 3.4 Hz, 1H), 5.50 (p, J = 1.6 | |
| | | | | Hz, 1H), 5.39 (t, J = 1.2 Hz, | |
| | | | | 1H), 4.81 (d, J = 6.1 Hz, 2H), | |
| | | | | 2.53 (s, 3H), 2.21 (dd, J = 1.6, | |
| | | | | 0.9 Hz, 3H). | |
| 38 | 10 | AA-4 | BB-2 | 1H NMR (400 MHz, DMSO- | 328.1 |
| | | | | d6) δ 8.82 (s, 1H), 7.64 (d, J = | |
| | | | | 1.8 Hz, 1H), 6.52 (d, J = 3.2 | |
| | | | | Hz, 1H), 6.45 (t, J = 2.6 Hz, | |
| | | | | 1H), 4.82 (s, 2H), 3.84 (p, J = | |
| | | | | 6.0 Hz, 1H), 3.37 (q, J = 7.0 | |
| | | | | Hz, 3H), 2.99 (d, J = 5.5 Hz, | |
| | | | | 2H), 2.44 (s, 3H), 1.21 (d, J = | |
| | | | | 6.5 Hz, 1H). | |
| 39 | 2 | AA-1 | BB-2 | 1H NMR (400 MHz, CD3OD) | 305.2 |
| | | | | δ 8.50 (s, 1H), 8.41 (s, 1H), | |
| | | | | 7.46 (m, 1H), 7.32 (m, 1H), | |
| | | | | 7.12 (m, 1H), 6.98 (m, 1H), | |
| | | | | 4.88 (d, J = 4.9 Hz, 2H), 3.70 | |
| | | | | (m, 2H), 3.30 (d, J = 6.6 Hz, | |
| | | | | 2H), 1.39 (d, J = 6.3 Hz, 3H). | |
| 40 | 1 | | BB-10 | 1H NMR (300 MHz, DMSO- | 276 |
| | | | | d6) δ 8.61 (s, 1H), 7.81 (t, J = | |
| | | | | 6.1 Hz, 1H), 7.68 (d, J = 1.2 | |
| | | | | Hz, 1H), 7.41 (dd, J = 5.1, 1.3 | |
| | | | | Hz, 1H), 7.15 (dd, J = 3.5, 1.2 | |
| | | | | Hz, 1H), 6.99 (dd, J = 5.1, 3.4 | |
| | | | | Hz, 1H), 4.82 (d, J = 6.1 Hz, | |
| | | | | 2H), 2.95 (qd, J = 7.4, 1.2 Hz, | |
| | | | | 2H), 1.32 (t, J = 7.5 Hz, 3H). | |
| 41 | | tert-butyl (R)-(1- | | 1H NMR (400 MHz, DMSO- | 335.1 |
| | | oxopropan-2- | | d6) δ 8.56 (s, 1H), 7.71 (t, J = | |
| | | yl)carbamate | | 6.3 Hz, 1H), 7.39 (dd, J = 5.1, | |
| | | | | 1.3 Hz, 1H), 7.13 (dd, J = 3.4, | |
| | | | | 1.2 Hz, 1H), 6.98 (dd, J = 5.1, | |
| | | | | 3.4 Hz, 1H), 4.79 (dd, J = 10.1, | |
| | | | | 6.7 Hz, 3H), 3.00 (p, J = 6.6 | |
| | | | | Hz, 1H), 2.44 (s, 3H), 0.94 | |
| | | | | (d, J = 6.5 Hz, 3H). | |
| 42 | | tert-butyl (R)-(1- | | 1H NMR (400 MHz, CD3OD) | 335 |
| | | oxopropan-2- | | δ 8.49 (s, 1H), 7.29 (m, 1H), | |
| | | yl)carbamate | | 7.11 (m, 1H), 6.97 (m, 1H), | |
| | | | | 5.27 (d, J = 4.4 Hz, 1H), 4.88 | |
| | | | | (s, 2H), 3.47 − 3.45 (m, 1H), 2.50 | |
| | | | | (s, 3H), 1.22 (d, J = 6.8 Hz, 3H) | |
| 43 | 5 | | BB-8 | 1H-NMR (300 MHz, Methanol- | 302.2 |
| | | | | d4) δ 8.66 (s, 1H), 6.94 (t, J = | |
| | | | | 2.1 Hz, 2H), 6.10 (t, J = 2.2 Hz, | |
| | | | | 2H), 5.60 (s, 2H), 3.37 (d, J = | |
| | | | | 6.6 Hz, 1H), 3.16 − 2.97 (m, | |
| | | | | 2H), 2.50 (s, 3H), 1.22 (d, J = | |
| | | | | 6.4 Hz, 3H). | |
| 44 | 5 | | BB-8 | 1H-NMR (300 MHz, Methanol- | 302.2 |
| | | | | d4) δ 8.95 (d, J = 0.8 Hz, 1H), | |
| | | | | 8.54 (s, 1H), 7.99 − 7.89 (m, | |
| | | | | 1H), 4.99 (d, J = 1.0 Hz, 2H), | |
| | | | | 3.37 (d, J = 6.7 Hz, 1H), 3.16 − | |
| | | | | 2.96 (m, 2H), 2.50 (s, 3H), 1.22 | |
| | | | | (d, J = 6.4 Hz, 3H). | |
| 45 | 5 | | BB-8 | 1H-NMR (300 MHz, Methanol- | 337.2 |
| | | | | d4) δ 8.48 (s, 1H), 6.96 (d, J = | |
| | | | | 1.5 Hz, 1H), 6.78 (t, J = 1.6 Hz, | |
| | | | | 1H), 4.86 − 4.79 (m, 2H), | |
| | | | | 3.15 − 2.96 (m, 2H), 2.50 (s, 3H), | |
| | | | | 1.21 (d, J = 6.4 Hz, 3H). | |
| 46 | 5 | | BB-8 | 1H NMR (400 MHz, Methanol- | 304.2 |
| | | | | d4) δ 8.59 (s, 1H), 8.16 (s, 1H), | |
| | | | | 7.15 (s, 1H), 4.79 (d, J = 1.0 | |
| | | | | Hz, 2H), 3.39 (q, J = 6.7 Hz, | |
| | | | | 1H), 3.08 (qd, J = 14.5, 6.9 Hz, | |
| | | | | 2H), 2.49 (s, 3H), 1.33 (s, 0H), | |
| | | | | 1.23 (dd, J = 6.5, 1.5 Hz, 3H). | |
| 48 | 10 | AA-19 | BB-2 | 1H-NMR (300 MHz, Methanol- | 351.2 |
| | | | | d4) δ 8.49 (s, 1H), 7.37 − | |
| | | | | 7.27 (m, 1H), 7.18 − 7.08 (m, | |
| | | | | 1H), 7.04 − 6.94 (m, 1H), 4.89 | |
| | | | | (s, 1H), 4.72 (h, J = 4.6 Hz, | |
| | | | | 1H), 4.68 − 4.47 (m, 1H), | |
| | | | | 3.44 − 3.34 (m, 1H), 3.16 − 3.06 | |
| | | | | (m, 2H), 2.50 (s, 3H), 2.11 − | |
| | | | | 1.68 (m, 2H). | |
| 49 | 1 | AA-1 | BB-2 | 1H NMR (400 MHz, DMSO- | 319.1 |
| | | | | d6) δ 8.54 (s, 1H), 7.67 (t, J = | |
| | | | | 6.3 Hz, 1H), 7.40 (dd, J = 5.1, | |
| | | | | 1.3 Hz, 1H), 7.13 (dd, J = 3.4, | |
| | | | | 1.2 Hz, 1H), 6.98 (dd, J = 5.0, | |
| | | | | 3.4 Hz, 1H), 4.79 (d, J = 6.2 | |
| | | | | Hz, 2H), 3.16 − 3.08 (m, 1H), | |
| | | | | 3.00 (dd, J = 16.2, 6.6 Hz, 1H), | |
| | | | | 2.88 (dd, J = 6.6, 1.8 Hz, 2H), | |
| | | | | 2.39 (s, 3H), 1.15 − 0.98 (m, | |
| | | | | 3H). | |
| 50 | 1 | AA-13 | BB-13 | 1H-NMR (300 MHz, Methanol- | 387.2 |
| | | | | d4) δ 8.50 (s, 1H), 7.37 − | |
| | | | | 7.27 (m, 1H), 7.17 − 7.09 (m, | |
| | | | | 1H), 7.04 − 6.94 (m, 1H), 4.90 | |
| | | | | (s, 1H), 3.62 − 3.49 (m, 1H), | |
| | | | | 3.26 − 3.03 (m, 2H), 2.51 (s, | |
| | | | | 3H), 2.49 − 2.28 (m, 2H). | |
| 51 | 2 | | BB-8 | 1H NMR (300 MHz, DMSO- | 333 |
| | | | | d6) δ 9.19 (s, 1H), 8.14 (dd, J = | |
| | | | | 3.8, 1.2 Hz, 1H), 8.00 (dd, J = | |
| | | | | 5.0, 1.1 Hz, 1H), 7.30 (dd, J = | |
| | | | | 5.0, 3.8 Hz, 1H), 3.50 (t, J = 6.5 | |
| | | | | Hz, 1H), 3.31 − 3.06 (m, 2H), | |
| | | | | 1.22 (d, J = 6.4 Hz, 4H). | |
| 52 | 1 | AA-9 | tert-butyl (furan- | 1H-NMR(300 MHz, Methanol- | 343.3 |
| | | | 2-ylmethyl)(7- | d4) δ 8.57 (s, 1H), 7.47 (t, J = | |
| | | | methylthieno[3,2- | 1.4 Hz, 1H), 6.38 (d, J = 1.4 | |
| | | | c]pyridazin-4- | Hz, 2H), 4.68 (s, 2H), 3.50 − | |
| | | | yl)carbamate | 3.36 (m, 1H), 3.32 − 3.22 (m, | |
| | | | | 1H), 3.16 − 3.06 (m, 1H), 2.50 | |
| | | | | (s, 3H), 1.51 (t, J = 6.7 Hz, | |
| | | | | 2H), 0.9 − 0.8 (dm, 1H), 0.66 − | |
| | | | | 0.49 (m, 2H), 0.25 − 0.05 (m, | |
| | | | | 2H). | |
| 53 | 1 | AA-4 | BB-15 | 1H NMR (300 MHz, DMSO- | 321 |
| | | | | d6) δ 8.44 (s, 1H), 7.38 (m, | |
| | | | | 1H), 7.31 (m, 1H), 7.11 (m, | |
| | | | | 1H), 6.98 (m, 1H), 4.87 (d, J = | |
| | | | | 4.9 Hz, 2H), 3.61 (m, 1H), 3.51 | |
| | | | | (m, 1H), 3.30 (d, J = 6.6 Hz, | |
| | | | | 2H), 3.02 (m, 1H). | |
| 54 | | | BB-2 | 1H NMR (300 MHz, DMSO- | 320.2 |
| | | | | d6) δ 8.54 (s, 1H), 7.67 (t, J = | |
| | | | | 6.3 Hz, 1H), 7.39 (dd, J = 5.1, | |
| | | | | 1.3 Hz, 1H), 7.12 (dd, J = 3.5, | |
| | | | | 1.2 Hz, 1H), 6.98 (dd, J = 5.1, | |
| | | | | 3.5 Hz, 1H), 5.95 (s, 1H), 4.79 | |
| | | | | (d, J = 6.2 Hz, 2H), 1.60 (s, | |
| | | | | 6H). | |
| 55 | | | BB-2 | 1H NMR (400 MHz, Methanol- | 337.1 |
| | | | | d4) δ 8.53 (s, 1H), 7.30 (dd, J = | |
| | | | | 5.1, 1.2 Hz, 1H), 7.15 − 7.09 | |
| | | | | (m, 1H), 6.98 (dd, J = 5.1, 3.5 | |
| | | | | Hz, 1H), 5.80 (dd, J = 45.8, 6.1 | |
| | | | | Hz, 1H), 3.39 (dq, J = 13.0, 6.5 | |
| | | | | Hz, 1H), 2.55 (d, J = 1.8 Hz, | |
| | | | | 3H), 1.25 (dd, J = 6.6, 1.4 Hz, | |
| | | | | 3H). | |
| 56 | 4 | AA-1 | BB-14 | −(300 MHz, Methanol-d4) δ | 337 |
| | | | | 8.43 (s, 1H), 6.78 − 6.68 (m, | |
| | | | | 1H), 6.59 (t, J = 1.9 Hz, 1H), | |
| | | | | 4.58 (d, J = 1.2 Hz, 2H), 3.40 − | |
| | | | | 3.27 (m, 1H) 3.15 − 2.95 (m, | |
| | | | | 2H), 2.49 (s, 3H), 1.21 (d, J = | |
| | | | | 6.4 Hz, 3H). | |
| 57 | 1 | None | BB-9 | 1H NMR (400 MHz, | 276 |
| | | | | Chloroform-d) δ 7.31 − 7.25 | |
| | | | | (m, 1H), 7.23 (s, 1H), 7.06 − | |
| | | | | 6.95 (m, 2H), 5.12 (d, J = 6.0 | |
| | | | | Hz, 2H), 4.55 (s, 1H), 2.66 (s, | |
| | | | | 3H), 2.59 (d, J = 1.4 Hz, 3H), | |
| | | | | 1.26 (s, 1H), 0.59 (s, 1H), 0.07 | |
| | | | | (s, 1H). | |
| 58 | 10 | AA-4 | BB-2 | 1H NMR (400 MHz, DMSO- | 365.1 |
| | | | | d6) δ 8.55 (s, 1H), 7.68 (t, J = | |
| | | | | 6.3 Hz, 1H), 7.40 (dd, J = 5.1, | |
| | | | | 1.3 Hz, 1H), 7.13 (d, J = 3.5 | |
| | | | | Hz, 1H), 6.98 (dd, J = 5.1, 3.5 | |
| | | | | Hz, 1H), 4.79 (d, J = 6.2 Hz, | |
| | | | | 2H), 3.12 (d, J = 11.3 Hz, 2H), | |
| | | | | 2.92 − 2.83 (m, 1H), 2.59 (dd, | |
| | | | | J = 13.1, 5.2 Hz, 1H), 2.47 | |
| | | | | (d, J = 6.4 Hz, 1H), 2.41 (s, 3H), | |
| | | | | 2.08 (s, 3H). | |
| 59 | 11 | AA-4 | BB-2 | 1H NMR (400 MHz, DMSO- | 381 |
| | | | | d6) δ 8.55 (d, J = 1.8 Hz, 1H), | |
| | | | | 7.70 (td, J = 6.2, 3.5 Hz, 1H), | |
| | | | | 7.39 (dd, J = 5.1, 1.3 Hz, 1H), | |
| | | | | 7.13 (d, J = 3.5 Hz, 1H), 6.98 | |
| | | | | (dd, J = 5.1, 3.4 Hz, 1H), 4.79 | |
| | | | | (d, J = 6.2 Hz, 2H), 3.16 − 2.96 | |
| | | | | (m, 2H), 2.82 (dd, J = 6.4, 1.9 | |
| | | | | Hz, 1H), 2.77 − 2.72 (m, 1H), | |
| | | | | 2.55 (d, J = 15.6 Hz, 3H), 2.41 | |
| | | | | (s, 3H), 2.35 − 2.07 (m, 1H). | |
| 60 | 11 | AA-4 | BB-2 | 1H NMR (400 MHz, DMSO- | 397 |
| | | | | d6) δ 8.56 (s, 1H), 7.70 (t, J = | |
| | | | | 6.2 Hz, 1H), 7.40 (dd, J = 5.1, | |
| | | | | 1.3 Hz, 1H), 7.13 (dd, J = 3.5, | |
| | | | | 1.2 Hz, 1H), 6.98 (dd, J = 5.1, | |
| | | | | 3.5 Hz, 1H), 4.79 (d, J = 6.2 | |
| | | | | Hz, 2H), 3.51 (q, J = 8.8, 7.1 | |
| | | | | Hz, 1H), 3.27 − 3.12 (m, 3H), | |
| | | | | 3.06 (s, 3H), 3.03 − 2.95 (m, | |
| | | | | 1H), 2.41 (s, 3H), 2.02 (d, J = | |
| | | | | 39.9 Hz, 2H). | |
| 61 | 1 | AA-19 | BB-2 | 1H NMR (300 MHz, Methanol- | 349 |
| | | | | d4) δ 8.49 (s, 1H), 7.37 − 7.27 | |
| | | | | (m, 1H), 7.17 − 7.07 (m, 1H), | |
| | | | | 7.04 − 6.94 (m, 1H), 4.89 (s, | |
| | | | | 2H), 3.75 (t, J = 6.2 Hz, 2H), | |
| | | | | 3.44 − 3.34 (m, 1H), 3.15 − | |
| | | | | 3.050 (m, 2H), 2.50 (s, 3H), | |
| | | | | 1.89 − 1.74 (m, 1H), 1.71 − | |
| | | | | 1.61 (m, 1H). | |
| 62 | 1, 12 | AA-1 | BB-2 | 1H NMR (400 MHz, Methanol- | 397 |
| | | | | d4) δ 8.52 (s, 1H), 6.97 (d, J = | |
| | | | | 3.8 Hz, 1H), 6.95 − 6.89 (m, | |
| | | | | 1H), 4.82 (d, J = 1.0 Hz, 2H), | |
| | | | | 3.66 (dt, J = 7.8, 6.3 Hz, 1H), | |
| | | | | 3.37 − 3.27 (m, 1H), 3.22 (dd, | |
| | | | | J = 14.8, 8.0 Hz, 1H), 2.51 (s, | |
| | | | | 3H), 1.38 (d, J = 6.5 Hz, 3H). | |
| 63 | 4 | None | BB-3 | 1H NMR (300 MHz, Methanol- | 326 |
| | | | | d4) δ 8.63 (s, 1H), 8.09 (s, 1H), | |
| | | | | 7.34 (dd, J = 5.1, 1.2 Hz, 1H), | |
| | | | | 7.15 (dd, J = 3.5, 1.1 Hz, 1H), | |
| | | | | 7.01 (dd, J = 5.1, 3.5 Hz, 1H). | |
| 64 | 1 | AA-22 | BB-2 | (300 MHz, Methanol-d4) δ 8.55 | 329 |
| | | | | (s, 1H), 7.47 (t, J = 1.4 Hz, 1H), | |
| | | | | 6.38 (d, J = 1.5 Hz, 2H), 4.68 | |
| | | | | (s, 2H), 3.31 − 3.10 (m, 2H), | |
| | | | | 2.51 (s, 3H), 2.46 − 2.36 (m, | |
| | | | | 1H), 0.95 − 0.850 (m, 1H), | |
| | | | | 0.62 − 0.42 (m, 2H), 0.36 − 0.26 | |
| | | | | (m, 1H), 0.18 − 0.08 (m, 1H). | |
| 65 | | None | BB-2 | 1H NMR (300 MHz, DMSO- | 320.2 |
| | | | | d6) δ 8.54 (s, 1H), 7.67 (t, J = | |
| | | | | 6.3 Hz, 1H), 7.39 (dd, J = 5.1, | |
| | | | | 1.3 Hz, 1H), 7.12 (dd, J = 3.5, | |
| | | | | 1.2 Hz, 1H), 6.98 (dd, J = 5.1, | |
| | | | | 3.5 Hz, 1H), 5.95 (s, 1H), 4.79 | |
| | | | | (d, J = 6.2 Hz, 2H), 1.60 (s, | |
| | | | | 6H). | |
| 66 | 4 | AA-1 | BB-17 | 1H NMR (400 MHz, Methanol- | 397 |
| | | | | d4) δ 7.28 (dd, J = 5.1, 1.2 Hz, | |
| | | | | 1H), 7.01 (dq, J = 3.5, 1.1 Hz, | |
| | | | | 1H), 6.93 (dd, J = 5.1, 3.5 Hz, | |
| | | | | 1H), 5.12 (d, J = 1.1 Hz, 2H), | |
| | | | | 3.25 (p, J = 6.5 Hz, 1H), 3.08 − | |
| | | | | 2.90 (m, 2H), 2.42 (s, 3H), 1.16 | |
| | | | | (d, J = 6.4 Hz, 3H). | |
| 67 | 4 | none | BB-5 | 1H NMR (400 MHz, Methanol- | 282 |
| | | | | d4) δ 7.28 (dd, J = 5.1, 1.2 Hz, | |
| | | | | 1H), 7.01 (dq, J = 3.5, 1.1 Hz, | |
| | | | | 1H), 6.93 (dd, J = 5.1, 3.5 Hz, | |
| | | | | 1H), 5.12 (d, J = 1.1 Hz, 2H), | |
| | | | | 3.25 (p, J = 6.5 Hz, 1H), 3.08 − | |
| | | | | 2.90 (m, 2H), 2.42 (s, 3H), 1.16 | |
| | | | | (d, J = 6.4 Hz, 3H). | |
| 68 | 1 | AA-9 | BB-2 | 1H NMR (300 MHz, Methanol- | 359 |
| | | | | d4) δ 8.49 (s, 1H), 7.05 − | |
| | | | | 6.95.37 − 7.27 (m, 1H), 7.18 − | |
| | | | | 7.08 (m, 1H), 7.00 (m, 1H), | |
| | | | | 4.90 (d, J = 1.0 Hz, 2H), 3.31 − | |
| | | | | 3.13 (m, 2H), 3.06 − 2.96 (m, | |
| | | | | 1H), 2.50 (s, 3H), 1.44 (t, J = | |
| | | | | 6.7 Hz, 2H), 0.94 − 0.77 (m, | |
| | | | | 1H), 0.59 − 0.49 (m, 2H), | |
| | | | | 0.23 − 0.05 (m, 2H). | |
| 69 | 14 | tert-butyl (R)-2- | BB-11 | 1H NMR (300 MHz, DMSO- | 345.1 |
| | | (iodomethyl)pyrrolidine- | | d6) δ 8.55 − 8.40 (m, 1H), | |
| | | 1-carboxylate | | 7.34 − 7.25 (m, 1H), 7.10 (d, J = | |
| | | | | 3.4 Hz, 1H), 6.97 (dt, J = 5.2, 2.9 | |
| | | | | Hz, 1H), 4.87 (d, J = 6.3 Hz, | |
| | | | | 1H), 3.49 (p, J = 7.2 Hz, 1H), | |
| | | | | 3.33 (s, 1H), 3.29 − 3.06 (m, | |
| | | | | 2H), 2.96 (ddd, J = 10.9, 8.3, | |
| | | | | 6.3 Hz, 1H), 2.48 (d, J = 2.7 | |
| | | | | Hz, 3H), 2.02 (s, 1H), 2.00 − | |
| | | | | 1.82 (m, 1H), 1.59 (dt, J = 12.0, | |
| | | | | 8.5 Hz, 1H). | |
| 70 | 14 | tert-butyl (S)-2- | BB-11 | 1H NMR (300 MHz, DMSO- | 345 |
| | | (iodomethyl)pyrrolidine- | | d6) δ 8.55 − 8.40 (m, 1H), | |
| | | 1-carboxylate | | 7.34 − 7.25 (m, 1H), 7.10 (d, J = | |
| | | | | 3.4 Hz, 1H), 6.97 (dt, J = 5.2, 2.9 | |
| | | | | Hz, 1H), 4.87 (d, J = 6.3 Hz, | |
| | | | | 1H), 3.49 (p, J = 7.2 Hz, 1H), | |
| | | | | 3.33 (s, 1H), 3.29 − 3.06 (m, | |
| | | | | 2H), 2.96 (ddd, J = 10.9, 8.3, | |
| | | | | 6.3 Hz, 1H), 2.48 (d, J = 2.7 | |
| | | | | Hz, 3H), 2.02 (s, 1H), 2.00 − | |
| | | | | 1.82 (m, 1H), 1.59 (dt, J = 12.0, | |
| | | | | 8.5 Hz, 1H). | |
| 71 | 14 | tert-butyl (S)-2- | BB-11 | 1H NMR (400 MHz, DMSO- | 331.2 |
| | | (iodomethyl)azetidine-1- | | d6) δ 8.55 (d, J = 7.1 Hz, 1H), | |
| | | carboxylate | | 7.72 (dt, J = 19.6, 6.3 Hz, 1H), | |
| | | | | 7.39 (d, J = 5.0 Hz, 1H), 7.13 | |
| | | | | (d, J = 3.4 Hz, 1H), 6.98 (dd, | |
| | | | | J = 5.1, 3.5 Hz, 1H), 4.79 (d, J = | |
| | | | | 6.1 Hz, 2H), 4.08 (p, J = 7.1 | |
| | | | | Hz, 1H), 3.73 (d, J = 7.9 Hz, | |
| | | | | 1H), 3.20 (td, J = 8.1, 3.5 Hz, | |
| | | | | 1H), 3.09 (dd, J = 6.6, 3.0 Hz, | |
| | | | | 1H), 2.41 (d, J = 5.6 Hz, 3H), | |
| | | | | 2.22 (ddt, J = 11.3, 7.9, 4.0 Hz, | |
| | | | | 1H), 2.13 − 1.97 (m, 1H). | |
| 72 | 14 | tert-butyl (R)-2- | BB-11 | 1H NMR (400 MHz, DMSO- | 331.1 |
| | | (iodomethyl)azetidine-1- | | d6) δ 8.55 (d, J = 7.5 Hz, 1H), | |
| | | carboxylate | | 7.72 (dt, J = 19.7, 6.2 Hz, 1H), | |
| | | | | 7.39 (dd, J = 5.1, 1.2 Hz, 1H), | |
| | | | | 7.13 (d, J = 3.4 Hz, 1H), 6.98 | |
| | | | | (dd, J = 5.1, 3.4 Hz, 1H), 4.79 | |
| | | | | (d, J = 6.1 Hz, 2H), 4.51 (d, J = | |
| | | | | 66.2 Hz, 1H), 4.25 − 3.99 (m, | |
| | | | | 1H), 3.82 − 3.67 (m, 1H), | |
| | | | | 3.65 − 3.57 (m, 1H), 3.53 − 3.46 | |
| | | | | (m, 2H), 3.21 (s, 1H), 3.10 − 3.05 | |
| | | | | (m, 1H), 2.41 (d, J = 4.9 Hz, | |
| | | | | 3H), 2.23 (d, J = 10.1 Hz, 1H), | |
| | | | | 2.11 − 1.98 (m, 1H). | |
| 73 | 14 | tert-buty1 3- | BB-11 | 1H NMR (300 MHz, DMSO- | 331.1 |
| | | (iodomethyl)azetidine-1- | | d6) δ 8.56 (s, 1H), 7.73 (t, J = | |
| | | carboxylate | | 6.2 Hz, 1H), 7.40 (dd, J = 5.1, | |
| | | | | 1.3 Hz, 1H), 7.13 (dd, J = 3.4, | |
| | | | | 1.2 Hz, 1H), 6.98 (dd, J = 5.1, | |
| | | | | 3.4 Hz, 1H), 4.79 (d, J = 6.1 | |
| | | | | Hz, 2H), 3.94 (s, 1H), 3.78 (s, | |
| | | | | 2H), 3.27 − 3.18 (m, 2H), 3.01 | |
| | | | | (s, 1H), 2.88 (s, 1H), 2.41 (s, | |
| | | | | 3H). | |
| 74 | 5 | AA-1 | BB-2 | (300 MHz, Methanol-d4) δ 8.57 | 349 |
| | | | | (d, J = 5.5 Hz, 1H), 7.25 (d, J = | |
| | | | | 5.5 Hz, 1H), 6.96 (d, J = 5.5 | |
| | | | | Hz, 1H), 4.70 (s, 2H), 3.97 (s, | |
| | | | | 3H), 3.42 (h, J = 6.6 Hz, 1H), | |
| | | | | 3.21 − 3.00 (m, 2H), 2.49 (s, | |
| | | | | 3H), 1.26 (d, J = 6.4 Hz, 3H). | |
| 75 | 5 | AA-1 | BB-2 | (300 MHz, Methanol-d4) δ 8. | 349 |
| | | | | 8.46 (s, 1H), 6.81 − 6.71 (m, | |
| | | | | 1H), 6.29 (d, J = 1.8 Hz, 1H), | |
| | | | | 4.78 (d, J = 1.1 Hz, 2H), 3.76 | |
| | | | | (s, 3H), 3.36 (d, J = 6.6 Hz, | |
| | | | | 1H), 3.15 − 2.95 (m, 2H), 2.49 | |
| | | | | (s, 3H), 1.21 (d, J = 6.4 Hz, 3H). | |
| 76 | 5 | AA-1 | BB-2 | (300 MHz, Methanol-d4) δ 8.51 | 349 |
| | | | | (s, 1H), 6.79 − 6.69 (m, 1H), | |
| | | | | 6.09 (d, J = 3.8 Hz, 1H), 4.71 | |
| | | | | (d, J = 1.1 Hz, 2H), 3.83 (s, | |
| | | | | 3H), 3.38 (d, J = 6.7 Hz, 1H), | |
| | | | | 3.16 − 2.96 (m, 2H), 2.49 (s, | |
| | | | | 3H), 1.22 (d, J= 6.4 Hz, 3H). | |
| 77 | 2 | AA-1 | BB-2 | (300 MHz, Methanol-d4) δ 8.51 | 333 |
| | | | | (s, 1H), 6.79 − 6.69 (m, 1H), | |
| | | | | 6.09 (d, J = 3.8 Hz, 1H), 4.71 | |
| | | | | (d, J = 1.1 Hz, 2H), 3.83 (s, | |
| | | | | 3H), 3.38 (d, J = 6.7 Hz, 1H), | |
| | | | | 3.16 − 2.96 (m, 2H), 2.49 (s, | |
| | | | | 3H), 1.22 (d, J = 6.4 Hz, 3H). | |
| 78 | 5 | | BB-14 | 1H-NMR (300 MHz, Methanol- | 333.2 |
| | | | | d4) δ 8.43 (s, 1H), 7.19 (d, J = | |
| | | | | 5.1 Hz, 1H), 6.87 (d, J = 5.1 | |
| | | | | Hz, 1H), 4.80 (s, 2H), 3.45 − | |
| | | | | 3.350 (m, 1H), 3.19 − 2.98 (m, | |
| | | | | 2H), 2.50 (s, 3H), 2.34 (s, 3H), | |
| | | | | 1.24 (d, J = 6.4 Hz, 3H). | |
| 79 | 5 | | BB-14 | 1H-NMR (300 MHz, Methanol- | 333.2 |
| | | | | d4) δ 8.49 (s, 1H), 6.96 − 6.84 | |
| | | | | (m, 2H), 4.82 (s, 2H), 3.38 (S, | |
| | | | | 1H), 3.17 − 2.98 (m, 2H), 2.50 | |
| | | | | (s, 3H), 2.21 (d, J = 1.1 Hz, | |
| | | | | 3H), 1.23 (d, J = 6.3 Hz, 3H). | |
| 80 | 15 | | | 1H NMR (300 MHz, DMSO- | 345.1 |
| | | | | d6) δ 8.52 (s, 1H), 7.63 (t, J = | |
| | | | | 6.3 Hz, 1H), 7.40 (dd, J = 5.1, | |
| | | | | 1.3 Hz, 1H), 7.13 (dd, J = 3.4, | |
| | | | | 1.2 Hz, 1H), 6.98 (dd, J = 5.1, | |
| | | | | 3.5 Hz, 1H), 4.78 (d, J = 6.1 | |
| | | | | Hz, 2H), 3.26 − 3.16 (m, 2H), | |
| | | | | 3.03 − 2.95 (m, 2H), 2.16 − | |
| | | | | 2.00 (m, 1H), 1.52 − 1.34 (m, | |
| | | | | 2H), 1.10 (d, J = 6.2 Hz, 3H), | |
| | | | | 0.94 − 0.81 (m, 2H). | |
| 82 | 2 | AA-1 | BB-2 | 1H NMR (300 MHz, Methanol- | 305 |
| | | | | d4) δ 8.59 (s, 1H), 7.39 (dd, J = | |
| | | | | 5.5, 1.5 Hz, 1H), 7.12 − 7.00 | |
| | | | | (m, 2H), 3.51 (dt, J = 7.8, 6.5 | |
| | | | | Hz, 1H), 3.27 − 3.05 (m, 2H), | |
| | | | | 2.52 (s, 3H), 1.30 (d, J = 6.5 | |
| | | | | Hz, 3H). | |
| 86 | | | | 1H NMR (400 MHz, Methanol- | 349 |
| | | | | d4) δ 7.38 (dd, J = 5.1, 1.2 Hz, | |
| | | | | 1H), 7.13 (dt, J = 3.6, 1.1 Hz, | |
| | | | | 1H), 6.99 (dd, J = 5.1, 3.5 Hz, | |
| | | | | 1H), 5.19 (d, J = 1.0 Hz, 2H), | |
| | | | | 4.21 (s, 3H), 3.63 (dt, J = 7.8, | |
| | | | | 6.4 Hz, 1H), 3.30 − 3.16 (m, | |
| | | | | 1H), 2.42 (s, 3H), 1.34 (d, J = | |
| | | | | 6.5 Hz, 3H). | |
| 87 | 1, 8 | | BB-2 | 1H NMR (400 MHz, Methanol- | 340 |
| | | | | d4) δ 7.56 (q, J = 1.2 Hz, 1H), | |
| | | | | 7.29 (dd, J = 5.0, 1.2 Hz, 1H), | |
| | | | | 7.02 (dq, J = 3.5, 1.1 Hz, 1H), | |
| | | | | 6.93 (dd, J = 5.1, 3.5 Hz, 1H), | |
| | | | | 5.14 (d, J = 1.1 Hz, 2H), 2.48 | |
| | | | | (d, J = 1.2 Hz, 3H). | |
| 88 | | | BB-11 | 1H NMR (300 MHz, DMSO- | 332 |
| | | | | d6) δ 9.24 (s, 1H), 8.70 (s, 1H), | |
| | | | | 7.48 (dd, J = 5.1, 1.3 Hz, 1H), | |
| | | | | 7.19 (dd, J = 3.5, 1.2 Hz, 1H), | |
| | | | | 7.02 (dd, J = 5.1, 3.4 Hz, 1H), | |
| | | | | 4.98 (d, J = 6.0 Hz, 2H), 3.13 (t, | |
| | | | | J = 7.0 Hz, 2H), 2.90 (t, J = 7.0 | |
| | | | | Hz, 2H), 2.39 (s, 3H), 2.13 (s, | |
| | | | | 3H). | |
| 89 | 1 | | BB-2 | 1H NMR (400 MHz, DMSO- | 276 |
| | | | | d6) δ 12.74 (s, 1H), 11.11 (s, | |
| | | | | 1H), 9.25 (d, J = 0.9 Hz, 1H), | |
| | | | | 8.18 − 8.11 (m, 2H), 8.01 (dd, | |
| | | | | J = 5.0, 1.2 Hz, 1H), 7.90 (d, J = | |
| | | | | 1.3 Hz, 1H), 7.31 (dd, J = 5.0, | |
| | | | | 3.8 Hz, 1H), 2.57 (d, J = 1.3 | |
| | | | | Hz, 3H). | |
| 90 | | | | 1H NMR (400 MHz, Methanol- | 321 |
| | | | | d4) δ 7.89 (d, J = 4.7 Hz, 1H), | |
| | | | | 7.33 (td, J = 5.1, 1.2 Hz, 1H), | |
| | | | | 7.18 − 7.10 (m, 2H), 6.99 (dt, | |
| | | | | J = 5.1, 3.3 Hz, 1H), 4.82 (dd, J = | |
| | | | | 28.4, 1.1 Hz, 2H), 3.36 (d, J = | |
| | | | | 6.5 Hz, 1H), 3.11 − 3.02 (m, | |
| | | | | 2H), 1.22 (d, J = 6.4 Hz, 3H). | |
| 91 | 1 | AA-4 | BB-4 | 1H NMR (400 MHz, Methanol- | 355.1 |
| | | | | d4) δ 8.56 (s, 1H), 7.32 (dd, J = | |
| | | | | 5.1, 1.2 Hz, 1H), 7.13 (dd, J = | |
| | | | | 3.5, 1.1 Hz, 1H), 6.98 (dd, J = | |
| | | | | 5.1, 3.5 Hz, 1H), 4.89 (d, J = | |
| | | | | 1.0 Hz, 2H), 3.60 (dd, J = 10.9, | |
| | | | | 4.5 Hz, 1H), 3.50 (dd, J = 10.9, | |
| | | | | 5.9 Hz, 1H), 3.29 − 3.20 (m, | |
| | | | | 2H), 3.11 − 3.01 (m, 1H). | |
| 92 | 14 | 4-methoxybenzyl (3- | BB-11 | 1H NMR (400 MHz, DMSO- | 347 |
| | | (bromomethyl)oxetan-3- | | d6) δ 8.55 (s, 1H), 7.73 (t, J = | |
| | | yl)carbamate | | 6.2 Hz, 1H), 7.39 (dd, J = 5.1, | |
| | | | | 1.3 Hz, 1H), 7.13 (d, J = 3.5 | |
| | | | | Hz, 1H), 6.98 (dd, J = 5.1, 3.4 | |
| | | | | Hz, 1H), 4.78 (d, J = 6.2 Hz, | |
| | | | | 2H), 4.47 (d, J = 6.1 Hz, 2H), | |
| | | | | 4.39 (d, J = 6.1 Hz, 2H), 2.45 | |
| | | | | (s, 3H). | |
| 93 | 1 | AA-20 | BB-2 | 1H-NMR (300 MHz, Methanol- | 361.2 |
| | | | | d4) δ 8.50 (s, 1H), 7.35 − 7.30 | |
| | | | | (m, 1H), 7.16 − 7.11 (m, | |
| | | | | 1H), 7.03 − 6.97 (m, 1H), 4.90 | |
| | | | | (s, 2H), 4.85 − 4.750 (m, 1H), | |
| | | | | 4.73 − 4.62 (m, 2H), 4.41 (t, J = | |
| | | | | 6.5 Hz, 1H), 3.69 − 3.50 (m, | |
| | | | | 1H), 3.20 − 3.10 (m, 1H), | |
| | | | | 3.08 − 2.98 (m, 2H), 2.50 (s, 3H). | |
| 94 | 1 | AA-4 | BB-4 | 1H NMR (400 MHz, Methanol- | 399 |
| | | | | d4) δ 8.56 (s, 1H), 7.31 (dd, J = | |
| | | | | 5.1, 1.2 Hz, 1H), 7.12 (dd, J = | |
| | | | | 3.5, 1.2 Hz, 1H), 6.98 (dd, J = | |
| | | | | 5.1, 3.5 Hz, 1H), 4.89 (s, 2H), | |
| | | | | 3.61 (dd, J = 10.8, 4.4 Hz, 1H), | |
| | | | | 3.55 − 3.47 (m, 1H), 3.29 − | |
| | | | | 3.22 (m, 2H), 3.07 (q, J = 7.0 | |
| | | | | Hz, 1H). | |
| 95 | 1 | AA-4 | BB-4 | 1H NMR (400 MHz, Methanol- | 385 |
| | | | | d4) δ 8.63 (s, 1H), 7.46 (dd, J = | |
| | | | | 1.8, 0.9 Hz, 1H), 6.41 − 6.34 | |
| | | | | (m, 2H), 4.68 (s, 2H), 3.67 − | |
| | | | | 3.55 (m, 1H), 3.50 (dd, J = | |
| | | | | 10.8, 6.0 Hz, 1H), 3.28 − 3.21 | |
| | | | | (m, 2H), 3.10 − 2.99 (m, 1H). | |
| 96 | 4 | AA-4 | BB-5 | 1H NMR (400 MHz, Methanol- | 339.2 |
| | | | | d4) δ 8.64 (s, 1H), 7.46 (dd, J = | |
| | | | | 1.8, 0.9 Hz, 1H), 6.38 (qd, J = | |
| | | | | 3.2, 1.3 Hz, 2H), 4.68 (s, 2H), | |
| | | | | 3.64 − 3.56 (m, 1H), 3.50 (dd, | |
| | | | | J = 10.9, 5.9 Hz, 1H), 3.30 − 3.20 | |
| | | | | (m, 2H), 3.11 − 3.00 (m, 1H). | |
| 97 | 1 | AA-1 | BB-12 | 1H NMR (400 MHz, DMSO- | 337.2 |
| | | | | d6) δ 7.84 (d, J = 6.4 Hz, 1H), | |
| | | | | 7.39 (dd, J = 5.1, 1.3 Hz, 1H), | |
| | | | | 7.02 (d, J = 3.7 Hz, 1H), 4.89 | |
| | | | | (d, J = 6.7 Hz, 2H), 3.08 (q, J = | |
| | | | | 6.4 Hz, 1H), 2.81 (d, J = 6.5 | |
| | | | | Hz, 2H), 2.33 (d, J = 2.8 Hz, | |
| | | | | 3H), 1.05 (dd, J = 27.1, 6.5 Hz, | |
| | | | | 3H). | |
| 98 | | | BB-11 | 1H NMR (400 MHz, DMSO- | 301 |
| | | | | d6) δ 8.65 (d, J = 4.9 Hz, 1H), | |
| | | | | 7.92 (t, J = 6.2 Hz, 1H), 7.42 | |
| | | | | (dd, J = 5.1, 1.3 Hz, 1H), 7.13 | |
| | | | | (dd, J = 3.4, 1.3 Hz, 1H), 6.98 | |
| | | | | (dd, J = 5.1, 3.5 Hz, 1H), 4.82 | |
| | | | | (d, J = 6.2 Hz, 2H), 4.45 (dp, | |
| | | | | J = 87.5, 6.6 Hz, 2H), 2.41 (s, | |
| | | | | 3H). | |
| 99 | See | tert-butyl (R)-(3- | BB-11 | 1H NMR (400 MHz, DMSO- | 333.2 |
| | Synthesis of | (4,4,5,5-tetramethyl- | | d6) δ 8.55 (d, J = 4.9 Hz, 1H), | |
| | Compounds | 1,3,2-dioxaborolan-2- | | 7.68 (t, J = 6.2 Hz, 1H), 7.40 | |
| | 25 and 26 | yl)but-3-en-2- | | (dd, J = 5.1, 1.3 Hz, 1H), 7.13 | |
| | | yl)carbamat | | (dd, J = 3.4, 1.3 Hz, 1H), 6.98 | |
| | | | | (dd, J = 5.1, 3.5 Hz, 1H), 4.78 | |
| | | | | (d, J = 6.2 Hz, 2H), 3.51 (p, J = | |
| | | | | 6.4 Hz, 1H), 2.96 (p, J = 6.4 | |
| | | | | Hz, 3H), 2.41 (s, 3H), 1.71 (d, | |
| | | | | J = 6.9 Hz, 2H), 1.08 (d, J = 6.4 | |
| | | | | Hz, 3H). | |
| 100 | 1 | AA-1 | BB-4 | 1H NMR (300 MHz, Methanol- | 367 |
| | | | | d4) δ 8.53 (m, 1H), 7.45 (s, | |
| | | | | 1H), 6.37 (m, 1H), 4.67 (s, 2H), | |
| | | | | 3.39 (q, J = 6.5 Hz, 1H), 3.11 | |
| | | | | (d, J = 6.8 Hz, 2H), 1.21 (d, J = | |
| | | | | 6.4 Hz, 3H). | |
| 101 | 4 | AA-4 | BB-5 | 1H NMR (400 MHz, Methanol- | 323.1 |
| | | | | d4) δ 8.64 (s, 1H), 7.46 (dd, J = | |
| | | | | 1.9, 0.9 Hz, 1H), 6.41 − 6.34 | |
| | | | | (m, 2H), 4.68 (s, 2H), 3.38 (s, | |
| | | | | 1H), 3.12 (d, J = 6.7 Hz, 2H), | |
| | | | | 1.20 (d, J = 6.3 Hz, 3H). | |
| 102 | See | tert-butyl (R)-(3- | BB-11 | 1H NMR (400 MHz, DMSO- | 333.2 |
| | Synthesis of | (4,4,5,5-tetramethyl- | | d6) δ 8.55 (d, J = 4.9 Hz, 1H), | |
| | Compounds | 1,3,2-dioxaborolan-2- | | 7.68 (t, J = 6.2 Hz, 1H), 7.40 | |
| | 25 and 26 | yl)but-3-en-2- | | (dd, J = 5.1, 1.3 Hz, 1H), 7.13 | |
| | | yl)carbamat | | (dd, J = 3.4, 1.3 Hz, 1H), 6.98 | |
| | | | | (dd, J = 5.1, 3.5 Hz, 1H), 4.78 | |
| | | | | (d, J = 6.2 Hz, 2H), 3.18 (p, J = | |
| | | | | 6.9 Hz, 1H), 2.96 (p, J = 6.4 | |
| | | | | Hz, 1H), 2.41 (s, 3H), 1.31 (d, | |
| | | | | J = 6.9 Hz, 3H), 0.97 (d, J = 6.4 | |
| | | | | Hz, 3H). | |
| 104 | 1 | AA-21 | BB-13 | 1H-NMR (300 MHz, Methanol- | 333.2 |
| | | | | d4) δ 8.48 (s, 1H), 7.37 − 7.27 | |
| | | | | (m, 1H), 7.17 − 7.09 (m, 1H), | |
| | | | | 7.04 − 6.95 (m, 1H), 4.72 (s, | |
| | | | | 2H), 3.12 − 3.02 (m, 1H), | |
| | | | | 2.93 − 2.79 (m, 2H), 2.77 − 2.67 | |
| | | | | (m, 1H), 2.47 (s, 3H), 2.17 − 2.07 | |
| | | | | (m, 1H), 1.05 (d, J = 6.7 Hz, | |
| | | | | 3H). | |
| 105 | | | BB-11 | 1H NMR (400 MHz, Methanol- | 319.2 |
| | | | | d4) δ 8.46 (s, 1H), 7.30 (dd, J = | |
| | | | | 5.1, 1.2 Hz, 1H), 7.10 (dt, J = | |
| | | | | 3.4, 1.1 Hz, 1H), 6.97 (dd, J = | |
| | | | | 5.1, 3.5 Hz, 1H), 4.87 (d, J = | |
| | | | | 0.9 Hz, 2H), 3.89 (s, 2H), 2.48 | |
| | | | | (s, 3H). | |
| 107 | 15 | | | 1H NMR (400 MHz, Methanol- | 343 |
| | | | | d4) δ 8.50 (s, 1H), 7.31 (dd, J = | |
| | | | | 5.1, 1.2 Hz, 1H), 7.11 (dt, J = | |
| | | | | 3.5, 1.1 Hz, 1H), 6.98 (dd, J = | |
| | | | | 5.1, 3.5 Hz, 1H), 3.42 (q, J = | |
| | | | | 6.6 Hz, 1H), 3.17 (d, J = 6.9 | |
| | | | | Hz, 2H), 2.17 (s, 3H), 1.21 (d, | |
| | | | | J = 6.4 Hz, 3H). | |
| 110 | 1 | AA-7 | BB-13 | 1H-NMR (300 MHz, Methanol- | 351 |
| | | | | d4) δ 8.49 (s, 1H), 7.37 − 7.27 | |
| | | | | (m, 1H), 7.17 − 7.09 (m, 1H), | |
| | | | | 7.04 − 6.94 (m, 1H), 4.88 (s, | |
| | | | | 2H), 4.76 − 4.50 (m, 1H), | |
| | | | | 3.29 − 3.19 (m, 2H), 3.11 − 2.92 | |
| | | | | (m, 1H), 2.50 (s, 3H), 1.52 − 1.35 | |
| | | | | (m, 3H). | |
| 111 | 1 | AA-8 | BB-13 | 1H-NMR (300 MHz, Methanol- | 351 |
| | | | | d4) δ 8.49 (s, 1H), 7.37 − 7.27 | |
| | | | | (m, 1H), 7.18 − 7.08 (m, 1H), | |
| | | | | 7.04 − 6.94 (m, 1H), 4.76 − | |
| | | | | 4.46 (m, 1H), 3.27 − 2.97 (m, | |
| | | | | 3H), 2.50 (s, 3H), 1.46 − 1.36 | |
| | | | | (m, 3H). | |
| 112 | 1 | AA-1 | BB-4 | 1H NMR (400 MHz, Methanol- | 399 |
| | | | | d4) δ 8.51 (s, 1H), 7.34 (dd, J = | |
| | | | | 5.0, 1.2 Hz, 1H), 7.13 − 7.07 | |
| | | | | (m, 1H), 6.97 (dd, J = 5.1, 3.5 | |
| | | | | Hz, 1H), 4.74 − 4.57 (m, 3H), | |
| | | | | 4.11 (d, J = 26.9 Hz, 1H), | |
| | | | | 3.17 − 2.98 (m, 2H), 2.76 (dd, | |
| | | | | J = 17.9, 9.8 Hz, 1H), 1.39 | |
| | | | | (dd, J = 13.8, 6.5 Hz, 3H). | |
| 113 | 1 | AA-1 | BB-4 | 1H NMR (400 MHz, Methanol- | 399 |
| | | | | d4) δ 8.51 (s, 1H), 7.34 (dd, J = | |
| | | | | 5.0, 1.2 Hz, 1H), 7.13 − 7.07 | |
| | | | | (m, 1H), 6.97 (dd, J = 5.1, 3.5 | |
| | | | | Hz, 1H), 4.74 − 4.57 (m, 3H), | |
| | | | | 4.11 (d, J = 26.9 Hz, 1H), | |
| | | | | 3.17 − 2.98 (m, 2H), 2.76 (dd, | |
| | | | | J = 17.9, 9.8 Hz, 1H), 1.39 | |
| | | | | (dd, J = 13.8, 6.5 Hz, 3H). | |
| 114 | | | BB-13 | 1H NMR (300 MHz, DMSO- | 291.2 |
| | | | | d6) δ 8.56 (s, 1H), 7.73 (t, J = | |
| | | | | 6.2 Hz, 1H), 7.40 (dd, J = 5.1, | |
| | | | | 1.3 Hz, 1H), 7.13 (dd, J = 3.5, | |
| | | | | 1.2 Hz, 1H), 6.98 (dd, J = 5.1, | |
| | | | | 3.5 Hz, 1H), 5.80 (t, J = 5.6 Hz, | |
| | | | | 1H), 4.80 (t, J = 5.8 Hz, 4H), | |
| | | | | 2.38 (s, 3H). | |
| 115 | 15 | | | 1H NMR (400 MHz, Methanol- | 313 |
| | | | | d4) δ 8.58 (d, J = 26.9 Hz, 1H), | |
| | | | | 7.46 (dd, J = 1.8, 0.9 Hz, 1H), | |
| | | | | 6.47 − 6.26 (m, 2H), 4.67 (s, 1H), | |
| | | | | 3.59 − 3.44 (d, J = 6.9 Hz, 2H), | |
| | | | | 1.25 (d, J = 6.4 Hz, 3H). | |
| 116 | 15 | | | 1H NMR (400 MHz, DMSO- | 328.8 |
| | | | | d6) δ 8.61 (d, J = 5.8 Hz, 1H), | |
| | | | | 7.96 (t, J = 6.1 Hz, 1H), 7.41 | |
| | | | | (dd, J = 5.0, 1.3 Hz, 1H), 7.15 | |
| | | | | (dd, J = 3.4, 1.2 Hz, 1H), 6.99 | |
| | | | | (dd, J = 5.1, 3.5 Hz, 1H), 4.80 | |
| | | | | (d, J = 6.1 Hz, 2H), 4.54 (s, | |
| | | | | 1H), 3.21 (q, J = 6.3 Hz, 1H), | |
| | | | | 3.19 − 2.96 (m, 2H), 1.05 (d, | |
| | | | | J = 6.2 Hz, 3H). | |
| 125 | | | | 1H NMR (300 MHz, Methanol- | 355 |
| | | | | d4) δ 8.58 (s, 1H), 7.31 (dd, J = | |
| | | | | 5.1, 1.3 Hz, 1H), 7.16 − 7.09 | |
| | | | | (m, 1H), 6.98 (dd, J = 5.1, 3.5 | |
| | | | | Hz, 1H), 4.89 (s, 10H), 4.59 (s, | |
| | | | | 4H), 3.59 (ddd, J = 18.2, 13.4, | |
| | | | | 6.8 Hz, 1H), 2.64 (t, J = 2.0 Hz, | |
| | | | | 3H), 1.22 (d, J = 6.7 Hz, 3H). | |
| 127 | 1 | AA-23 | BB-13 | 1H-NMR (400 MHz, Methanol- | 395 |
| | | | | d4) δ 8.47 (d, J = 1.1 Hz, 1H), | |
| | | | | 7.30 (d, J = 5.3 Hz, 1H), 7.11 | |
| | | | | (d, J = 3.5 Hz, 1H), 7.02 − 6.92 | |
| | | | | (m, 1H), 4.87 (s, 2H), 3.22 − | |
| | | | | 3.12 (m, 1H), 3.06 − 2.96 (m, | |
| | | | | 1H), 2.48 (s, 3H), 1.74 (dm, | |
| | | | | 3H), 1.58 − 1.48 (m, 1H), | |
| | | | | 1.46 − 1.33 (m, 1H), 1.13 − 1.03 | |
| | | | | (m, 1H). | |
| 128 | 1 | AA-23 | BB-13 | 1H-NMR (400 MHz, Methanol- | 395 |
| | | | | d4) δ 8.47 (d, J = 1.1 Hz, 1H), | |
| | | | | 7.30 (d, J = 5.3 Hz, 1H), 7.11 | |
| | | | | (d, J = 3.5 Hz, 1H), 7.02 − 6.92 | |
| | | | | (m, 1H), 4.87 (s, 2H), 3.22 − | |
| | | | | 3.12 (m, 1H), 3.06 − 2.96 (m, | |
| | | | | 1H), 2.48 (s, 3H), 1.74 (dm, | |
| | | | | 3H), 1.58 − 1.48 (m, 1H), | |
| | | | | 1.46 − 1.33 (m, 1H), 1.13 − 1.03 | |
| | | | | (m, 1H). | |
| 129 | 1 | AA-23 | BB-13 | 1H NMR (400 MHz, Methanol- | 394.9 |
| | | | | d4) δ 8.47 (s, 1H), 7.35 − 7.250 | |
| | | | | (m, 1H), 7.15 − 7.050 (m, 1H), | |
| | | | | 7.02 − 6.92 (m, 1H), 4.87 (s, | |
| | | | | 2H), 3.26 (t, J = 6.5 Hz, 1H), | |
| | | | | 3.2 − 3.1 (m, 1H), 3.04 − 2.94 | |
| | | | | (m, 1H), 2.48 (s, 3H), 1.84 − | |
| | | | | 1.58 (m, 3H), 1.57 − 1.45 (m, | |
| | | | | 1H), 1.17 − 1.00 (m, 1H). | |
| 130 | 1 | AA-23 | BB-13 | 1H NMR (400 MHz, Methanol- | 395 |
| | | | | d4) δ 8.47 (s, 1H), 7.35 − 7.250 | |
| | | | | (m, 1H), 7.15 − 7.050 (m, 1H), | |
| | | | | 7.02 − 6.92 (m, 1H), 4.87 (s, | |
| | | | | 2H), 3.26 (t, J = 6.5 Hz, 1H), | |
| | | | | 3.2 − 3.1 (m, 1H), 3.04 − 2.94 | |
| | | | | (m, 1H), 2.48 (s, 3H), 1.84 − | |
| | | | | 1.58 (m, 3H), 1.57 − 1.45 (m, | |
| | | | | 1H), 1.17 − 1.00 (m, 1H). | |
| 131 | 1 | AA-11 | BB-13 | 1H NMR (400 MHz, Methanol- | 375 |
| | | | | d4) δ 8.46 (s, 1H), 7.30 (dd, J = | |
| | | | | 5.1, 1.2 Hz, 1H), 7.10 (dq, J = | |
| | | | | 3.4, 1.1 Hz, 1H), 6.97 (dd, J = | |
| | | | | 5.1, 3.5 Hz, 1H), 3.52 (dd, J = | |
| | | | | 9.5, 4.4 Hz, 1H), 3.43 (dd, J = | |
| | | | | 9.5, 5.8 Hz, 1H), 3.34 (s, 1H), | |
| | | | | 3.25 (ddd, J = 7.1, 5.9, 4.4 Hz, | |
| | | | | 1H), 3.12 (dd, J = 14.6, 6.8 Hz, | |
| | | | | 1H), 2.98 (dd, J = 14.5, 7.2 Hz, | |
| | | | | 1H), 2.46 (s, 3H), 0.62 − 0.52 | |
| | | | | (m, 2H), 0.52 − 0.42 (m, 2H). | |
| 132 | 1 | AA-14 | BB-13 | 1H NMR (400 MHz, Methanol- | 403 |
| | | | | d4) δ 8.47 (s, 1H), 7.33 − 7.27 | |
| | | | | (m, 1H), 7.13 − 7.08 (m, 1H), | |
| | | | | 6.97 (dd, J = 5.1, 3.4 Hz, 1H), | |
| | | | | 4.61 (s, 2H), 4.04 (dd, J = 9.6, | |
| | | | | 4.7 Hz, 1H), 3.98 (dd, J = 9.6, | |
| | | | | 5.6 Hz, 1H), 3.40 (p, J = 5.7 | |
| | | | | Hz, 1H), 3.19 (dd, J = 14.8, 6.3 | |
| | | | | Hz, 1H), 3.05 (dd, J = 14.7, 7.7 | |
| | | | | Hz, 1H), 2.48 (s, 3H). | |
| 133 | 1 | AA-12 | BB-13 | 1H NMR (400 MHz, Methanol- | 384.9 |
| | | | | d4) δ 9.32 (s, 1H), 8.05 (t, J = | |
| | | | | 58.4 Hz, 1H), 7.41 (dd, J = 5.2, | |
| | | | | 1.3 Hz, 1H), 7.23 (dd, J = 3.6, | |
| | | | | 1.1 Hz, 1H), 7.03 (dd, J = 5.1, | |
| | | | | 3.5 Hz, 1H), 5.06 (s, 2H), 3.80 | |
| | | | | (dd, J = 11.2, 2.8 Hz, 1H), 3.64 | |
| | | | | (td, J = 12.0, 11.1, 5.2 Hz, 2H), | |
| | | | | 3.53 (dd, J = 14.7, 8.1 Hz, 1H), | |
| | | | | 3.39 (dd, J = 14.8, 6.2 Hz, 1H), | |
| | | | | 2.54 (s, 3H). | |
| 135 | 1 | AA-7 | BB-4 | 1H NMR (400 MHz, Methanol- | 416.9 |
| | | | | d4) δ 8.59 (d, J = 6.4 Hz, 1H), | |
| | | | | 7.32 (dd, J = 5.1, 1.3 Hz, 1H), | |
| | | | | 7.13 (dd, J = 3.6, 1.4 Hz, 1H), | |
| | | | | 6.98 (dd, J = 5.2, 3.5 Hz, 1H), | |
| | | | | 4.90 (s, 3H), 3.73 (s, 1H), 3.51 | |
| | | | | (dd, J = 15.2, 4.7 Hz, 1H), | |
| | | | | 3.27 − 3.16 (m, 1H), 1.59 − 1.44 | |
| | | | | (m, 3H). | |
| 136 | 1 | AA-5 | BB-4 | 1H NMR (400 MHz, Methanol- | 415 |
| | | | | d4) δ 8.69 − 8.56 (m, 1H), 7.34 | |
| | | | | (dd, J = 5.1, 1.2 Hz, 1H), 7.15 | |
| | | | | (dd, J = 3.5, 1.2 Hz, 1H), 7.00 | |
| | | | | (dd, J = 5.2, 3.5 Hz, 1H), 4.94 | |
| | | | | (s, 2H), 3.86 − 3.77 (m, 1H), | |
| | | | | 3.61 (dd, J = 10.6, 3.1 Hz, 1H), | |
| | | | | 3.53 − 3.47 (m, 2H), 3.44 (s, | |
| | | | | 4H). | |
| 137 | 1 | AA-11 | BB-4 | 1H NMR (400 MHz, Methanol- | 438.9 |
| | | | | d4) δ 8.56 (s, 1H), 7.31 (dd, J = | |
| | | | | 5.1, 1.2 Hz, 1H), 7.12 (dt, J = | |
| | | | | 3.5, 1.1 Hz, 1H), 6.98 (dd, J = | |
| | | | | 5.1, 3.5 Hz, 1H), 4.89 (s, 2H), | |
| | | | | 3.54 (dd, J = 9.5, 4.4 Hz, 1H), | |
| | | | | 3.46 (dd, J = 9.5, 5.7 Hz, 1H), | |
| | | | | 3.42 − 3.35 (m, 1H), 3.35 − | |
| | | | | 3.32 (m, 1H), 3.23 (dd, J = | |
| | | | | 14.5, 6.6 Hz, 1H), 3.08 (dd, J = | |
| | | | | 14.5, 7.3 Hz, 1H), 0.63 − 0.53 | |
| | | | | (m, 2H), 0.51 − 0.40 (m, 2H). | |
| 139 | | AA-19 | BB-13 | 1H NMR (400 MHz, Methanol- | 399 |
| | | | | d4) δ 8.46 (s, 1H), 7.35 − 7.250 | |
| | | | | (m, 1H), 7.13 − 7.07 (m, 1H), | |
| | | | | 7.02 − 6.92 (m, 1H), 6.36 (t, J = | |
| | | | | 75.7 Hz, 1H), 4.87 (s, 2H), 4.01 | |
| | | | | (t, J = 6.3 Hz, 2H), 3.29 − 3.26 | |
| | | | | (m, 1H), 3.16 − 2.93 (m, 2H), | |
| | | | | 2.48 (s, 3H), 2.02 − 1.65 (m, | |
| | | | | 2H). | |
| 140 | See | | BB-4 | 1H NMR (400 MHz, DMSO- | 420.8 |
| | Synthesis of | | | d6) δ 8.78 (s, 1H), 8.31 (t, J = | |
| | Compound | | | 6.0 Hz, 1H), 7.43 (dd, J = 5.0, | |
| | 125 | | | 1.3 Hz, 1H), 7.18 (d, J = 3.5 | |
| | | | | Hz, 1H), 7.00 (dd, J = 5.1, 3.4 | |
| | | | | Hz, 1H), 4.86 (d, J = 6.0 Hz, | |
| | | | | 2H), 1.90 (s, 2H), 1.16 (d, J = | |
| | | | | 6.6 Hz, 3H). | |
| 141 | 1 | AA-3 | BB-13 | 1H NMR (400 MHz, Methanol- | 337 |
| | | | | d4) δ 8.47 (s, 1H), 7.30 (dd, J = | |
| | | | | 5.1, 1.3 Hz, 1H), 7.11 (dt, J = | |
| | | | | 3.5, 1.1 Hz, 1H), 6.97 (dd, J = | |
| | | | | 5.1, 3.5 Hz, 1H), 4.37 (dddd, | |
| | | | | J = 47.4, 27.2, 9.3, 4.7 Hz, 2H), | |
| | | | | 3.37 (ddd, J = 12.1, 7.2, 4.7 Hz, | |
| | | | | 1H), 3.17 (dd, J = 14.6, 6.6 Hz, | |
| | | | | 1H), 3.04 (dd, J = 14.7, 7.5 Hz, | |
| | | | | 1H), 2.47 (s, 3H). | |
| 142 | | | BB-13 | 1H NMR (400 MHz, Methanol- | 343 |
| | | | | d4) δ 8.46 (d, J = 1.8 Hz, 1H), | |
| | | | | 7.35 − 7.250 (m, 1H), 7.10 (s, | |
| | | | | 1H), 7.02 − 6.92 (m, 1H), 4.87 | |
| | | | | (s, 2H), 3.29 − 3.19 (m, 2H), | |
| | | | | 3.09 − 2.99 (m, 1H), 2.53 − | |
| | | | | 2.43 (m, 4H), 2.43 − 2.33 (m, | |
| | | | | 2H). | |

### Specific Examples of General Methods

### Specific Example of General Method 1, Synthesis of 6-[(2S)-2-aminopropyl]-N-(furan-2-ylmethyl)-7-methylthieno[3,2-c]pyridazin-4-amine Compound 2

### Step 1

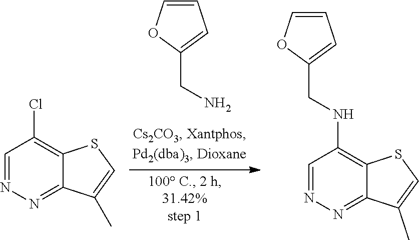 [SMILES: Cc1csc2c(Cl)cnnc12 ; NCc1ccco1 ; Cc1csc2c(NCc3ccco3)cnnc12]

To a solution of furfurylamine (630 mg, 6.487 mmol, 1 equiv) and 4-chloro-7-methylthieno[3,2-c]pyridazine (1000 mg, 5.416 mmol, 0.83 equiv) in Dioxane (15 mL, 177.059 mmol, 27.29 equiv) were added Cs₂CO₃ (3170.39 mg, 9.730 mmol, 1.5 equiv), Xantphos (563.04 mg, 0.973 mmol, 0.15 equiv) and Pd₂(dba)₃ (594.04 mg, 0.649 mmol, 0.1 equiv). After stirring for 2 h at 100° C. under a nitrogen atmosphere, the resulting mixture was concentrated under reduced pressure. The residue was purified by reverse flash chromatography with the following conditions: (column, C18; mobile phase, MeCN in water, 10% to 100% gradient in 15 min; detector, UV 254 nm) to afford N-(furan-2-ylmethyl)-7-methylthieno[3,2-c]pyridazin-4-amine (500 mg, 31.42%) as a white solid.

### Step 2

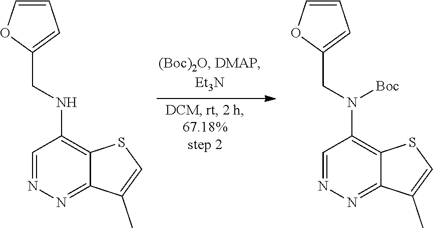 [SMILES: Cc1csc2c(NCc3ccco3)cnnc12 ; Cc1csc2c(N(Cc3ccco3)C(=O)OC(C)(C)C)cnnc12]

To a solution of N-(furan-2-ylmethyl)-7-methylthieno[3,2-c]pyridazin-4-amine (370 mg, 1.508 mmol, 1 equiv) and (Boc)₂O (493.80 mg, 2.262 mmol, 1.5 equiv) in DCM (5.5 mL, 86.518 mmol, 57.36 equiv) were added DMAP (276.42 mg, 2.262 mmol, 1.5 equiv) and Et₃N (305.27 mg, 3.016 mmol, 2 equiv). After stirring for 2 h at room temperature under a nitrogen atmosphere, the resulting mixture was concentrated under reduced pressure. The residue was purified by reverse flash chromatography with the following conditions: (column, C₁₈; mobile phase, MeCN in water, 10% to 100% gradient in 15 min; detector, UV 254 nm. to afford tert-butyl N-(furan-2-ylmethyl)-N-{7-methylthieno[3,2-c]pyridazin-4-yl}carbamate (350 mg, 67.18%) as a brown solid.

### Step 3

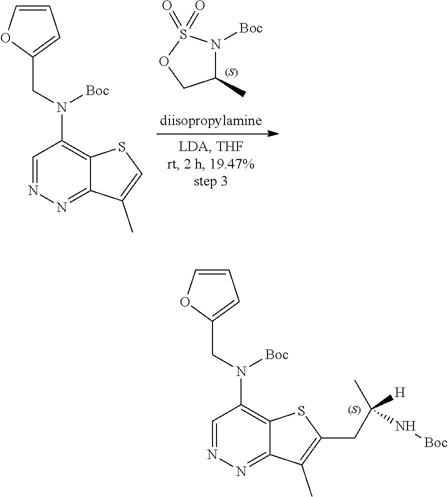

A solution of diisopropylamine (175.77 mg, 1.738 mmol, 2 equiv) in THF was treated with n-BuLi (111.27 mg, 1.738 mmol, 2 equiv) for 15 min at −78° C. under nitrogen atmosphere followed by the addition of tert-butyl N-(furan-2-ylmethyl)-N-{7-methylthieno[3,2-c]pyridazin-4-yl}carbamate (300 mg, 0.869 mmol, 1 equiv) dropwise at −78° C. To the above mixture was added tert-butyl (4S)-4-methyl-2,2-dioxo-1, 2lambda6,3-oxathiazolidine-3-carboxylate (206.07 mg, 0.869 mmol, 1 equiv) dropwise over 3 min at −78° C. The resulting mixture was stirred for additional 2 h at room temperature. The residue was purified by reverse flash chromatography with the following conditions: (column, C18; mobile phase, MeCN in water, 10% to 100% gradient in 15 min; detector, UV 254 nm.) to afford tert-butyl N-{6-[(2S)-2-[(tert-butoxycarbonyl) amino]propyl]-7-methylthieno[3,2-c]pyridazin-4-yl}-N-(furan-2-ylmethyl)carbamate (85 mg, 19.47%) as a white solid.

### Step 4

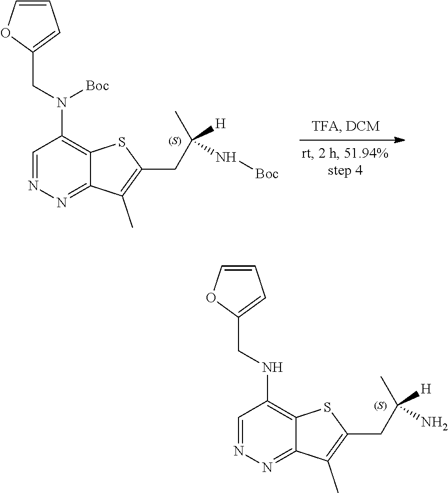

To a solution of tert-butyl N-{6-[(2S)-2-[(tert-butoxycarbonyl)amino]propyl]-7-methylthieno[3,2-c]pyridazin-4-yl}-N-(furan-2-ylmethyl)carbamate (80 mg, 0.159 mmol, 1 equiv) in TFA (1 mL, 13.463 mmol, 84.59 equiv) and DCM (3 mL, 47.192 mmol, 296.50 equiv). After stirring for 2 h at room temperature under a nitrogen atmosphere, the resulting mixture was concentrated under reduced pressure. The residue was purified by reverse flash chromatography with the following conditions: (column, C₁₈; mobile phase, MeCN in Water (10 mmol/L NH₄HCO₃), 10% to 50% gradient in 10 min; detector, UV 254 nm) to afford 6-[(2S)-2-aminopropyl]-N-(furan-2-ylmethyl)-7-methylthieno[3,2-c]pyridazin-4-amine (25 mg, 51.94%). LC-MS (ES, m/z): [M+H]⁺=303.3

¹H NMR (300 MHz, DMSO-d₆) δ 8.62 (s, 1H), 7.62-7.51 (m, 2H), 6.41 (d, J=1.8 Hz, 2H), 4.59 (d, J=6.1 Hz, 2H), 3.17 (q, J=6.4 Hz, 1H), 2.90 (d, J=6.8 Hz, 2H), 2.40 (s, 3H), 1.14-1.02 (m, 3H).

### Specific Example of General Synthesis Method 2, synthesis of (2R)-2-amino-3-{7-methyl-4-[(thiophen-2-ylmethyl)amino]thieno[3,2-c]pyridazin-6-yl}propan-1-ol, Compound 3

### Step 1

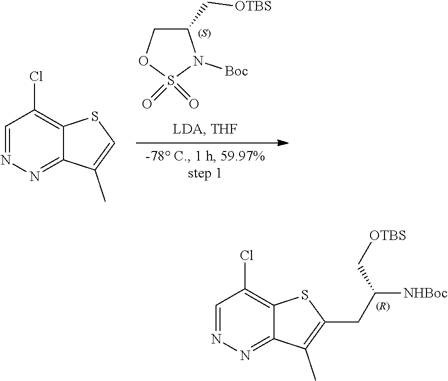

Into a 40-mL vial, to a solution of 4-chloro-7-methylthieno[3,2-c]pyridazine (BB-2300 mg, 1.625 mmol, 1 equiv) in THF (10 mL) was added LDA (1.62 mL, 3.250 mmol, 2 equiv) dropwise at −78 degrees C. under N₂ atmosphere. The reaction mixture was stirred at −78 degrees C. for 30 min. Then a solution of tert-butyl (4S)-4-{[(tert-butyldimethylsilyl)oxy]methyl}-2,2-dioxo-1,2lambda6,3-oxathiazolidine-3-carboxylate (AA-4, 776.30 mg, 2.113 mmol, 1.3 equiv) in 2 mL THE was added dropwise and the mixture was stirred for another 1 h −78 degrees C. The reaction was quenched by the addition of sat. Citric acid (10%) (3 mL) at 0° C., and then the mixture was extracted with EtOAc (3*15 mL). The combined organic extracts were washed with brine (15 mL), sat. sodium hyposulfite (aq.) (15 mL), dried over anhydrous Na₂SO₄, and concentrated under vacuum. The residue was purified by reverse flash chromatography with the following conditions: column, C₁₈; mobile phase, MeCN in Water (10 mmol/L NH₄HCO₃), 30% to 100% gradient in 20 min; detector, UV 254 nm. This resulted in tert-butyl N-[(2R)-1-[(tert-butyldimethylsilyl)oxy]-3-{4-chloro-7-methylthieno[3,2-c]pyridazin-6-yl}propan-2-yl]carbamate (460 mg, 59.97%) as a yellow solid.

### Step 2

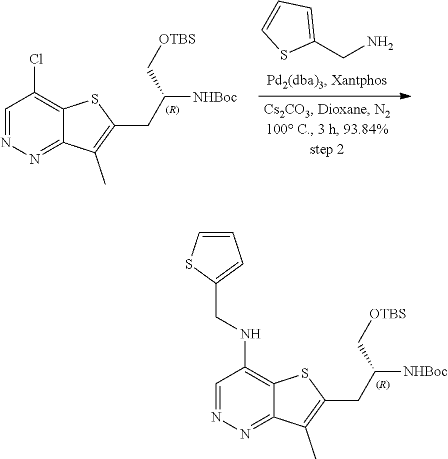

Into a 8-mL vial purged and maintained with an inert atmosphere of nitrogen, were added tert-butyl N-[(2R)-1-[(tert-butyldimethylsilyl)oxy]-3-{4-chloro-7-methylthieno[3,2-c]pyridazin-6-yl}propan-2-yl]carbamate (220 mg, 0.466 mmol, 1 equiv), 1-(thiophen-2-yl)methanamine (158.22 mg, 1.398 mmol, 3 equiv), Pd₂(dba)₃ (42.67 mg, 0.047 mmol, 0.1 equiv), Xantphos (53.93 mg, 0.093 mmol, 0.2 equiv) and Cs₂CO₃ (303.65 mg, 0.932 mmol, 2 equiv) in Dioxane (2 mL) at room temperature. The resulting mixture was stirred for 3 h at 100 degrees C. under nitrogen atmosphere. The residue was purified by silica gel column chromatography, eluted with PE/EA (1:5) to afford tert-butyl N-[(2R)-1-[(tert-butyldimethylsilyl)oxy]-3-{7-methyl-4-[(thiophen-2-ylmethyl)amino]thieno[3,2-c]pyridazin-6-yl}propan-2-yl]carbamate (240 mg, 93.84%) as a yellow solid.

### Step 3

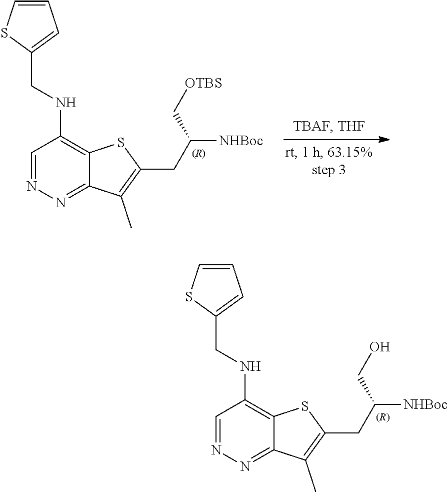

To a solution of tert-butyl N-[(2R)-1-[(tert-butyldimethylsilyl)oxy]-3-{7-methyl-4-[(thiophen-2-ylmethyl)amino]thieno[3,2-c]pyridazin-6-yl}propan-2-yl]carbamate (220 mg, 0.401 mmol, 1 equiv) in THE (2 mL) was added TBAF (0.48 mL, 0.481 mmol, 1.2 equiv) dropwise at 0° C. and stirred for 1 h at room temperature. The residue was purified by silica gel column chromatography, eluted with CH₂Cl₂/MeOH (5:1) to afford tert-butyl N-[(2R)-1-hydroxy-3-{7-methyl-4-[(thiophen-2-ylmethyl)amino]thieno[3,2-c]pyridazin-6-yl}propan-2-yl]carbamate (110 mg, 63.15%) as a yellow solid.

### Step 4

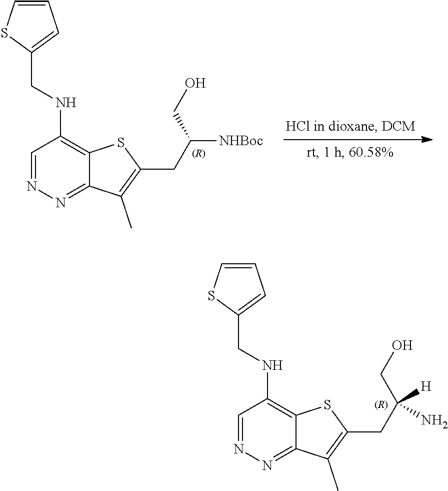

To a solution of tert-butyl N-[(2R)-1-hydroxy-3-{7-methyl-4-[(thiophen-2-ylmethyl)amino]thieno[3,2-c]pyridazin-6-yl}propan-2-yl]carbamate (110 mg, 0.253 mmol, 1 equiv) in DCM (1 mL) was added HCl (gas) in 1,4-dioxane (1 mL) dropwise at 0° C. The resulting mixture was stirred for 1 h at room temperature. The mixture/residue was basified pH 8 with TEA. The resulting mixture was concentrated under vacuum. The residue was purified by reverse flash chromatography with the following conditions: column, C₁₈; mobile phase, ACN in water, 0% to 100% gradient in 40 min; detector, UV 254 nm. This resulted in (2R)-2-amino-3-{7-methyl-4-[(thiophen-2-ylmethyl)amino]thieno[3,2-c]pyridazin-6-yl}propan-1-ol (51.6 mg, 60.58%).

LC-MS (ES, m/z): [M+H]⁺=335

¹H-NMR300 MHz, DMSO-d₆) δ 8.55 (s, 1H), 7.69 (t, J=6.1 Hz, 1H), 7.45-7.350 (m, 1H), 7.19-7.09 (m, 1H), 7.04-6.94 (m, 1H), 4.80 (d, J=6.1 Hz, 2H), 3.39-3.29 (m, 5H), 3.07-2.97 (m, 2H), 2.84-2.74 (m, 1H), 2.40 (s, 3H).

### Specific Example of General Synthesis Method 3. Synthesis of 6-[(2S)-2-aminopropyl]-7-methyl-N-(thiophen-2-ylmethyl)thieno[3,2-c]pyridazin-4-amine Compound 4

### Step 1

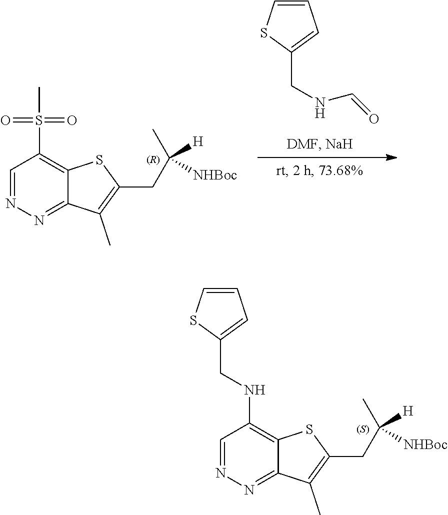

Into a 8 mL vial were added tert-butyl N-[(2S)-1-{4-methanesulfonyl-7-methylthieno[3,2-c]pyridazin-6-yl}propan-2-yl]carbamate (100 mg, 0.259 mmol, 1 equiv), N-(thiophen-2-ylmethyl)formamide (54.94 mg, 0.389 mmol, 1.5 equiv), DMF (4 mL) and NaH (18.68 mg, 0.777 mmol, 3 equiv) at 0° C. The resulting mixture was stirred for 2 h at room temperature under nitrogen atmosphere. The residue was purified by reverse flash chromatography with the following conditions: column, C18; mobile phase, MeCN in Water (10 mmol/L NH₄HCO₃), 20% to 60% gradient in 20 min; detector, UV 254 nm. This resulted in tert-butyl N-[(2S)-1-{7-methyl-4-[(thiophen-2-ylmethyl)amino]thieno[3,2-c]pyridazin-6-yl}propan-2-yl]carbamate (80 mg, 73.68%) as a light yellow solid.

### Step 2

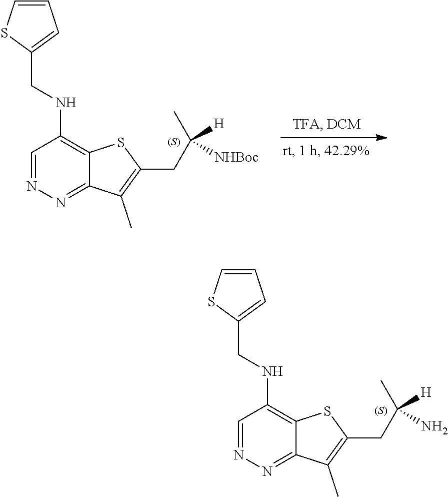

Into a 8 mL vial were added tert-butyl N-[(2S)-1-{7-methyl-4-[(thiophen-2-ylmethyl)amino]thieno [3,2-c]pyridazin-6-yl}propan-2-yl]carbamate (80 mg, 0.191 mmol, 1 equiv), DCM (2 mL) and TFA (1 mL) at room temperature. The resulting mixture was stirred for 1 h at room temperature. The resulting mixture was concentrated under vacuum. The residue was purified by reverse flash chromatography with the following conditions: column, C₁₈; mobile phase, MeCN in Water (10 mmol/L NH₄HCO₃), 10% to 50% gradient in 10 min; detector, UV 254 nm. This resulted in 6-[(2S)-2-aminopropyl]-7-methyl-N-(thiophen-2-ylmethyl)thieno[3,2-c]pyridazin-4-amine (30 mg, 49.29%). LC-MS (ES, m/z): [M+H]⁺=319

¹H NMR (300 MHz, DMSO-d₆) δ 8.55 (s, 1H), 7.69 (t, J=6.2 Hz, 1H), 7.40 (dd, J=5.1, 1.3 Hz, 1H), 7.14 (dd, J=3.5, 1.2 Hz, 1H), 6.99 (dd, J=5.1, 3.4 Hz, 1H), 4.80 (d, J=6.1 Hz, 2H), 3.14 (h, J=6.3 Hz, 1H), 2.88 (d, J=6.4 Hz, 2H), 2.40 (s, 3H), 1.05 (d, J=6.3 Hz, 3H).

### Specific Example of General Synthesis Method 4, Synthesis of 7-bromo-N-(thiophen-2-ylmethyl)thieno[3,2-c]pyridazin-4-amine Compound 63

### Step 2

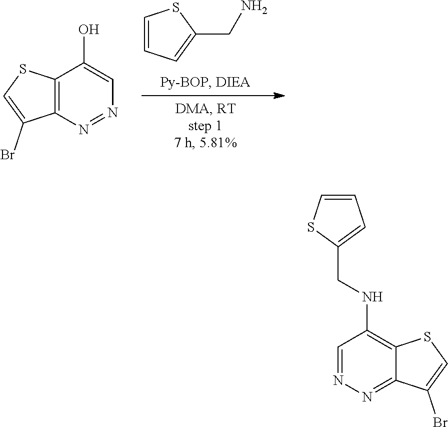

Into a 8-mL vial purged and maintained with an inert atmosphere of nitrogen, was placed 7-bromothieno[3,2-c]pyridazin-4-ol (100 mg, 0.433 mmol, 1 equiv), PyBOP (337.82 mg, 0.649 mmol, 1.5 equiv), DIEA (223.74 mg, 1.732 mmol, 4 equiv), DMA (2 mL). The resulting solution was stirred for 1 h at room temperature. Then 1-(thiophen-2-yl)methanamine (171.43 mg, 1.516 mmol, 3.5 equiv) was added into the above mixture for one time at room temperature. Subsequently, the resulting solution was stirred for 6 h at 25 degrees C. Then concentrate the reaction solution under vacuum. The residue was applied on the HP-Flash with acetonitrile/NH₄HCO₃ (4:1). The desired fractions were collected and concentrated under reduced pressure to remove most of the acetonitrile. Then the mixture was lyophilizied to dryness. This resulted in 8.2 mg (5.81%) of 7-bromo-N-(thiophen-2-ylmethyl)thieno[3,2-c]pyridazin-4-amine.

LCMS C₁₁H₈BrN₃S₂ calc. [M+H]⁺ 325.93 found 326.05.

¹H NMR (300 MHz, Methanol-d₄) δ 8.63 (s, 1H), 8.09 (s, 1H), 7.34 (dd, J=5.1, 1.2 Hz, 1H), 7.15 (dd, J=3.5, 1.1 Hz, 1H), 7.01 (dd, J=5.1, 3.5 Hz, 1H).

### Specific Example of General Synthesis Method 5, Synthesis of 6-[(2S)-2-aminopropyl]-N-[(3-methoxythiophen-2-yl)methyl]-7-methylthieno[3,2-c]pyridazin-4-amine, Compound 74

### Step 1

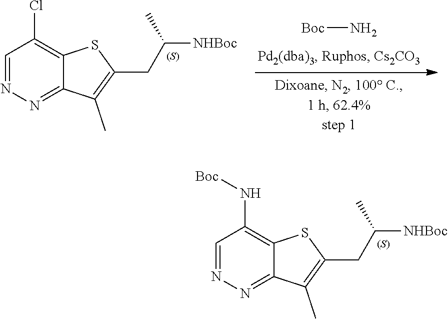

Into a 20-mL vial purged and maintained with an inert atmosphere of nitrogen, were added tert-butyl N-[(2S)-1-{4-chloro-7-methylthieno[3,2-c]pyridazin-6-yl}propan-2-yl]carbamate (700 mg, 2.048 mmol, 1 equiv), tert-butyl carbamate (719.65 mg, 6.144 mmol, 3 equiv), Pd₂(dba)₃ (187.51 mg, 0.205 mmol, 0.1 equiv), Ruphos (191.11 mg, 0.410 mmol, 0.2 equiv) and Cs₂CO₃ (1334.35 mg, 4.096 mmol, 2 equiv) in Dioxane (7 mL) at room temperature. The resulting mixture was stirred for 1 h at 100 degrees C. under nitrogen atmosphere. The residue was purified by silica gel column chromatography, eluted with PE/EA (1:5) to afford tert-butyl N-[(2S)-1-{4-[(tert-butoxycarbonyl)amino]-7-methylthieno[3,2-c]pyridazin-6-yl}propan-2-yl]carbamate (BB-14, 540 mg, 62.41%) as a yellow solid.

### Step 2

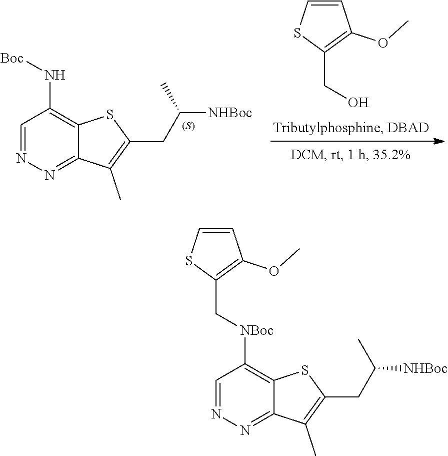

To a stirred solution of tert-butyl N-[(2S)-1-{4-[(tert-butoxycarbonyl)amino]-7-methylthieno[3,2-c]pyridazin-6-yl}propan-2-yl]carbamate (350 mg, 0.828 mmol, 1 equiv), (3-methoxythiophen-2-yl)methanol (238.87 mg, 1.656 mmol, 2 equiv) and Tributylphosphine (620.70 uL, 2.485 mmol, 3.00 equiv) in DCM (3.5 mL) was added di-tert-butyl dicarboxylate DBAD (381.47 mg, 1.656 mmol, 2 equiv) dissolved in DCM (1.5 mL) dropwise at 0° C. under nitrogen atmosphere. The resulting mixture was stirred for 1 h at room temperature under nitrogen atmosphere. The resulting mixture was concentrated under reduced pressure. The residue was purified by reverse flash chromatography with the following conditions: column, Cis; mobile phase, MeCN in Water (0.1% FA), 20% to 100% gradient in 20 min; detector, UV 254 nm. This resulted in tert-butyl N-{6-[(2S)-2-[(tert-butoxycarbonyl)amino]propyl]-7-methylthieno[3,2-c]pyridazin-4-yl}-N-[(3-methoxythiophen-2-yl)methyl]carbamate (160 mg, 35.20%) as a yellow solid.

### Step 3

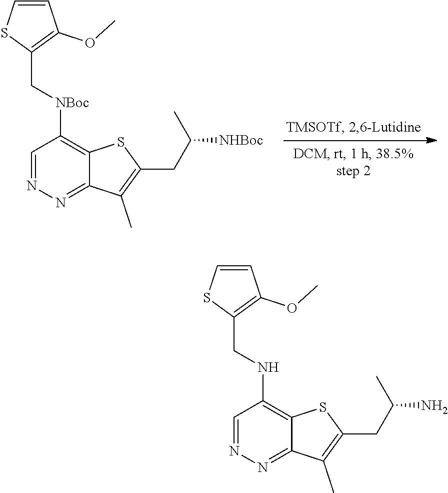 [SMILES: COc1ccsc1CN(C(=O)OC(C)(C)C)c1cnnc2c(C)c(C[C@H](C)NC(=O)OC(C)(C)C)sc12 ; COc1ccsc1CNc1cnnc2c(C)c(C[C@H](C)N)sc12]

To a stirred solution of tert-butyl N-{6-[(2S)-2-[(tert-butoxycarbonyl)amino]propyl]-7-methylthieno [3,2-c]pyridazin-4-yl}-N-[(3-methoxythiophen-2-yl)methyl]carbamate (140 mg, 0.255 mmol, 1 equiv) and 2,6-Lutidine (0.30 mL, 2.550 mmol, 10 equiv) in DCM (2 mL) was added TMSOTf (0.28 mL, 1.547 mmol, 6.06 equiv) dropwise at 0° C. The resulting mixture was stirred for 1 h at room temperature. The resulting mixture was concentrated under vacuum. The residue was purified by reverse flash chromatography with the following conditions: Column: XBridge Prep OBD C₁₈ Column, 30*150 mm, 5 m; Mobile Phase A: Water (10 mmol/L NH₄HCO₃), Mobile Phase B: ACN; Flow rate: 60 mL/min; Gradient: 2% B to 30% B in 2 min, 30% B to 50% B in 8 min, 50% B; Wave Length: 254/220 nm. This resulted in 6-[(2S)-2-aminopropyl]-N-[(3-methoxythiophen-2-yl)methyl]-7-methylthieno[3,2-c]pyridazin-4-amine (35.2 mg, 38.52%). LC-MS (ES, m/z): [M+H]⁺=349

1H-NMR (300 MHz, Methanol-d₄) δ 8.57 (d, J=5.5 Hz, 1H), 7.25 (d, J=5.5 Hz, 1H), 6.96 (d, J=5.5 Hz, 1H), 4.70 (s, 2H), 3.97 (s, 3H), 3.42 (h, J=6.6 Hz, 1H), 3.21-3.00 (m, 2H), 2.49 (s, 3H), 1.26 (d, J=6.4 Hz, 3H).

### Specific Example of General Synthesis Method 10, Synthesis of 6-[(2R)-2-amino-3-(methylsulfanyl)propyl]-7-methyl-N-(thiophen-2-ylmethyl)thieno[3,2-c]pyridazin-4-amine Compound 58

### Step 1

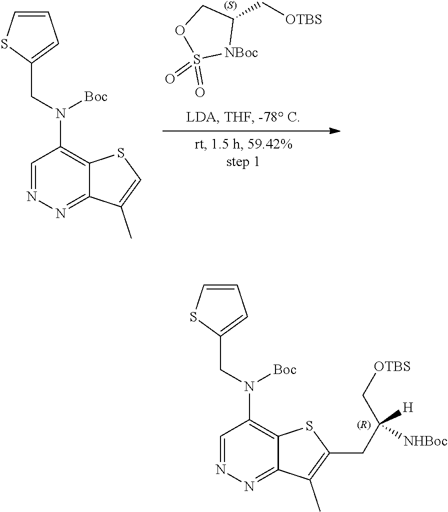

A solution of tert-butyl N-{7-methylthieno[3,2-c]pyridazin-4-yl}-N-(thiophen-2-ylmethyl)carbamate (1.5 g, 4.150 mmol, 1 equiv) in THF (15 mL, 185.142 mmol, 44.62 equiv) was treated with LDA (3 mL) for 15 min at −78° C. under nitrogen atmosphere followed by the addition of tert-butyl (4S)-4-{[(tert-butyldimethylsilyl)oxy]methyl}-2,2-dioxo-1,2lambda6,3-oxathiazolidine-3-carboxylate (1.53 g, 4.150 mmol, 1 equiv) dropwise at −78° C. The resulting mixture was stirred for additional 1.5 h at room temperature. The residue was purified by reverse flash chromatography with the following conditions: (column, C₁₈; mobile phase, MeCN in Water (0.1% FA), 10% to 100% gradient in 10 min; detector, UV 254 nm.) to afford tert-butyl N-{6-[(2R)-2-[(tert-butoxycarbonyl)amino]-3-[(tert-butyldimethylsilyl)oxy]propyl]-7-methylthieno[3,2-c]pyridazin-4-yl}-N-(thiophen-2-ylmethyl)carbamate (1.6 g, 59.42%) as a light yellow solid.

### Step 2

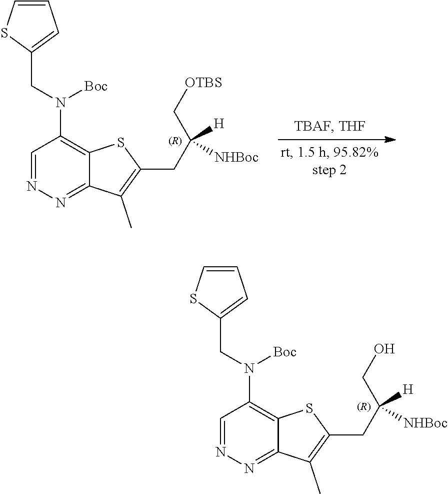

A solution of tert-butyl N-{6-[(2R)-2-[(tert-butoxycarbonyl)amino]-3-[(tert-butyldimethylsilyl)oxy]propyl]-7-methylthieno[3,2-c]pyridazin-4-yl}-N-(thiophen-2-ylmethyl)carbamate (1.9 g, 2.928 mmol, 1 equiv) and TBAF (5.5 mL) in THF (18.49 mL, 228.179 mmol, 77.93 equiv) was stirred for 1.5 h at room temperature under air atmosphere. The residue was purified by silica gel column chromatography, eluted with CH₂Cl₂/MeOH (10:1) to afford tert-butyl N-{6-[(2R)-2-[(tert-butoxycarbonyl)amino]-3-hydroxypropyl]-7-methylthieno[3,2-c]pyridazin-4-yl}-N-(thiophen-2-ylmethyl)carbamate (1.5 g, 95.82%) as a light yellow solid.

### Step 3

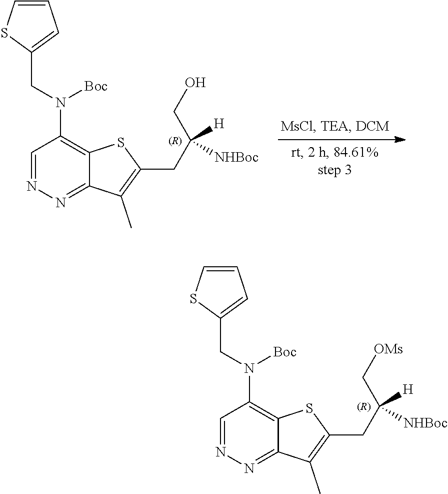

A solution of tert-butyl N-{6-[(2R)-2-[(tert-butoxycarbonyl)amino]-3-hydroxypropyl]-7-methylthieno [3,2-c]pyridazin-4-yl}-N-(thiophen-2-ylmethyl)carbamate (1.65 g, 3.086 mmol, 1 equiv) in DCM (18 mL, 283.151 mmol, 91.76 equiv) was treated with TEA (0.47 g, 4.629 mmol, 1.5 equiv) for 3 min at room temperature under nitrogen atmosphere followed by the addition of MsCl (0.42 g, 3.703 mmol, 1.2 equiv) dropwise at 0° C. The resulting mixture was stirred for additional 2 h at room temperature. The resulting mixture was extracted with EtOAc (2×30 mL) dried over anhydrous Na₂SO₄. After filtration, the filtrate was concentrated under reduced pressure to afford tert-butyl N-{6-[(2R)-2-[(tert-butoxycarbonyl)amino]-3-(methanesulfonyloxy)propyl]-7-methylthieno[3,2-c]pyridazin-4-yl}-N-(thiophen-2-ylmethyl)carbamate (1.6 g, 84.61%) as a brown solid.

### Step 4

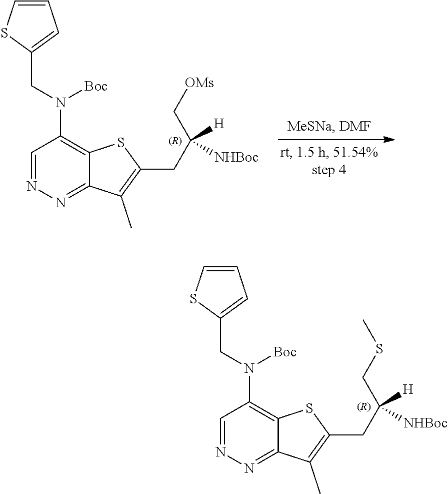

A solution of tert-butyl N-{6-[(2R)-2-[(tert-butoxycarbonyl)amino]-3-(methanesulfonyloxy)propyl]-7-methylthieno[3,2-c]pyridazin-4-yl}-N-(thiophen-2-ylmethyl)carbamate (800 mg, 1.306 mmol, 1 equiv) and MeSNa (182.77 mg, 2.612 mmol, 2 equiv) in DMF (12 mL, 155.058 mmol, 118.77 equiv) was stirred for 2 h at room temperature under nitrogen atmosphere. The residue was purified by reverse flash chromatography with the following conditions: (column, Cis; mobile phase, MeCN in Water (0.1% FA), 10% to 100% gradient in 20 min; detector, UV 254 nm.) to afford tert-butyl N-{6-[(2R)-2-[(tert-butoxycarbonyl)amino]-3-(methylsulfanyl)propyl]-7-methylthieno[3,2-c]pyridazin-4-yl}-N-(thiophen-2-ylmethyl)carbamate (380 mg, 51.54%) as a brown solid.

### Step 5

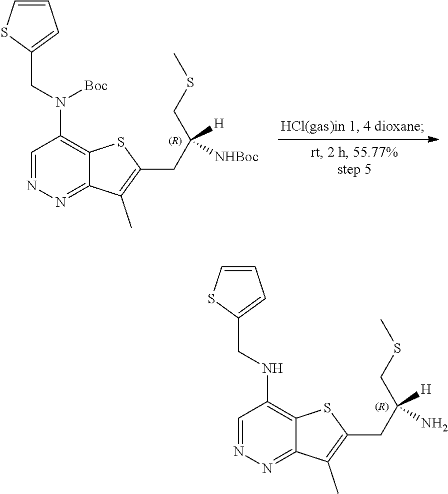

A solution of tert-butyl N-{6-[(2R)-2-[(tert-butoxycarbonyl)amino]-3-(methylsulfanyl)propyl]-7-methylthieno[3,2-c]pyridazin-4-yl}-N-(thiophen-2-ylmethyl)carbamate (50 mg, 0.089 mmol, 1 equiv) in HCl (gas) in 1,4-dioxane (2 mL, 65.826 mmol, 743.54 equiv) was stirred for 2 h at room temperature under air atmosphere. The residue was purified by reverse flash chromatography with the following conditions: (column, C₁₈; mobile phase, MeCN in Water (10 mmol/L NH₄HCO₃), 10% to 50% gradient in 10 min; detector, UV 254 nm.) to afford 6-[(2R)-2-amino-3-(methylsulfanyl)propyl]-7-methyl-N-(thiophen-2-ylmethyl)thieno[3,2-c]pyridazin-4-amine (18 mg, 55.77%). LC-MS ES, m/z): [M+H]⁺=365.1. ¹H NMR (400 MHz, DMSO-d₆) δ 8.55 (s, 1H), 7.68 (t, J=6.3 Hz, 1H), 7.40 (dd, J=5.1, 1.3 Hz, 1H), 7.13 (d, J 3.5 Hz, 1H), 6.98 (dd, J=5.1, 3.5 Hz, 1H), 4.79 (d, J=6.2 Hz, 2H), 3.12 (d, J=11.3 Hz, 2H), 2.92-2.83 (m, 1H), 2.59 (dd, J=13.1, 5.2 Hz, 1H), 2.47 (d, J=6.4 Hz, 1H), 2.41 (s, 3H), 2.08 (s, 3H).

Specific Example of General Synthesis Scheme 11, Synthesis of 6-[(2R)-2-amino-3-methanesulfonylpropyl]-7-methyl-N-(thiophen-2-ylmethyl)thieno[3,2-c]pyridazin-4-amine Compound 60.

### Step 1

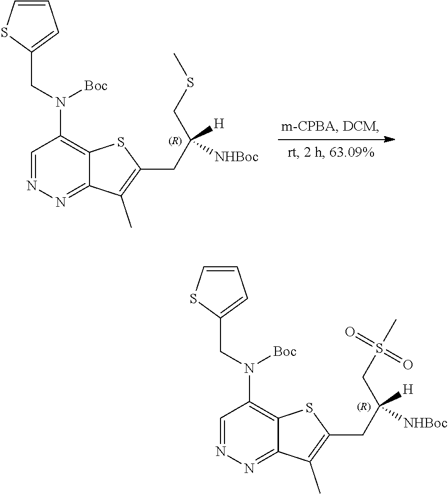

A solution of tert-butyl N-{6-[(2R)-2-[(tert-butoxycarbonyl)amino]-3-(methylsulfanyl)propyl]-7-methylthieno[3,2-c]pyridazin-4-yl}-N-(thiophen-2-ylmethyl)carbamate (150 mg, 0.266 mmol, 1 equiv) and m-CPBA (91.66 mg, 0.532 mmol, 2 equiv) in DCM (3 mL, 47.192 mmol, 177.69 equiv) was stirred for 2 h at room temperature under nitrogen atmosphere. The residue was purified by reverse flash chromatography with the following conditions: (column, Cig; mobile phase, MeCN in Water (10 mmol/L NH₄HCO₃), 10% to 100% gradient in 15 min; detector, UV 254 nm.) to afford tert-butyl N-{6-[(2R)-2-[(tert-butoxycarbonyl) amino]-3-methanesulfonylpropyl]-7-methylthieno[3,2-c]pyridazin-4-yl}-N-(thiophen-2-ylmethyl)carbamate (100 mg, 63.09%) as a white solid.

### Step 2

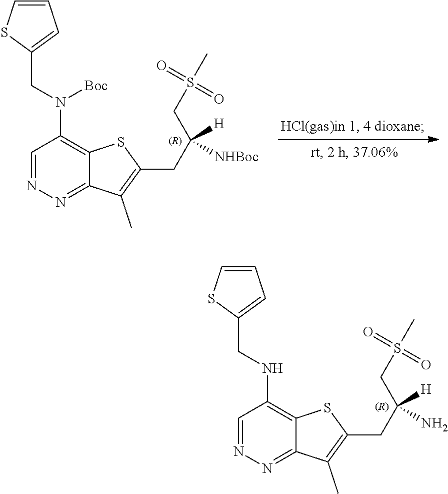

A solution of tert-butyl N-{6-[(2R)-2-[(tert-butoxycarbonyl)amino]-3-methanesulfonylpropyl]-7-methylthieno[3,2-c]pyridazin-4-yl}-N-(thiophen-2-ylmethyl)carbamate (80 mg, 0.134 mmol, 1 equiv) in HCl (gas) in 1,4-dioxane (3 mL, 98.738 mmol, 736.56 equiv) was stirred for 2 h at room temperature under air atmosphere. The residue was purified by reverse flash chromatography with the following conditions: (column, C₁₈; mobile phase, MeCN in Water (10 mmol/L NH₄HCO₃), 10% to 50% gradient in 10 min; detector, UV 254 nm.) to afford 6-[(2R)-2-amino-3-methanesulfonylpropyl]-7-methyl-N-(thiophen-2-ylmethyl)thieno[3,2-c]pyridazin-4-amine (20 mg, 37.62%). LC-MS (ES, m/z): [M+H]⁺=397.0. ¹H NMR (400 MHz, DMSO-d₆) δ 8.56 (s, 1H), 7.70 (t, J=6.2 Hz, 1H), 7.40 (dd, J=5.1, 1.3 Hz, 1H), 7.13 (dd, J 3.5, 1.2 Hz, 1H), 6.98 (dd, J=5.1, 3.5 Hz, 1H), 4.79 (d, J=6.2 Hz, 2H), 3.51 (q, J=8.8, 7.1 Hz, 1H), 3.27-3.12 (m, 3H), 3.06 (s, 3H), 3.03-2.95 (m, 1H), 2.41 (s, 3H), 2.02 (d, J=39.9 Hz, 2H).

### Specific Example of General Method 12, Synthesis of 6-[(2S)-2-aminopropyl]-N-[(5-bromothiophen-2-yl)methyl]-7-methylthieno[3,2-c]pyridazin-4-amine Compound 62

### Step 1

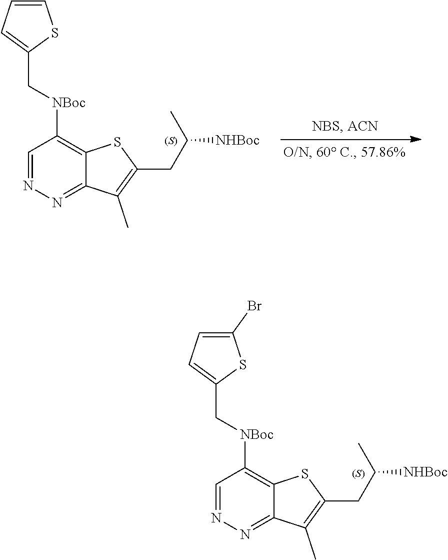

Into a 8 mL vial were added tert-butyl N-{6-[(2S)-2-[(tert-butoxycarbonyl)amino]propyl]-7-methylthieno[3,2-c]pyridazin-4-yl}-N-(thiophen-2-ylmethyl)carbamate (150 mg, 0.289 mmol, 1 equiv), ACN (3 mL) and NBS (61.77 mg, 0.347 mmol, 1.2 equiv) at room temperature The resulting mixture was stirred for overnight at 60° C. The residue was purified by reverse flash chromatography with the following conditions: column, Cis; mobile phase, MeCN in Water (10 mmol/L NH₄HCO₃), 10% to 100% gradient in 10 min; detector, UV 254 nm. This resulted in tert-butyl N-[(5-bromothiophen-2-yl)methyl]-N-{6-[(2S)-2-[(tert-butoxycarbonyl)amino]propyl]-7-methylthieno[3,2-c]pyridazin-4-yl}carbamate (100 mg, 57.86%) as a yellow solid.

### Step 2

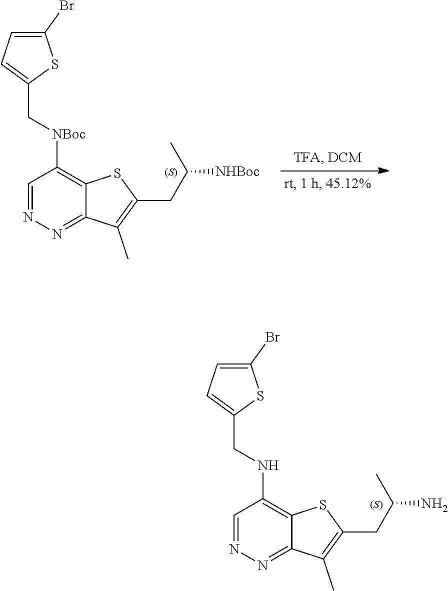

Into a 8 mL vial were added tert-butyl N-[(5-bromothiophen-2-yl)methyl]-N-{6-[(2S)-2-[(tert-butoxycarbonyl)amino]propyl]-7-methylthieno[3,2-c]pyridazin-4-yl}carbamate (50 mg, 0.084 mmol, 1 equiv), DCM (1 mL) and TFA (0.3 mL) at 0° C. The resulting mixture was stirred for 1 h at room temperature. The resulting mixture was concentrated under reduced pressure. The residue was purified by reverse flash chromatography with the following conditions: column, Cis; mobile phase, MeCN in Water (10 mmol/L NH₄HCO₃), 10% to 50% gradient in 10 min; detector, UV 254 nm. This resulted in 6-[(2S)-2-aminopropyl]-N-[(5-bromothiophen-2-yl)methyl]-7-methylthieno[3,2-c]pyridazin-4-amine (15 mg, 45.12%). LCMS-[M+H]⁺ 397 ¹H NMR (400 MHz, Methanol-d₄) δ 8.52 (s, 1H), 6.97 (d, J=3.8 Hz, 1H), 6.95-6.89 (m, 1H), 4.82 (d, J=1.0 Hz, 2H), 3.66 (dt, J=7.8, 6.3 Hz, 1H), 3.37-3.27 (m, 1H), 3.22 (dd, J=14.8, 8.0 Hz, 1H), 2.51 (s, 3H), 1.38 (d, J=6.5 Hz, 3H).

### Specific Example of General Synthesis Scheme 13, Synthesis of 7-methyl-6-(prop-1-en-2-yl)-N-(thiophen-2-ylmethyl)thieno[3,2-c]pyridazin-4-amine Compound 37

### Step 1

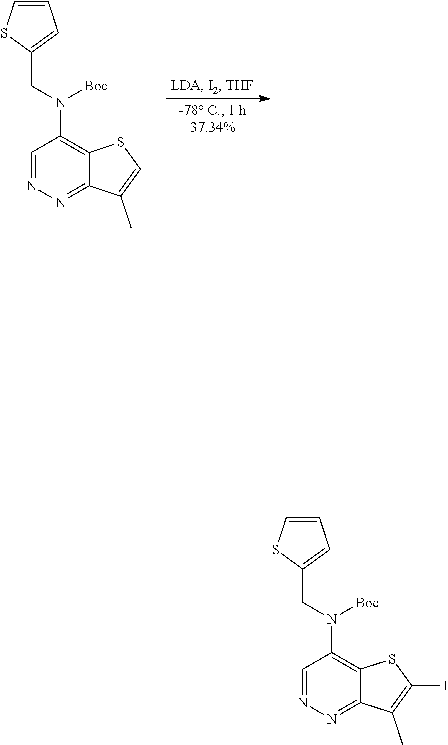 [SMILES: Cc1csc2c(N(Cc3cccs3)C(=O)OC(C)(C)C)cnnc12 ; Cc1c(I)sc2c(N(Cc3cccs3)C(=O)OC(C)(C)C)cnnc12]

In a 50-mL round bottom flask, to a solution of tert-butyl N-{7-methylthieno[3,2-c]pyridazin-4-yl}-N-(thiophen-2-ylmethyl)carbamate (1 g, 2.766 mmol, 1 equiv) in THE (10 mL) was added dropwise n-butyllithium solution (2 M in THF, 2.77 mL, 5.532 mmol) at −78 degrees C. under N₂ atmosphere. The reaction mixture was stirred at −78 degrees C. for 30 mins. Then a solution of I₂ (0.70 g, 2.766 mmol, 1 equiv) in 3 mL THE was added dropwise and the mixture was stirred for another 60 mins. The reaction was quenched with water/sat. NH₄Cl (0.5 mL), and then the mixture was extracted with EtOAc (2*50 mL). The combined organic extracts were washed with brine (100 mL), dried over anhydrous Na₂SO₄, and concentrated under vacuum to yield a crude product which was directly purified by silica gel column chromatography, eluted with PE/EA (5:1) to afford 6-iodo-7-methyl-N-(thiophen-2-ylmethyl)thieno [3,2-c]pyridazin-4-amine (400 mg, 37.34%) as a light yellow oil.

### Step 2

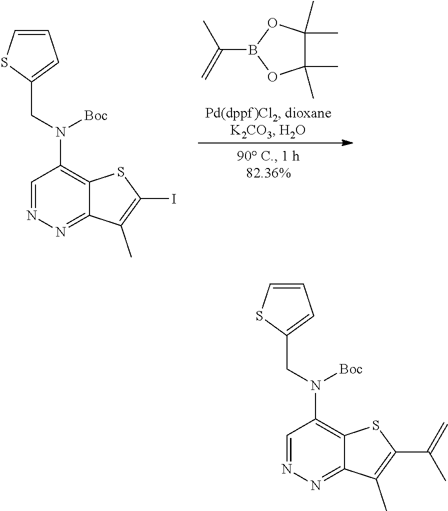 [SMILES: Cc1c(I)sc2c(N(Cc3cccs3)C(=O)OC(C)(C)C)cnnc12 ; C=C(C)B1OC(C)(C)C(C)(C)O1 ; C=C(C)c1sc2c(N(Cc3cccs3)C(=O)OC(C)(C)C)cnnc2c1C]

To a solution of tert-butyl N-{6-iodo-7-methylthieno[3,2-c]pyridazin-4-yl}-N-(thiophen-2-ylmethyl)carbamate (1.4 g, 2.873 mmol, 1 equiv) and 4,4,5,5-tetramethyl-2-(prop-1-en-2-yl)-1,3,2-dioxaborolane (675.79 mg, 4.022 mmol, 1.4 equiv) in dioxane (15 mL, 177.059 mmol, 61.64 equiv) and H₂O (3 mL, 166.528 mmol, 57.97 equiv) were added K₂CO₃ (794.00 mg, 5.746 mmol, 2 equiv) and Pd(dppf)Cl₂ (210.19 mg, 0.287 mmol, 0.1 equiv). After stirring for 1 h at 90° C. under a nitrogen atmosphere, the resulting mixture was concentrated under reduced pressure. The residue was purified by silica gel column chromatography, eluted with PE/EA (5:1) to afford tert-butyl N-[7-methyl-6-(prop-1-en-2-yl)thieno [3,2-c]pyridazin-4-yl]-N-(thiophen-2-ylmethyl)carbamate (950 mg, 82.36%) as a light yellow oil.

### Step 3

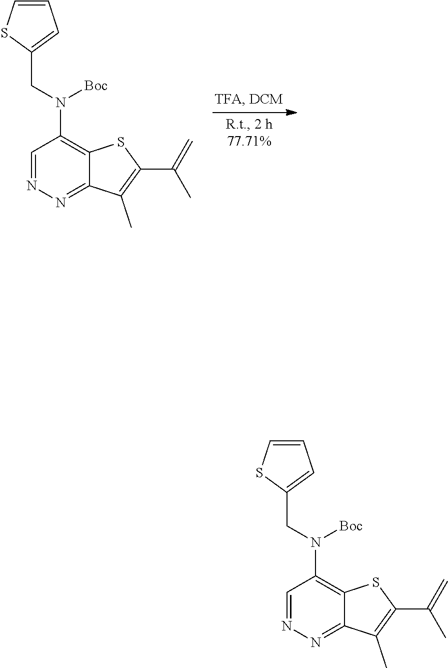 [SMILES: C=C(C)c1sc2c(N(Cc3cccs3)C(=O)OC(C)(C)C)cnnc2c1C ; C=C(C)c1sc2c(N(Cc3cccs3)C(=O)OC(C)(C)C)cnnc2c1C]

To a stirred mixture of tert-butyl N-[7-methyl-6-(prop-1-en-2-yl)thieno[3,2-c]pyridazin-4-yl]-N-(thiophen-2-ylmethyl)carbamate (60 mg, 0.149 mmol, 1 equiv) in DCM (1 mL) was added TFA (0.5 mL, 6.732 mmol, 45.05 equiv) dropwise at room temperature. The resulting mixture was stirred for 2 h at room temperature. The resulting mixture was concentrated under reduced pressure. The residue was basified to pH >7 with TEA. The resulting mixture was concentrated under reduced pressure. The residue was purified by reverse flash chromatography with the following conditions: column, Cis; mobile phase, MeCN in Water (10 mmol/L NH₄HCO₃), 10% to 50% gradient in 10 min; detector, UV 254 nm. This resulted in 7-methyl-6-(prop-1-en-2-yl)-N-(thiophen-2-ylmethyl)thieno[3,2-c]pyridazin-4-amine (35 mg, 77.71%). LC-MS [M+H]⁺=302.2.

¹H NMR (300 MHz, DMSO-d₆) δ 8.60 (s, 1H), 7.79 (t, J=6.2 Hz, 1H), 7.41 (dd, J=5.1, 1.3 Hz, 1H), 7.14 (dd, J=3.5, 1.2 Hz, 1H), 6.99 (dd, J=5.1, 3.4 Hz, 1H), 5.50 (p, J=1.6 Hz, 1H), 5.39 (t, J=1.2 Hz, 1H), 4.81 (d, J=6.1 Hz, 2H), 2.53 (s, 3H), 2.21 (dd, J=1.6, 0.9 Hz, 3H).

### Specific Examples of General Method 14, Synthesis of in 7-methyl-6-[(2R)-pyrrolidin-2-ylmethyl]-N-(thiophen-2-ylmethyl)thieno[3,2-c]pyridazin-4-amine (Compound 69)

### Step 1

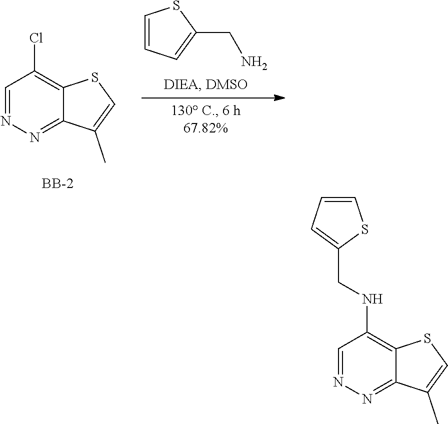

A mixture of 4-chloro-7-methylthieno[3,2-c]pyridazine (25 g, 135.399 mmol, 1 equiv), DIEA (35.00 g, 270.798 mmol, 2 equiv) and 1-(thiophen-2-yl)methanamine (61.30 g, 541.596 mmol, 4 equiv) in DMSO (250 mL) was stirred for 6 h at 130° C. under nitrogen atmosphere. The mixture was allowed to cool down to room temperature. The mixture was purified by reversed-phase flash chromatography with the following conditions: column, C₁₈ silica gel; mobile phase, MeCN in Water (10 mmol/L NH4HCO3), 10% to 60% gradient in 20 min; detector, UV 254 nm. This resulted in 7-methyl-N-(thiophen-2-ylmethyl)thieno[3,2-c]pyridazin-4-amine (24 g, 67.82%) as a brown yellow solid.

### Step 2

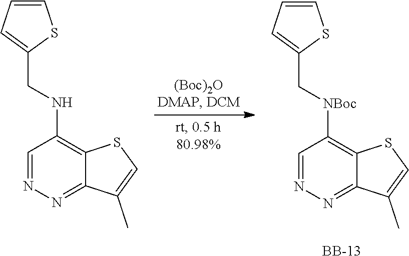 [SMILES: Cc1csc2c(NCc3cccs3)cnnc12 ; Cc1csc2c(N(Cc3cccs3)C(=O)OC(C)(C)C)cnnc12 ; "BB-13"]

To a stirred mixture of 7-methyl-N-(thiophen-2-ylmethyl)thieno[3,2-c]pyridazin-4-amine (25 g, 95.654 mmol, 1 equiv) and DMAP (16.36 g, 133.916 mmol, 1.4 equiv) in DCM (250 mL) was added di-tert-butyl dicarbonate (27.14 g, 124.350 mmol, 1.3 equiv) dropwise at room temperature under nitrogen atmosphere. The resulting mixture was stirred for 30 min at room temperature under nitrogen atmosphere. The reaction was quenched with water (0.1 mL) at room temperature. The resulting mixture was concentrated under reduced pressure. The residue was purified by reversed-phase flash chromatography with the following conditions: column, C₁₈ silica gel; mobile phase, MeCN in Water (10 mmol/L NH4HCO3), 20% to 100% gradient in 20 min; detector, UV 254 nm. This resulted in tert-butyl N-{7-methylthieno[3,2-c]pyridazin-4-yl}-N-(thiophen-2-ylmethyl)carbamate (BB-13) 28 g, 80.98%) as an off-white solid.

### Step 3

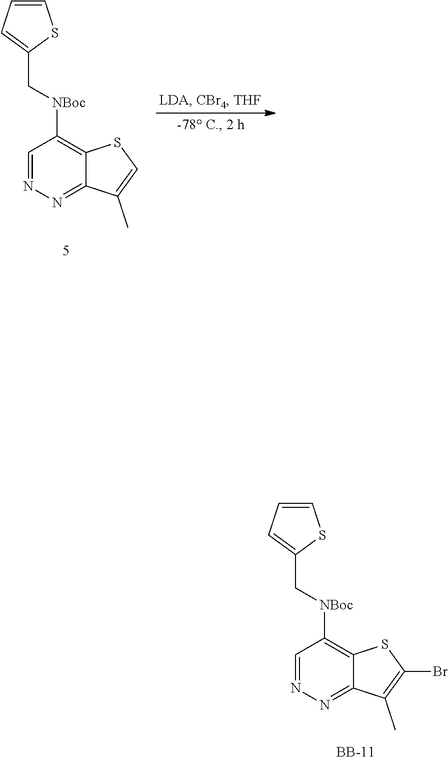 [SMILES: Cc1csc2c(N(Cc3cccs3)C(=O)OC(C)(C)C)cnnc12 ; "5" ; Cc1c(Br)sc2c(N(Cc3cccs3)C(=O)OC(C)(C)C)cnnc12 ; "BB-11"]

In a 25-mL round bottom flask, to a solution of tert-butyl N-{7-methylthieno[3,2-c]-163-yridazine-4-yl}-N-(thiophen-2-ylmethyl)carbamate (1.5 g, 4.150 mmol, 1 equiv) in THF (15 mL) was added dropwise n-butyllithium solution (2 M in THE, 4.15 mL, 8.300 mmol) at −78 degrees C. under N2 atmosphere. The reaction mixture was stirred at −78 degrees C. for 15 mins. Then a solution of CBr₄ (1.38 g, 4.150 mmol, 1 equiv) in 2 mL THE was added dropwise and the mixture was stirred for another 60 mins. The reaction was quenched with sat. NH₄Cl (0.2 mL), and then the mixture was extracted with EtOAc (2*25 mL). The combined organic extracts were washed with brine (40 mL), dried over anhydrous Na₂SO₄, and concentrated under vacuum to yield a crude product which was directly purified by silica gel column chromatography, eluted with PE/EA (8:1) to afford tert-butyl N-{6-bromo-7-methylthieno[3,2-c]-164-yridazine-4-yl}-N-(thiophen-2-ylmethyl)carbamate (BB-11, 1.2 g, 65.67%) as a light yellow solid.

### Step 4

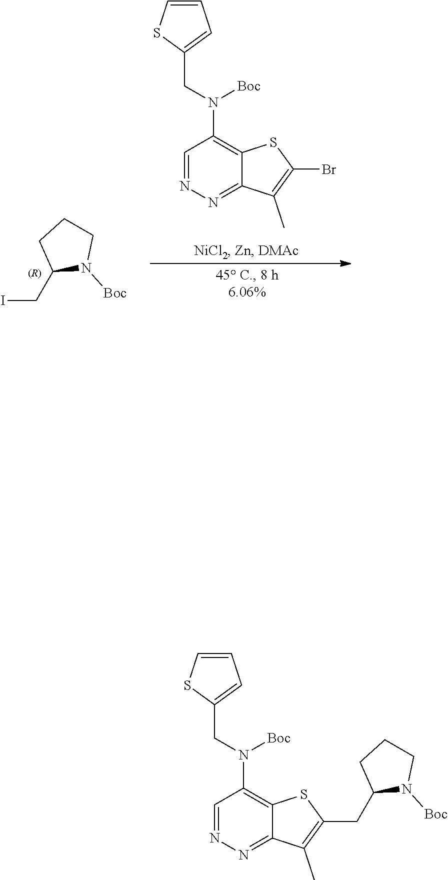

A mixture of pyridine-2-carboximidamide (44.01 mg, 0.363 mmol, 0.4 equiv), NiCl₂ (353.13 mg, 2.724 mmol, 3 equiv) and Zn (475.09 mg, 7.264 mmol, 8 equiv) in DMAc (5 mL) was stirred for 5 min at room temperature under nitrogen atmosphere. Then a solution of tert-butyl N-{6-bromo-7-methylthieno[3,2-c]pyridazin-4-yl}-N-(thiophen-2-ylmethyl)carbamate (400 mg, 0.908 mmol, 1 equiv) and tert-butyl (2R)-2-(iodomethyl)pyrrolidine-1-carboxylate (847.91 mg, 2.724 mmol, 3 equiv) in 3 mL DMAc was added dropwise and the resulting mixture was stirred for 8 h at 45° C. under nitrogen atmosphere. The residue was purified by reverse flash chromatography with the following conditions: column, C₁₈; mobile phase, MeCN in Water (10 mmol/L NH₄HCO₃), 20% to 90% gradient in 20 min; detector, UV 254 nm. This resulted in tert-butyl (2R)-2-({4-[(tert-butoxycarbonyl)(thiophen-2-ylmethyl)amino]-7-methylthieno[3,2-c]pyridazin-6-yl}methyl)pyrrolidine-1-carboxylate (30 mg, 6.06%) as a light yellow solid.

### Step 5

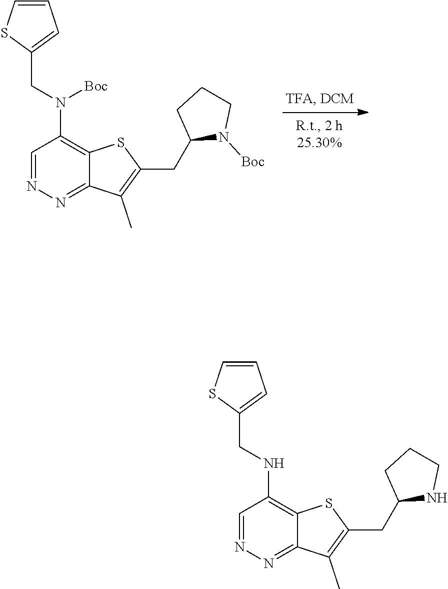 [SMILES: Cc1c(C[C@H]2CCCN2C(=O)OC(C)(C)C)sc2c(N(Cc3cccs3)C(=O)OC(C)(C)C)cnnc12 ; Cc1c(C[C@H]2CCCN2)sc2c(NCc3cccs3)cnnc12]

To a stirred solution of tert-butyl (2R)-2-({4-[(tert-butoxycarbonyl)(thiophen-2-ylmethyl)amino]-7-methylthieno[3,2-c]pyridazin-6-yl}methyl)pyrrolidine-1-carboxylate (25 mg, 0.046 mmol, 1 equiv) in DCM (0.5 mL, 7.865 mmol, 171.38 equiv) was added TFA (0.5 mL, 6.732 mmol, 146.67 equiv) dropwise at room temperature under nitrogen atmosphere. The resulting mixture was stirred for 2 h at room temperature under nitrogen atmosphere. The resulting mixture was concentrated under reduced pressure. The residue was purified by reverse flash chromatography with the following conditions: column, Cig; mobile phase, MeCN in Water (10 mmol/L NH₄HCO₃), 10% to 50% gradient in 10 min; detector, UV 254 nm. This resulted in 7-methyl-6-[(2R)-pyrrolidin-2-ylmethyl]-N-(thiophen-2-ylmethyl)thieno[3,2-c]pyridazin-4-amine (4 mg, 25.30%). LC-MS (ES, m/z): [M+H]⁺=345.1. H-NMR ¹H NMR (300 MHz, DMSO-d₆) δ 8.55-8.40 (m, 1H), 7.34-7.25 (m, 1H), 7.10 (d, J=3.4 Hz, 1H), 6.97 (dt, J=5.2, 2.9 Hz, 1H), 4.87 (d, J=6.3 Hz, 1H), 3.49 (p, J=7.2 Hz, 1H), 3.33 (s, 1H), 3.29-3.06 (m, 2H), 2.96 (ddd, J=10.9, 8.3, 6.3 Hz, 1H), 2.48 (d, J=2.7 Hz, 3H), 2.02 (s, 1H), 2.00-1.82 (m, 1H), 1.59 (dt, J=12.0, 8.5 Hz, 1H).

### Synthesis of (S)-6-(2-aminopropyl)-7-methoxy-N-(thiophen-2-ylmethyl)thieno[3,2-c]pyridazin-4-amine Compound 22

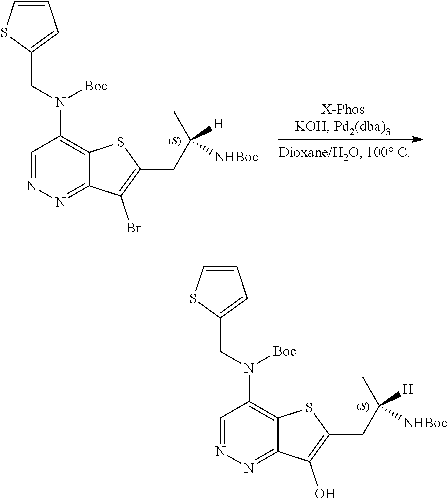

### Step 1. tert-butyl (S)-(6-(2-((tert-butoxycarbonyl)amino)propyl)-7-hydroxythieno[3,2-c]pyridazin-4-yl)(thiophen-2-ylmethyl)carbamate

Tert-butyl (S)-(7-bromo-6-(2-((tert-butoxycarbonyl)amino)propyl)thieno[3,2-c]pyridazin-4-yl)(thiophen-2-ylmethyl)carbamate (prepared according to general synthesis scheme 1 from BB-4 and AA-1, 500 mg, 0.857 mmol, 1 equiv), X-Phos (81.69 mg, 0.171 mmol, 0.2 equiv), KOH (96.14 mg, 1.714 mmol, 2 equiv) and Pd₂(dba)₃ (78.46 mg, 0.086 mmol, 0.1 equiv) in dioxane (9 mL) and water (1 mL) were stirred for 1 h at 100° C. under a nitrogen atmosphere. The reaction mixture was quenched with water and extracted with ethyl acetate. The combined organic layers were dried over anhydrous sodium sulfate and concentrated. The residue was purified by a silica gel chromatography (dichloromethane/methanol 10:1) yielding the product yellow solid (300 mg, 67%).

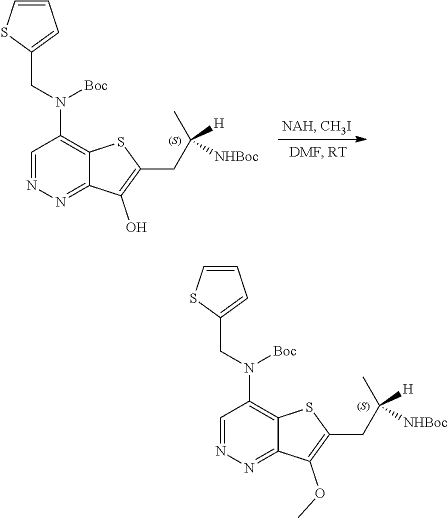

### Step 2. tert-butyl (S)-(6-(2-((tert-butoxycarbonyl)amino)propyl)-7-methoxythieno[3,2-c]pyridazin-4-yl)(thiophen-2-ylmethyl)carbamate

A mixture of tert-butyl (S)-(6-(2-((tert-butoxycarbonyl)amino)propyl)-7-hydroxythieno[3,2-c]pyridazin-4-yl)(thiophen-2-ylmethyl)carbamate (300 mg, 0.576 mmol, 1 equiv) and NaH (27.65 mg, 1.152 mmol, 2 equiv) in DMF (5 mL) was stirred for 0.5 h at 25° C., CH₃I (122.68 mg, 0.864 mmol, 1.5 equiv) was added and stirring was continued for 1 h at this temperature. The reaction mixture was quenched through addition of water and extracted with ethyl acetate. The combined organic layers were dried over anhydrous sodium sulfate, the solvents were evaporated, and the raw product purified by silica gel chromatography (dichloromethane/methanol=10:1). Yellow solid (100 mg, 33%)

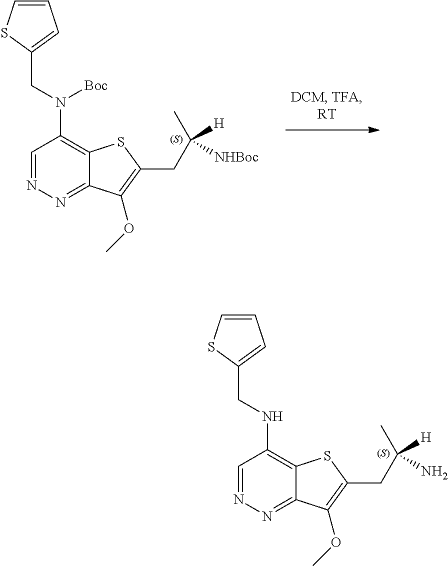

### Step 3. (S)-6-(2-aminopropyl)-7-methoxy-N-(thiophen-2-ylmethyl)thieno[3,2-c]pyridazin-4-amine

A solution of tert-butyl (S)-(6-(2-((tert-butoxycarbonyl)amino)propyl)-7-methoxythieno[3,2-c]pyridazin-4-yl)(thiophen-2-ylmethyl)carbamate (100 mg, 0.187 mmol, 1 equiv) in TFA (1 mL) and DCM (2 mL) was stirred for 2 h at room temperature. The volatile components were removed in vacuo and the residue was purified by reverse phase flash chromatography (C18 column, mobile phase: 10% to 100% MeCN in 10M aqueous NH4HCO3 solution, detector: UV 254 nm). The product containing fractions were collected, concentrated, and freeze dried providing the product (4.3 mg (6.9%). LCMS: m/z 335.00 [M+H]⁺. ¹H NMR (400 MHz, Methanol-d₄) δ 8.50 (d, J=12.4 Hz, 1H), 7.30 (dd, J=5.1, 1.2 Hz, 1H), 7.11 (dd, J 3.5, 1.2 Hz, 1H), 6.97 (dd, J=5.1, 3.5 Hz, 1H), 4.11 (s, 2H), 3.37-3.32 (m, 1H), 3.14-2.88 (m, 2H), 1.24-1.13 (m, 3H).

### Synthesis of Compound 90 6-[(2S)-2-aminopropyl]-4-[(thiophen-2-ylmethyl) amino]thieno[3,2-c]pyridazin-7-ol

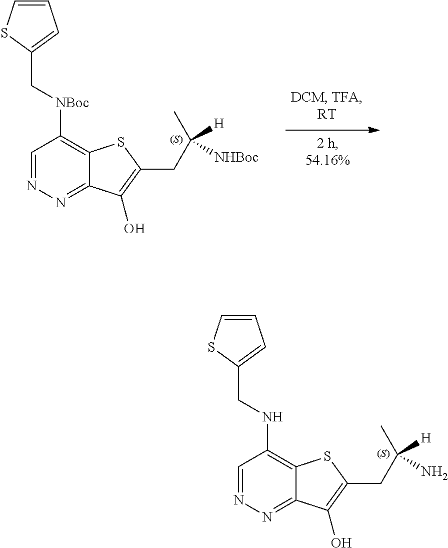

Into a 8-mL vial purged and maintained with an inert atmosphere of nitrogen, was placed tert-butyl N-{6-[(2S)-2-[(tert-butoxycarbonyl)amino]propyl]-7-hydroxythieno[3,2-c]pyridazin-4-yl}-N-(thiophen-2-ylmethyl)carbamate (30 mg, 0.058 mmol, 1 equiv), DCM (2 mL), TFA (1 mL). The resulting solution was stirred for 2 hr at room temperature. Then concentrate the reaction solution under vacuum. The residue was applied on the HP-Flash with acetonitrile/NH₄HCO₃ (4:1). The desired fractions were collected and concentrated under reduced pressure to remove most of the acetonitrile. Then the mixture was lyophilized to dryness. This resulted in 10 mg (54.16%) of 6-[(2S)-2-aminopropyl]-4-[(thiophen-2-ylmethyl) amino]thieno[3,2-c]pyridazin-7-ol.

LCMSC₁₄H₁₆N₄OS₂ calc. [M+H]⁺ 321.08 found 320.95.

¹H NMR (400 MHz, Methanol-d₄) δ 7.89 (d, J=4.7 Hz, 1H), 7.33 (td, J=5.1, 1.2 Hz, 1H), 7.18-7.10 (m, 2H), 6.99 (dt, J=5.1, 3.3 Hz, 1H), 4.82 (dd, J=28.4, 1.1 Hz, 2H), 3.36 (d, J=6.5 Hz, 1H), 3.11-3.02 (m, 2H), 1.22 (d, J=6.4 Hz, 3H).

### Synthesis of (S)-6-(2-aminopropyl)-4-((thiophen-2-ylmethyl)amino)thieno[3,2-c]pyridazine-7-carbonitrile Compound 24

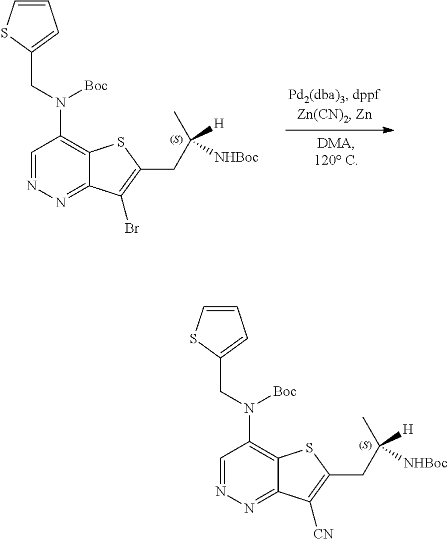

### Step 1: tert-butyl (S)-(6-(2-((tert-butoxycarbonyl)amino)propyl)-7-cyanothieno[3,2-c]pyridazin-4-yl)(thiophen-2-ylmethyl)carbamate

Tert-butyl (S)-(7-bromo-6-(2-((tert-butoxycarbonyl)amino)propyl)thieno[3,2-c]pyridazin-4-yl)(thiophen-2-ylmethyl)carbamate (prepared according to general synthesis scheme 1 from BB-4 and AA-1, 500 mg, 0.857 mmol, 1 equiv), Pd₂(dba)₃ (78.5 mg, 0.086 mmol, 0.1 equiv), dppf (94.7 mg, 0.171 mmol, 0.2 equiv), zinc cyanide (100.6 mg, 0.857 mmol, 1 equiv) and Zn powder (6.7 mg, 0.103 mmol, 0.12 equiv) in DMA (12 mL) were stirred under a nitrogen atmosphere for 1 h at 120° C. The reaction mixture was quenched with water and extracted with ethyl acetate. The combined organic layers were dried over anhydrous sodium sulfate and concentrated. The residue was chromatographed (silica gel column, dichloromethane/methanol=10:1) providing the product as yellow solid (200 mg, 44%).

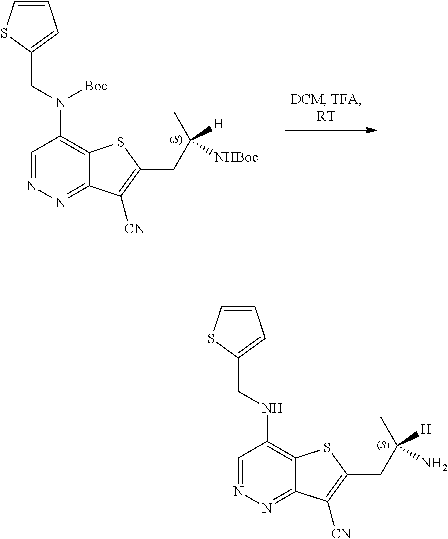

### Step 2. (S)-6-(2-aminopropyl)-4-((thiophen-2-ylmethyl)amino)thieno[3,2-c]pyridazine-7-carbonitrile

Tert-butyl (S)-(6-(2-((tert-butoxycarbonyl)amino)propyl)-7-cyanothieno[3,2-c]pyridazin-4-yl)(thiophen-2-ylmethyl)carbamate (100 mg, 0.189 mmol, 1 equiv) was stirred in DCM (2 mL) and TFA (1 mL) for 2 h at room temperature. The reaction mixture was concentrated and the raw product purified by reverse phase flash chromatography (C18 column, mobile phase: 10% to 100% MeCN in 10M aqueous NH4HCO3 solution, detector: UV 254 nm). White solid (4.6 mg, 7.4%). LCMS: m/z 330.16 [M+H]⁺ 329.08. ¹H NMR (300 MHz, Methanol-d₄) δ 8.70-8.58 (m, 1H), 7.35 (dd, J=5.1, 1.2 Hz, 1H), 7.15 (dd, J=3.6, 1.1 Hz, 1H), 7.06-6.96 (m, 1H), 3.44-3.37 (m, 1H), 3.29-3.10 (m, 2H), 1.24 (d, J=6.3 Hz, 3H).

### Synthesis of 6-((2R,3S)-3-aminobutan-2-yl)-N-(thiophen-2-ylmethyl)thieno[3,2-c]pyridazin-4-amine and 6-((2S,3S)-3-aminobutan-2-yl)-N-(thiophen-2-ylmethyl)thieno[3,2-c]pyridazin-4-amine Compounds 25 and 26

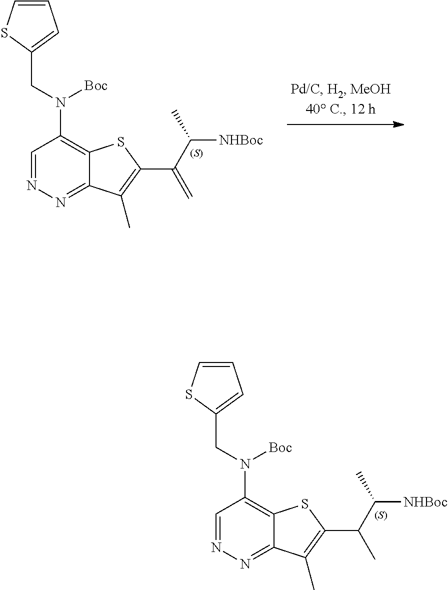

### Step 1. Tert-butyl (6-((3S)-3-((tert-butoxycarbonyl)amino)butan-2-yl)-7-methylthieno[3,2-c]-168-yridazine-4-yl)(thiophen-2-ylmethyl)carbamate

Tert-butyl (S)-(6-(3-((tert-butoxycarbonyl)amino)but-1-en-2-yl)-7-methylthieno[3,2-c]pyridazin-4-yl)(thiophen-2-ylmethyl)carbamate (prepared from BB-11 and tert-butyl (S)-(3-(4,4,5,5-tetramethyl-1,3,2-dioxaborolan-2-yl)but-3-en-2-yl)carbamate in analogy to step 2 of Compound 37, 400 mg, 0.754 mmol, 1 equiv) in MeOH (8 mL) was hydrogenated at 40° C. for 12 h with Pd/C (10%, 100 mg) as catalyst using a balloon as hydrogen source. The mixture was filtered through a pad of Celite, the solvent evaporated and the residue purified by silica gel column chromatography (PE/EA=1:1) affording tert-butyl (6-((3S)-3-((tert-butoxycarbonyl)amino)butan-2-yl)-7-methylthieno[3,2-c]pyridazin-4-yl)(thiophen-2-ylmethyl)carbamate (260 mg, 65%) as a colorless oil. The two diastereomers obtained were separated through preparative SFC. Prep SFC conditions: column: (R, R)-WHELK-01-Kromasil, 4.6 mm×250 mm, 5 m; mobile phase A: Hex with 0.5% 2M NH3-MeOH, mobile phase B: EtOH; flow rate: 40 mL/min; gradient: 20% B to 20% B in 18 min; wave length: 208/224 nm; sample dissolved in MeOH:DCM=1:1; injection volume: 0.9 mL. Faster eluting diastereomer: White solid (80 mg); retention time of 8 min. Slower eluting diastereomer: White solid (100 mg); retention time of 13 min.

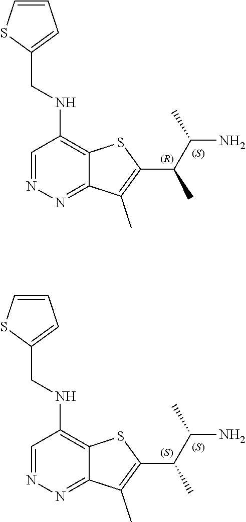

### Step 2: 6-((2R,3S)-3-aminobutan-2-yl)-N-(thiophen-2-ylmethyl)thieno[3,2-c]pyridazin-4-amine and 6-((2S,3S)-3-aminobutan-2-yl)-N-(thiophen-2-ylmethyl)thieno[3,2-c]pyridazin-4-amine

The title compounds were obtained from the two diastereomers of tert-butyl (6-((3S)-3-((tert-butoxycarbonyl)amino)butan-2-yl)-7-methylthieno[3,2-c]pyridazin-4-yl)(thiophen-2-ylmethyl)carbamate through treatment with TFA in DCM.

Compound 25 was obtained from the faster eluting step 1 diastereomer: (20 mg). LCMS (m/z): [M+H]⁺=333.1. ¹H NMR (400 MHz, DMSO-d₆) δ 8.55 (d, J=6.8 Hz, 1H), 7.69 (dt, J=12.4, 6.1 Hz, 1H), 7.40 (dt, J=5.0, 1.5 Hz, 1H), 7.14 (dd, J=3.3, 1.5 Hz, 1H), 6.98 (td, J=4.2, 3.4, 1.4 Hz, 1H), 4.79 (t, J=5.4 Hz, 2H), 3.14 (t, J=7.0 Hz, 1H), 3.04-2.81 (m, 1H), 2.41 (d, J=2.8 Hz, 3H), 1.30 (d, J=6.6 Hz, 3H), 0.95 (d, J=6.2 Hz, 3H).

Compound 26 was obtained from the slower eluting step 1 diastereomer: (15 mg). LCMS (m/z): [M+H]⁺=333.1. ¹H NMR (400 MHz, DMSO-d₆) δ 8.55 (d, J=2.2 Hz, 1H), 7.69 (s, 1H), 7.48-7.31 (m, 1H), 7.13 (d, J=3.4 Hz, 1H), 6.98 (dd, J=5.1, 3.4 Hz, 1H), 2.95 (dt, J 19.7, 9.7 Hz, 3H), 2.38 (d, J=9.3 Hz, 3H), 1.84-1.47 (m, 2H), 1.09 (dd, J=11.9, 6.5 Hz, 3H).

### Synthesis of Compound 41 and Compound 42

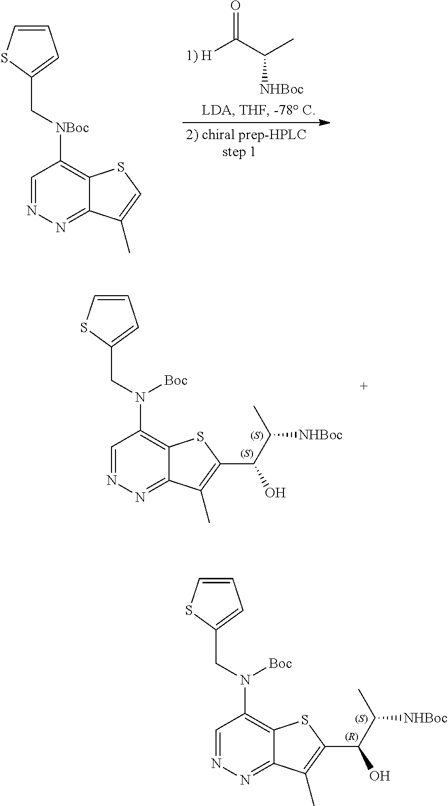

In a 25-mL round bottom flask, to a solution of tert-butyl N-{7-methylthieno[3,2-c]pyridazin-4-yl}-N-(thiophen-2-ylmethyl)carbamate (200 mg, 0.553 mmol, 1 equiv) in THE (20 mL) was added dropwise LDA (1M, 1.0 mL, 1.0 mmol, 1.8 equiv) at −78° C. under N₂ atmosphere. The reaction mixture was stirred at −78° C. for 30 min. Then a solution of tert-butyl (S)-(1-oxopropan-2-yl)carbamate (143.75 mg, 0.830 mmol, 1.5 equiv) in THE (1 mL) was added dropwise and the mixture was stirred for another 30 min. Subsequently, the resulting solution was stirred for 1 h at 25 degrees C. The residue was purified by reverse flash chromatography directly with the following conditions: column, C₁₈; mobile phase, MeCN in Water (10 mmol/L NH₄HCO₃), 10% to 100% gradient in 10 min; detector, UV 254 nm.

The crude product was purified by Prep-Chiral-HPLC with the following conditions, column, CHIRALPAK IF-3; mobile phase, Hex (0.1% DEA):EtOH=80:20, Flow rate: 16 ml/min, Temperature: Ambient, The first eluting peak afforded 80 mg of a yellow solid which was carried forward in the synthesis of Compound 41. The second eluting peak afforded 80 mg of a yellow solid which was carried forward in the synthesis of Compound 42. The absolute configuration at oxygen was not established.

### Synthesis of Compound 41 and Compound 42

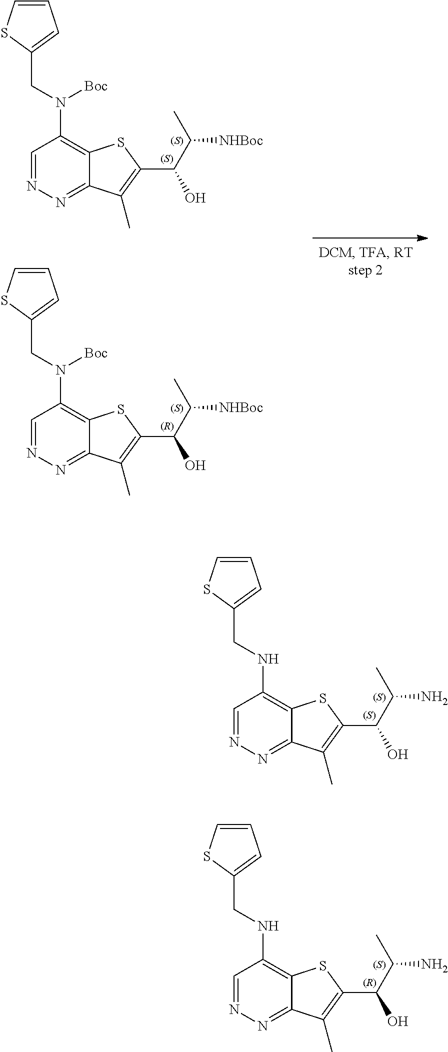

For Compound 41: Into a 8-mL vial purged and maintained with an inert atmosphere of nitrogen, was placed tert-butyl N-{6-[(2S)-2-[(tert-butoxycarbonyl)amino]-1-hydroxypropyl]-7-methylthieno[3,2-c]pyridazin-4-yl}-N-(thiophen-2-ylmethyl)carbamate (50 mg, 0.094 mmol, 1 equiv), DCM (2 mL, 31.461 mmol, 336.44 equiv), TFA (1 mL, 13.463 mmol, 143.97 equiv). The resulting solution was stirred for 2 hr at room temperature. Then concentrate the reaction solution under vacuum. The residue was purified by reverse flash chromatography with the following conditions: column, C18 silica gel; mobile phase, MeCN in Water (10 mmol/L NH₄HCO₃), 10% to 100% gradient in 10 min; detector, UV 254 nm. The desired fractions were collected and concentrated under reduced pressure to remove most of the acetonitrile. Then the mixture was lyophilizied to dryness. This resulted in 15.7 mg.

LCMS C₁₅H₁₈N₄OS₂ calc. [M+H]⁺ 335.09 found 335.10.

¹H NMR (400 MHz, DMSO-d₆) δ 8.56 (s, 1H), 7.71 (t, J=6.3 Hz, 1H), 7.39 (dd, J=5.1, 1.3 Hz, 1H), 7.13 (dd, J=3.4, 1.2 Hz, 1H), 6.98 (dd, J=5.1, 3.4 Hz, 1H), 4.80-4.77 (m, 3H), 3.00 (p, J=6.6 Hz, 1H), 2.44 (s, 3H), 0.94 (d, J=6.5 Hz, 3H).

For Compound 42: Into a 8-mL vial purged and maintained with an inert atmosphere of nitrogen, was placed (2S)-2-amino-1-{7-methyl-4-[(thiophen-2-ylmethyl)amino]thieno[3,2-c]pyridazin-6-yl}propan-1-ol (20 mg, 0.060 mmol, 1 equiv), DCM (2 mL, 31.461 mmol, 526.13 equiv), TFA (1 mL, 13.463 mmol, 225.14 equiv). The resulting solution was stirred for 1 h at 25 degrees C. Then concentrate the reaction solution under vacuum. The residue was purified by reverse flash chromatography with the following conditions: column, C18 silica gel; mobile phase, MeCN in Water (10 mmol/L NH4HCO3), 10% to 100% gradient in 10 min; detector, UV 254 nm. The desired fractions were collected and concentrated under reduced pressure to remove most of the acetonitrile. Then the mixture was lyophilized to dryness. This resulted in 4.6 mg (23.00%) of Compound 42. LCMS C₁₅H₁₈N₄OS₂ calc. [M+H]⁺ 335.09 found 335.05.

¹H NMR (400 MHz, CD₃OD) δ 8.49 (s, 1H), 7.29 (m, 1H), 7.11 (m, 1H), 6.97 (m, 1H), 5.27 (d, J=4.4 Hz, 1H), 4.88 (s, 2H), 3.47-3.45 (m, 1H), 2.50 (s, 3H), 1.22 (d, J=6.8 Hz, 3H).

### Synthesis of tert-butyl N-[(2S)-1-{4-[(tert-butoxycarbonyl)(thiophen-2-ylmethyl)amino]-7-methylthieno[3,2-c]pyridazin-6-yl}-1,1-difluoropropan-2-yl]carbamate

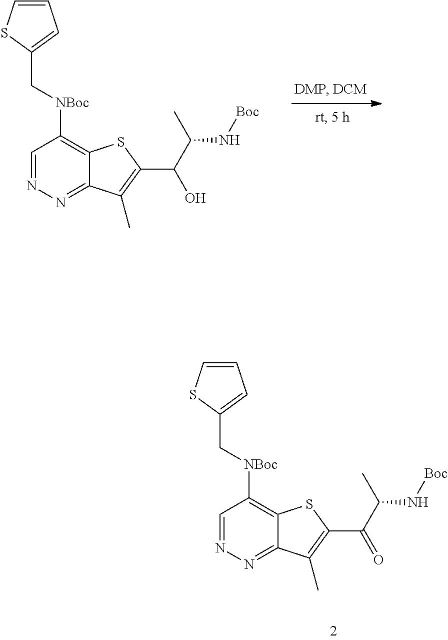 [SMILES: Cc1c(C(O)[C@H](C)NC(=O)OC(C)(C)C)sc2c(N(Cc3cccs3)C(=O)OC(C)(C)C)cnnc12 ; Cc1c(C(=O)[C@H](C)NC(=O)OC(C)(C)C)sc2c(N(Cc3cccs3)C(=O)OC(C)(C)C)cnnc12]

### Compound 125

To a stirred solution tert-butyl N-{6-[(2S)-2-[(tert-butoxycarbonyl)amino]-1-hydroxypropyl]-7-methylthieno[3,2-c]pyridazin-4-yl}-N-(thiophen-2-ylmethyl)carbamate (described above) (200 mg, 0.374 mmol, 1 equiv) in DCM (5 mL) was added Dess-Martin (237.98 mg, 0.561 mmol, 1.5 equiv) in portions at 0° C. under air atmosphere. The resulting mixture was stirred for additional 5 h at room temperature. The reaction was quenched by the addition of 1M Na₂SO₃ (3 mL) at 0° C. The mixture was basified to pH 8 with saturated NaHCO₃ (aq.). The resulting mixture was extracted with CH₂Cl₂ (3×50 mL). The combined organic layers were washed with brine (1×7 mL), dried over anhydrous Na₂SO₄. After filtration, the filtrate was concentrated under reduced pressure. The residue was purified by silica gel column chromatography, eluted with PE/EA (1:1) to afford tert-butyl N-{6-[(2S)-2-[(tert-butoxycarbonyl)amino]propanoyl]-7-methylthieno[3,2-c]pyridazin-4-yl}-N-(thiophen-2-ylmethyl)carbamate (110 mg, 55.21%) as a light yellow solid. LC-MS (ES, m/z): [M+H]⁺=533

### Step 2

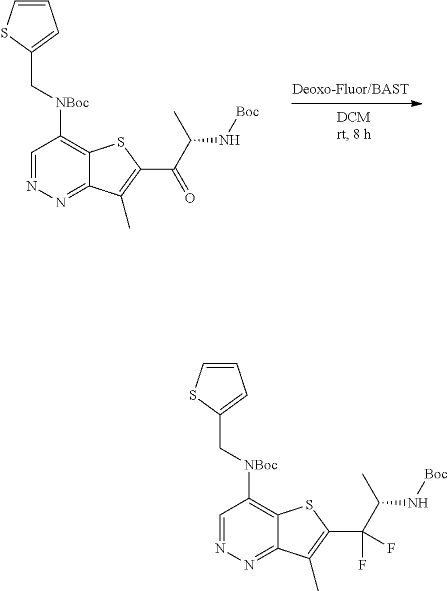 [SMILES: Cc1c(C(=O)[C@H](C)NC(=O)OC(C)(C)C)sc2c(N(Cc3cccs3)C(=O)OC(C)(C)C)cnnc12 ; Cc1c(C(F)(F)[C@H](C)NC(=O)OC(C)(C)C)sc2c(N(Cc3cccs3)C(=O)OC(C)(C)C)cnnc12]

A solution tert-butyl N-{6-[(2S)-2-[(tert-butoxycarbonyl)amino]propanoyl]-7-methylthieno[3,2-c]pyridazin-4-yl}-N-(thiophen-2-ylmethyl)carbamate (100 mg, 0.188 mmol, 1 equiv) in DAST (3.5 mL, 3.572 mmol) was stirred for overnight at room temperature under air atmosphere. To the above mixture was added BAST (186.90 mg, 0.846 mmol, 4.5 equiv) in portions over 0.5 min at 0° C. The resulting mixture was stirred for additional 8 h at room temperature. The reaction was quenched with 50 mL sat. NaHCO₃ (aq.) at 0° C. The resulting mixture was extracted with EtOAc (3×50 mL). The combined organic layers were washed with brine (2×10 mL), dried over anhydrous Na₂SO₄. After filtration, the filtrate was concentrated under reduced pressure. The residue was purified by silica gel column chromatography, eluted with PE/EA (4:1) to afford tert-butyl N-[(2S)-1-{4-[(tert-butoxycarbonyl)(thiophen-2-ylmethyl)amino]-7-methylthieno[3,2-c]pyridazin-6-yl}-1,1-difluoropropan-2-yl]carbamate (23 mg, 22.09%) as a light yellow solid. LC-MS (ES, m/z): [M+H]⁺=555

### Step 3

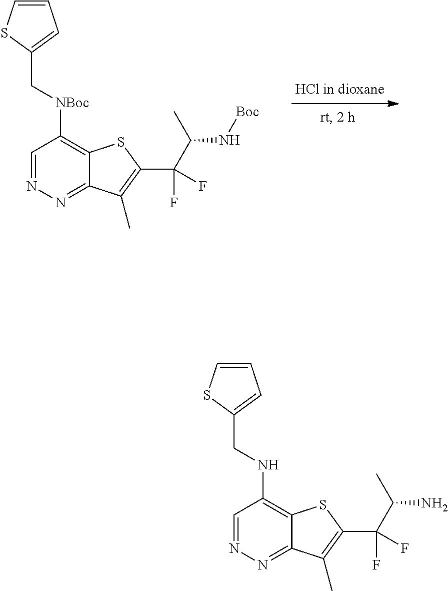 [SMILES: Cc1c(C(F)(F)[C@H](C)NC(=O)OC(C)(C)C)sc2c(N(Cc3cccs3)C(=O)OC(C)(C)C)cnnc12 ; Cc1c(C(F)(F)[C@H](C)N)sc2c(NCc3cccs3)cnnc12]

A solution tert-butyl N-[(2S)-1-{4-[(tert-butoxycarbonyl)(thiophen-2-ylmethyl)amino]-7-methylthieno[3,2-c]pyridazin-6-yl}-1,1-difluoropropan-2-yl]carbamate (23 mg, 0.041 mmol, 1 equiv) in DCM (1 mL) was stirred for 5 min at 0° C. under air atmosphere. To the above mixture was added HCl (gas) in 1,4-dioxane (1 mL, 32.913 mmol, 793.73 equiv) dropwise 0.5 min at 0° C. The resulting mixture was stirred for additional 2 h at room temperature. The resulting mixture was concentrated under reduced pressure. The residue was dissolved in MeOH (2 mL). The crude product was purified by Prep-HPLC with the following conditions (Column: XBridge Prep C₁₈ OBD Column 30*100 mm, 5 m 13 nm; Mobile Phase A: Water (10 mmol/L NH₄HCO₃+0.05NH₃·H₂O), Mobile Phase B: ACN; Flow rate: 50 mL/min mL/min; Gradient: 20% B to 60% B in 9 min; Wave Length: 254 nm/220 nm nm; RT1 (min): 6.65) to afford 6-[(2S)-2-amino-1,1-difluoropropyl]-7-methyl-N-(thiophen-2-ylmethyl)thieno[3,2-c]pyridazin-4-amine (5 mg, 34.02%). LCMS (ES, m/z): [M+H]⁺=355

¹H NMR (300 MHz, Methanol-d₄) δ 8.58 (s, 1H), 7.31 (dd, J=5.1, 1.3 Hz, 1H), 7.16-7.09 (m, 1H), 6.98 (dd, J=5.1, 3.5 Hz, 1H), 4.89 (s, 10H), 4.59 (s, 4H), 3.59 (ddd, J=18.2, 13.4, 6.8 Hz, 1H), 2.64 (t, J=2.0 Hz, 3H), 1.22 (d, J=6.7 Hz, 3H).

### Synthesis of 2-{7-methyl-4-[(thiophen-2-ylmethyl)amino]thieno [3,2-c]pyridazin-6-yl}propan-2-ol and 2-{7-methyl-4-[(thiophen-2-ylmethyl)amino]thieno[3,2-c]pyridazin-6-yl}propan-1-ol

### Step 1

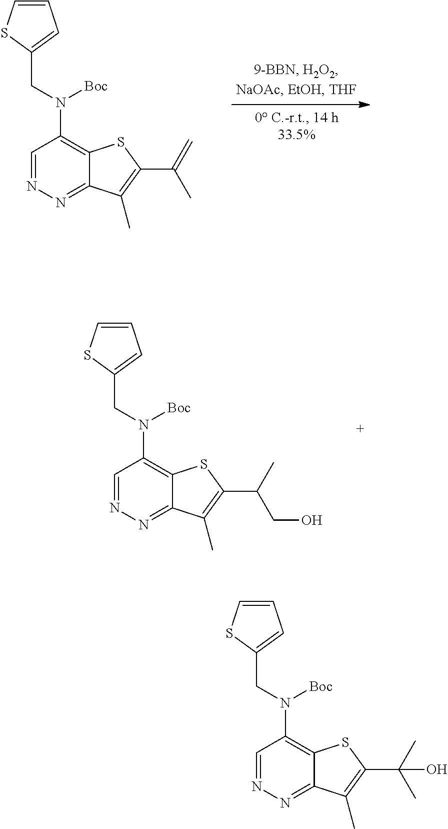 [SMILES: C=C(C)c1sc2c(N(Cc3cccs3)C(=O)OC(C)(C)C)cnnc2c1C ; Cc1c(C(C)CO)sc2c(N(Cc3cccs3)C(=O)OC(C)(C)C)cnnc12 ; Cc1c(C(C)(C)O)sc2c(N(Cc3cccs3)C(=O)OC(C)(C)C)cnnc12]

To a stirred solution of tert-butyl N-[7-methyl-6-(prop-1-en-2-yl)thieno[3,2-c]pyridazin-4-yl]-N-(thiophen-2-ylmethyl)carbamate (obtained in step 2 of the synthesis of compound 38) 100 mg, 0.250 mmol, 1 equiv) in THE (2 mL) was added 9-borabicyclo[3.3.1]nonane (2.00 mL, 1.000 mmol, 4 equiv) dropwise at 0° C. under nitrogen atmosphere. The resulting mixture was stirred for 14 h at room temperature under nitrogen atmosphere. To the above mixture was added EtOH (0.2 mL, 1.721 mmol, 13.82 equiv), sat. NaOAc (0.50 mL) and H₂O₂ (0.3 mL, 1.288 mmol, 10.34 equiv, 30%) dropwise at 0° C. The resulting mixture was stirred for additional 5 h at room temperature. The residue was purified by reverse flash chromatography with the following conditions: column, C₁₈; mobile phase, MeCN in Water (10 mmol/L NH₄HCO₃), 10% to 50% gradient in 10 min; detector, UV 254 nm. This resulted in tert-butyl N-[6-(1-hydroxypropan-2-yl)-7-methylthieno[3,2-c]pyridazin-4-yl]-N-(thiophen-2-ylmethyl)carbamate (35 mg, 33.50%) as a light yellow oil.

### Step 2. Synthesis of 2-{7-methyl-4-[(thiophen-2-ylmethyl)amino]thieno[3,2-c]pyridazin-6-yl}propan-1-ol Compound 54

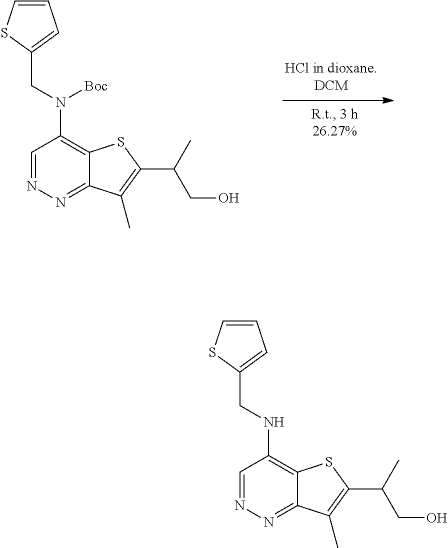 [SMILES: Cc1c(C(C)CO)sc2c(N(Cc3cccs3)C(=O)OC(C)(C)C)cnnc12 ; Cc1c(C(C)CO)sc2c(NCc3cccs3)cnnc12]

To a stirred mixture of tert-butyl N-[6-(1-hydroxypropan-2-yl)-7-methylthieno[3,2-c]pyridazin-4-yl]-N-(thiophen-2-ylmethyl)carbamate (50 mg, 0.119 mmol, 1 equiv) in DCM (1 mL, 15.731 mmol, 132.00 equiv) was added HCl (gas) in 1,4-dioxane (1 mL, 4.000 mmol, 33.56 equiv) dropwise at room temperature under nitrogen atmosphere. The resulting mixture was stirred for 3 h at room temperature. The resulting mixture was concentrated under reduced pressure. The mixture was basified to pH >7 with TEA. The resulting mixture was concentrated under reduced pressure. The residue was purified by reverse flash chromatography with the following conditions: column, C₁₈; mobile phase, MeCN in Water (10 mmol/L NH₄HCO₃), 15% to 60% gradient in 20 min; detector, UV 254 nm. This resulted in 2-{7-methyl-4-[(thiophen-2-ylmethyl)amino]thieno[3,2-c]pyridazin-6-yl}propan-1-ol (10 mg, 26.27%) as a white solid. LC-MS (ES, m/z): [M+H]⁺=320.25.

¹H NMR (300 MHz, DMSO-d₆) δ 8.55 (s, 1H), 7.69 (t, J=6.2 Hz, 1H), 7.40 (dd, J 5.1, 1.2 Hz, 1H), 7.13 (d, J=3.4 Hz, 1H), 6.98 (dd, J=5.1, 3.4 Hz, 1H), 4.97 (s, 1H), 4.80 (d, J 6.2 Hz, 2H), 3.56 (s, 2H), 3.52-3.42 (m, 1H), 2.42 (s, 3H), 1.61 (s, 1H), 1.29 (d, J=6.7 Hz, 3H).

### Step 3. Synthesis of 2-{7-methyl-4-[(thiophen-2-ylmethyl)amino]thieno [3,2-c]pyridazin-6-yl}propan-2-ol (Compound 65)

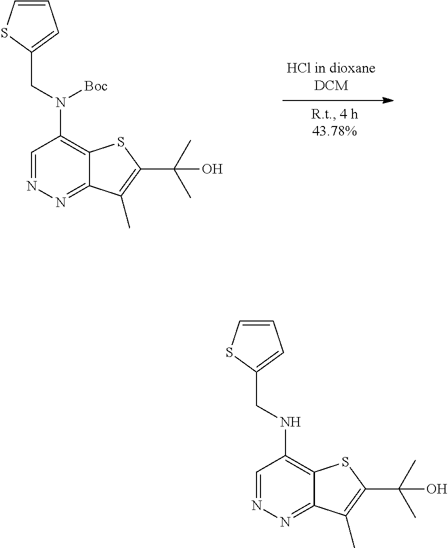 [SMILES: Cc1c(C(C)(C)O)sc2c(N(Cc3cccs3)C(=O)OC(C)(C)C)cnnc12 ; Cc1c(C(C)(C)O)sc2c(NCc3cccs3)cnnc12]

To a stirred mixture of tert-butyl N-[6-(2-hydroxypropan-2-yl)-7-methylthieno[3,2-c]pyridazin-4-yl]-N-(thiophen-2-ylmethyl)carbamate (60 mg, 0.143 mmol, 1 equiv) in DCM (1 mL, 15.731 mmol, 110.00 equiv) was added HCl (gas) in 1,4-dioxane (1.5 mL, 49.369 mmol, 345.22 equiv) dropwise at room temperature. The resulting mixture was stirred for 4 h at room temperature. The resulting mixture was concentrated under vacuum. The residue was purified by reverse flash chromatography with the following conditions: column, C₁₈; mobile phase, MeCN in Water (10 mmol/L NH₄HCO₃), 10% to 60% gradient in 20 min; detector, UV 254 nm. This resulted in 2-{7-methyl-4-[(thiophen-2-ylmethyl)amino]thieno [3,2-c]pyridazin-6-yl}propan-2-ol (20 mg, 43.78%).

LC-MSES, m/z): [M+H]⁺=320.20.

¹H NMR (300 MHz, DMSO-d₆) δ 8.54 (s, 1H), 7.67 (t, J=6.3 Hz, 1H), 7.39 (dd, J=5.1, 1.3 Hz, 1H), 7.12 (dd, J=3.5, 1.2 Hz, 1H), 6.98 (dd, J=5.1, 3.5 Hz, 1H), 5.95 (s, 1H), 4.79 (d, J=6.2 Hz, 2H), 1.60 (s, 6H).

### Synthesis of 4-{7-methyl-4-[(thiophen-2-ylmethyl)amino]thieno[3,2-c]pyridazin-6-yl}butan-2-one (Compound 88)

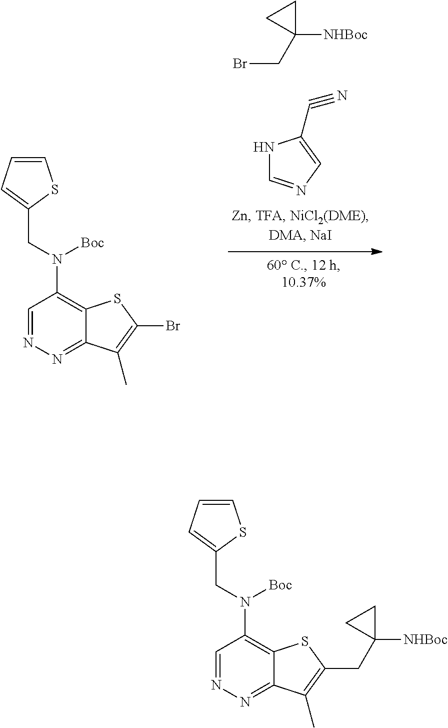 [SMILES: CC(C)(C)OC(=O)NC1(CBr)CC1 ; Cc1c(Br)sc2c(N(Cc3cccs3)C(=O)OC(C)(C)C)cnnc12 ; N#Cc1cnc[nH]1 ; Cc1c(CC2(NC(=O)OC(C)(C)C)CC2)sc2c(N(Cc3cccs3)C(=O)OC(C)(C)C)cnnc12]

### Step 1

Into a 4 mL vial were added tert-butyl N-[1-(bromomethyl)cyclopropyl]carbamate (BB-11, 170.40 mg, 0.681 mmol, 1.5 equiv), tert-butyl N-{6-bromo-7-methylthieno[3,2-c]pyridazin-4-yl}-N-(thiophen-2-ylmethyl) carbamate (200 mg, 0.454 mmol, 1 equiv), 3H-imidazole-4-carbonitrile (4.23 mg, 0.045 mmol, 0.1 equiv), Zn (118.77 mg, 1.816 mmol, 4 equiv), 1,2-dimethoxyethane dihydrochloride nickel (9.98 mg, 0.045 mmol, 0.1 equiv), NaI (17.02 mg, 0.114 mmol, 0.25 equiv) and DMA (4 mL) at room temperature under nitrogen atmosphere. To the above mixture was added TFA (5.18 mg, 0.045 mmol, 0.1 equiv) dropwise at room temperature under nitrogen atmosphere. The resulting mixture was stirred for 12 h at 60° C. under nitrogen atmosphere. The residue was purified by reverse flash chromatography with the following conditions: column, C18; mobile phase, MeCN in Water (0.1% FA), 20% to 90% gradient in 20 min; detector, UV 254 nm. This resulted in tert-butyl N-[6-({1-[(tert-butoxycarbonyl)amino]cyclopropyl}methyl)-7-methylthieno [3,2-c]pyridazin-4-yl]-N-(thiophen-2-ylmethyl)carbamate (25 mg, 10.37%) as a light yellow oil.

### Step 2

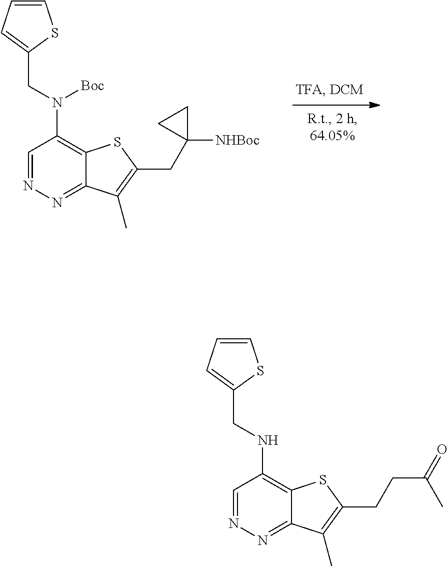 [SMILES: Cc1c(CC2(NC(=O)OC(C)(C)C)CC2)sc2c(N(Cc3cccs3)C(=O)OC(C)(C)C)cnnc12 ; CC(=O)CCc1sc2c(NCc3cccs3)cnnc2c1C]

To a stirred solution of tert-butyl N-[6-({1-[(tert-butoxycarbonyl)amino]cyclopropyl}methyl)-7-methylthieno[3,2-c]pyridazin-4-yl]-N-(thiophen-2-ylmethyl)carbamate (30 mg, 0.057 mmol, 1 equiv) in DCM (0.4 mL, 6.292 mmol, 111.31 equiv) was added TFA (0.4 mL, 5.385 mmol, 95.26 equiv) dropwise at room temperature under nitrogen atmosphere. The resulting mixture was stirred for 2 h at room temperature under nitrogen atmosphere. The resulting mixture was concentrated under reduced pressure. The residue was purified by reverse flash chromatography with the following conditions: column, Cis; mobile phase, MeCN in Water (0.1% FA), 10% to 50% gradient in 10 min; detector, UV 254 nm. This resulted in 4-{7-methyl-4-[(thiophen-2-ylmethyl)amino]thieno[3,2-c]pyridazin-6-yl}butan-2-one (12 mg, 64.05%).

LC-MS (ES, m/z): [M+H]⁺=332.05.

¹H NMR (300 MHz, DMSO-d₆) δ 9.24 (s, 1H), 8.70 (s, 1H), 7.48 (dd, J=5.1, 1.3 Hz, 1H), 7.19 (dd, J=3.5, 1.2 Hz, 1H), 7.02 (dd, J=5.1, 3.4 Hz, 1H), 4.98 (d, J=6.0 Hz, 2H), 3.13 (t, J 7.0 Hz, 2H), 2.90 (t, J=7.0 Hz, 2H), 2.39 (s, 3H), 2.13 (s, 3H).

### Synthesis of 2-{7-methyl-4-[(thiophen-2-ylmethyl)amino]thieno[3,2-c]pyridazin-6-yl}acetonitrile Compound 98

### Step 1

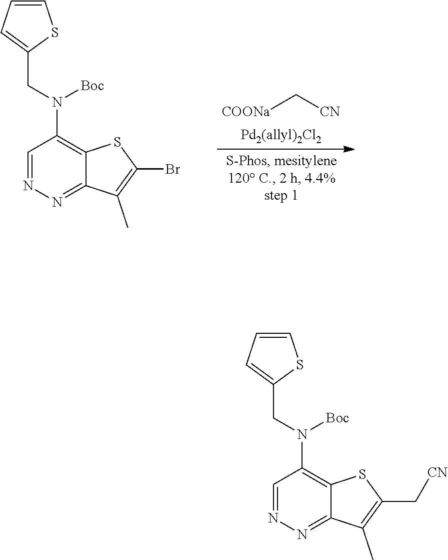

To a solution of tert-butyl N-{6-bromo-7-methylthieno[3,2-c]pyridazin-4-yl}-N-(thiophen-2-ylmethyl) carbamate (1 g, 2.271 mmol, 1 equiv) and 2-sodioacetonitrile; carbon dioxide (0.49 g, 4.542 mmol, 2 equiv) in mesitylene (15 mL) were added Pd₂(allyl)₂Cl₂ (0.08 g, 0.227 mmol, 0.1 equiv) and S-Phos (0.19 g, 0.454 mmol, 0.2 equiv). After stirring for 2 h at 120° C. under a nitrogen atmosphere, the resulting mixture was concentrated under reduced pressure. The residue was purified by reverse flash chromatography with the following conditions: (column, Cis; mobile phase, MeCN in Water (10 mmol/L NH₄HCO₃), 10% to 50% gradient in 10 min; detector, UV 254 nm.) to afford tert-butyl N-[6-(cyanomethyl)-7-methylthieno[3,2-c]pyridazin-4-yl]-N-(thiophen-2-ylmethyl)carbamate (40 mg, 4.40%) as a light yellow solid.

### Step 2

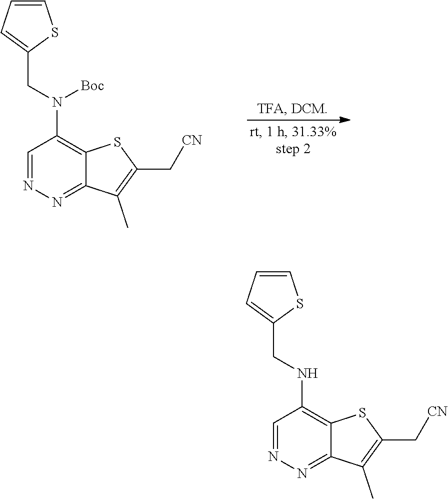 [SMILES: Cc1c(CC#N)sc2c(N(Cc3cccs3)C(=O)OC(C)(C)C)cnnc12 ; Cc1c(CC#N)sc2c(NCc3cccs3)cnnc12]

A solution of tert-butyl N-[6-(cyanomethyl)-7-methylthieno[3,2-c]pyridazin-4-yl]-N-(thiophen-2-ylmethyl)carbamate (40 mg, 0.100 mmol, 1 equiv) in TFA (0.5 mL, 6.732 mmol, 67.40 equiv), DCM (1.5 mL, 23.596 mmol, 236.27 equiv) was stirred for 1 h at room temperature under air atmosphere. The residue was purified by reverse flash chromatography with the following conditions: (column, Cis; mobile phase, MeCN in Water (10 mmol/L NH₄HCO₃), 10% to 50% gradient in 10 min; detector, UV 254 nm) to afford 2-{7-methyl-4-[(thiophen-2-ylmethyl)amino]thieno[3,2-c]pyridazin-6-yl}acetonitrile (9.4 mg, 31.33%).

LC-MS (ES, m/z): [M+H]⁺=300.0.

¹H NMR (400 MHz, DMSO-d₆) δ 8.65 (d, J=4.9 Hz, 1H), 7.92 (t, J=6.2 Hz, 1H), 7.42 (dd, J 5.1, 1.3 Hz, 1H), 7.13 (dd, J=3.4, 1.3 Hz, 1H), 6.98 (dd, J=5.1, 3.5 Hz, 1H), 4.82 (d, J=6.2 Hz, 2H), 4.45 (dp, J=87.5, 6.6 Hz, 2H), 2.41 (s, 3H).

### Synthesis of 6-[(2S)-2-amino-4-fluorobutyl]-7-methyl-N-(thiophen-2-ylmethyl)thieno[3,2-c]pyridazin-4-amine Compound 48

### Step 1

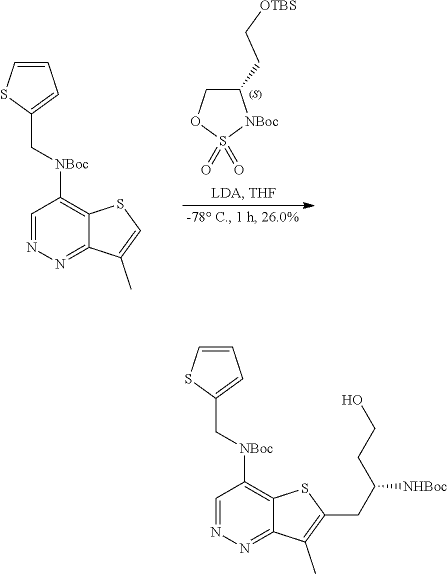

Into a 50-mL three-necked bottle, to a solution of tert-butyl N-{7-methylthieno[3,2-c]pyridazin-4-yl}-N-(thiophen-2-ylmethyl)carbamate (2.3 g, 6.363 mmol, 1 equiv) in THE (20 mL) was added LDA (4.14 mL, 8.272 mmol, 1.3 equiv) dropwise at −78° C. under N₂ atmosphere. The reaction mixture was stirred at −78° C. for 30 mins. Then a solution of tert-butyl (4S)-4-{2-[(tert-butyldimethylsilyl)oxy]ethyl}-2,2-dioxo-1,2lambda6,3-oxathiazolidine-3-carboxylate (3.64 g, 9.545 mmol, 1.5 equiv) in 15 mL THE was added dropwise and the mixture was stirred for another 1 h −78 degrees C. The residue was purified by reversed-phase flash chromatography with the following conditions: column, C₁₈ silica gel; mobile phase, MeCN in Water (10 mmol/L NH₄HCO₃), 0% to 100% gradient in 30 min; detector, UV 254 nm. This resulted in tert-butyl N-{6-[(2S)-2-[(tert-butoxycarbonyl)amino]-4-hydroxybutyl]-7-methylthieno[3,2-c]pyridazin-4-yl}-N-(thiophen-2-ylmethyl)carbamate (910 mg, 26.06%) as a yellow solid.

### Step 2

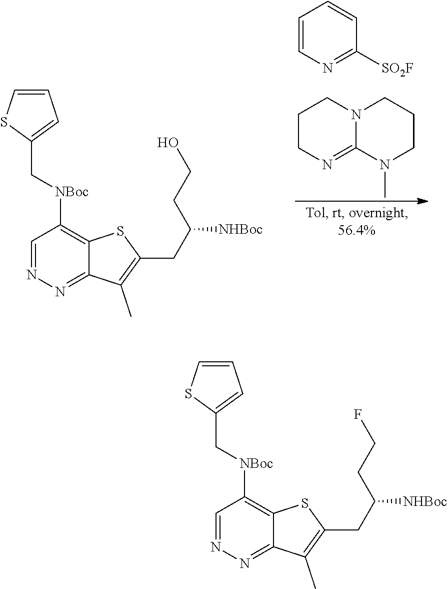

To a stirred mixture of tert-butyl N-{6-[(2S)-2-[(tert-butoxycarbonyl)amino]-4-hydroxybutyl]-7-methylthieno[3,2-c]pyridazin-4-yl}-N-(thiophen-2-ylmethyl)carbamate (150 mg, 0.273 mmol, 1 equiv) and pyridine-2-sulfonyl fluoride (48.46 mg, 0.300 mmol, 1.1 equiv) in toluene (2 mL) was added 1-methyl-2H,3H,4H,6H,7H,8H-pyrimido[1,2-a][1,3]diazine (83.77 mg, 0.546 mmol, 2 equiv). The resulting mixture was stirred for overnight at room temperature. The resulting mixture was concentrated under reduced pressure. The residue was purified by reversed-phase flash chromatography with the following conditions: column, C₁₈ silica gel; mobile phase, MeCN in Water (10 mmol/L NH₄HCO₃), 30% to 100% gradient in 15 min; detector, UV 254 nm. This resulted in tert-butyl N-[(2S)-1-{4-[(tert-butoxycarbonyl)(thiophen-2-ylmethyl)amino]-7-methylthieno[3,2-c]pyridazin-6-yl}-4-fluorobutan-2-yl]carbamate (85 mg, 56.46%) as a yellow solid.

### Step 3

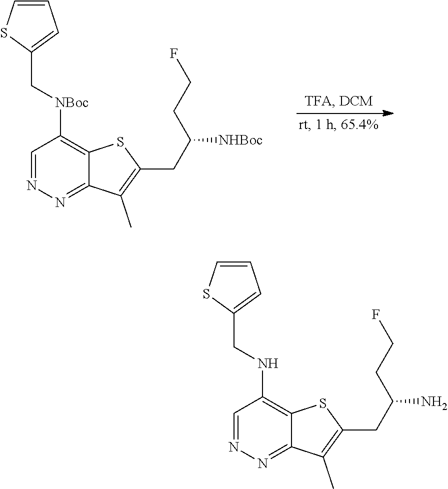 [SMILES: Cc1c(C[C@H](CCF)NC(=O)OC(C)(C)C)sc2c(N(Cc3cccs3)C(=O)OC(C)(C)C)cnnc12 ; Cc1c(C[C@@H](N)CCF)sc2c(NCc3cccs3)cnnc12]

To a stirred solution of tert-butyl N-[(2S)-1-{4-[(tert-butoxycarbonyl)(thiophen-2-ylmethyl)amino]-7-methylthieno[3,2-c]pyridazin-6-yl}-4-fluorobutan-2-yl]carbamate (85 mg, 0.154 mmol, 1 equiv) in DCM (0.6 mL) was added TFA (0.2 mL) dropwise at 0° C. The resulting mixture was stirred for 1 h at room temperature. The resulting mixture was concentrated under vacuum. The residue was purified by reversed-phase flash chromatography with the following conditions: column, C₁₈ silica gel; mobile phase, MeCN in Water (10 mmol/L NH₄HCO₃), 0% to 100% gradient in 20 min; detector, UV 254 nm. This resulted in 6-[(2S)-2-amino-4-fluorobutyl]-7-methyl-N-(thiophen-2-ylmethyl)thieno[3,2-c]pyridazin-4-amine (35.8 mg, 65.45%).

LC-MS (ES, m/z): [M+H]⁺=351

1H-NMR (400 MHz, Methanol-d₄) δ 8.47 (s, 1H), 7.35-7.250 (m, 1H), 7.15-7.050 (m, 1H), 7.02-6.92 (m, 1H), 4.87 (s, 2H), 4.73-4.49 (m, 2H), 3.38-3.33 (m, 1H), 3.20-2.97 (m, 2H), 2.53-2.43 (m, 3H), 2.08-1.65 (m, 2H).

### Synthesis of 6-[(2S)-2-amino-4-(difluoromethoxy)butyl]-7-methyl-N-(thiophen-2-ylmethyl)thieno[3,2-c]pyridazin-4-amine Compound 139

### Step 1

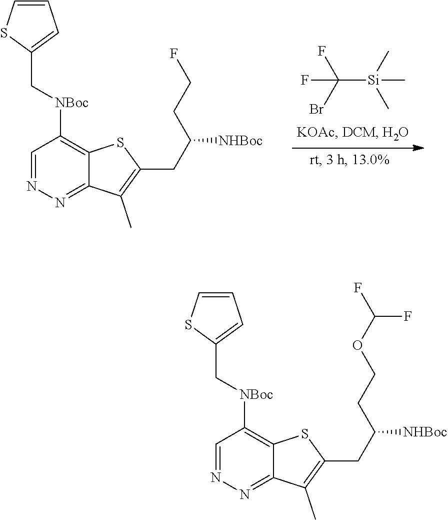

To a stirred mixture of tert-butyl N-{6-[(2S)-2-[(tert-butoxycarbonyl)amino]-4-hydroxybutyl]-7-methylthieno[3,2-c]pyridazin-4-yl}-N-(thiophen-2-ylmethyl)carbamate (described above) (350 mg, 0.638 mmol, 1 equiv) and (bromodifluoromethyl)trimethylsilane (388.64 mg, 1.914 mmol, 3 equiv) in DCM (3.5 mL) and H₂O (3.5 mL) was added KOAc (375.60 mg, 3.828 mmol, 6 equiv). The resulting mixture was stirred for 3 h at room temperature. The resulting mixture was extracted with CH₂Cl₂ (3×5 mL). The combined organic layers were dried over anhydrous Na₂SO₄. After filtration, the filtrate was concentrated under reduced pressure. The residue was purified by reversed-phase flash chromatography with the following conditions: column, C₁₈ silica gel; mobile phase, MeCN in Water (10 mmol/L NH₄HCO₃), 20% to 100% gradient in 20 min; detector, UV 254 nm. This resulted in tert-butyl N-[(2S)-1-{4-[(tert-butoxycarbonyl)(thiophen-2-ylmethyl)amino]-7-methylthieno[3,2-c]pyridazin-6-yl}-4-(difluoromethoxy)butan-2-yl]carbamate (50 mg, 13.09%) as a white solid.

### Step 2

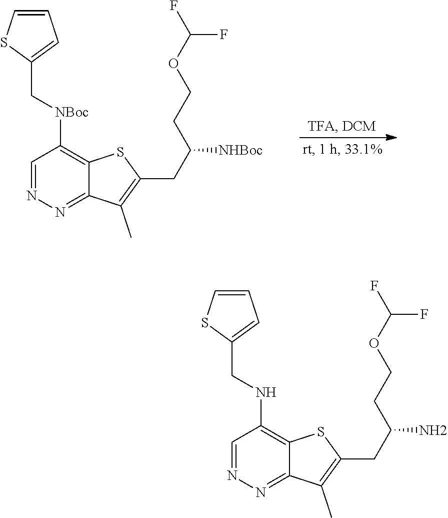 [SMILES: Cc1c(C[C@H](CCOC(F)F)NC(=O)OC(C)(C)C)sc2c(N(Cc3cccs3)C(=O)OC(C)(C)C)cnnc12 ; Cc1c(C[C@@H](N)CCOC(F)F)sc2c(NCc3cccs3)cnnc12]

To a stirred solution of tert-butyl N-[(2S)-1-{4-[(tert-butoxycarbonyl)(thiophen-2-ylmethyl)amino]-7-methylthieno[3,2-c]pyridazin-6-yl}-4-(difluoromethoxy)butan-2-yl]carbamate (50 mg, 0.084 mmol, 1 equiv) in DCM (0.6 mL) was added TFA (0.2 mL) dropwise at 0° C. The resulting mixture was stirred for 1 h at room temperature. The resulting mixture was concentrated under vacuum. The residue was purified by reversed-phase flash chromatography with the following conditions: column, C₁₈ silica gel; mobile phase, MeCN in Water (10 mmol/L NH₄HCO₃), 0% to 100% gradient in 20 min; detector, UV 254 nm. This resulted in 6-[(2S)-2-amino-4-(difluoromethoxy)butyl]-7-methyl-N-(thiophen-2-ylmethyl)thieno[3,2-c]pyridazin-4-amine (11.6 mg, 33.15%).

LC-MS (ES, m/z): [M+H]⁺=399

¹H-NMR (400 MHz, Methanol-d₄) δ 8.46 (s, 1H), 7.35-7.250 (m, 1H), 7.13-7.07 (m, 1H), 7.02-6.92 (m, 1H), 6.36 (t, J=75.7 Hz, 1H), 4.87 (s, 2H), 4.01 (t, J=6.3 Hz, 2H), 3.29-3.26 (m, 1H), 3.16-2.93 (m, 2H), 2.48 (s, 3H), 2.02-1.65 (m, 2H).

### Synthesis of 6-[(2S)-2-aminopent-4-yn-1-yl]-7-methyl-N-(thiophen-2-ylmethyl)thieno[3,2-c]pyridazin-4-amine Compound 142

### Step 1

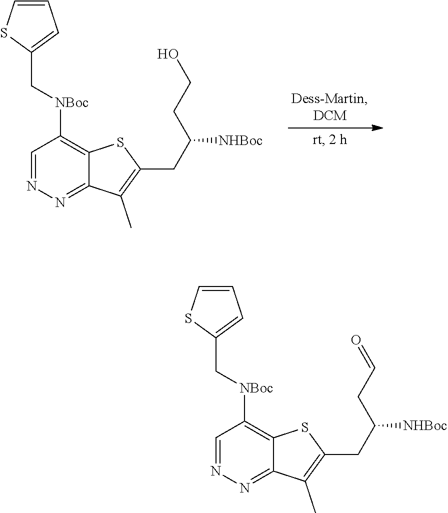 [SMILES: Cc1c(C[C@H](CCO)NC(=O)OC(C)(C)C)sc2c(N(Cc3cccs3)C(=O)OC(C)(C)C)cnnc12 ; Cc1c(C[C@H](CC=O)NC(=O)OC(C)(C)C)sc2c(N(Cc3cccs3)C(=O)OC(C)(C)C)cnnc12]

To a stirred solution of tert-butyl N-{6-[(2S)-2-[(tert-butoxycarbonyl)amino]-4-hydroxybutyl]-7-methylthieno[3,2-c]pyridazin-4-yl}-N-(thiophen-2-ylmethyl)carbamate (360 mg, 0.656 mmol, 1 equiv) in DCM (4 mL) was added Dess-Martin (417.40 mg, 0.984 mmol, 1.5 equiv) at 0° C. The resulting mixture was stirred for 2 h at room temperature. The resulting mixture was washed with 2×10 mL of saturated NaHCO₃ (aq.), dried over anhydrous Na₂SO₄. After filtration, the filtrate was concentrated under reduced pressure. The crude product was used in the next step directly without further purification.

### Step 2

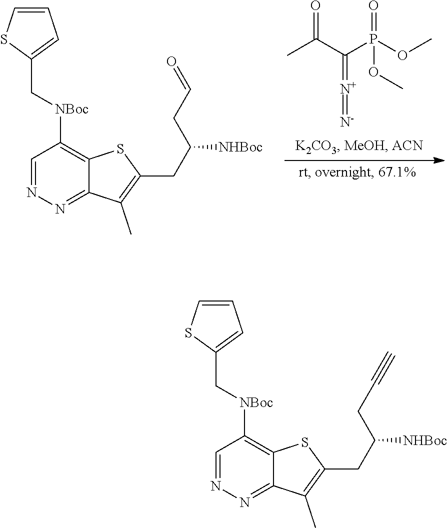 [SMILES: Cc1c(C[C@H](CC=O)NC(=O)OC(C)(C)C)sc2c(N(Cc3cccs3)C(=O)OC(C)(C)C)cnnc12 ; COP(=O)(OC)C(=[N+]=[N-])C(C)=O ; C#CC[C@@H](Cc1sc2c(N(Cc3cccs3)C(=O)OC(C)(C)C)cnnc2c1C)NC(=O)OC(C)(C)C]

Into a 20-mL vial were added tert-butyl N-{6-[(2S)-2-[(tert-butoxycarbonyl)amino]-4-oxobutyl]-7-methylthieno[3,2-c]pyridazin-4-yl}-N-(thiophen-2-ylmethyl)carbamate (360 mg, 0.658 mmol, 1 equiv) and K₂CO₃ (182.01 mg, 1.316 mmol, 2 equiv) in MeOH (4 mL). To the above mixture was added seyferth-gilbert homologation (139.15 mg, 0.724 mmol, 1.1 equiv) dissolved in ACN (4 mL) dropwise at 0° C. The resulting mixture was stirred for overnight at room temperature. The resulting mixture was concentrated under reduced pressure. The residue was purified by reversed-phase flash chromatography with the following conditions: column, C₁₈ silica gel; mobile phase, MeCN in Water (10 mmol/L NH₄HCO₃), 0% to 100% gradient in 30 min; detector, UV 254 nm. This resulted in tert-butyl N-{6-[(2S)-2-[(tert-butoxycarbonyl)amino]pent-4-yn-1-yl]-7-methylthieno[3,2-c]pyridazin-4-yl}-N-(thiophen-2-ylmethyl)carbamate (240 mg, 67.16%) as a light yellow solid.

### Step 3

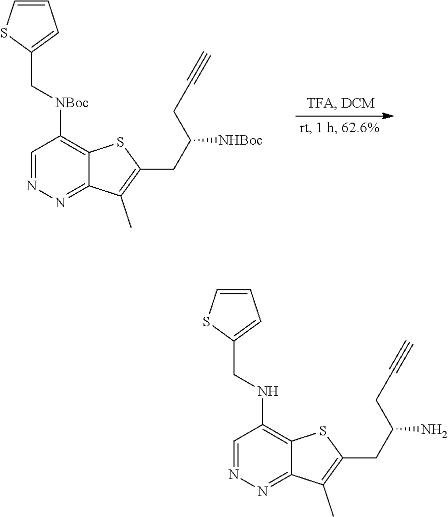 [SMILES: C#CC[C@@H](Cc1sc2c(N(Cc3cccs3)C(=O)OC(C)(C)C)cnnc2c1C)NC(=O)OC(C)(C)C ; C#CC[C@H](N)Cc1sc2c(NCc3cccs3)cnnc2c1C]

To a stirred solution of tert-butyl N-{6-[(2S)-2-[(tert-butoxycarbonyl)amino]pent-4-yn-1-yl]-7-methylthieno[3,2-c]pyridazin-4-yl}-N-(thiophen-2-ylmethyl)carbamate (100 mg, 0.184 mmol, 1 equiv) in DCM (0.6 mL) was added TFA (0.2 mL) dropwise at 0° C. The resulting mixture was stirred for 1 h at room temperature. The resulting mixture was concentrated under vacuum. The residue was purified by reversed-phase flash chromatography with the following conditions: column, C₁₈ silica gel; mobile phase, MeCN in Water (10 mmol/L NH₄HCO₃), 0% to 100% gradient in 20 min; detector, UV 254 nm. This resulted in 6-[(2S)-2-aminopent-4-yn-1-yl]-7-methyl-N-(thiophen-2-ylmethyl)thieno[3,2-c]pyridazin-4-amine (39.7 mg, 62.66%).

LC-MS (ES, m/z): [M+H]⁺=343

¹H-NMR (400 MHz, Methanol-d₄) δ 8.46 (d, J=1.8 Hz, 1H), 7.35-7.250 (m, 1H), 7.10 (s, 1H), 7.02-6.92 (m, 1H), 4.87 (s, 2H), 3.29-3.19 (m, 2H), 3.09-2.99 (m, 1H), 2.53-2.43 (m, 4H), 2.43-2.33 (m, 2H).

### Synthetic Scheme for Compounds 127-130

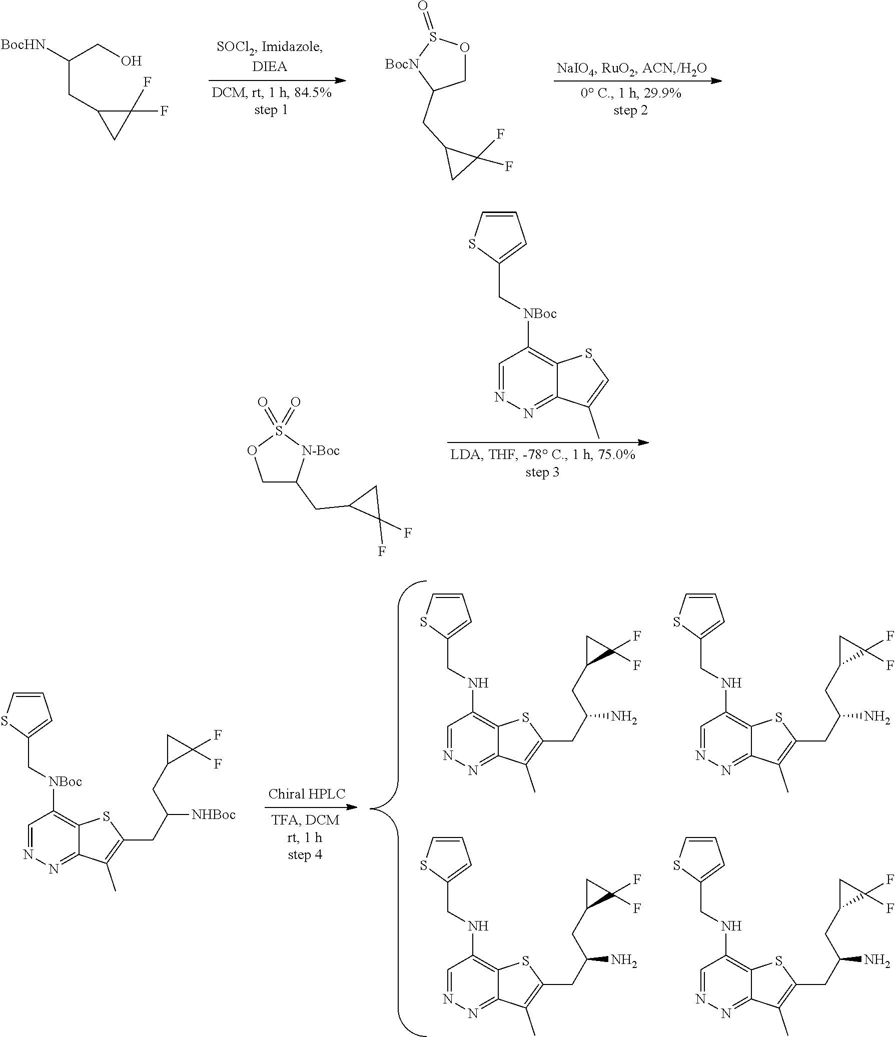

### Synthesis of Compounds 127-130

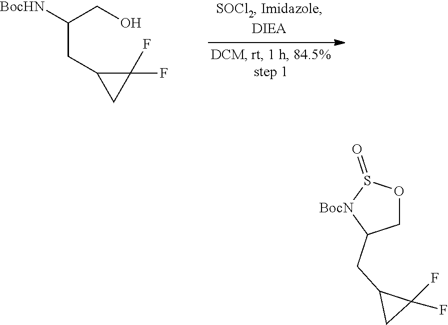 [SMILES: CC(C)(C)OC(=O)NC(CO)CC1CC1(F)F ; CC(C)(C)OC(=O)N1C(CC2CC2(F)F)COS1=O]

To a solution of Imidazole (791.13 mg, 11.620 mmol, 4 equiv) in DCM (22 mL) were added SOCl₂ (0.32 mL, 4.357 mmol, 1.5 equiv) followed by DIEA (1.01 mL, 5.810 mmol, 2 equiv) dropwise at 0° C. To the above mixture was added tert-butyl N-[(1S)-1-cyclopropyl-2-hydroxyethyl]carbamate (1.6 g, 7.950 mmol, 1 equiv) dissloved in DCM (4 mL) dropwise over 30 min at 0° C. The resulting mixture was stirred for additional 1 h at room temperature. The residue was washed with 0.5M HCl (3×30 mL). The resulting mixture was concentrated under reduced pressure to afford tert-butyl 4-[(2,2-difluorocyclopropyl)methyl]-2-oxo-1,2lambda4,3-oxathiazolidine-3-carboxylate (730 mg, 84.51%) as a yellow oil.

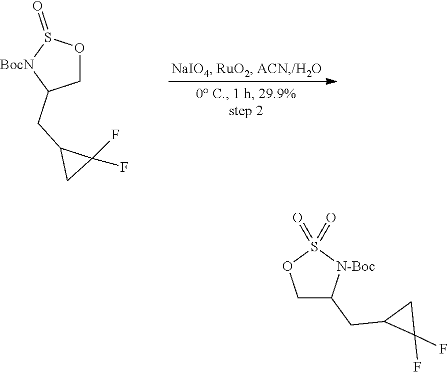

To a solution of tert-butyl 4-[(2,2-difluorocyclopropyl)methyl]-2-oxo-1,2lambda4,3-oxathiazolidine-3-carboxylate (730 mg, 2.455 mmol, 1 equiv) in ACN (11 mL) and H₂O (6 mL) was added NaIO₄ (630.19 mg, 2.946 mmol, 1.2 equiv) and ruthenium(iv) oxide hydrate (7.42 mg, 0.049 mmol, 0.02 equiv) at 0° C. The resulting mixture was stirred for 1 h at 0° C. The resulting mixture was filtered. The filtrate was extracted with EtOAc (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (2:1) to afford tert-butyl 4-[(2,2-difluorocyclopropyl)methyl]-2,2-dioxo-1,2lambda6,3-oxathiazolidine-3-carboxylate (230 mg, 29.90%) as a white solid.

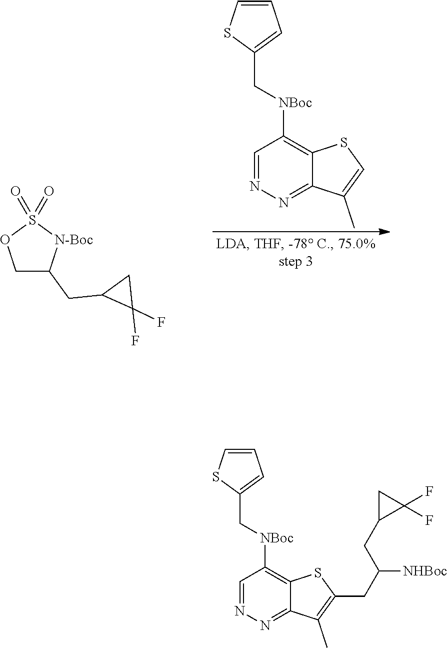

Into a 8-mL vial, to a solution of tert-butyl N-{7-methylthieno[3,2-c]pyridazin-4-yl}-N-(thiophen-2-ylmethyl)carbamate (170 mg, 0.470 mmol, 1 equiv) in THF (3 mL) was added LDA (0.47 mL, 0.940 mmol, 2 equiv) dropwise at −78 degrees C. under N₂ atmosphere. The reaction mixture was stirred at −78 degrees C. for 30 mins. Then a solution of tert-butyl 4-[(2,2-difluorocyclopropyl)methyl]-2,2-dioxo-1,2lambda6,3-oxathiazolidine-3-carboxylate (221.03 mg, 0.705 mmol, 1.5 equiv) in 1 mL THF was added dropwise and the mixture was stirred for another 1 h −78 degrees C. The residue was purified by reversed-phase flash chromatography with the following conditions: column, C₁₈; mobile phase, MeCN in Water (10 mmol/L NH₄HCO₃), 0% to 100% gradient in 20 min; detector, UV 254 nm. This resulted in tert-butyl N-[(2R)-1-{4-[(tert-butoxycarbonyl)(thiophen-2-ylmethyl)amino]-7-methylthieno[3,2-c]pyridazin-6-yl}-3-[(1S)-2,2-difluorocyclopropyl]propan-2-yl]carbamate (210 mg, 75.08%) as a yellow solid.

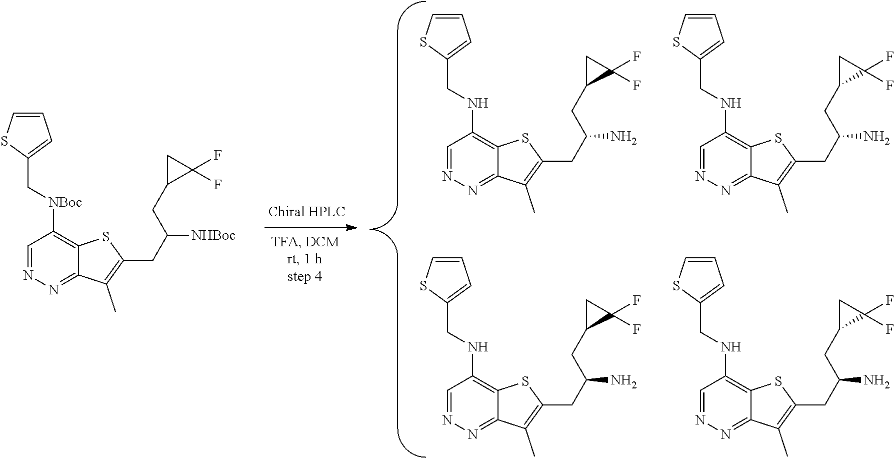 [SMILES: Cc1c(CC(CC2CC2(F)F)NC(=O)OC(C)(C)C)sc2c(N(Cc3cccs3)C(=O)OC(C)(C)C)cnnc12 ; Cc1c(C[C@@H](N)C[C@H]2CC2(F)F)sc2c(NCc3cccs3)cnnc12 ; Cc1c(C[C@H](N)C[C@H]2CC2(F)F)sc2c(NCc3cccs3)cnnc12 ; Cc1c(C[C@@H](N)C[C@@H]2CC2(F)F)sc2c(NCc3cccs3)cnnc12 ; Cc1c(C[C@H](N)C[C@@H]2CC2(F)F)sc2c(NCc3cccs3)cnnc12]

To a stirred solution of tert-butyl N-(1-{4-[(tert-butoxycarbonyl)(thiophen-2-ylmethyl)amino]-7-methylthieno[3,2-c]pyridazin-6-yl}-3-(2,2-difluorocyclopropyl)propan-2-yl)carbamate (210 mg, 0.353 mmol, 1 equiv) in DCM (3 mL) was added TFA (1 mL) dropwise at 0° C. The resulting mixture was stirred for 1 h at room temperature. The mixture was basified to pH 8 with saturated NaHCO₃ (aq.). The resulting mixture was extracted with EtOAc (3×10 mL). The combined organic layers were washed with brine (10 mL), dried over anhydrous Na₂SO₄. After filtration, the filtrate was concentrated under reduced pressure. The crude product (130 mg) was purified by Prep-Chiral-HPLC with the following conditions (Column: CHIRALPAK IF-3, 4.6*50 mm, 3 m; Mobile Phase A: Hex (0.1% DEA):EtOH=50:50; Flow rate: 1 mL/min; Gradient: isocratic) to afford 6-[(2S)-2-amino-3-[(1S)-2,2-difluorocyclopropyl]propyl]-7-methyl-N-(thiophen-2-ylmethyl)thieno[3,2-c]pyridazin-4-amine (6.6 mg, 4.43%), 6-[(2S)-2-amino-3-[(1R)-2,2-difluorocyclopropyl]propyl]-7-methyl-N-(thiophen-2-ylmethyl)thieno[3,2-c]pyridazin-4-amine (4.5 mg, 3.16%), 6-[(2R)-2-amino-3-[(1S)-2,2-difluorocyclopropyl]propyl]-7-methyl-N-(thiophen-2-ylmethyl)thieno[3,2-c]pyridazin-4-amine (7.8 mg, 5.42%) and 6-[(2R)-2-amino-3-[(1R)-2,2-difluorocyclopropyl]propyl]-7-methyl-N-(thiophen-2-ylmethyl)thieno[3,2-c]pyridazin-4-amine (7 mg, 4.93%).

### Compound 127

LC-MS-(ES, m/z): [M+H]⁺=395

H-NMR-(400 MHz, Methanol-d₄) δ 8.47 (d, J=1.1 Hz, 1H), 7.30 (d, J=5.3 Hz, 1H), 7.11 (d, J 3.5 Hz, 1H), 7.02-6.92 (m, 1H), 4.87 (s, 2H), 3.22-3.12 (m, 1H), 3.06-2.96 (m, 1H), 2.48 (s, 3H), 1.74 (dm, 3H), 1.58-1.48 (m, 1H), 1.46-1.33 (m, 1H), 1.13-1.03 (m, 1H).

### Compound 128

LC-MS-(ES, m/z): [M+H]⁺=395

(400 MHz, Methanol-d₄) δ 8.47 (d, J=1.1 Hz, 1H), 7.30 (d, J=5.3 Hz, 1H), 7.11 (d, J=3.5 Hz, 1H), 7.02-6.92 (m, 1H), 4.87 (s, 2H), 3.22-3.12 (m, 1H), 3.06-2.96 (m, 1H), 2.48 (s, 3H), 1.74 (dm, 3H), 1.58-1.48 (m, 1H), 1.46-1.33 (m, 1H), 1.13-1.03 (m, 1H).

### Compound 129

LC-MS-(ES, m/z): [M+H]⁺=395

(400 MHz, Methanol-d₄) δ 8.47 (s, 1H), 7.35-7.250 (m, 1H), 7.15-7.050 (m, 1H), 7.02-6.92 (m, 1H), 4.87 (s, 2H), 3.26 (t, J=6.5 Hz, 1H), 3.2-3.1 (m, 1H), 3.04-2.94 (m, 1H), 2.48 (s, 3H), 1.84-1.58 (m, 3H), 1.57-1.45 (m, 1H), 1.17-1.00 (m, 1H).

### Compound 130

LC-MS-(ES, m/z): [M+H]⁺=395

H-NMR-(400 MHz, Methanol-d₄) δ 8.47 (s, 1H), 7.35-7.250 (m, 1H), 7.15-7.050 (m, 1H), 7.02-6.92 (m, 1H), 4.87 (s, 2H), 3.26 (t, J=6.5 Hz, 1H), 3.2-3.1 (m, 1H), 3.04-2.94 (m, 1H), 2.48 (s, 3H), 1.84-1.58 (m, 3H), 1.57-1.45 (m, 1H), 1.17-1.00 (m, 1H).

### Example 2: ATXN3 Quantitative Splicing Assay

Human neuroblastoma SK-N-MC cells were plated in 384-well plates at 20,000 cells/well. Twenty-four hours after plating, cells were treated with compounds for 24 h at appropriate concentrations ranging from 30 μM to 0.6 nM (0.3% DMSO). Treated cells were lysed in 15 μL of lysis buffer, and cDNA was synthesized using the Fast Advanced Cells-to-Ct kit. Two μL of each cDNA was used in qPCR reactions to confirm the exon 4 skipped transcripts of ATXN3. A second set of primers/probe E4E5 was used to detect the transcripts containing exon 4. The third set of primers/probe E8E9 was used to detect total gene level of ATXN3. The qPCR reactions were prepared in 384-well plates in 10 μL volume, using TaqMan™ Fast Advanced Master Mix with primers and probes shown in the table below. Reactions were run in a Quant Studio 6 qPCR instrument with default settings.

The primers and probes are listed below in Table 4.

TABLE 4

| Target | | | |
|---|---|---|---|
| Sequence | Forward Primer | Probe | Reverse Primer |
| ATXN3 | SEQ ID NO: 2 | SEQ ID NO: 3 | SEQ ID NO: 4 |
| E4skpping- | 5′ GCAGCC | 5′ TTCTCTAT | 5′ CTGGACC |
| FAM | TTCTGGAAA | TCAGAAATGA | CGTCAAGA |
| | TATGG 3′ | AAGATCATT 3′ | GAGAA 3′ |
| ATXN3 | SEQ ID NO: 5 | SEQ ID NO: 6 | SEQ ID NO: 7 |
| E4E5-Cy5 | 5′ TGTTCAA | 5′ AGGCTCAG | 5′ ACCCGTC |
| | CAGTCCAGAG | GATCGATCCT | AAGAGAGA |
| | TATCAG 3′ | ATAAATGAAAGA | ATTCAAG 3′ |
| | | 3′ | |
| ATXN3 | SEQ ID NO: 8 | SEQ ID NO: 9 | SEQ ID NO: 10 |
| E8E9- | 5′ GATGAGG | 5′ ATGTTTCT | 5′ CCTGATG |
| total-FAM | AGGATTTGC | GGAACTACCTT | TCTGTGTCA |
| | AGAGG 3′ | GCATACTTAGC | TATCTTGA 3′ |
| | | TG 3′ | |
| TBP-YAK | SEQ ID NO: 11 | SEQ ID NO: 12 | SEQ ID NO: 13 |
| (endogenous | 5′ TCGGAG | 5′ CCGCAGC | 5′ AAGTGCAA |
| control) | AGTTCTGGG | TGCAAAATAT | TGGTCTTTAG |
| | ATT 3′ | TGTATCCACA 3′ | GT 3′ |

### Example 3: ATXN3 Total Protein Assay

Human neuroblastoma SK-N-MC cells were seeded at 10,000 cells/well in 384 well plates one day prior to compound treatment. The concentrations of compounds were tested at appropriate doses ranging from 30 μM to 0.6 nM. After incubation for 48 hours, the cells were lysed with 25 μL of lysis buffer containing protease inhibitors, and total ATXN3 protein levels were assessed by Mesoscale Discovery (MSD) assay developed with one pair of anti-ATXN3 antibodies. The capture and detect antibodies were raised in mouse and rabbit respectively. Anti-rabbit MSD-ST antibody was used for secondary antibody.

ATXN3 recombinant protein was used for standards. The readouts were captured with 35 μL of MSD read buffer and multi-array 384-well high binding plates.

One plate replica was carried out for parallel viability testing by CellTiter Glo® 2.0 with a seeding density of 4,000 cells/well. Compounds were incubated for 48 hours. The viability readouts were carried out by Envision according to the manufacturer's instructions.

Compounds were tested as outlined in Examples 2 and 3 above and the results are shown below in Table 5.

TABLE 5
* IC₅₀/EC₅₀ range (nM): 0.01 ≤ A ≤ 100; 101 ≤ B ≤ 500;
501 ≤ C ≤ 5000; 5001 ≤ D ≤ 10000; 10001 ≤ E ≤40,000.

| | ATXN3 Potency - | ATXN3 Potency - |
|---|---|---|
| | Protein | Splicing |
| Compound | IC50 (nM) | E4E5 IC50 (nM) |
| 1 | C | C |
| 2 | C | C |
| 3 | C | C |
| 4 | B | C |
| 5 | E | E |
| 6 | C | C |
| 7 | C | C |
| 8 | C | C |
| 9 | E | E |
| 10 | C | C |
| 11 | E | E |
| 12 | E | E |
| 15 | C | C |
| 16 | C | C |
| 17 | E | D |
| 18 | E | E |
| 19 | E | E |
| 20 | D | D |
| 21 | E | E |
| 22 | C | C |
| 23 | D | D |
| 24 | C | C |
| 25 | E | E |
| 26 | E | E |
| 27 | D | C |
| 28 | E | E |
| 29 | C | C |
| 30 | C | C |
| 31 | C | C |
| 32 | E | E |
| 33 | E | E |
| 34 | E | E |
| 35 | B | B |
| 36 | B | B |
| 37 | E | E |
| 38 | D | D |
| 39 | C | C |
| 40 | E | E |
| 41 | E | E |
| 42 | D | D |
| 43 | D | D |
| 44 | E | E |
| 45 | C | C |
| 46 | E | E |
| 48 | C | C |
| 49 | E | E |
| 50 | C | C |
| 51 | E | E |
| 52 | C | C |
| 53 | C | C |
| 54 | E | E |
| 55 | C | C |
| 56 | E | E |
| 57 | E | E |
| 58 | C | C |
| 59 | E | D |
| 60 | D | D |
| 61 | C | C |
| 62 | D | D |
| 63 | E | E |
| 64 | D | D |
| 65 | E | E |
| 66 | E | E |
| 67 | E | E |
| 68 | C | C |
| 69 | E | E |
| 70 | E | |
| 71 | E | E |
| 72 | E | E |
| 73 | E | E |
| 74 | E | E |
| 75 | E | E |
| 76 | E | E |
| 77 | E | |
| 78 | E | |
| 79 | E | |
| 80 | C | C |
| 82 | E | E |
| 86 | E | E |
| 87 | E | E |
| 88 | E | |
| 89 | E | E |
| 90 | E | E |
| 91 | B | B |
| 92 | E | E |
| 93 | C | D |
| 94 | B | B |
| 95 | B | C |
| 96 | B | B |
| 97 | B | B |
| 98 | E | E |
| 99 | E | E |
| 100 | B | B |
| 101 | B | B |
| 102 | E | E |
| 104 | E | E |
| 105 | E | E |
| 107 | B | B |
| 110 | B | B |
| 111 | C | C |
| 112 | E | E |
| 113 | E | E |
| 114 | E | E |
| 115 | B | A |
| 116 | B | A |
| 117 | D | D |
| 125 | C | C |
| 127 | E | E |
| 128 | E | E |
| 129 | C | D |
| 130 | C | C |
| 131 | D | C |
| 132 | E | E |
| 133 | C | C |
| 135 | B | B |
| 136 | B | B |
| 137 | C | C |
| 139 | C | C |
| 140 | C | C |
| 141 | C | C |
| 142 | C | C |

1. A compound of Formula (I), or a pharmaceutically acceptable salt thereof: 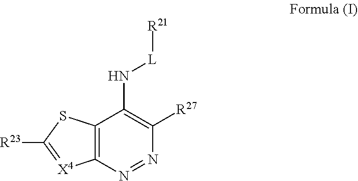
wherein,
X⁴ is selected from the group consisting of N, and CR²⁴;
L is absent or selected from the group consisting of C₁₋₆ alkylene, C₂₋₆ alkenylene, and C₂₋₆ alkynylene, wherein the C₁₋₆ alkylene, C₂₋₆ alkenylene, and C₂₋₆ alkynylene are each optionally substituted by 1, 2, 3, or 4 independently selected R²⁰ groups;
R²¹ is selected from the group consisting of 5 membered heteroaryl, and 5 membered heterocycloalkyl, each of which is unsubstituted or substituted with 1, 2, or 3, independently selected R^{1A} groups; each R^{1A} is independently selected from halo, CN, NO₂, C₁₋₆ alkyl, C₂₋₆ alkenyl, C₂₋₆ alkynyl, C₁₋₆ haloalkyl, C₁₋₆ alkoxy, —C(═O)OH, —C(═O)C₁₋₆ alkyl, —C(═O)C₁₋₆ haloalkyl, and —C(═O)C₁₋₆ alkoxy;
R²³ is selected from the group consisting of H, azido, halo, CN, NO₂, C₁₋₆ alkyl, C₂₋₆ alkenyl, C₂₋₆ alkynyl, C₁₋₆ heteroalkyl, —(C₁₋₆ alkylene)-C₃₋₁₀ cycloalkyl, —(C₁₋₆ alkylene)-4-10 membered heterocycloalkyl, —(C₁₋₆ heteroalkylene)-C₃₋₁₀ cycloalkyl, —(C₁₋₆ heteroalkylene)-4-10 membered heterocycloalkyl, C₃₋₁₀ cycloalkyl, C₆₋₁₀ aryl, 5-10 membered heteroaryl, 4-10 membered heterocycloalkyl, OR^{a3}, SR^{a3}, C(═O)R^{b3}, C(═O)OR^{b3}, NR^{c3}R^{d3}, C(═O)NR^{c3}R^{d3}, —OC(═O)NR^{c3}R^{d3} NR^{c3}C(═O)R^{b3}, NR^{c3}C(═O)OR^{b3}, NR^{c3}C(═O)NR^{c3}R^{d3} NR^{c3}S(═O)₂R^{b3}, NR^{c3}S(═O)₂NR^{c3}R^{d3} S(O)NR^{c3}R^{d3}, and S(O)₂NR^{c3}R^{d3}, wherein the C₁₋₆ alkyl, C₂₋₆ alkenyl, C₂₋₆ alkynyl, C₁₋₆ heteroalkyl, C₁₋₆ alkylene, C₁₋₆ heteroalkylene, C₃₋₁₀ cycloalkyl, C₆₋₁₀ aryl, 5-10 membered heteroaryl, and 4-10 membered heterocycloalkyl are each optionally substituted by 1, 2, 3, or 4 independently selected R²⁰ groups;
R²⁴ is selected from the group consisting of H, azido, halo, CN, NO₂, C₁₋₆ alkyl, C₂₋₆ alkenyl, C₂₋₆ alkynyl, C₃₋₁₀ cycloalkyl, C₆₋₁₀ aryl, 5-10 membered heteroaryl, 4-10 membered heterocycloalkyl, OR^{a4}, C(═O)R^{b4}, C(═O)OR^{b4}, NR^{c4}R^{d4}, C(═O)NR^{c4}R^{d4}, —OC(═O)NR^{c4}R^{d4}, NR^{c4}C(═O)R^{b4}, NR^{c4}C(═O)OR^{b4}, NR^{c4}C(═O)NR^{c4}R^{d4} NR^{c4}S(═O)₂R¹⁴, NR^{c4}S(═O)₂NR^{c4}R^{d4}, S(O)NR^{c4}R^{d4}, and S(O)₂NR^{c4}R^{d4}, wherein the C₁₋₆ alkyl, C₃₋₁₀ cycloalkyl, C₂₋₆ alkenyl, C₂₋₆ alkynyl, C₆₋₁₀ aryl, 5-10 membered heteroaryl, and 4-10 membered heterocycloalkyl are each optionally substituted by 1, 2, 3, or 4 independently selected R²⁰ groups;
R²⁷ is selected from the group consisting of H, halogen, alkyl, heteroalkyl, CN, NO₂, OR^{a7}, C(═O)R^{b7}, C(═O)OR^{b7}, NR^{c7}R^{d7}, C(═O)NR^{c7}R^{d7}, —OC(═O)NR^{c7}R^{d7} NR^{c7}C(═O)R^{b7}, NR^{c7}C(═O)OR^{b7}, NR^{c7}C(═O)NR^{c7}R^{d7} NR^{c7}S(═O)₂R^{b7}, and NR^{c7}S(═O)₂NR^{c7}R^{d7};
each R^{a3}, R^{b3}, R^{c3}, R^{d3}, R^{a4}, R^{b4}, R^{c4}, R^{d4}, R^{a7}, R^{b7}, R^{c7}, and R^{d7} is independently selected from the group consisting of H, C₁₋₆ alkyl, C₂₋₆ alkenyl, C₂₋₆ alkynyl, C₁₋₆ hydroxyalkyl, C₁₋₆ haloalkyl, C₁₋₆ alkoxy, —(C₁₋₆ alkylene)-C₁₋₆ alkoxy, C₃₋₁₀ cycloalkyl, —(C₁₋₆ alkylene)-C₃₋₁₀ cycloalkyl, C₆₋₁₀ aryl, 5-10 membered heteroaryl, 4-10 membered heterocycloalkyl, wherein the C₁₋₆ alkyl, C₂₋₆ alkenyl, C₂₋₆ alkynyl, C₃₋₁₀ cycloalkyl, —(C₁₋₆ alkylene)-C₃₋₁₀ cycloalkyl, C₆₋₁₀ aryl, 5-10 membered heteroaryl, and 4-10 membered heterocycloalkyl are each optionally substituted by 1, 2, 3, or 4 independently selected R²⁰ groups;
or R^{c3} and R^{d3} together with the N atom to which they are connected, come together to form a 5-10 membered heteroaryl or 4-10 membered heterocycloalkyl ring, each optionally substituted by 1, 2, 3, or 4 independently selected R²⁰ groups;
or R^{c4} and R^{d4} together with the N atom to which they are connected, come together to form a 5-10 membered heteroaryl or 4-10 membered heterocycloalkyl ring, each optionally substituted by 1, 2, 3, or 4 independently selected R²⁰ groups;
or R^{c7} and R^{d7} together with the N atom to which they are connected, come together to form a 5-10 membered heteroaryl or 4-10 membered heterocycloalkyl ring, each optionally substituted by 1, 2, 3, or 4 independently selected R²⁰ groups; and
each R²⁰ is independently selected from the group consisting of OH, SH, CN, NO₂, halo, oxo, C₁₋₄ alkyl, C₂₋₄ alkenyl, C₂₋₄ alkynyl, C₁₋₄ haloalkyl, C₁₋₄ cyanoalkyl, C₁₋₄ hydroxyalkyl, C₁₋₄ alkoxy, —(C₁₋₄ alkyl)-(C₁₋₄ alkoxy), —(C₁₋₄ alkoxy)-(C₁₋₄ alkoxy), C₁₋₄ haloalkoxy, C₃₋₆ cycloalkyl, phenyl, 5-6 membered heteroaryl, 4-6 membered heterocycloalkyl, amino, C₁₋₄ alkylamino, di(C₁₋₄ alkyl)amino, carbamyl, C₁₋₄ alkylcarbamyl, di(C₁₋₄ alkyl)carbamyl, carbamoyl, C₁₋₄ alkylcarbamoyl, di(C₁₋₄ alkyl)carbamoyl, C₁₋₄ alkylcarbonyl, C₁₋₄ alkoxycarbonyl, C₁₋₄ alkylcarbonylamino, C₁₋₄ alkylsulfonylamino, aminosulfonyl, C₁₋₄ alkylaminosulfonyl, di(C₁₋₄ alkyl)aminosulfonyl, aminosulfonylamino, C₁₋₄ alkylaminosulfonylamino, di(C₁₋₄ alkyl)aminosulfonylamino, aminocarbonylamino, C₁₋₄ alkylaminocarbonylamino, di(C₁₋₄ alkyl)aminocarbonylamino, and amidinyl.

2. The compound of claim 1, or a pharmaceutically acceptable salt thereof, wherein R²¹ is 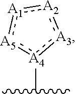
wherein [symbol] represents a single or a double bond; each of A₁, A₂, A₃, and A₅ is independently selected from the group consisting of O, S, N, NH, NR^{1A}, C, CH, CR^{1A}, CH₂, and CHR^{1A}; and A4 is selected from the group consisting of N, C, CH and CR^{1A}.

3. The compound of claim 2, or a pharmaceutically acceptable salt thereof, wherein R²¹ is selected from the group consisting of

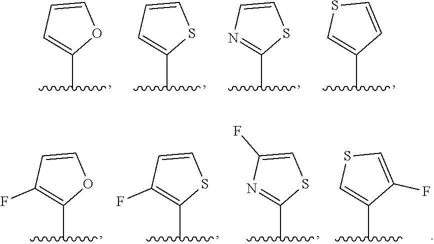

4. The compound of claim 1, or a pharmaceutically acceptable salt thereof, wherein L is —CH₂—.

5. The compound of claim 1, wherein R²³ is substituted or unsubstituted C₁₋₆ alkyl or substituted or unsubstituted C₁₋₆ heteroalkyl.

6. The compound of claim 5, or a pharmaceutically acceptable salt, wherein R²³ is CH₂CHNH₂CH₃, CH₂CHNH₂CH₂OH, CH₂CHNH₂CH₂CH₃, CH₂CHNH₂CH₂CH₂OH, CH₂CHNH₂CH₂CH₂F, CH₂CHNH₂CH₂CHF₂, or CH₂CHNH₂CH₂CH(CH₃)₂.

7. (canceled)

8. (canceled)

9. (canceled)

10. (canceled)

11. (canceled)

12. (canceled)

13. The compound of claim 1, or a pharmaceutically acceptable salt thereof, wherein X⁴ is N.

14. The compound of claim 1, or a pharmaceutically acceptable salt thereof, wherein X⁴ is CR²⁴, wherein R²⁴ is selected from the group consisting of hydrogen, OH, halo, CN, substituted or unsubstituted C₁₋₆ alkyl, substituted or unsubstituted C₁₋₆ alkoxyl, substituted or unsubstituted C₃₋₆ cycloalkyl, substituted or unsubstituted C₂₋₄ alkenyl, and substituted or unsubstituted C₂₋₄ alkynyl.

15. The compound of claim 1, or a pharmaceutically acceptable salt thereof, wherein X⁴ is CCl.

16. The compound of claim 1, or a pharmaceutically acceptable salt thereof, wherein X⁴ is CBr.

17. The compound of claim 1, or a pharmaceutically acceptable salt thereof, wherein X⁴ is CF.

18. The compound of claim 1, or a pharmaceutically acceptable salt thereof, wherein X⁴ is CCN.

19. The compound of claim 1, or a pharmaceutically acceptable salt thereof, wherein X⁴ is CCH₃.

20. The compound of claim 1, or a pharmaceutically acceptable salt thereof, wherein X⁴ is C-cyclopropyl.

21. A compound, or a pharmaceutically acceptable salt thereof, wherein the compound is selected from Table 1.

22. A pharmaceutical composition comprising a compound of claim 1, or a pharmaceutically acceptable salt thereof, and a pharmaceutically acceptable excipient or carrier.

23. (canceled)

24. A method of modulating splicing of a Ataxin3 (ATXN3) pre-mRNA, comprising contacting a compound or salt of claim 1 to the ATXN3 pre-mRNA with a splice site sequence or cells comprising the ATXN3 pre-mRNA, wherein the compound binds to the ATXN3 pre-mRNA and modulates splicing of the ATXN3 pre-mRNA in a cell of a subject to produce a spliced product of the ATXN3 pre-mRNA.

25. (canceled)

26. The compound of claim 1, or a pharmaceutically acceptable salt thereof, wherein X is CR²⁴, and R²⁴ is C₂₋₄ alkynyl.
